(19) United States
(12) Patent Application Publication
(10) Pub. No.: US 2023/0107304 A1
(43) Pub. Date: Apr. 6, 2023

(54) METHODS OF TREATING CANCER AND TUMOR-RELATED WEIGHT LOSS AND CACHEXIA

(71) Applicant: NGM Biopharmaceuticals, Inc., South San Francisco, CA (US)

(72) Inventors: Daniel Chan, San Francisco, CA (US); Alexander Mark DePaoli, Santa Barbara, CA (US); Josh Lichtman, Berkeley, CA (US); Bich Ngoc Tran-Muchowski, San Francisco, CA (US); Yi Qun Xiao, Foster City, CA (US); Jiping Zha, Foster City, CA (US); Jian Luo, Albany, CA (US)

(21) Appl. No.: 17/929,518

(22) Filed: Sep. 2, 2022

Related U.S. Application Data
(60) Provisional application No. 63/242,936, filed on Sep. 10, 2021.

(51) Int. Cl. C07K 16/28; A61K 39/395; A61K 31/337; A61K 31/7068; A61P 1/14; A61P 1/18; A61P 1/00; A61P 13/08; A61P 13/10; A61P 35/00

(52) U.S. Cl. CPC C07K 16/2863; A61K 39/3955; A61K 31/337; A61K 31/7068; A61P 1/14; A61P 1/18; A61P 1/00; A61P 13/08; A61P 13/10; A61P 35/00; C07K 2317/565; A61K 2039/505

(57) ABSTRACT

The present disclosure relates generally to methods of treating (i) a tumor (e.g., a solid tumor, an advanced solid tumor, a cancer), (ii) tumor-related weight loss, or (iii) tumor-related cachexia in a human patient, the method comprising administering to the human patient an anti-GDNF family receptor alpha-like (GFRAL) antibody, wherein the antibody inhibits GFRAL binding to Ret proto-oncogene (RET). The present disclosure also relates generally to methods of treating pancreatic cancer (e.g., an advanced pancreatic cancer, a metastatic pancreatic cancer, a pancreatic adenocarcinoma, a metastatic pancreatic adenocarcinoma, an MSI-H pancreatic cancer, a pancreatic ductal adenocarcinoma, a metastatic pancreatic ductal adenocarcinoma) in a human patient, comprising administering to the human patient (i) an anti-GFRAL antibody, wherein the antibody inhibits GFRAL binding to RET; (ii) paclitaxel; and (iii) gemcitabine.

### I. CROSS-REFERENCE TO RELATED APPLICATION

This application claims the priority benefit of U.S. Provisional Application No. 63/242,936, filed Sep. 10, 2021, the content of which is incorporated by reference in its entirety herein.

### II. SEQUENCE LISTING

The instant application contains a Sequence Listing which has been submitted electronically in XML file format and is hereby incorporated by reference in its entirety. Said XML copy, created on Aug. 29, 2022, is named 47702-0108001_SL.xml and is 546,728 bytes in size.

### III. FIELD

The present disclosure relates generally to methods of treating (i) a tumor (e.g., cancer) or (ii) tumor-related weight loss or cachexia in a human patient, the method comprising administering to the human patient an anti-glial cell line-derived neurotrophic factor family receptor alpha-like (GFRAL) antibody, wherein the anti-GFRAL antibody inhibits GFRAL binding to Ret proto-oncogene (RET). The present disclosure also relates generally to methods of treating pancreatic cancer (e.g., advanced pancreatic cancer, pancreatic adenocarcinoma, metastatic pancreatic cancer, metastatic pancreatic adenocarcinoma, microsatellite instability high pancreatic cancer, pancreatic ductal adenocarcinoma, metastatic pancreatic ductal adenocarcinoma) in a human patient, comprising administering to the human patient (i) an anti-GFRAL antibody, wherein the antibody inhibits GFRAL binding to RET; (ii) paclitaxel; and (iii) gemcitabine.

### IV. BACKGROUND OF THE INVENTION

Growth differentiation factor 15 (GDF15) is a protein belonging to the transforming growth factor beta (TGF-β) superfamily. GDF15 is also known as TGF-PL, MIC-1, PDF, PLAB, NAG-1, and PTGFB. GDF15 mRNA is reported to be most abundant in the liver, with lower levels seen in some other tissues. Its expression in liver can be significantly up-regulated in injury of organs such as liver, kidney, heart and lung.

GDF15 is reported to play a role in regulating inflammatory and apoptotic pathways in injured tissues and during disease processes. It has been reported that GDF15 is a mediator of cachexia in various diseases. However, cachexia is a complex and incompletely understood syndrome. In addition, at least some tumors over-express and secrete GDF15, and elevated serum GDF15 levels have been associated with various cancers. GDF-15 has been described as a negative regulator of macrophage activation by suppressing the release of TNF-α, IL-1, IL-2 and MCS-F, thus inhibiting the positive feedback of local inflammatory signaling similar to the effects of TGF-β. Monoclonal antibodies against GDF15 have been disclosed as potential therapeutic agents for the treatment of cachexia and of cancer.

There is a significant unmet need for therapeutic agents effective to treat weight loss associated with a number of diseases and conditions, including wasting diseases such as cachexia or sarcopenia and inflammatory conditions such as systemic inflammation or an acute inflammatory response. There is also a significant unmet need for therapeutic agents effective to treat tumors, including in which involuntary body weight loss and/or muscle mass loss is involved.

### V. BRIEF SUMMARY OF THE INVENTION

This disclosure relates to dosing human patients in need thereof with therapeutically effective doses of an anti-GDNF Family Receptor Alpha-Like (GFRAL) antibody.

In a first aspect, this disclosure features a method for treating a tumor (e.g., a solid tumor, an advanced solid tumor, a cancer, e.g., a prostate cancer (e.g., a metastatic castration-resistant prostate cancer (mCRPC), a castration-sensitive prostate cancer, a castration-resistant prostate cancer; an MSI-H prostate cancer, a metastatic prostate cancer, a metastatic castrate-resistant, chemo-refractory prostate cancer; an advanced prostate cancer where the patient has exhausted all standard therapies with elevated baseline serum GDF15; an advanced prostate cancer where the patient has previously received one or more (e.g., 1, 2, 3, or more) lines of cancer therapies (e.g., prostate cancer therapies known in the art or described herein. e.g., leuprolide, abiraterone, enzalutamide, leuprolide acetate (LUPRON), zoledronic acid (ZOMETA), docetaxel, cabazitaxel, radium 223, mitoxantrone, Oxaliplatin & pemetrexed); a chemo-refractory prostate cancer; a metastatic chemo-refractory prostate cancer), a bladder cancer, a melanoma, a small cell lung cancer (SCLC) (including an extensive-stage SCLC), a non-small cell lung cancer (NSCLC), a pancreatic cancer (e.g., an advanced pancreatic cancer, a metastatic pancreatic cancer, a pancreatic adenocarcinoma, a metastatic pancreatic adenocarcinoma, an MSI-H pancreatic cancer, a pancreatic ductal adenocarcinoma, a metastatic pancreatic ductal adenocarcinoma), a colorectal cancer, a gastric cancer, an esophageal cancer, an ovarian cancer, a head and neck squamous cell carcinoma, or an MSI-H (microsatellite instability high) cancer (e.g., an MSI-H prostate cancer, MSI-H ovarian cancer. MSI-H ovarian cancer, MSI-H HNSCC, MSI-H SLCLC (e.g., an MSI-H extensive-stage SCLC)) in a human patient. In some instances, the cancer is an MSI-H cancer selected from a castration-resistant prostate cancer, a bladder cancer, a melanoma, a SCLC (e.g., an extensive-stage SCLC), a pancreatic cancer (e.g., an advanced pancreatic cancer, a metastatic pancreatic cancer, a pancreatic adenocarcinoma, a metastatic pancreatic adenocarcinoma, an MSI-H pancreatic cancer, a pancreatic ductal adenocarcinoma, a metastatic pancreatic ductal adenocarcinoma), a colorectal cancer, a gastric cancer, an esophageal cancer, an ovarian cancer, a head and neck squamous cell carcinoma). The method comprises administering to the human patient an anti-GFRAL antibody at a dose of about 30 mg about every 3 weeks. In some cases, the method comprises administering to the human patient an anti-GFRAL antibody at a dose of 30 mg every 3 weeks. In some instances, the anti-GFRAL antibody inhibits GFRAL binding to RET.

In a second aspect, this disclosure features a method for treating a tumor (e.g., a solid tumor, an advanced solid tumor, a cancer, e.g., a prostate cancer (e.g., an mCRPC, a castration-sensitive prostate cancer, a castration-resistant prostate cancer; an MSI-H prostate cancer, a metastatic prostate cancer, a metastatic castrate-resistant, chemo-refractory prostate cancer; an advanced prostate cancer where the patient has exhausted all standard therapies with elevated baseline serum GDF15; an advanced prostate cancer where the patient has previously received one or more (e.g., 1, 2, 3, or more) lines of cancer therapies (e.g., prostate cancer therapies known in the art or described herein, e.g., leuprolide, abiraterone, enzalutamide, leuprolide acetate (LUPRON), zoledronic acid (ZOMETA), docetaxel, cabazitaxel, radium 223, mitoxantrone, Oxaliplatin & pemetrexed); a chemo-refractory prostate cancer; a metastatic chemo-refractory prostate cancer), a bladder cancer, a melanoma, a small cell lung cancer (SCLC) (including an extensive-stage SCLC), a non-small cell lung cancer (NSCLC), a pancreatic cancer (e.g., an advanced pancreatic cancer, a metastatic pancreatic cancer, a pancreatic adenocarcinoma, a metastatic pancreatic adenocarcinoma, an MSI-H pancreatic cancer, a pancreatic ductal adenocarcinoma, a metastatic pancreatic ductal adenocarcinoma), a colorectal cancer, a gastric cancer, an esophageal cancer, an ovarian cancer, a head and neck squamous cell carcinoma, or an MSI-H (microsatellite instability high) cancer (e.g., an MSI-H prostate cancer, MSI-H ovarian cancer, MSI-H ovarian cancer, MSI-H HNSCC, MSI-H SLCLC (e.g., an MSI-H extensive-stage SCLC)) in a human patient. In some instances, the cancer is an MSI-H cancer selected from a castration-resistant prostate cancer, a bladder cancer, a melanoma, a SCLC (e.g., an extensive-stage SCLC), a pancreatic cancer (e.g., an advanced pancreatic cancer, a metastatic pancreatic cancer, a pancreatic adenocarcinoma, a metastatic pancreatic adenocarcinoma, an MSI-H pancreatic cancer, a pancreatic ductal adenocarcinoma, a metastatic pancreatic ductal adenocarcinoma), a colorectal cancer, a gastric cancer, an esophageal cancer, an ovarian cancer, a head and neck squamous cell carcinoma). The method comprises administering to the human patient an anti-GFRAL antibody at a dose of about 100 mg about every 3 weeks. The method comprises administering to the human patient an anti-GFRAL antibody at a dose of 100 mg every 3 weeks. In some instances, the anti-GFRAL antibody inhibits GFRAL binding to RET.

In some cases in the above methods, the human patient has a relapsed or a refractory cancer or tumor after receiving one or more anti-cancer therapies. In some instances, the one or more anti-cancer therapies comprise a standard of care therapy for the respective cancer or tumor. In one case, the human patient has metastatic castration-resistant, chemo-refractory prostate cancer. In some cases, the human patient has an MSI-H cancer or tumor. In some cases, the human patient has castration-resistant prostate cancer, and has received one or more lines of cancer therapies (e.g., leuprolide acetate (LUPRON), zoledronic acid (ZOMETA), docetaxel, cabazitaxel, radium 223, mitoxantrone, Oxaliplatin and pemetrexed). In some cases, the human patient has an advanced prostate cancer (e.g., castration-resistant prostate cancer) and the patient has previously received one, two, three, or more lines of cancer therapies (e.g., leuprolide acetate (LUPRON), zoledronic acid (ZOMETA), docetaxel, cabazitaxel, radium 223, mitoxantrone, Oxaliplatin and pemetrexed).

In some instances, the tumor is prostate cancer. In some instances, the tumor is advanced prostate cancer. In some instances, the prostate cancer is a castration-sensitive prostate cancer. In some instances, the prostate cancer is an mCRPC. In some instances, the prostate cancer is a castration-resistant prostate cancer. In some instances, the prostate cancer is an MSI-H prostate cancer. In some instances, the prostate cancer is a metastatic prostate cancer. In some instances, the human patient has prostate cancer and poor survival. In some instances, the cancer is an mCRPC. In some instances, the tumor is chemo-refractory prostate cancer. In some instances, the tumor is metastatic chemo-refractory prostate cancer. In some instances, the tumor is metastatic castrate-resistant, chemo-refractory prostate cancer. In some instances, the tumor is advanced prostate cancer, wherein the human patient has exhausted all standard therapies, and wherein the human patient has an elevated baseline serum GDF15 (compared to a human or a panel of humans not having prostate cancer). In some instances, the tumor is advanced prostate cancer, wherein the human patient has previously received one or more (e.g., 1, 2, 3, or more) lines of therapies (e.g., leuprolide acetate (LUPRON), zoledronic acid (ZOMETA), docetaxel, cabazitaxel, radium 223, mitoxantrone, Oxaliplatin and pemetrexed).

In some instances, the above methods stabilizes, slows down, or prevents progression of the tumor or cancer. In certain instances, the human patient has tumor-related weight loss or cachexia. In certain instances, the method reduces tumor-related weight loss or cachexia. In some instances, the above methods reduce the serum level of GDF15 in the patients. In some instances, the human patient has castration-resistant prostate cancer, and the methods reduce the serum PSA level in the patient.

In some cases in the above methods, at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more doses of the anti-GFRAL antibody are administered to the human patient. In some cases in the above methods, the human patient is at least 35, 40, 45, 50, 55, 60, 65, 70, 75, 80, or 85 years of age.

In a third aspect, the disclosure provides a method for treating tumor-related weight loss or cachexia in a human patient. The method comprises administering to the human patient an anti-GFRAL antibody at a dose of about 30 mg once about every 3 weeks. In some cases, the method comprises administering to the human patient an anti-GFRAL antibody at a dose of 30 mg once every 3 weeks. In some cases, the antibody inhibits GFRAL binding to RET.

In a fourth aspect, the disclosure provides a method for treating tumor-related weight loss or cachexia in a human patient. The method comprises administering to the human patient an anti-GFRAL antibody at a dose of about 100 mg once about every 3 weeks. In some cases, the method comprises administering to the human patient an anti-GFRAL antibody at a dose of 100 mg once every 3 weeks. In some cases, the antibody inhibits GFRAL binding to RET

In some instances in the above methods, the human patient is receiving a concomitant anti-cancer therapy. In some instances, the concomitant anti-cancer therapy is a standard of care anti-cancer therapy for the tumor or cancer being treated. In certain cases, the concomitant anti-cancer therapy is a chemotherapy, a radiation therapy, a hormonal therapy, or a surgical therapy. In some cases in the above methods, the human patient has a relapsed or a refractory cancer or tumor after receiving one or more anti-cancer therapies. In some cases, the human patient has castration-resistant prostate cancer, and has received one or more lines of hormone therapies (e.g., leuprolide acetate (LUPRON), zoledronic acid (ZOMETA), docetaxel, cabazitaxel, radium 223, mitoxantrone, Oxaliplatin and pemetrexed). In some cases, the human patient has an advanced prostate cancer (e.g., castration-resistant prostate cancer) and the patient has previously received one or more (e.g., 1, 2, 3, or more) lines of cancer therapies (e.g., leuprolide acetate (LUPRON), zoledronic acid (ZOMETA), docetaxel, cabazitaxel, radium 223, mitoxantrone, Oxaliplatin and pemetrexed).

In some cases in all of the above methods, the tumor is a solid tumor. In some cases, the tumor is an advanced solid tumor. In some cases, the tumor is a cancer. In certain cases, the cancer is advanced or metastatic. In certain cases, the cancer is selected from a group consisting of a prostate cancer (e.g., an mCRPC, a castration-sensitive prostate cancer, a castration-resistant prostate cancer, an MSI-H prostate cancer; a metastatic prostate cancer, a metastatic castrate-resistant, chemo-refractory prostate cancer; an advanced prostate cancer where the patient has exhausted all standard therapies with elevated baseline serum GDF15; an advanced prostate cancer where the patient has previously received one or more (e.g., 1, 2, 3, or more) lines of cancer therapies; a chemo-refractory prostate cancer; a metastatic chemo-refractory prostate cancer), a bladder cancer, a melanoma, a SCLC (e.g., an extensive-stage SCLC), an NSCLC, a pancreatic cancer (e.g., an advanced pancreatic cancer, a metastatic pancreatic cancer, a pancreatic adenocarcinoma, a metastatic pancreatic adenocarcinoma, an MSI-H pancreatic cancer, a pancreatic ductal adenocarcinoma, a metastatic pancreatic ductal adenocarcinoma), a colorectal cancer, a gastric cancer, an esophageal cancer, an ovarian cancer, and a head neck squamous cell carcinoma, or an MSI-H cancer (e.g., an MSI-H prostate cancer, an MSI-H ovarian cancer, an MSI-H ovarian cancer, an MSI-H HNSCC, an MSI-H SLCLC (e.g., an MSI-H extensive-stage SCLC). In some instances, the cancer is an MSI-H cancer selected from a castration-resistant prostate cancer, a bladder cancer, a melanoma, a SCLC (e.g., an extensive-stage SCLC), a pancreatic cancer (e.g., an advanced pancreatic cancer, a metastatic pancreatic cancer, a pancreatic adenocarcinoma, a metastatic pancreatic adenocarcinoma, an MSI-H pancreatic cancer, a pancreatic ductal adenocarcinoma, a metastatic pancreatic ductal adenocarcinoma), a colorectal cancer, a gastric cancer, an esophageal cancer, an ovarian cancer, a head and neck squamous cell carcinoma). In one instance, the cancer is a pancreatic adenocarcinoma. In certain cases, the administering is performed subcutaneously. In certain cases, the administering is performed intravenously. In some cases in all of the above methods, the human patient has an elevated serum level of GDF15 prior to the administering of the anti-GFRAL antibody compared to the serum level of GDF15 in said human prior to developing the tumor. In some cases in all of the above methods, the human patient has an elevated blood/serum level of GDF15 relative to a blood/serum level of GDF15 in a human not having the tumor or cancer. In some cases in all of the above methods, the human patient has an elevated level of GDF15 in the cancer tissue relative to the same non-cancerous tissue (e.g., in a cancerous prostate tissue relative to a non-cancerous prostate tissue). In certain cases, the human patient has a blood/serum GDF level of at least about 500 pg/mL, at least about 600 pg/mL, at least about 700 pg/mL, at least about 800 pg/mL, at least about 900 pg/mL, at least about 950 pg/mL, at least about 1000 pg/mL, at least about 1200 pg/mL, at least about 1300, at least about 1400 pg/mL, at least about 1500 pg/mL, at least about 1600 pg/mL, at least about 1700 pg/m, at least about 1800 pg/mL, at least about 1900 pg/mL, at least about 2000 pg/mL. In one case, the human patient has a blood/serum GDF level of at least about 950 pg/mL (e.g., prior to the administering of the anti-GFRAL antibody). In another case, the human patient has a blood/serum GDF level of at least about 1300 pg/mL (e.g., prior to the administering of the anti-GFRAL antibody). In some instances, the human patient has a reduced serum GDF15 level after the administering of the anti-GFRAL antibody as compared to the serum GDF15 level in the human patient prior to the administering of the anti-GFRAL antibody. “About” in the context of GDF levels means+/−10% of the recited value. In one case, the human patient has a relapsed or a refractory cancer or tumor after receiving one or more anti-cancer therapies. In certain cases, the human patient has castration-resistant prostate cancer, and has received one or more lines of hormone therapies (e.g., leuprolide acetate (LUPRON), zoledronic acid (ZOMETA), docetaxel, cabazitaxel, radium 223, mitoxantrone, Oxaliplatin and pemetrexed). In some cases of the above methods, the method further comprises determining the level of beta hydroxybutyrate (BHB) in serum during or following the treatment period. In some instances, the level of BHB in serum is reduced following treatment with the anti-GFRAL antibody. In some cases in the above methods, at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more doses of the anti-GFRAL antibody are administered to the human patient. In some cases in the above methods, the human patient is at least 35, 40, 45, 50, 55, 60, 65, 70, 75, 80, or 85 years of age.

In some instances, the tumor is a prostate cancer. In some instances, the tumor is an advanced prostate cancer. In some instances, the human patient has prostate cancer and poor survival. In some instances, the prostate cancer is an mCRPC. In some instances, the prostate cancer is a castration-sensitive prostate cancer. In some instances, the prostate cancer is a castration-resistant prostate cancer tumor. In some instances, the prostate cancer is an MSI-H prostate cancer. In some instances, the prostate cancer is a metastatic prostate cancer. In some instances, the tumor is a chemo-refractory prostate cancer. In some instances, the tumor is a metastatic chemo-refractory prostate cancer. In some instances, the tumor is a metastatic castrate-resistant, chemo-refractory prostate cancer. In some instances, the tumor is an advanced prostate cancer, wherein the human patient has exhausted all standard therapies, and wherein the human patient has an elevated baseline serum GDF15 (compared to a human or a panel of humans not having prostate cancer). In some instances, the tumor is an advanced prostate cancer, wherein the human patient has previously received one or more (e.g., 1, 2, 3, or more) lines of therapies (e.g., leuprolide acetate (LUPRON), zoledronic acid (ZOMETA), docetaxel, cabazitaxel, radium 223, mitoxantrone, Oxaliplatin and pemetrexed).

In a fifth aspect, the disclosure relates to a method for treating pancreatic cancer in a human patient. The method comprises administering to the human patient an anti-GFRAL antibody at a dose of about 30 mg once about every 4 weeks; paclitaxel (e.g., nab-paclitaxel); and gemcitabine. In some cases, the method comprises administering to the human patient an anti-GFRAL antibody at a dose of 30 mg once every 4 weeks; paclitaxel (e.g., nab-paclitaxel); and gemcitabine. In some cases, the anti-GFRAL antibody inhibits GFRAL binding to RET. In some instances, the paclitaxel (e.g., nab-paclitaxel) is administered to the human patient at a dose of 125 mg/m² once every week. In some instances, the gemcitabine is administered to the human patient at a dose of 1000 mg/m² gemcitabine once every week. In some instances, the pancreatic cancer is an advanced pancreatic cancer. In some instances, the pancreatic cancer is a metastatic pancreatic cancer. In some instances, the pancreatic cancer is a pancreatic adenocarcinoma. In some instances, the pancreatic cancer is a metastatic pancreatic adenocarcinoma. In some instances, the pancreatic cancer is an MSI-H pancreatic cancer. In some instances, the pancreatic cancer is a pancreatic ductal adenocarcinoma. In some instances, the pancreatic cancer is a metastatic pancreatic ductal adenocarcinoma.

In a sixth aspect, the disclosure relates to a method for treating pancreatic cancer in a human patient. The method comprises administering to the human patient an anti-GFRAL antibody at a dose of about 100 mg once about every 4 weeks; paclitaxel (e.g., nab-paclitaxel); and gemcitabine. In some cases, the method comprises administering to the human patient an anti-GFRAL antibody at a dose of 100 mg once every 4 weeks; paclitaxel (e.g., nab-paclitaxel); and gemcitabine. In some cases, the anti-GFRAL antibody inhibits GFRAL binding to RET. In some instances, the paclitaxel (e.g., nab-paclitaxel) is administered to the human patient at a dose of 125 mg/m² once every week. In some instances, the gemcitabine is administered to the human patient at a dose of 1000 mg/m² gemcitabine once every week. In some instances, the pancreatic cancer is an advanced pancreatic cancer. In some instances, the pancreatic cancer is a metastatic pancreatic cancer. In some instances, the pancreatic cancer is a pancreatic adenocarcinoma. In some instances, the pancreatic cancer is a metastatic pancreatic adenocarcinoma. In some instances, the pancreatic cancer is an MSI-H pancreatic cancer. In some instances, the pancreatic cancer is a pancreatic ductal adenocarcinoma. In some instances, the pancreatic cancer is a metastatic pancreatic ductal adenocarcinoma.

In some instances, the paclitaxel is administered to the human patient twice during the 4 week interval during which the anti-GFRAL antibody is administered to the human patient, and gemcitabine is administered to the human patient twice during the 4 week interval during which the anti-GFRAL antibody is administered to the human patient. In certain instances, the paclitaxel is administered to the human patient three times during the 4 week interval during which the anti-GFRAL antibody is administered to the human patient, and the gemcitabine is administered to the human patient three times during the 4 week interval during which the anti-GFRAL antibody is administered to the human patient. In some cases, the paclitaxel is nab-paclitaxel. In cases wherein the paclitaxel is nab-paclitaxel. In some instances, the nab-paclitaxel is administered to the human patient at a dose of 125 mg/m² once every week for three weeks during the 4 week interval during which the anti-GFRAL antibody is administered to the human patient. In some instances, the gemcitabine is administered to the human patient at a dose of 1000 mg/m² once every week for three weeks during the 4 week interval during which the anti-GFRAL antibody is administered to the human patient. In some cases, the human patient has pancreatic tumor-related weight loss or cachexia. In certain cases, the method reduces pancreatic tumor-related weight loss or cachexia in the human patient. In some cases, the pancreatic cancer is pancreatic adenocarcinoma. In certain cases, the pancreatic cancer is metastatic. In some cases, the pancreatic cancer is metastatic pancreatic adenocarcinoma. In some instances, the pancreatic cancer is an advanced pancreatic cancer. In some instances, the pancreatic cancer is an MSI-H pancreatic cancer. In some instances, the pancreatic cancer is a pancreatic ductal adenocarcinoma. In some instances, the pancreatic cancer is a metastatic pancreatic ductal adenocarcinoma. In some cases, the administering of the anti-GRFAL antibody is performed subcutaneously, whereas the administering of the paclitaxel and gemcitabine is performed intravenously. In some cases, the administering of the anti-GRFAL, the paclitaxel, and the gemcitabine is each performed intravenously. In some cases, the human patient has an elevated serum level of GDF15 prior to the administering of the anti-GFRAL antibody compared to the serum level of GDF15 in said human prior to developing the cancer. In some cases, the human patient has an elevated blood/serum level of GDF15 relative to a blood/serum level of GDF15 in a human not having the cancer. In some cases, the human patient has an elevated level of GDF15 in the cancer tissue relative to the same non-cancerous tissue (e.g., in a cancerous prostate tissue relative to a non-cancerous prostate tissue). In certain cases, the human patient has a blood/serum GDF level of at least about 500 pg/mL, at least about 600 pg/mL, at least about 700 pg/mL, at least about 800 pg/mL, at least about 900 pg/mL, at least about 950 pg/mL, at least about 1000 pg/mL, at least about 1200 pg/mL, at least about 1300, at least about 1400 pg/mL, at least about 1500 pg/mL, at least about 1600 pg/mL, at least about 1700 pg/m, at least about 1800 pg/mL, at least about 1900 pg/mL, at least about 2000 pg/mL. In one case, the human patient has a blood/serum GDF level of at least about 950 pg/mL. In another case, the human patient has a blood/serum GDF level of at least about 1300 pg/mL. In some instances, the human patient has a reduced serum GDF15 level after the administering of the anti-GFRAL antibody as compared to the serum GDF15 level in the human patient prior to the administering of the anti-GFRAL antibody. In one case, the human patient has a relapsed or a refractory cancer or tumor after receiving one or more anti-cancer therapies. In some cases in the above methods, at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more doses of the anti-GFRAL antibody are administered to the human patient. In some cases in the above methods, the human patient is at least 35, 40, 45, 50, 55, 60, 65, 70, 75, 80, or 85 years of age. In some cases, the method further comprises determining the levels of the tumor biomarker CA19-9. In certain cases, the levels of the tumor biomarker CAl9-9 are reduced relative to the level prior to treatment.

In some instances in all of the above methods, the anti-GFRAL antibody specifically binds within amino acid residues 220-316 of the amino acid sequence set forth in SEQ ID NO: 1797. In certain cases, the anti-GFRAL antibody specifically binds to one or more (e.g., 1, 2, 3, 4, 5, 6, 7, 8, 9, 10, 11, 12, 13, 14, 15, 16, 17, 18, 19) residues selected from the group consisting of: Met214, Pro216, Pro217, Gln290, Cys291, Thr292, Cys293, Arg294, Thr295, Ile296, Thr297, Gln298, Ser299, Glu301, Lys305, Gln308, His309, His312, and Ser315 of the amino acid sequence set forth in SEQ ID NO: 1797. In some cases, the anti-GFRAL antibody specifically binds to at least Thr297, Gln298, and Ser299 of the amino acid sequence set forth in SEQ ID NO:1797. In some cases, the antibody comprises a heavy chain variable region (VH) and a light chain variable region (VL), and wherein the VH comprises a VH complementarity determining region (CDR)1, a VH CDR2, and a VH CDR3 from the amino acid sequence set forth in SEQ ID NO:1982, and the VL comprises a VL CDR1, a VL CDR2, and a VL CDR3 from the amino acid sequence set forth in SEQ ID NO:1997. In some cases the CDRS are based on one of the following definitions: Exemplary, IMGT, Kabat, Chothia, Contact, or AbM. In some instances, the antibody comprises a VH comprising the three VH CDRs and a VL comprising the three VL CDRs of any one of the six CDR definitions provided in Table 1. In certain cases, the VH comprises an amino acid sequence that has at least 90% sequence identity to the amino acid sequence set forth in SEQ ID NO:1982 and the VL comprises an amino acid sequence that has at least 90% sequence identity to the amino acid sequence set forth in SEQ ID NO: 1997. In other cases, the VH comprises an amino acid sequence that has at least 95% sequence identity to the amino acid sequence set forth in SEQ ID NO:1982 and the VL comprises an amino acid sequence that has at least 95% sequence identity to the amino acid sequence set forth in SEQ ID NO:1997. In yet other cases, the VH comprises the amino acid sequence set forth in SEQ ID NO:1982 and the VL comprises the amino acid sequence set forth in SEQ ID NO:1997. In some cases, the antibody is a human IgG1, human IgG2, or human IgG4 antibody. In certain cases, the antibody is a human IgG1 antibody. In certain instances, the antibody comprises a human kappa light chain constant region. In some instances, the antibody comprises a human lambda light chain constant region. In certain instances, the antibody comprises a heavy chain comprising an amino acid sequence with 80% identity to the amino acid sequence set forth in SEQ ID NO:2010 and a light chain comprising an amino acid sequence with 80% identity to the amino acid sequence set forth in SEQ ID NO:2012. In other instances, the antibody comprises a heavy chain comprising an amino acid sequence with 90% identity to the amino acid sequence set forth in SEQ ID NO:2010 and a light chain comprising an amino acid sequence with 90% identity to the amino acid sequence set forth in SEQ ID NO: 2012. In yet other instances, the antibody comprises a heavy chain comprising an amino acid sequence with 95% identity to the amino acid sequence set forth in SEQ ID NO: 2010 and a light chain comprising an amino acid sequence with 95% identity the amino acid sequence set forth in SEQ ID NO: 2012. In some cases, the antibody comprises a heavy chain comprising the amino acid sequence set forth in SEQ ID NO: 2010 and a light chain comprising the amino acid sequence set forth in SEQ ID NO: 2012. In other cases, the antibody comprises a heavy chain consisting of the amino acid sequence set forth in SEQ ID NO: 2010 and a light chain consisting of the amino acid sequence set forth in SEQ ID NO: 2012.

The disclosure also features a method of predicting the efficacy of an anti-GFRAL antibody therapy in a human subject. The method comprises determining the blood or serum level of beta hydroxybutyrate (BHB) following treatment comprising the anti-GFRAL antibody relative to the blood or serum level of BHB in an untreated patient or the serum level of BHB prior to treatment in the human subject. The treatment is likely to be effective (and so can be continued if needed) if the blood or serum levels of BHB are reduced following treatment.

Also provided herein is a method for treating a tumor (e.g., a solid tumor, an advanced solid tumor, a cancer) in a human patient, the method comprising administering to the human patient an anti-GFRAL antibody at a dose of 30 mg once every 3 weeks, wherein the anti-GFRAL antibody comprises: (i) a VH comprising a VH CDR1 comprising the amino acid sequence set forth in SEQ ID NO:48, a VH CDR2 comprising the amino acid sequence set forth in SEQ ID NO:137, and a VH CDR3 comprising the amino acid sequence set forth in SEQ ID NO:225, and (ii) a VL comprising a VL CDR1 comprising the amino acid sequence set forth in SEQ ID NO:301, a VL CDR2 comprising the amino acid sequence set forth in SEQ ID NO:376, and a VL CDR3 comprising the amino acid sequence set forth in SEQ ID NO:426. In some instances, the administering is performed subcutaneously. In some instances, the administering is performed intravenously. In some instances, the human patient has an elevated level of GDF15 (e.g., compared to a level of GDF15 in a human or a panel of humans (e.g., 2, 3, 4, 5, 10, 15, 20, 25, or more) not having the tumor). In some instances, the tumor is a solid tumor. In some instances, the tumor is an advanced solid tumor. In some instances, the tumor is a cancer. In some instances, the cancer is metastatic. In some instances, the cancer is advanced. In some instances, the cancer is a prostate cancer (e.g., an mCRPC, a castration-sensitive prostate cancer, a castration-resistant prostate cancer, an MSI-H prostate cancer; a metastatic prostate cancer, a metastatic castrate-resistant, chemo-refractory prostate cancer; an advanced prostate cancer where the patient has exhausted all standard therapies with elevated baseline serum GDF15; an advanced prostate cancer where the patient has previously received one or more (e.g., 1, 2, 3, or more) lines of cancer therapies; a chemo-refractory prostate cancer; a metastatic chemo-refractory prostate cancer), a bladder cancer, a melanoma, an SCLC (e.g., an extensive-stage SCLC), an NSCLC, a pancreatic cancer (e.g., an advanced pancreatic cancer, a metastatic pancreatic cancer, a pancreatic adenocarcinoma, a metastatic pancreatic adenocarcinoma, an MSI-H pancreatic cancer, a pancreatic ductal adenocarcinoma, a metastatic pancreatic ductal adenocarcinoma), a colorectal cancer, a gastric cancer, an esophageal cancer, an ovarian cancer, a head neck squamous cell carcinoma, or an MSI-H cancer (e.g., an MSI-H prostate cancer, an MSI-H ovarian cancer, an MSI-H ovarian cancer, an MSI-H HNSCC, an MSI-H SLCLC (e.g., an MSI-H extensive-stage SCLC). In some instances, the cancer is an MSI-H cancer selected from a castration-resistant prostate cancer, a bladder cancer, a melanoma, a SCLC (e.g., an extensive-stage SCLC), a pancreatic cancer (e.g., an advanced pancreatic cancer, a metastatic pancreatic cancer, a pancreatic adenocarcinoma, a metastatic pancreatic adenocarcinoma, an MSI-H pancreatic cancer, a pancreatic ductal adenocarcinoma, a metastatic pancreatic ductal adenocarcinoma), a colorectal cancer, a gastric cancer, an esophageal cancer, an ovarian cancer, a head and neck squamous cell carcinoma). In some instances, the cancer is a pancreatic cancer (e.g., an advanced pancreatic cancer, a metastatic pancreatic cancer, a pancreatic adenocarcinoma, a metastatic pancreatic adenocarcinoma, an MSI-H pancreatic cancer, a pancreatic ductal adenocarcinoma, a metastatic pancreatic ductal adenocarcinoma). In some instances, the cancer is a pancreatic adenocarcinoma. In some instances, the cancer is a metastatic pancreatic cancer. In some instances, the cancer is a metastatic pancreatic adenocarcinoma. In some instances, the cancer is a metastatic pancreatic cancer. In some instances, the pancreatic cancer is an advanced pancreatic cancer. In some instances, the pancreatic cancer is an MSI-H pancreatic cancer. In some instances, the pancreatic cancer is a pancreatic ductal adenocarcinoma. In some instances, the pancreatic cancer is a metastatic pancreatic ductal adenocarcinoma. In some instances, the human patient has a prostate cancer (e.g., an mCRPC, a castration-sensitive prostate cancer, a castration-resistant prostate cancer, an MSI-H prostate cancer; a metastatic prostate cancer, a metastatic castrate-resistant, chemo-refractory prostate cancer; an advanced prostate cancer where the patient has exhausted all standard therapies with elevated baseline serum GDF15; an advanced prostate cancer where the patient has previously received one or more (e.g., 1, 2, 3, or more) lines of cancer therapies; a chemo-refractory prostate cancer; a metastatic chemo-refractory prostate cancer). In some instances, the cancer is advanced prostate cancer. In some instances, the human patient has prostate cancer and poor survival. In some instances, the prostate cancer is an mCRPC. In some instances, the cancer is chemo-refractory prostate cancer. In some instances, the cancer is metastatic chemo-refractory prostate cancer. In some instances, the cancer is metastatic castrate-resistant, chemo-refractory prostate cancer. In some instances, the cancer is advanced prostate cancer, wherein the human patient has exhausted all standard therapies, and wherein the human patient has an elevated baseline serum GDF15 (compared to a human or a panel of humans not having prostate cancer). In some instances, the cancer is advanced prostate cancer (e.g., castration-resistant prostate cancer), wherein the human patient has previously received one or more (e.g., 1, 2, 3, or more) lines of therapies (e.g., leuprolide acetate (LUPRON), zoledronic acid (ZOMETA), docetaxel, cabazitaxel, radium 223, mitoxantrone, Oxaliplatin and pemetrexed). In some instances, the human patient has castration-resistant prostate cancer. In some instances, the human patient has castration-resistant prostate cancer and previously failed after one or more lines of hormone therapies. In some cases, the human patient has an advanced prostate cancer (e.g., an mCRPC, castration-resistant prostate cancer) and the patient has previously received one or more (e.g., 1, 2, 3, or more) lines of cancer therapies (e.g., leuprolide acetate (LUPRON), zoledronic acid (ZOMETA), docetaxel, cabazitaxel, radium 223, mitoxantrone, Oxaliplatin and pemetrexed). In some instances, the human patient previously failed to be treated after treatment with one or more (e.g., 1, 2, 3, 4, 5) other cancer therapies (i.e., other than an anti-GFRAL antibody described herein) prior to the administering of the anti-GFRAL antibody.

Also provided herein is a method for treating a tumor (e.g., a solid tumor, an advanced solid tumor, a cancer) in a human patient, the method comprising administering to the human patient an anti-GFRAL antibody at a dose of 30 mg once every 3 weeks, wherein the anti-GFRAL antibody comprises a VH comprising the amino acid sequence set forth in SEQ ID NO:1982 and a VL comprising the amino acid sequence set forth in SEQ ID NO:1997. In some instances, the administering is performed subcutaneously. In some instances, the administering is performed intravenously. In some instances, the human patient has an elevated level of GDF15 (e.g., compared to a level of GDF15 in a human or a panel of humans (e.g., 2, 3, 4, 5, 10, 15, 20, 25, or more) not having the tumor). In some instances, the tumor is a solid tumor. In some instances, the tumor is an advanced solid tumor. In some instances, the tumor is a cancer. In some instances, the cancer is metastatic. In some instances, the cancer is advanced. In some instances, the cancer is a prostate cancer (e.g., an mCRPC, a castration-sensitive prostate cancer, a castration-resistant prostate cancer, an MSI-H prostate cancer; a metastatic prostate cancer, a metastatic castrate-resistant, chemo-refractory prostate cancer; an advanced prostate cancer where the patient has exhausted all standard therapies with elevated baseline serum GDF15; an advanced prostate cancer where the patient has previously received one or more (e.g., 1, 2, 3, or more) lines of cancer therapies; a chemo-refractory prostate cancer; a metastatic chemo-refractory prostate cancer), a bladder cancer, a melanoma, an SCLC (e.g., an extensive-stage SCLC), an NSCLC, a pancreatic cancer (e.g., an advanced pancreatic cancer, a metastatic pancreatic cancer, a pancreatic adenocarcinoma, a metastatic pancreatic adenocarcinoma, an MSI-H pancreatic cancer, a pancreatic ductal adenocarcinoma, a metastatic pancreatic ductal adenocarcinoma), a colorectal cancer, a gastric cancer, an esophageal cancer, an ovarian cancer, a head neck squamous cell carcinoma or an MSI-H cancer (e.g., an MSI-H prostate cancer, an MSI-H ovarian cancer, an MSI-H ovarian cancer, an MSI-H HNSCC, an MSI-H SLCLC (e.g., an MSI-H extensive-stage SCLC). In some instances, the cancer is an MSI-H cancer selected from a castration-resistant prostate cancer, a bladder cancer, a melanoma, a SCLC (e.g., an extensive-stage SCLC), a pancreatic cancer (e.g., an advanced pancreatic cancer, a metastatic pancreatic cancer, a pancreatic adenocarcinoma, a metastatic pancreatic adenocarcinoma, an MSI-H pancreatic cancer, a pancreatic ductal adenocarcinoma, a metastatic pancreatic ductal adenocarcinoma), a colorectal cancer, a gastric cancer, an esophageal cancer, an ovarian cancer, a head and neck squamous cell carcinoma). In some instances, the cancer is a pancreatic cancer (e.g., an advanced pancreatic cancer, a metastatic pancreatic cancer, a pancreatic adenocarcinoma, a metastatic pancreatic adenocarcinoma, an MSI-H pancreatic cancer, a pancreatic ductal adenocarcinoma, a metastatic pancreatic ductal adenocarcinoma). In some instances, the cancer is a pancreatic adenocarcinoma. In some instances, the cancer is a metastatic pancreatic cancer. In some instances, the cancer is a metastatic pancreatic adenocarcinoma. In some instances, the pancreatic cancer is an advanced pancreatic cancer. In some instances, the pancreatic cancer is an MSI-H pancreatic cancer. In some instances, the pancreatic cancer is a pancreatic ductal adenocarcinoma. In some instances, the pancreatic cancer is a metastatic pancreatic ductal adenocarcinoma. In some instances, the human patient has a prostate cancer (e.g., an mCRPC, a castration-sensitive prostate cancer, a castration-resistant prostate cancer, an MSI-H prostate cancer; a metastatic prostate cancer, a metastatic castrate-resistant, chemo-refractory prostate cancer; an advanced prostate cancer where the patient has exhausted all standard therapies with elevated baseline serum GDF15; an advanced prostate cancer where the patient has previously received one or more (e.g., 1, 2, 3, or more) lines of cancer therapies; a chemo-refractory prostate cancer; a metastatic chemo-refractory prostate cancer). In some instances, the cancer is advanced prostate cancer. In some instances, the human patient has prostate cancer and poor survival. In some instances, the cancer is an mCRPC. In some instances, the cancer is chemo-refractory prostate cancer. In some instances, the cancer is metastatic chemo-refractory prostate cancer. In some instances, the cancer is metastatic castrate-resistant, chemo-refractory prostate cancer. In some instances, the cancer is advanced prostate cancer, wherein the human patient has exhausted all standard therapies, and wherein the human patient has an elevated baseline serum GDF15 (compared to a human or a panel of humans not having prostate cancer). In some instances, the cancer is advanced prostate cancer (e.g., castration-resistant prostate cancer), wherein the human patient has previously received one or more (e.g., 1, 2, 3, or more) lines of therapies (e.g., leuprolide acetate (LUPRON), zoledronic acid (ZOMETA), docetaxel, cabazitaxel, radium 223, mitoxantrone, Oxaliplatin and pemetrexed). In some instances, the human patient previously failed to be treated with one or more (e.g., 1, 2, 3, 4, 5) other cancer therapies (i.e., other than an anti-GFRAL antibody described herein) prior to the administering of the anti-GFRAL antibody.

Also provided herein is a method for treating a tumor (e.g., a solid tumor, an advanced solid tumor, a cancer) in a human patient, comprising administering to the human patient an anti-GFRAL antibody at a dose of 30 mg once every 3 weeks, wherein the anti-GFRAL antibody comprises a heavy chain comprising the amino acid sequence set forth in SEQ ID NO:2010 and a light chain comprising the amino acid sequence set forth in SEQ ID NO:2012. In some instances, the anti-GFRAL antibody comprises a heavy chain consisting of the amino acid sequence set forth in SEQ ID NO:2010 and a light chain comprising the amino acid sequence set forth in SEQ ID NO:2012. In some instances, the administering is performed subcutaneously. In some instances, the administering is performed intravenously. In some instances, the human patient has an elevated level of GDF15 (e.g., compared to a level of GDF15 in a human or a panel of humans (e.g., 2, 3, 4, 5, 10, 15, 20, 25, or more) not having the tumor). In some instances, the tumor is a solid tumor. In some instances, the tumor is an advanced solid tumor. In some instances, the tumor is a cancer. In some instances, the cancer is metastatic. In some instances, the cancer is advanced. In some instances, the cancer is a prostate cancer (e.g., an mCRPC, a castration-sensitive prostate cancer, a castration-resistant prostate cancer, an MSI-H prostate cancer; a metastatic prostate cancer, a metastatic castrate-resistant, chemo-refractory prostate cancer; an advanced prostate cancer where the patient has exhausted all standard therapies with elevated baseline serum GDF15; an advanced prostate cancer where the patient has previously received one or more (e.g., 1, 2, 3, or more) lines of cancer therapies; a chemo-refractory prostate cancer; a metastatic chemo-refractory prostate cancer), a bladder cancer, a melanoma, an SCLC (e.g., an extensive-stage SCLC), an NSCLC, a pancreatic cancer (e.g., an advanced pancreatic cancer, a metastatic pancreatic cancer, a pancreatic adenocarcinoma, a metastatic pancreatic adenocarcinoma, an MSI-H pancreatic cancer, a pancreatic ductal adenocarcinoma, a metastatic pancreatic ductal adenocarcinoma), a colorectal cancer, a gastric cancer, an esophageal cancer, an ovarian cancer, a head neck squamous cell carcinoma or an MSI-H cancer (e.g., an MSI-H prostate cancer, an MSI-H ovarian cancer, an MSI-H ovarian cancer, an MSI-H HNSCC, an MSI-H SLCLC (e.g., an MSI-H extensive-stage SCLC). In some instances, the cancer is an MSI-H cancer selected from a castration-resistant prostate cancer, a bladder cancer, a melanoma, a SCLC (e.g., an extensive-stage SCLC), a pancreatic cancer (e.g., an advanced pancreatic cancer, a metastatic pancreatic cancer, a pancreatic adenocarcinoma, a metastatic pancreatic adenocarcinoma, an MSI-H pancreatic cancer, a pancreatic ductal adenocarcinoma, a metastatic pancreatic ductal adenocarcinoma), a colorectal cancer, a gastric cancer, an esophageal cancer, an ovarian cancer, a head and neck squamous cell carcinoma). In some instances, the cancer is a pancreatic cancer (e.g., an advanced pancreatic cancer, a metastatic pancreatic cancer, a pancreatic adenocarcinoma, a metastatic pancreatic adenocarcinoma, an MSI-H pancreatic cancer, a pancreatic ductal adenocarcinoma, a metastatic pancreatic ductal adenocarcinoma). In some instances, the cancer is a pancreatic adenocarcinoma. In some instances, the cancer is a metastatic pancreatic cancer. In some instances, the cancer is a metastatic pancreatic adenocarcinoma. In some instances, the pancreatic cancer is an advanced pancreatic cancer. In some instances, the pancreatic cancer is an MSI-H pancreatic cancer. In some instances, the pancreatic cancer is a pancreatic ductal adenocarcinoma. In some instances, the pancreatic cancer is a metastatic pancreatic ductal adenocarcinoma. In some instances, the human patient has a prostate cancer (e.g., an mCRPC, a castration-sensitive prostate cancer, a castration-resistant prostate cancer, an MSI-H prostate cancer; a metastatic prostate cancer, a metastatic castrate-resistant, chemo-refractory prostate cancer; an advanced prostate cancer where the patient has exhausted all standard therapies with elevated baseline serum GDF15; an advanced prostate cancer where the patient has previously received one or more (e.g., 1, 2, 3, or more) lines of cancer therapies; a chemo-refractory prostate cancer; a metastatic chemo-refractory prostate cancer). In some instances, the cancer is advanced prostate cancer. In some instances, the human patient has prostate cancer and poor survival. In some instances, the cancer is an mCRPC. In some instances, the cancer is chemo-refractory prostate cancer. In some instances, the cancer is metastatic chemo-refractory prostate cancer. In some instances, the cancer is metastatic castrate-resistant, chemo-refractory prostate cancer. In some instances, the cancer is advanced prostate cancer, wherein the human patient has exhausted all standard therapies, and wherein the human patient has an elevated baseline serum GDF15 (compared to a human or a panel of humans not having prostate cancer). In some instances, the cancer is advanced prostate cancer (e.g., castration-resistant prostate cancer), wherein the human patient has previously received one or more (e.g., 1, 2, 3, or more) lines of therapies (e.g., leuprolide acetate (LUPRON), zoledronic acid (ZOMETA), docetaxel, cabazitaxel, radium 223, mitoxantrone, Oxaliplatin and pemetrexed). In some instances, the human patient previously failed to be treated with one or more (e.g., 1, 2, 3, 4, 5) other cancer therapies (i.e., other than an anti-GFRAL antibody described herein) prior to the administering of the anti-GFRAL antibody.

Also provided herein is a method for treating a tumor (e.g., a solid tumor, an advanced solid tumor, a cancer) in a human patient, the method comprising administering to the human patient an anti-GFRAL antibody at a dose of 100 mg once every 3 weeks, wherein the anti-GFRAL antibody comprises: (i) a VH comprising a VH CDR1 comprising the amino acid sequence set forth in SEQ ID NO:48, a VH CDR2 comprising the amino acid sequence set forth in SEQ ID NO:137, and a VH CDR3 comprising the amino acid sequence set forth in SEQ ID NO:225, and (ii) a VL comprising a VL CDR1 comprising the amino acid sequence set forth in SEQ ID NO:301, a VL CDR2 comprising the amino acid sequence set forth in SEQ ID NO:376, and a VL CDR3 comprising the amino acid sequence set forth in SEQ ID NO:426. In some instances, the administering is performed subcutaneously. In some instances, the administering is performed intravenously. In some instances, the human patient has an elevated level of GDF15 (e.g., compared to a level of GDF15 in a human or a panel of humans (e.g., 2, 3, 4, 5, 10, 15, 20, 25, or more) not having the tumor). In some instances, the tumor is a solid tumor. In some instances, the tumor is an advanced solid tumor. In some instances, the tumor is a cancer. In some instances, the cancer is metastatic. In some instances, the cancer is advanced. In some instances, the cancer is a prostate cancer (e.g., an mCRPC, a castration-sensitive prostate cancer, a castration-resistant prostate cancer, an MSI-H prostate cancer; a metastatic prostate cancer, a metastatic castrate-resistant, chemo-refractory prostate cancer; an advanced prostate cancer where the patient has exhausted all standard therapies with elevated baseline serum GDF15; an advanced prostate cancer where the patient has previously received one or more (e.g., 1, 2, 3, or more) lines of cancer therapies; a chemo-refractory prostate cancer; a metastatic chemo-refractory prostate cancer), a bladder cancer, a melanoma, an SCLC (e.g., an extensive-stage SCLC), an NSCLC, a pancreatic cancer (e.g., an advanced pancreatic cancer, a metastatic pancreatic cancer, a pancreatic adenocarcinoma, a metastatic pancreatic adenocarcinoma, an MSI-H pancreatic cancer, a pancreatic ductal adenocarcinoma, a metastatic pancreatic ductal adenocarcinoma), a colorectal cancer, a gastric cancer, an esophageal cancer, an ovanan cancer, a head neck squamous cell carcinoma or an MSI-H cancer (e.g., an MSI-H prostate cancer, an MSI-H ovarian cancer, an MSI-H ovarian cancer, an MSI-H HNSCC, an MSI-H SLCLC (e.g., an MSI-H extensive-stage SCLC). In some instances, the cancer is an MSI-H cancer selected from a castration-resistant prostate cancer, a bladder cancer, a melanoma, a SCLC (e.g., an extensive-stage SCLC), a pancreatic cancer (e.g., an advanced pancreatic cancer, a metastatic pancreatic cancer, a pancreatic adenocarcinoma, a metastatic pancreatic adenocarcinoma, an MSI-H pancreatic cancer, a pancreatic ductal adenocarcinoma, a metastatic pancreatic ductal adenocarcinoma), a colorectal cancer, a gastric cancer, an esophageal cancer, an ovarian cancer, a head and neck squamous cell carcinoma). In some instances, the cancer is a pancreatic cancer (e.g., an advanced pancreatic cancer, a metastatic pancreatic cancer, a pancreatic adenocarcinoma, a metastatic pancreatic adenocarcinoma, an MSI-H pancreatic cancer, a pancreatic ductal adenocarcinoma, a metastatic pancreatic ductal adenocarcinoma). In some instances, the cancer is a pancreatic adenocarcinoma. In some instances, the cancer is a metastatic pancreatic cancer. In some instances, the cancer is a metastatic pancreatic adenocarcinoma. In some instances, the pancreatic cancer is an advanced pancreatic cancer. In some instances, the pancreatic cancer is an MSI-H pancreatic cancer. In some instances, the pancreatic cancer is a pancreatic ductal adenocarcinoma. In some instances, the pancreatic cancer is a metastatic pancreatic ductal adenocarcinoma. In some instances, the human patient has a prostate cancer (e.g., an mCRPC, a castration-sensitive prostate cancer, a castration-resistant prostate cancer, an MSI-H prostate cancer; a metastatic prostate cancer, a metastatic castrate-resistant, chemo-refractory prostate cancer; an advanced prostate cancer where the patient has exhausted all standard therapies with elevated baseline serum GDF15; an advanced prostate cancer where the patient has previously received one or more (e.g., 1, 2, 3, or more) lines of cancer therapies; a chemo-refractory prostate cancer; a metastatic chemo-refractory prostate cancer). In some instances, the cancer is advanced prostate cancer. In some instances, the human patient has prostate cancer and poor survival. In some instances, the cancer is an mCRPC. In some instances, the cancer is chemo-refractory prostate cancer. In some instances, the cancer is metastatic chemo-refractory prostate cancer. In some instances, the cancer is metastatic castrate-resistant, chemo-refractory prostate cancer. In some instances, the cancer is advanced prostate cancer, wherein the human patient has exhausted all standard therapies, and wherein the human patient has an elevated baseline serum GDF15 (compared to a human or a panel of humans not having prostate cancer). In some instances, the cancer is advanced prostate cancer (e.g., castration-resistant prostate cancer), wherein the human patient has previously received one or more (e.g., 1, 2, 3, or more) lines of therapies (e.g., leuprolide acetate (LUPRON), zoledronic acid (ZOMETA), docetaxel, cabazitaxel, radium 223, mitoxantrone, Oxaliplatin and pemetrexed). In some instances, the human patient previously failed to be treated with one or more (e.g., 1, 2, 3, 4, 5) other cancer therapies (i.e., other than an anti-GFRAL antibody described herein) prior to the administering of the anti-GFRAL antibody.

Also provided herein is a method for treating a tumor (e.g., a solid tumor, an advanced solid tumor, a cancer) in a human patient, the method comprising administering to the human patient an anti-GFRAL antibody at a dose of 100 mg once every 3 weeks, wherein the anti-GFRAL antibody comprises a VH comprising the amino acid sequence set forth in SEQ ID NO:1982 and a VL comprising the amino acid sequence set forth in SEQ ID NO:1997. In some instances, the administering is performed subcutaneously. In some instances, the administering is performed intravenously. In some instances, the human patient has an elevated level of GDF15 (e.g., compared to a level of GDF15 in a human or a panel of humans (e.g., 2, 3, 4, 5, 10, 15, 20, 25, or more) not having the tumor). In some instances, the tumor is a solid tumor. In some instances, the tumor is an advanced solid tumor. In some instances, the tumor is a cancer. In some instances, the cancer is metastatic. In some instances, the cancer is advanced. In some instances, the cancer is a prostate cancer (e.g., an mCRPC, a castration-sensitive prostate cancer, a castration-resistant prostate cancer, an MSI-H prostate cancer; a metastatic prostate cancer, a metastatic castrate-resistant, chemo-refractory prostate cancer; an advanced prostate cancer where the patient has exhausted all standard therapies with elevated baseline serum GDF15; an advanced prostate cancer where the patient has previously received one or more (e.g., 1, 2, 3, or more) lines of cancer therapies; a chemo-refractory prostate cancer; a metastatic chemo-refractory prostate cancer), a bladder cancer, a melanoma, an SCLC (e.g., an extensive-stage SCLC), an NSCLC, a pancreatic cancer (e.g., an advanced pancreatic cancer, a metastatic pancreatic cancer, a pancreatic adenocarcinoma, a metastatic pancreatic adenocarcinoma, an MSI-H pancreatic cancer, a pancreatic ductal adenocarcinoma, a metastatic pancreatic ductal adenocarcinoma), a colorectal cancer, a gastric cancer, an esophageal cancer, an ovarian cancer, a head neck squamous cell carcinoma or an MSI-H cancer (e.g., an MSI-H prostate cancer, an MSI-H ovarian cancer, an MSI-H ovarian cancer, an MSI-H HNSCC, an MSI-H SLCLC (e.g., an MSI-H extensive-stage SCLC). In some instances, the cancer is an MSI-H cancer selected from a castration-resistant prostate cancer, a bladder cancer, a melanoma, a SCLC (e.g., an extensive-stage SCLC), a pancreatic cancer (e.g., an advanced pancreatic cancer, a metastatic pancreatic cancer, a pancreatic adenocarcinoma, a metastatic pancreatic adenocarcinoma, an MSI-H pancreatic cancer, a pancreatic ductal adenocarcinoma, a metastatic pancreatic ductal adenocarcinoma), a colorectal cancer, a gastric cancer, an esophageal cancer, an ovarian cancer, a head and neck squamous cell carcinoma). In some instances, the cancer is a pancreatic cancer (e.g., an advanced pancreatic cancer, a metastatic pancreatic cancer, a pancreatic adenocarcinoma, a metastatic pancreatic adenocarcinoma, an MSI-H pancreatic cancer, a pancreatic ductal adenocarcinoma, a metastatic pancreatic ductal adenocarcinoma). In some instances, the cancer is a pancreatic adenocarcinoma. In some instances, the cancer is a metastatic pancreatic cancer. In some instances, the cancer is a metastatic pancreatic adenocarcinoma. In some instances, the pancreatic cancer is an advanced pancreatic cancer. In some instances, the pancreatic cancer is an MSI-H pancreatic cancer. In some instances, the pancreatic cancer is a pancreatic ductal adenocarcinoma. In some instances, the pancreatic cancer is a metastatic pancreatic ductal adenocarcinoma. In some instances, the human patient has a prostate cancer (e.g., an mCRPC, a castration-sensitive prostate cancer, a castration-resistant prostate cancer, an MSI-H prostate cancer; a metastatic prostate cancer, a metastatic castrate-resistant, chemo-refractory prostate cancer; an advanced prostate cancer where the patient has exhausted all standard therapies with elevated baseline serum GDF15; an advanced prostate cancer where the patient has previously received one or more (e.g., 1, 2, 3, or more) lines of cancer therapies; a chemo-refractory prostate cancer; a metastatic chemo-refractory prostate cancer). In some instances, the cancer is advanced prostate cancer. In some instances, the human patient has prostate cancer and poor survival. In some instances, the cancer is an mCRPC. In some instances, the cancer is chemo-refractory prostate cancer. In some instances, the cancer is metastatic chemo-refractory prostate cancer. In some instances, the cancer is metastatic castrate-resistant, chemo-refractory prostate cancer. In some instances, the cancer is advanced prostate cancer, wherein the human patient has exhausted all standard therapies, and wherein the human patient has an elevated baseline serum GDF15 (compared to a human or a panel of humans not having prostate cancer). In some instances, the cancer is advanced prostate cancer (e.g., castration-resistant prostate cancer), wherein the human patient has previously received one or more (e.g., 1, 2, 3, or more) lines of therapies (e.g., leuprolide acetate (LUPRON), zoledronic acid (ZOMETA), docetaxel, cabazitaxel, radium 223, mitoxantrone, Oxaliplatin and pemetrexed). In some instances, the human patient previously failed to be treated with one or more (e.g., 1, 2, 3, 4, 5) other cancer therapies (i.e., other than an anti-GFRAL antibody described herein) prior to the administering of the anti-GFRAL antibody.

Also provided herein is a method for treating a tumor (e.g., a solid tumor, an advanced solid tumor, a cancer) in a human patient, the method comprising administering to the human patient an anti-GFRAL antibody at a dose of 100 mg once every 3 weeks, wherein the anti-GFRAL antibody comprises a heavy chain comprising the amino acid sequence set forth in SEQ ID NO:2010 and a light chain comprising the amino acid sequence set forth in SEQ ID NO:2012. In some instances, the anti-GFRAL antibody comprises a heavy chain consisting of the amino acid sequence set forth in SEQ ID NO:2010 and a light chain comprising the amino acid sequence set forth in SEQ ID NO:2012. In some instances, the administering is performed subcutaneously. In some instances, the administering is performed intravenously. In some instances, the human patient has an elevated level of GDF15 (e.g., compared to a level of GDF15 in a human or a panel of humans (e.g., 2, 3, 4, 5, 10, 15, 20, 25, or more) not having the tumor). In some instances, the tumor is a solid tumor. In some instances, the tumor is an advanced solid tumor. In some instances, the tumor is a cancer. In some instances, the cancer is metastatic. In some instances, the cancer is advanced. In some instances, the cancer is a prostate cancer (e.g., an mCRPC, a castration-sensitive prostate cancer, a castration-resistant prostate cancer, an MSI-H prostate cancer; a metastatic prostate cancer, a metastatic castrate-resistant, chemo-refractory prostate cancer; an advanced prostate cancer where the patient has exhausted all standard therapies with elevated baseline serum GDF15; an advanced prostate cancer where the patient has previously received one or more (e.g., 1, 2, 3, or more) lines of cancer therapies; a chemo-refractory prostate cancer; a metastatic chemo-refractory prostate cancer), a bladder cancer, a melanoma, an SCLC (e.g., an extensive-stage SCLC), an NSCLC, a pancreatic cancer (e.g., an advanced pancreatic cancer, a metastatic pancreatic cancer, a pancreatic adenocarcinoma, a metastatic pancreatic adenocarcinoma, an MSI-H pancreatic cancer, a pancreatic ductal adenocarcinoma, a metastatic pancreatic ductal adenocarcinoma), a colorectal cancer, a gastric cancer, an esophageal cancer, an ovarian cancer, a head neck squamous cell carcinoma or an MSI-H cancer (e.g., an MSI-H prostate cancer, an MSI-H ovarian cancer, an MSI-H ovarian cancer, an MSI-H HNSCC, an MSI-H SLCLC (e.g., an MSI-H extensive-stage SCLC). In some instances, the cancer is an MSI-H cancer selected from a castration-resistant prostate cancer, a bladder cancer, a melanoma, a SCLC (e.g., an extensive-stage SCLC), a pancreatic cancer (e.g., an advanced pancreatic cancer, a metastatic pancreatic cancer, a pancreatic adenocarcinoma, a metastatic pancreatic adenocarcinoma, an MSI-H pancreatic cancer, a pancreatic ductal adenocarcinoma, a metastatic pancreatic ductal adenocarcinoma), a colorectal cancer, a gastric cancer, an esophageal cancer, an ovarian cancer, a head and neck squamous cell carcinoma). In some instances, the cancer is a pancreatic cancer (e.g., an advanced pancreatic cancer, a metastatic pancreatic cancer, a pancreatic adenocarcinoma, a metastatic pancreatic adenocarcinoma, an MSI-H pancreatic cancer, a pancreatic ductal adenocarcinoma, a metastatic pancreatic ductal adenocarcinoma). In some instances, the cancer is a pancreatic adenocarcinoma. In some instances, the cancer is a metastatic pancreatic cancer. In some instances, the cancer is a metastatic pancreatic adenocarcinoma. In some instances, the pancreatic cancer is an advanced pancreatic cancer. In some instances, the pancreatic cancer is an MSI-H pancreatic cancer. In some instances, the pancreatic cancer is a pancreatic ductal adenocarcinoma. In some instances, the pancreatic cancer is a metastatic pancreatic ductal adenocarcinoma. In some instances, the human patient has a prostate cancer (e.g., an mCRPC, a castration-sensitive prostate cancer, a castration-resistant prostate cancer, an MSI-H prostate cancer; a metastatic prostate cancer, a metastatic castrate-resistant, chemo-refractory prostate cancer; an advanced prostate cancer where the patient has exhausted all standard therapies with elevated baseline serum GDF15; an advanced prostate cancer where the patient has previously received one or more (e.g., 1, 2, 3, or more) lines of cancer therapies; a chemo-refractory prostate cancer; a metastatic chemo-refractory prostate cancer). In some instances, the cancer is advanced prostate cancer. In some instances, the human patient has prostate cancer and poor survival. In some instances, the cancer is an mCRPC. In some instances, the cancer is chemo-refractory prostate cancer. In some instances, the cancer is metastatic chemo-refractory prostate cancer. In some instances, the cancer is metastatic castrate-resistant, chemo-refractory prostate cancer. In some instances, the cancer is advanced prostate cancer, wherein the human patient has exhausted all standard therapies, and wherein the human patient has an elevated baseline serum GDF15 (compared to a human or a panel of humans not having prostate cancer). In some instances, the cancer is advanced prostate cancer (e.g., castration-resistant prostate cancer), wherein the human patient has previously received one or more (e.g., 1, 2, 3, or more) lines of therapies (e.g., leuprolide acetate (LUPRON), zoledronic acid (ZOMETA), docetaxel, cabazitaxel, radium 223, mitoxantrone, Oxaliplatin and pemetrexed). In some instances, the human patient previously failed to be treated with one or more (e.g., 1, 2, 3, 4, 5) other cancer therapies (i.e., other than an anti-GFRAL antibody described herein) prior to the administering of the anti-GFRAL antibody.

Also provided herein is a method for treating tumor-related weight loss or cachexia in a human patient, the method comprising administering to the human patient an anti-GFRAL antibody at a dose of 30 mg once every 3 weeks, wherein the anti-GFRAL antibody comprises. (i) a VH comprising a VH CDR1 comprising the amino acid sequence set forth in SEQ ID NO:48, a VH CDR2 comprising the amino acid sequence set forth in SEQ ID NO:137, and a VH CDR3 comprising the amino acid sequence set forth in SEQ ID NO:225; and (ii) a VL comprising a VL CDR1 comprising the amino acid sequence set forth in SEQ ID NO:301, a VL CDR2 comprising the amino acid sequence set forth in SEQ ID NO:376, and a VL CDR3 comprising the amino acid sequence set forth in SEQ ID NO:426. In some instances, the administering is performed subcutaneously. In some instances, the administering is performed intravenously. In some instances, the human patient has an elevated level of GDF15 (e.g., compared to a level of GDF15 in a human or a panel of humans (e.g., 2, 3, 4, 5, 10, 15, 20, 25, or more) not having the tumor). In some instances, the tumor is a solid tumor. In some instances, the tumor is an advanced solid tumor. In some instances, the tumor is a cancer. In some instances, the cancer is metastatic. In some instances, the cancer is advanced. In some instances, the cancer is a prostate cancer (e.g., an mCRPC, a castration-sensitive prostate cancer, a castration-resistant prostate cancer, an MSI-H prostate cancer; a metastatic prostate cancer, a metastatic castrate-resistant, chemo-refractory prostate cancer; an advanced prostate cancer where the patient has exhausted all standard therapies with elevated baseline serum GDF15; an advanced prostate cancer where the patient has previously received one or more (e.g., 1, 2, 3, or more) lines of cancer therapies; a chemo-refractory prostate cancer; a metastatic chemo-refractory prostate cancer), a bladder cancer, a melanoma, an SCLC (e.g., an extensive-stage SCLC), an NSCLC, a pancreatic cancer (e.g., an advanced pancreatic cancer, a metastatic pancreatic cancer, a pancreatic adenocarcinoma, a metastatic pancreatic adenocarcinoma, an MSI-H pancreatic cancer, a pancreatic ductal adenocarcinoma, a metastatic pancreatic ductal adenocarcinoma), a colorectal cancer, a gastric cancer, an esophageal cancer, an ovarian cancer, a head neck squamous cell carcinoma or an MSI-H cancer (e.g., an MSI-H prostate cancer, an MSI-H ovarian cancer, an MSI-H ovarian cancer, an MSI-H HNSCC, an MSI-H SLCLC (e.g., an MSI-H extensive-stage SCLC). In some instances, the cancer is an MSI-H cancer selected from a castration-resistant prostate cancer, a bladder cancer, a melanoma, a SCLC (e.g., an extensive-stage SCLC), a pancreatic cancer (e.g., an advanced pancreatic cancer, a metastatic pancreatic cancer, a pancreatic adenocarcinoma, a metastatic pancreatic adenocarcinoma, an MSI-H pancreatic cancer, a pancreatic ductal adenocarcinoma, a metastatic pancreatic ductal adenocarcinoma), a colorectal cancer, a gastric cancer, an esophageal cancer, an ovarian cancer, a head and neck squamous cell carcinoma). In some instances, the cancer is a pancreatic cancer (e.g., an advanced pancreatic cancer, a metastatic pancreatic cancer, a pancreatic adenocarcinoma, a metastatic pancreatic adenocarcinoma, an MSI-H pancreatic cancer, a pancreatic ductal adenocarcinoma, a metastatic pancreatic ductal adenocarcinoma). In some instances, the cancer is a pancreatic adenocarcinoma. In some instances, the cancer is a metastatic pancreatic cancer. In some instances, the cancer is a metastatic pancreatic adenocarcinoma. In some instances, the pancreatic cancer is an advanced pancreatic cancer. In some instances, the pancreatic cancer is an MSI-H pancreatic cancer. In some instances, the pancreatic cancer is a pancreatic ductal adenocarcinoma. In some instances, the pancreatic cancer is a metastatic pancreatic ductal adenocarcinoma. In some instances, the human patient has a prostate cancer (e.g., an mCRPC, a castration-sensitive prostate cancer, a castration-resistant prostate cancer, an MSI-H prostate cancer; a metastatic prostate cancer, a metastatic castrate-resistant, chemo-refractory prostate cancer; an advanced prostate cancer where the patient has exhausted all standard therapies with elevated baseline serum GDF15; an advanced prostate cancer where the patient has previously received one or more (e.g., 1, 2, 3, or more) lines of cancer therapies; a chemo-refractory prostate cancer; a metastatic chemo-refractory prostate cancer). In some instances, the cancer is advanced prostate cancer. In some instances, the human patient has prostate cancer and poor survival. In some instances, the cancer is an mCRPC. In some instances, the cancer is chemo-refractory prostate cancer. In some instances, the cancer is metastatic chemo-refractory prostate cancer. In some instances, the cancer is metastatic castrate-resistant, chemo-refractory prostate cancer. In some instances, the cancer is advanced prostate cancer, wherein the human patient has exhausted all standard therapies, and wherein the human patient has an elevated baseline serum GDF15 (compared to a human or a panel of humans not having prostate cancer). In some instances, the cancer is advanced prostate cancer (e.g., castration-resistant prostate cancer), wherein the human patient has previously received one or more (e.g., 1, 2, 3, or more) lines of therapies (e.g., leuprolide acetate (LUPRON), zoledronic acid (ZOMETA), docetaxel, cabazitaxel, radium 223, mitoxantrone, Oxaliplatin and pemetrexed). In some instances, the human patient previously failed to be treated with one or more (e.g., 1, 2, 3, 4, 5) other cancer therapies (i.e., other than an anti-GFRAL antibody described herein) prior to the administering of the anti-GFRAL antibody.

Also provided herein is a method for treating tumor-related weight loss or cachexia in a human patient, the method comprising administering to the human patient an anti-GFRAL antibody at a dose of 30 mg once every 3 weeks, wherein the anti-GFRAL antibody comprises a VH comprising the amino acid sequence set forth in SEQ ID NO:1982 and a VL comprising the amino acid sequence set forth in SEQ ID NO:1997. In some instances, the administering is performed subcutaneously. In some instances, the administering is performed intravenously. In some instances, the human patient has an elevated level of GDF15 (e.g., compared to a level of GDF15 in a human or a panel of humans (e.g., 2, 3, 4, 5, 10, 15, 20, 25, or more) not having the tumor). In some instances, the tumor is a solid tumor. In some instances, the tumor is an advanced solid tumor. In some instances, the tumor is a cancer. In some instances, the cancer is metastatic. In some instances, the cancer is advanced. In some instances, the cancer is a prostate cancer (e.g., an mCRPC, a castration-sensitive prostate cancer, a castration-resistant prostate cancer, an MSI-H prostate cancer; a metastatic prostate cancer, a metastatic castrate-resistant, chemo-refractory prostate cancer; an advanced prostate cancer where the patient has exhausted all standard therapies with elevated baseline serum GDF15; an advanced prostate cancer where the patient has previously received one or more (e.g., 1, 2, 3, or more) lines of cancer therapies; a chemo-refractory prostate cancer; a metastatic chemo-refractory prostate cancer), a bladder cancer, a melanoma, an SCLC (e.g., an extensive-stage SCLC), an NSCLC, a pancreatic cancer (e.g., an advanced pancreatic cancer, a metastatic pancreatic cancer, a pancreatic adenocarcinoma, a metastatic pancreatic adenocarcinoma, an MSI-H pancreatic cancer, a pancreatic ductal adenocarcinoma, a metastatic pancreatic ductal adenocarcinoma), a colorectal cancer, a gastric cancer, an esophageal cancer, an ovarian cancer, a head neck squamous cell carcinoma or an MSI-H cancer (e.g., an MSI-H prostate cancer, an MSI-H ovarian cancer, an MSI-H ovarian cancer, an MSI-H HNSCC, an MSI-H SLCLC (e.g., an MSI-H extensive-stage SCLC). In some instances, the cancer is an MSI-H cancer selected from a castration-resistant prostate cancer, a bladder cancer, a melanoma, a SCLC (e.g., an extensive-stage SCLC), a pancreatic cancer (e.g., an advanced pancreatic cancer, a metastatic pancreatic cancer, a pancreatic adenocarcinoma, a metastatic pancreatic adenocarcinoma, an MSI-H pancreatic cancer, a pancreatic ductal adenocarcinoma, a metastatic pancreatic ductal adenocarcinoma), a colorectal cancer, a gastric cancer, an esophageal cancer, an ovarian cancer, a head and neck squamous cell carcinoma). In some instances, the cancer is a pancreatic cancer (e.g., an advanced pancreatic cancer, a metastatic pancreatic cancer, a pancreatic adenocarcinoma, a metastatic pancreatic adenocarcinoma, an MSI-H pancreatic cancer, a pancreatic ductal adenocarcinoma, a metastatic pancreatic ductal adenocarcinoma). In some instances, the cancer is a pancreatic adenocarcinoma. In some instances, the cancer is a metastatic pancreatic cancer. In some instances, the cancer is a metastatic pancreatic adenocarcinoma. In some instances, the pancreatic cancer is an advanced pancreatic cancer. In some instances, the pancreatic cancer is an MSI-H pancreatic cancer. In some instances, the pancreatic cancer is a pancreatic ductal adenocarcinoma. In some instances, the pancreatic cancer is a metastatic pancreatic ductal adenocarcinoma. In some instances, the human patient has a prostate cancer (e.g., an mCRPC, a castration-sensitive prostate cancer, a castration-resistant prostate cancer, an MSI-H prostate cancer; a metastatic prostate cancer, a metastatic castrate-resistant, chemo-refractory prostate cancer; an advanced prostate cancer where the patient has exhausted all standard therapies with elevated baseline serum GDF15; an advanced prostate cancer where the patient has previously received one or more (e.g., 1, 2, 3, or more) lines of cancer therapies; a chemo-refractory prostate cancer; a metastatic chemo-refractory prostate cancer). In some instances, the cancer is advanced prostate cancer. In some instances, the human patient has prostate cancer and poor survival. In some instances, the cancer is an mCRPC. In some instances, the cancer is chemo-refractory prostate cancer. In some instances, the cancer is metastatic chemo-refractory prostate cancer. In some instances, the cancer is metastatic castrate-resistant, chemo-refractory prostate cancer. In some instances, the cancer is advanced prostate cancer, wherein the human patient has exhausted all standard therapies, and wherein the human patient has an elevated baseline serum GDF15 (compared to a human or a panel of humans not having prostate cancer). In some instances, the cancer is advanced prostate cancer (e.g., castration-resistant prostate cancer), wherein the human patient has previously received one or more (e.g., 1, 2, 3, or more) lines of therapies (e.g., leuprolide acetate (LUPRON), zoledronic acid (ZOMETA), docetaxel, cabazitaxel, radium 223, mitoxantrone, Oxaliplatin and pemetrexed). In some instances, the human patient previously failed to be treated with one or more (e.g., 1, 2, 3, 4, 5) other cancer therapies (i.e., other than an anti-GFRAL antibody described herein) prior to the administering of the anti-GFRAL antibody.

Also provided herein is a method for treating tumor-related weight loss or cachexia in a human patient, the method comprising administering to the human patient an anti-GFRAL antibody at a dose of 30 mg once every 3 weeks, wherein the anti-GFRAL antibody comprises a heavy chain comprising the amino acid sequence set forth in SEQ ID NO:2010 and a light chain comprising the amino acid sequence set forth in SEQ ID NO:2012. In some instances, the anti-GFRAL antibody comprises a heavy chain consisting of the amino acid sequence set forth in SEQ ID NO:2010 and a light chain comprising the amino acid sequence set forth in SEQ ID NO:2012. In some instances, the administering is performed subcutaneously. In some instances, the administering is performed intravenously. In some instances, the human patient has an elevated level of GDF15 (e.g., compared to a level of GDF15 in a human or a panel of humans (e.g., 2, 3, 4, 5, 10, 15, 20, 25, or more) not having the tumor). In some instances, the tumor is a solid tumor. In some instances, the tumor is an advanced solid tumor. In some instances, the tumor is a cancer. In some instances, the cancer is metastatic. In some instances, the cancer is advanced. In some instances, the cancer is a prostate cancer (e.g., an mCRPC, a castration-sensitive prostate cancer, a castration-resistant prostate cancer, an MSI-H prostate cancer; a metastatic prostate cancer, a metastatic castrate-resistant, chemo-refractory prostate cancer; an advanced prostate cancer where the patient has exhausted all standard therapies with elevated baseline serum GDF15; an advanced prostate cancer where the patient has previously received one or more (e.g., 1, 2, 3, or more) lines of cancer therapies; a chemo-refractory prostate cancer; a metastatic chemo-refractory prostate cancer), a bladder cancer, a melanoma, an SCLC (e.g., an extensive-stage SCLC), an NSCLC, a pancreatic cancer (e.g., an advanced pancreatic cancer, a metastatic pancreatic cancer, a pancreatic adenocarcinoma, a metastatic pancreatic adenocarcinoma, an MSI-H pancreatic cancer, a pancreatic ductal adenocarcinoma, a metastatic pancreatic ductal adenocarcinoma), a colorectal cancer, a gastric cancer, an esophageal cancer, an ovarian cancer, a head neck squamous cell carcinoma or an MSI-H cancer (e.g., an MSI-H prostate cancer, an MSI-H ovarian cancer, an MSI-H ovarian cancer, an MSI-H HNSCC, an MSI-H SLCLC (e.g., an MSI-H extensive-stage SCLC). In some instances, the cancer is an MSI-H cancer selected from a castration-resistant prostate cancer, a bladder cancer, a melanoma, a SCLC (e.g., an extensive-stage SCLC), a pancreatic cancer (e.g., an advanced pancreatic cancer, a metastatic pancreatic cancer, a pancreatic adenocarcinoma, a metastatic pancreatic adenocarcinoma, an MSI-H pancreatic cancer, a pancreatic ductal adenocarcinoma, a metastatic pancreatic ductal adenocarcinoma), a colorectal cancer, a gastric cancer, an esophageal cancer, an ovarian cancer, a head and neck squamous cell carcinoma). In some instances, the cancer is a pancreatic cancer (e.g., an advanced pancreatic cancer, a metastatic pancreatic cancer, a pancreatic adenocarcinoma, a metastatic pancreatic adenocarcinoma, an MSI-H pancreatic cancer, a pancreatic ductal adenocarcinoma, a metastatic pancreatic ductal adenocarcinoma). In some instances, the cancer is a pancreatic adenocarcinoma. In some instances, the cancer is a metastatic pancreatic cancer. In some instances, the cancer is a metastatic pancreatic adenocarcinoma. In some instances, the pancreatic cancer is an advanced pancreatic cancer. In some instances, the pancreatic cancer is an MSI-H pancreatic cancer. In some instances, the pancreatic cancer is a pancreatic ductal adenocarcinoma. In some instances, the pancreatic cancer is a metastatic pancreatic ductal adenocarcinoma. In some instances, the human patient has a prostate cancer (e.g., an mCRPC, a castration-sensitive prostate cancer, a castration-resistant prostate cancer, an MSI-H prostate cancer; a metastatic prostate cancer, a metastatic castrate-resistant, chemo-refractory prostate cancer; an advanced prostate cancer where the patient has exhausted all standard therapies with elevated baseline serum GDF15; an advanced prostate cancer where the patient has previously received one or more (e.g., 1, 2, 3, or more) lines of cancer therapies; a chemo-refractory prostate cancer; a metastatic chemo-refractory prostate cancer). In some instances, the cancer is advanced prostate cancer. In some instances, the human patient has prostate cancer and poor survival. In some instances, the cancer is an mCRPC. In some instances, the cancer is chemo-refractory prostate cancer. In some instances, the cancer is metastatic chemo-refractory prostate cancer. In some instances, the cancer is metastatic castrate-resistant, chemo-refractory prostate cancer. In some instances, the cancer is advanced prostate cancer, wherein the human patient has exhausted all standard therapies, and wherein the human patient has an elevated baseline serum GDF15 (compared to a human or a panel of humans not having prostate cancer). In some instances, the cancer is advanced prostate cancer (e.g., castration-resistant prostate cancer), wherein the human patient has previously received one or more (e.g., 1, 2, 3, or more) lines of therapies (e.g., leuprolide acetate (LUPRON), zoledronic acid (ZOMETA), docetaxel, cabazitaxel, radium 223, mitoxantrone, Oxaliplatin and pemetrexed). In some instances, the human patient previously failed to be treated with one or more (e.g., 1, 2, 3, 4, 5) other cancer therapies (i.e., other than an anti-GFRAL antibody described herein) prior to the administering of the anti-GFRAL antibody.

Also provided herein is a method for treating tumor-related weight loss or cachexia in a human patient, the method comprising administering to the human patient an anti-GFRAL antibody at a dose of 100 mg once every 3 weeks, wherein the anti-GFRAL antibody comprises: (i) a VH comprising a VH CDR1 comprising the amino acid sequence set forth in SEQ ID NO:48, a VH CDR2 comprising the amino acid sequence set forth in SEQ ID NO:137, and a VH CDR3 comprising the amino acid sequence set forth in SEQ ID NO:225; and (ii) a VL comprising a VL CDR1 comprising the amino acid sequence set forth in SEQ ID NO:301, a VL CDR2 comprising the amino acid sequence set forth in SEQ ID NO:376, and a VL CDR3 comprising the amino acid sequence set forth in SEQ ID NO:426. In some instances, the administering is performed subcutaneously. In some instances, the administering is performed intravenously. In some instances, the human patient has an elevated level of GDF15 (e.g., compared to a level of GDF15 in a human or a panel of humans (e.g., 2, 3, 4, 5, 10, 15, 20, 25, or more) not having the tumor). In some instances, the tumor is a solid tumor. In some instances, the tumor is an advanced solid tumor. In some instances, the tumor is a cancer. In some instances, the cancer is metastatic. In some instances, the cancer is advanced. In some instances, the cancer is a prostate cancer (e.g., an mCRPC, a castration-sensitive prostate cancer; a castration-resistant prostate cancer; an MSI-H prostate cancer; a metastatic prostate cancer; an advanced prostate cancer where the patient has exhausted all standard therapies with elevated baseline serum GDF15; an advanced prostate cancer where the patient has previously received one or more (e.g., 1, 2, 3, or more) lines of cancer therapies; a chemo-refractory prostate cancer; a metastatic chemo-refractory prostate cancer), a bladder cancer, a melanoma, an SCLC (e.g., an extensive-stage SCLC), an NSCLC, a pancreatic cancer (e.g., an advanced pancreatic cancer, a metastatic pancreatic cancer, a pancreatic adenocarcinoma, a metastatic pancreatic adenocarcinoma, an MSI-H pancreatic cancer, a pancreatic ductal adenocarcinoma, a metastatic pancreatic ductal adenocarcinoma), a colorectal cancer, a gastric cancer, an esophageal cancer, an ovarian cancer, a head neck squamous cell carcinoma or an MSI-H cancer (e.g., an MSI-H prostate cancer, an MSI-H ovarian cancer, an MSI-H ovarian cancer, an MSI-H HNSCC, an MSI-H SLCLC (e.g., an MSI-H extensive-stage SCLC). In some instances, the cancer is an MSI-H cancer selected from a castration-resistant prostate cancer, a bladder cancer, a melanoma, a SCLC (e.g., an extensive-stage SCLC), a pancreatic cancer (e.g., an advanced pancreatic cancer, a metastatic pancreatic cancer, a pancreatic adenocarcinoma, a metastatic pancreatic adenocarcinoma, an MSI-H pancreatic cancer, a pancreatic ductal adenocarcinoma, a metastatic pancreatic ductal adenocarcinoma), a colorectal cancer, a gastric cancer, an esophageal cancer, an ovarian cancer, a head and neck squamous cell carcinoma). In some instances, the cancer is a pancreatic cancer (e.g., an advanced pancreatic cancer, a metastatic pancreatic cancer, a pancreatic adenocarcinoma, a metastatic pancreatic adenocarcinoma, an MSI-H pancreatic cancer, a pancreatic ductal adenocarcinoma, a metastatic pancreatic ductal adenocarcinoma). In some instances, the cancer is a pancreatic adenocarcinoma. In some instances, the cancer is a metastatic pancreatic cancer. In some instances, the cancer is a metastatic pancreatic adenocarcinoma. In some instances, the pancreatic cancer is an advanced pancreatic cancer. In some instances, the pancreatic cancer is an MSI-H pancreatic cancer. In some instances, the pancreatic cancer is a pancreatic ductal adenocarcinoma. In some instances, the pancreatic cancer is a metastatic pancreatic ductal adenocarcinoma. In some instances, the human patient has a prostate cancer (e.g., an mCRPC, a castration-sensitive prostate cancer, a castration-resistant prostate cancer, an MSI-H prostate cancer, a metastatic prostate cancer, a metastatic castrate-resistant, chemo-refractory prostate cancer; an advanced prostate cancer where the patient has exhausted all standard therapies with elevated baseline serum GDF15; an advanced prostate cancer where the patient has previously received one or more (e.g., 1, 2, 3, or more) lines of cancer therapies; a chemo-refractory prostate cancer; a metastatic chemo-refractory prostate cancer). In some instances, the cancer is advanced prostate cancer. In some instances, the human patient has prostate cancer and poor survival. In some instances, the cancer is an mCRPC. In some instances, the cancer is chemo-refractory prostate cancer. In some instances, the cancer is metastatic chemo-refractory prostate cancer. In some instances, the cancer is metastatic castrate-resistant, chemo-refractory prostate cancer. In some instances, the cancer is advanced prostate cancer, wherein the human patient has exhausted all standard therapies, and wherein the human patient has an elevated baseline serum GDF15 (compared to a human or a panel of humans not having prostate cancer). In some instances, the cancer is advanced prostate cancer (e.g., castration-resistant prostate cancer), wherein the human patient has previously received one or more (e.g., 1, 2, 3, or more) lines of therapies (e.g., leuprolide acetate (LUPRON), zoledronic acid (ZOMETA), docetaxel, cabazitaxel, radium 223, mitoxantrone, Oxaliplatin and pemetrexed). In some instances, the cancer is a metastatic pancreatic adenocarcinoma. In some instances, the human patient previously failed to be treated with one or more (e.g., 1, 2, 3, 4, 5) other cancer therapies (i.e., other than an anti-GFRAL antibody described herein) prior to the administering of the anti-GFRAL antibody.

Also provided herein is a method for treating tumor-related weight loss or cachexia in a human patient, the method comprising administering to the human patient an anti-GFRAL antibody at a dose of 100 mg once every 3 weeks, wherein the anti-GFRAL antibody comprises a VH comprising the amino acid sequence set forth in SEQ ID NO:1982 and a VL comprising the amino acid sequence set forth in SEQ ID NO:1997. In some instances, the administering is performed subcutaneously. In some instances, the administering is performed intravenously. In some instances, the human patient has an elevated level of GDF15 (e.g., compared to a level of GDF15 in a human or a panel of humans (e.g., 2, 3, 4, 5, 10, 15, 20, 25, or more) not having the tumor). In some instances, the tumor is a solid tumor. In some instances, the tumor is an advanced solid tumor. In some instances, the tumor is a cancer. In some instances, the cancer is metastatic. In some instances, the cancer is advanced. In some instances, the cancer is a prostate cancer (e.g., an mCRPC, a castration-sensitive prostate cancer, a castration-resistant prostate cancer, an MSI-H prostate cancer, a metastatic prostate cancer, a metastatic castrate-resistant, chemo-refractory prostate cancer; an advanced prostate cancer where the patient has exhausted all standard therapies with elevated baseline serum GDF15; an advanced prostate cancer where the patient has previously received one or more (e.g., 1, 2, 3, or more) lines of cancer therapies; a chemo-refractory prostate cancer; a metastatic chemo-refractory prostate cancer), a bladder cancer, a melanoma, an SCLC (e.g., an extensive-stage SCLC), an NSCLC, a pancreatic cancer (e.g., an advanced pancreatic cancer, a metastatic pancreatic cancer, a pancreatic adenocarcinoma, a metastatic pancreatic adenocarcinoma, an MSI-H pancreatic cancer, a pancreatic ductal adenocarcinoma, a metastatic pancreatic ductal adenocarcinoma), a colorectal cancer, a gastric cancer, an esophageal cancer, an ovarian cancer, a head neck squamous cell carcinoma or an MSI-H cancer (e.g., an MSI-H prostate cancer, an MSI-H ovarian cancer, an MSI-H ovarian cancer, an MSI-H HNSCC, an MSI-H SLCLC (e.g., an MSI-H extensive-stage SCLC). In some instances, the cancer is an MSI-H cancer selected from a castration-resistant prostate cancer, a bladder cancer, a melanoma, a SCLC (e.g., an extensive-stage SCLC), a pancreatic cancer (e.g., an advanced pancreatic cancer, a metastatic pancreatic cancer, a pancreatic adenocarcinoma, a metastatic pancreatic adenocarcinoma, an MSI-H pancreatic cancer, a pancreatic ductal adenocarcinoma, a metastatic pancreatic ductal adenocarcinoma), a colorectal cancer, a gastric cancer, an esophageal cancer, an ovarian cancer, a head and neck squamous cell carcinoma). In some instances, the cancer is a pancreatic cancer (e.g., an advanced pancreatic cancer, a metastatic pancreatic cancer, a pancreatic adenocarcinoma, a metastatic pancreatic adenocarcinoma, an MSI-H pancreatic cancer, a pancreatic ductal adenocarcinoma, a metastatic pancreatic ductal adenocarcinoma). In some instances, the cancer is a pancreatic adenocarcinoma. In some instances, the cancer is a metastatic pancreatic cancer. In some instances, the cancer is a metastatic pancreatic adenocarcinoma. In some instances, the pancreatic cancer is an advanced pancreatic cancer. In some instances, the pancreatic cancer is an MSI-H pancreatic cancer. In some instances, the pancreatic cancer is a pancreatic ductal adenocarcinoma. In some instances, the pancreatic cancer is a metastatic pancreatic ductal adenocarcinoma. In some instances, the human patient has a prostate cancer (e.g., an mCRPC, a castration-sensitive prostate cancer, a castration-resistant prostate cancer, an MSI-H prostate cancer, a metastatic prostate cancer, a metastatic castrate-resistant, chemo-refractory prostate cancer; an advanced prostate cancer where the patient has exhausted all standard therapies with elevated baseline serum GDF15; an advanced prostate cancer where the patient has previously received one or more (e.g., 1, 2, 3, or more) lines of cancer therapies; a chemo-refractory prostate cancer; a metastatic chemo-refractory prostate cancer). In some instances, the cancer is advanced prostate cancer. In some instances, the human patient has prostate cancer and poor survival. In some instances, the cancer is an mCRPC. In some instances, the cancer is chemo-refractory prostate cancer. In some instances, the cancer is metastatic chemo-refractory prostate cancer. In some instances, the cancer is metastatic castrate-resistant, chemo-refractory prostate cancer. In some instances, the cancer is advanced prostate cancer, wherein the human patient has exhausted all standard therapies, and wherein the human patient has an elevated baseline serum GDF15 (compared to a human or a panel of humans not having prostate cancer). In some instances, the cancer is advanced prostate cancer (e.g., castration-resistant prostate cancer), wherein the human patient has previously received one or more (e.g., 1, 2, 3, or more) lines of therapies (e.g., leuprolide acetate (LUPRON), zoledronic acid (ZOMETA), docetaxel, cabazitaxel, radium 223, mitoxantrone, Oxaliplatin and pemetrexed). In some instances, the human patient previously failed to be treated with one or more (e.g., 1, 2, 3, 4, 5) other cancer therapies (i.e., other than an anti-GFRAL antibody described herein) prior to the administering of the anti-GFRAL antibody.

Also provided herein is a method for treating tumor-related weight loss or cachexia in a human patient, the method comprising administering to the human patient an anti-GFRAL antibody at a dose of 100 mg once every 3 weeks, wherein the anti-GFRAL antibody comprises a heavy chain comprising the amino acid sequence set forth in SEQ ID NO:2010 and a light chain comprising the amino acid sequence set forth in SEQ ID NO:2012. In some instances, the anti-GFRAL antibody comprises a heavy chain consisting of the amino acid sequence set forth in SEQ ID NO:2010 and a light chain comprising the amino acid sequence set forth in SEQ ID NO:2012. In some instances, the administering is performed subcutaneously. In some instances, the administering is performed intravenously. In some instances, the human patient has an elevated level of GDF15 (e.g., compared to a level of GDF15 in a human or a panel of humans (e.g., 2, 3, 4, 5, 10, 15, 20, 25, or more) not having the tumor). In some instances, the tumor is a solid tumor. In some instances, the tumor is an advanced solid tumor. In some instances, the tumor is a cancer. In some instances, the cancer is metastatic. In some instances, the cancer is advanced. In some instances, the cancer is a prostate cancer (e.g., an mCRPC, a castration-sensitive prostate cancer, a castration-resistant prostate cancer, an MSI-H prostate cancer, a metastatic prostate cancer, a metastatic castrate-resistant, chemo-refractory prostate cancer; an advanced prostate cancer where the patient has exhausted all standard therapies with elevated baseline serum GDF15; an advanced prostate cancer where the patient has previously received one or more (e.g., 1, 2, 3, or more) lines of cancer therapies; a chemo-refractory prostate cancer; a metastatic chemo-refractory prostate cancer), a bladder cancer, a melanoma, an SCLC (e.g., an extensive-stage SCLC), an NSCLC, a pancreatic cancer (e.g., an advanced pancreatic cancer, a metastatic pancreatic cancer, a pancreatic adenocarcinoma, a metastatic pancreatic adenocarcinoma, an MSI-H pancreatic cancer, a pancreatic ductal adenocarcinoma, a metastatic pancreatic ductal adenocarcinoma), a colorectal cancer, a gastric cancer, an esophageal cancer, an ovarian cancer, a head neck squamous cell carcinoma or an MSI-H cancer (e.g., an MSI-H prostate cancer, an MSI-H ovanan cancer, an MSI-H ovarian cancer, an MSI-H HNSCC, an MSI-H SLCLC (e.g., an MSI-H extensive-stage SCLC). In some instances, the cancer is an MSI-H cancer selected from a castration-resistant prostate cancer, a bladder cancer, a melanoma, a SCLC (e.g., an extensive-stage SCLC), a pancreatic cancer (e.g., an advanced pancreatic cancer, a metastatic pancreatic cancer, a pancreatic adenocarcinoma, a metastatic pancreatic adenocarcinoma, an MSI-H pancreatic cancer, a pancreatic ductal adenocarcinoma, a metastatic pancreatic ductal adenocarcinoma), a colorectal cancer, a gastric cancer, an esophageal cancer, an ovarian cancer, a head and neck squamous cell carcinoma). In some instances, the cancer is a pancreatic cancer (e.g., an advanced pancreatic cancer, a metastatic pancreatic cancer, a pancreatic adenocarcinoma, a metastatic pancreatic adenocarcinoma, an MSI-H pancreatic cancer, a pancreatic ductal adenocarcinoma, a metastatic pancreatic ductal adenocarcinoma). In some instances, the cancer is a pancreatic adenocarcinoma. In some instances, the cancer is a metastatic pancreatic cancer. In some instances, the cancer is a metastatic pancreatic adenocarcinoma. In some instances, the human patient has a prostate cancer (e.g., an mCRPC, a castration-sensitive prostate cancer, a castration-resistant prostate cancer, an MSI-H prostate cancer, a metastatic prostate cancer, a metastatic castrate-resistant, chemo-refractory prostate cancer; an advanced prostate cancer where the patient has exhausted all standard therapies with elevated baseline serum GDF15; an advanced prostate cancer where the patient has previously received one or more (e.g., 1, 2, 3, or more) lines of cancer therapies; a chemo-refractory prostate cancer; a metastatic chemo-refractory prostate cancer). In some instances, the cancer is advanced prostate cancer. In some instances, the human patient has prostate cancer and poor survival. In some instances, the cancer is an mCRPC. In some instances, the cancer is chemo-refractory prostate cancer. In some instances, the cancer is metastatic chemo-refractory prostate cancer. In some instances, the cancer is metastatic castrate-resistant, chemo-refractory prostate cancer. In some instances, the cancer is advanced prostate cancer, wherein the human patient has exhausted all standard therapies, and wherein the human patient has an elevated baseline serum GDF15 (compared to a human or a panel of humans not having prostate cancer). In some instances, the cancer is advanced prostate cancer (e.g., castration-resistant prostate cancer), wherein the human patient has previously received one or more (e.g., 1, 2, 3, or more) lines of therapies (e.g., leuprolide acetate (LUPRON), zoledronic acid (ZOMETA), docetaxel, cabazitaxel, radium 223, mitoxantrone, Oxaliplatin and pemetrexed). In some instances, the human patient previously failed to be treated with one or more (e.g., 1, 2, 3, 4, 5) other cancer therapies (i.e., other than an anti-GFRAL antibody described herein) prior to the administering of the anti-GFRAL antibody.

Also provided herein is a method for treating pancreatic cancer (e.g., an advanced pancreatic cancer, a metastatic pancreatic cancer, a pancreatic adenocarcinoma, a metastatic pancreatic adenocarcinoma, an MSI-H pancreatic cancer, a pancreatic ductal adenocarcinoma, a metastatic pancreatic ductal adenocarcinoma) in a human patient, the method comprising administering to the human patient: (a) an anti-GFRAL antibody at a dose of 30 mg once every 4 weeks; (b) paclitaxel (e.g., nab-paclitaxel), and (c) gemcitabine; wherein the anti-GFRAL antibody comprises: (i) a VH comprising a VH CDR1 comprising the amino acid sequence set forth in SEQ ID NO:48, a VH CDR2 comprising the amino acid sequence set forth in SEQ ID NO:137, and a VH CDR3 comprising the amino acid sequence set forth in SEQ ID NO:225; and (ii) a VL comprising a VL CDR1 comprising the amino acid sequence set forth in SEQ ID NO:301, a VL CDR2 comprising the amino acid sequence set forth in SEQ ID NO:376, and a VL CDR3 comprising the amino acid sequence set forth in SEQ ID NO:426. In some instances, the anti-GFRAL antibody is administered to the human patient subcutaneously. In some instances, the anti-GFRAL antibody is administered to the human patient intravenously. In some instances, the paclitaxel (e.g., nab-paclitaxel) is administered to the human patient at a dose of 125 mg/m² once every week. In some instances, the paclitaxel (e.g., nab-paclitaxel) is administered to the human patient intravenously. In some instances, the paclitaxel is nab-paclitaxel. In some instances, the gemcitabine is administered to the human patient at a dose of 1000 mg/m² once every week. In some instances, the gemcitabine is administered to the human patient intravenously. In some instances, the human patient has an elevated level of GDF15 (e.g., compared to a level of GDF15 in a human or a panel of humans (e.g., 2, 3, 4, 5, 10, 15, 20, 25, or more) not having pancreatic cancer). In some instances, the pancreatic cancer is advanced pancreatic cancer. In some instances, the pancreatic cancer is metastatic pancreatic cancer. In some instances, the pancreatic cancer is pancreatic adenocarcinoma. In some instances, the pancreatic cancer is an advanced pancreatic cancer. In some instances, the pancreatic cancer is an MSI-H pancreatic cancer. In some instances, the pancreatic cancer is a pancreatic ductal adenocarcinoma. In some instances, the pancreatic cancer is a metastatic pancreatic ductal adenocarcinoma. In some instances, the pancreatic cancer is metastatic pancreatic adenocarcinoma. In some instances, the human patient previously failed to be treated with one or more (e.g., 1, 2, 3, 4, 5) other cancer therapies (i.e., other than an anti-GFRAL antibody described herein) prior to the administering of the anti-GFRAL antibody, paclitaxel, and gemcitabine.

Also provided herein is a method for treating pancreatic cancer (e.g., an advanced pancreatic cancer, a metastatic pancreatic cancer, a pancreatic adenocarcinoma, a metastatic pancreatic adenocarcinoma, an MSI-H pancreatic cancer, a pancreatic ductal adenocarcinoma, a metastatic pancreatic ductal adenocarcinoma) in a human patient, the method comprising administering to the human patient: (a) an anti-GFRAL antibody at a dose of 30 mg once every 4 weeks; (b) paclitaxel (e.g., nab-paclitaxel), and (c) gemcitabine; wherein the anti-GFRAL antibody comprises: a VH comprising the amino acid sequence set forth in SEQ ID NO:1982 and a VL comprising the amino acid sequence set forth in SEQ ID NO:1997. In some instances, the anti-GFRAL antibody is administered to the human patient subcutaneously. In some instances, the anti-GFRAL antibody is administered to the human patient intravenously. In some instances, the paclitaxel (e.g., nab-paclitaxel) is administered to the human patient at a dose of 125 mg/m² once every week. In some instances, the paclitaxel (e.g., nab-paclitaxel) is administered to the human patient intravenously. In some instances, the paclitaxel is nab-paclitaxel. In some instances, the gemcitabine is administered to the human patient at a dose of 1000 mg/m² once every week. In some instances, the gemcitabine is administered to the human patient intravenously. In some instances, the human patient has an elevated level of GDF15 (e.g., compared to a level of GDF15 in a human or a panel of humans (e.g., 2, 3, 4, 5, 10, 15, 20, 25, or more) not having pancreatic cancer). In some instances, the pancreatic cancer is advanced pancreatic cancer. In some instances, the pancreatic cancer is metastatic pancreatic cancer. In some instances, the pancreatic cancer is pancreatic adenocarcinoma. In some instances, the pancreatic cancer is metastatic pancreatic adenocarcinoma. In some instances, the pancreatic cancer is an advanced pancreatic cancer. In some instances, the pancreatic cancer is an MSI-H pancreatic cancer. In some instances, the pancreatic cancer is a pancreatic ductal adenocarcinoma. In some instances, the pancreatic cancer is a metastatic pancreatic ductal adenocarcinoma. In some instances, the human patient previously failed to be treated with one or more (e.g., 1, 2, 3, 4, 5) other cancer therapies (i.e., other than an anti-GFRAL antibody described herein) prior to the administering of the anti-GFRAL antibody, paclitaxel, and gemcitabine.

Also provided herein is a method for treating pancreatic cancer (e.g., an advanced pancreatic cancer, a metastatic pancreatic cancer, a pancreatic adenocarcinoma, a metastatic pancreatic adenocarcinoma, an MSI-H pancreatic cancer, a pancreatic ductal adenocarcinoma, a metastatic pancreatic ductal adenocarcinoma) in a human patient, the method comprising administering to the human patient: (a) an anti-GFRAL antibody at a dose of 30 mg once every 4 weeks; (b) paclitaxel (e.g., nab-paclitaxel), and (c) gemcitabine; wherein the anti-GFRAL antibody comprises: a heavy chain comprising the amino acid sequence set forth in SEQ ID NO:2010 and a light chain comprising the amino acid sequence set forth in SEQ ID NO:2012. In some instances, the anti-GFRAL antibody comprises a heavy chain consisting of the amino acid sequence set forth in SEQ ID NO:2010 and a light chain comprising the amino acid sequence set forth in SEQ ID NO:2012. In some instances, the anti-GFRAL antibody is administered to the human patient subcutaneously. In some instances, the anti-GFRAL antibody is administered to the human patient intravenously. In some instances, the paclitaxel (e.g., nab-paclitaxel) is administered to the human patient at a dose of 125 mg/m² once every week. In some instances, the paclitaxel (e.g., nab-paclitaxel) is administered to the human patient intravenously. In some instances, the paclitaxel is nab-paclitaxel. In some instances, the gemcitabine is administered to the human patient at a dose of 1000 mg/m² once every week. In some instances, the gemcitabine is administered to the human patient intravenously. In some instances, the human patient has an elevated level of GDF15 (e.g., compared to a level of GDF15 in a human or a panel of humans (e.g., 2, 3, 4, 5, 10, 15, 20, 25, or more) not having pancreatic cancer). In some instances, the pancreatic cancer is advanced pancreatic cancer. In some instances, the pancreatic cancer is metastatic pancreatic cancer. In some instances, the pancreatic cancer is pancreatic adenocarcinoma. In some instances, the pancreatic cancer is metastatic pancreatic adenocarcinoma. In some instances, the pancreatic cancer is an advanced pancreatic cancer. In some instances, the pancreatic cancer is an MSI-H pancreatic cancer. In some instances, the pancreatic cancer is a pancreatic ductal adenocarcinoma. In some instances, the pancreatic cancer is a metastatic pancreatic ductal adenocarcinoma. In some instances, the human patient previously failed to be treated with one or more (e.g., 1, 2, 3, 4, 5) other cancer therapies (i.e., other than an anti-GFRAL antibody described herein) prior to the administering of the anti-GFRAL antibody, paclitaxel, and gemcitabine.

Also provided herein is a method for treating pancreatic cancer (e.g., an advanced pancreatic cancer, a metastatic pancreatic cancer, a pancreatic adenocarcinoma, a metastatic pancreatic adenocarcinoma, an MSI-H pancreatic cancer, a pancreatic ductal adenocarcinoma, a metastatic pancreatic ductal adenocarcinoma) in a human patient, comprising administering to the human patient: (a) an anti-GFRAL antibody at a dose of 100 mg once every 4 weeks; (b) paclitaxel (e.g., nab-paclitaxel), and (c) gemcitabine; wherein the anti-GFRAL antibody comprises: (i) a VH comprising a VH CDR1 comprising the amino acid sequence set forth in SEQ ID NO:48, a VH CDR2 comprising the amino acid sequence set forth in SEQ ID NO:137, and a VH CDR3 comprising the amino acid sequence set forth in SEQ ID NO:225; and (ii) a VL comprising a VL CDR1 comprising the amino acid sequence set forth in SEQ ID NO:301, a VL CDR2 comprising the amino acid sequence set forth in SEQ ID NO:376, and a VL CDR3 comprising the amino acid sequence set forth in SEQ ID NO:426. In some instances, the anti-GFRAL antibody is administered to the human patient subcutaneously. In some instances, the anti-GFRAL antibody is administered to the human patient intravenously. In some instances, the paclitaxel (e.g., nab-paclitaxel) is administered to the human patient at a dose of 125 mg/m² once every week. In some instances, the paclitaxel (e.g., nab-paclitaxel) is administered to the human patient intravenously. In some instances, the paclitaxel is nab-paclitaxel. In some instances, the gemcitabine is administered to the human patient at a dose of 1000 mg/m² once every week. In some instances, the gemcitabine is administered to the human patient intravenously. In some instances, the human patient has an elevated level of GDF15 (e.g., compared to a level of GDF15 in a human or a panel of humans (e.g., 2, 3, 4, 5, 10, 15, 20, 25, or more) not having pancreatic cancer). In some instances, the pancreatic cancer is advanced pancreatic cancer. In some instances, the pancreatic cancer is metastatic pancreatic cancer. In some instances, the pancreatic cancer is pancreatic adenocarcinoma. In some instances, the pancreatic cancer is metastatic pancreatic adenocarcinoma. In some instances, the pancreatic cancer is an advanced pancreatic cancer. In some instances, the pancreatic cancer is an MSI-H pancreatic cancer. In some instances, the pancreatic cancer is a pancreatic ductal adenocarcinoma. In some instances, the pancreatic cancer is a metastatic pancreatic ductal adenocarcinoma. In some instances, the human patient previously failed to be treated with one or more (e.g., 1, 2, 3, 4, 5) other cancer therapies (i.e., other than an anti-GFRAL antibody described herein) prior to the administering of the anti-GFRAL antibody, paclitaxel, and gemcitabine.

Also provided herein is a method for treating pancreatic cancer (e.g., an advanced pancreatic cancer, a metastatic pancreatic cancer, a pancreatic adenocarcinoma, a metastatic pancreatic adenocarcinoma, an MSI-H pancreatic cancer, a pancreatic ductal adenocarcinoma, a metastatic pancreatic ductal adenocarcinoma) in a human patient, the method comprising administering to the human patient: (a) an anti-GFRAL antibody at a dose of 100 mg once every 4 weeks; (b) paclitaxel (e.g., nab-paclitaxel), and (c) gemcitabine; wherein the anti-GFRAL antibody comprises: a VH comprising the amino acid sequence set forth in SEQ ID NO:1982 and a VL comprising the amino acid sequence set forth in SEQ ID NO:1997. In some instances, the anti-GFRAL antibody is administered to the human patient subcutaneously. In some instances, the anti-GFRAL antibody is administered to the human patient intravenously. In some instances, the paclitaxel (e.g., nab-paclitaxel) is administered to the human patient at a dose of 125 mg/m² once every week. In some instances, the paclitaxel (e.g., nab-paclitaxel) is administered to the human patient intravenously. In some instances, the paclitaxel is nab-paclitaxel. In some instances, the gemcitabine is administered to the human patient at a dose of 1000 mg/m² once every week. In some instances, the gemcitabine is administered to the human patient intravenously. In some instances, the human patient has an elevated level of GDF15 (e.g., compared to a level of GDF15 in a human or a panel of humans (e.g., 2, 3, 4, 5, 10, 15, 20, 25, or more) not having pancreatic cancer). In some instances, the pancreatic cancer is advanced pancreatic cancer. In some instances, the pancreatic cancer is metastatic pancreatic cancer. In some instances, the pancreatic cancer is pancreatic adenocarcinoma. In some instances, the pancreatic cancer is metastatic pancreatic adenocarcinoma. In some instances, the pancreatic cancer is an advanced pancreatic cancer. In some instances, the pancreatic cancer is an MSI-H pancreatic cancer. In some instances, the pancreatic cancer is a pancreatic ductal adenocarcinoma. In some instances, the pancreatic cancer is a metastatic pancreatic ductal adenocarcinoma. In some instances, the human patient previously failed to be treated with one or more (e.g., 1, 2, 3, 4, 5) other cancer therapies (i.e., other than an anti-GFRAL antibody described herein) prior to the administering of the anti-GFRAL antibody, paclitaxel, and gemcitabine.

Also provided herein is a method for treating pancreatic cancer (e.g., an advanced pancreatic cancer, a metastatic pancreatic cancer, a pancreatic adenocarcinoma, a metastatic pancreatic adenocarcinoma, an MSI-H pancreatic cancer, a pancreatic ductal adenocarcinoma, a metastatic pancreatic ductal adenocarcinoma) in a human patient, comprising administering to the human patient: (a) an anti-GFRAL antibody at a dose of 100 mg once every 4 weeks; (b) paclitaxel (e.g., nab-paclitaxel), and (c) gemcitabine; wherein the anti-GFRAL antibody comprises: a heavy chain comprising the amino acid sequence set forth in SEQ ID NO:2010 and a light chain comprising the amino acid sequence set forth in SEQ ID NO:2012. In some instances, the anti-GFRAL antibody comprises a heavy chain consisting of the amino acid sequence set forth in SEQ ID NO:2010 and a light chain comprising the amino acid sequence set forth in SEQ ID NO:2012. In some instances, the anti-GFRAL antibody is administered to the human patient subcutaneously. In some instances, the anti-GFRAL antibody is administered to the human patient intravenously. In some instances, the paclitaxel (e.g., nab-paclitaxel) is administered to the human patient at a dose of 125 mg/m² once every week. In some instances, the paclitaxel (e.g., nab-paclitaxel) is administered to the human patient intravenously. In some instances, the paclitaxel is nab-paclitaxel. In some instances, the gemcitabine is administered to the human patient at a dose of 1000 mg/m² once every week. In some instances, the gemcitabine is administered to the human patient intravenously. In some instances, the human patient has an elevated level of GDF15 (e.g., compared to a level of GDF15 in a human or a panel of humans (e.g., 2, 3, 4, 5, 10, 15, 20, 25, or more) not having pancreatic cancer). In some instances, the pancreatic cancer is advanced pancreatic cancer. In some instances, the pancreatic cancer is metastatic pancreatic cancer. In some instances, the pancreatic cancer is pancreatic adenocarcinoma. In some instances, the pancreatic cancer is metastatic pancreatic adenocarcinoma. In some instances, the pancreatic cancer is an advanced pancreatic cancer. In some instances, the pancreatic cancer is an MSI-H pancreatic cancer. In some instances, the pancreatic cancer is a pancreatic ductal adenocarcinoma. In some instances, the pancreatic cancer is a metastatic pancreatic ductal adenocarcinoma. In some instances, the human patient previously failed to be treated with one or more (e.g., 1, 2, 3, 4, 5) other cancer therapies (i.e., other than an anti-GFRAL antibody described herein) prior to the administering of the anti-GFRAL antibody, paclitaxel, and gemcitabine.

In some instances of the foregoing methods, the human patient has a reduced level of GDF15 (e.g., compared to a baseline level of GDF15 prior to the administering of the anti-GFRAL antibody) after the administering of the anti-GFRAL antibody (e.g., a level that is reduced by at least 10%, at least 20%, at least 30%, at least 40%, at least 50%, at least 60%, at least 70%, at least 80%, at least 90%, or at least 95% relative to the baseline level).

In some instances of the foregoing methods, the human patient is about 35 years of age, about 40 years of age, about 45 years of age, about 50 years of age, about 55 years of age, about 60 years of age, about 65 years of age, about 70 years of age, about 75 years of age, about 80 years of age, or about 85 years of age. In some instances of the foregoing methods, the human patient is at least 35 years of age, at least 40 years of age, at least 45 years of age, at least 50 years of age, at least 55 years of age, at least 60 years of age, at least 65 years of age, at least 70 years of age, at least 75 years of age, at least 80 years of age, or at least 85 years of age. In some instances of the foregoing methods, the human patient is 35-90 years of age, 35-80 years of age, 35-70 years of age, 45-90 years of age, 45-80 years of age, 45-70 years of age, 55-90 years of age, 55-80 years of age, 55 to 75 years of age, or 55-65 years of age.

### VI. BRIEF DESCRIPTION OF THE DRAWINGS

FIG. 1 is a graph showing dose dependent effect of the anti-GFRAL antibody on preventing or reducing cisplatin-induced body weight loss in rats. Values are mean f SEM from n=8 in each group. P values for group 3, group 4 and group 5 were calculated in comparison with group 2 by t-test using the Holm-Sidak method. ns=not significant. Day 21: lines are Group 1, Group 5, Group 4, Group 6, Group 3, and Group 2, respectively, from top to bottom.

FIG. 2A is a series of graphs showing the effect of the anti-GFRAL antibody on body weight gain in healthy human volunteers. The percentage change of body weight relative to the baseline body weight in each subject was plotted. Each line represents a subject.

FIG. 2B is a chart showing exemplary body weight changes on day 85.

FIG. 3 is a graph showing serum concentrations of the anti-GFRAL antibody following administration at a frequency of once every 3 weeks in subjects diagnosed with various cancers including colorectal cancer, pancreatic cancer, prostate cancer, bladder cancer, and esophageal cancer. Each line represents a subject. Top 7 lines at day 40 correspond to the 100 mg Antibody dose; the rest of the lines correspond to the 30 mg Antibody dose.

FIG. 4 is a graph showing lean body mass change in subjects undergoing the treatment of the anti-GFRAL antibody. Each line represents a subject. Square, diamond, circle, point-up triangle, and point-down triangle correspond to the 100 mg Antibody dose; the rest of the data points correspond to the 30 mg Antibody dose.

FIG. 5 is a graph showing serum concentrations of the anti-GFRAL antibody following administration of the antibody together with gemcitabine and abraxane in subjects diagnosed with metastatic pancreatic cancer. Each line represents a subject.

FIG. 6 is a graph showing tumor progression in 8 metastatic pancreatic cancer patients during the treatment of the anti-GFRAL antibody in combination with gemcitabine and abraxane. SD is an abbreviation for Stable Disease; PR is an abbreviation for Partial Response; NE is an abbreviation for Not Evaluable.

FIG. 7 is a graph showing percentage changes of CA19-9 levels in circulation from the baseline level in metastatic pancreatic cancer patients undergoing the combination therapy. Solid lines represent CA19-9 positive patients. The dotted line represents a CA19-9 negative patient. 4 patients received 30 mg of the anti-GFRAL antibody together with gemcitabine and abraxane, 2 patients received 100 mg of the anti-GFRAL antibody together with gemcitabine and abraxane.

FIG. 8 is a graph showing the percentage change of lean body mass in 6 metastatic pancreatic cancer patients undergoing the combination therapy. 4 patients received 30 mg of the anti-GFRAL antibody together with gemcitabine and abraxane. 2 patients received 100 mg of the anti-GFRAL antibody together with gemcitabine and abraxane.

FIG. 9 is a graph showing the percentage change of body weight in 6 metastatic pancreatic cancer patients undergoing the combination. 4 patients received 30 mg of the anti-GFRAL antibody together with gemcitabine and abraxane. 2 patients received 100 mg of the anti-GFRAL antibody together with gemcitabine and abraxane.

FIG. 10A shows alignments of VH sequences of humanized 5F12 antibodies. SEQ ID NOS are noted in parenthesis and bold text.

FIG. 10B shows alignments of VL sequences of humanized 5F12 antibodies. SEQ ID NOS are noted in parenthesis and bold text.

### VII. DETAILED DESCRIPTION OF THE INVENTION

The present disclosure relates generally to methods of (i) treating a tumor (e.g., a solid tumor, an advanced solid tumor, a cancer) in a human patient, or (ii) treating tumor-related weight loss or cachexia in a human patient, the method comprising administering to the human patient an antibody that binds to GFRAL (an “anti-GFRAL antibody”), at a dose of about 30 mg once about every 3 weeks or about 100 mg once about every 3 weeks, wherein the antibody inhibits GFRAL binding to RET. In some instances, the tumor is a solid tumor. In some instances, the tumor is an advanced solid tumor. In instances, the tumor is a cancer. In some instances, the cancer is an advanced solid tumor. In some instances, the cancer is a prostate cancer, a bladder cancer, a melanoma, a small cell lung cancer (SCLC) (including an extensive-stage SCLC), a non-small cell lung cancer (NSCLC), a pancreatic cancer, a colorectal cancer, a gastric cancer, an esophageal cancer, an ovarian cancer, a head and neck squamous cell carcinoma, or an MSI-H (microsatellite instability high) cancer. The prostate cancer can include, for example, a castration-sensitive prostate cancer, a castration-resistant prostate cancer, an MSI-H prostate cancer, a metastatic prostate cancer, a chemo-refractory prostate cancer, a prostate cancer where the patient has previously received one or more, two or more, or one or more (e.g., 1, 2, 3, or more) lines of standard prostate cancer therapies, or an advanced prostate cancer where the patient has exhausted all standard lines of therapies. A pancreatic cancer can include, for example, an advanced pancreatic cancer, a metastatic pancreatic cancer, a pancreatic adenocarcinoma, a metastatic pancreatic adenocarcinoma, an MSI-H pancreatic cancer, a pancreatic ductal adenocarcinoma, or a metastatic pancreatic ductal adenocarcinoma. In some instances, the pancreatic cancer is a pancreatic adenocarcinoma. In some instances, the pancreatic cancer is a metastatic pancreatic adenocarcinoma. In some instances, the pancreatic cancer is an advanced pancreatic cancer. In some instances, the pancreatic cancer is a metastatic pancreatic cancer. In some instances, the pancreatic cancer is an MSI-H pancreatic cancer. In some instances, the pancreatic cancer is a pancreatic ductal adenocarcinoma. In some instances, the pancreatic cancer is a metastatic pancreatic ductal adenocarcinoma. An MSI-H cancer can include, for example, an MSI-H prostate cancer, an MSI-H bladder cancer, an MSI-H melanoma, an MSI-H small cell lung cancer (SCLC) (including an extensive-stage SCLC), an MSI-H non-small cell lung cancer (NSCLC), an MSI-H pancreatic cancer, an MSI-H colorectal cancer, an MSI-H gastric cancer, an MSI-H esophageal cancer, an MSI-H ovarian cancer, or an MSI-H head and neck squamous cell carcinoma. In some instances, the patient has an elevated level of GDF15 in the serum compared to the baseline level of GDF15 thereof without the tumor. In some instances, the antibody is administered subcutaneously. In some instances, the antibody is administered intravenously. As used herein throughout, in the context of a dose, “about” means+/−10% of the recited dose. For instance, “about 30 mg” means “27 mg to 33 mg” and “about 100 mg” means “90 mg to 110 mg”. As used herein throughout, in the context of a duration of time, “about” means+/−one week. For instance, “about 3 weeks” means “2 weeks to 4 weeks” and “about 4 weeks” means “3 weeks to 5 weeks”. The present disclosure also relates generally to methods of treating pancreatic cancer (e.g., an advanced pancreatic cancer, a metastatic pancreatic cancer, a pancreatic adenocarcinoma, a metastatic pancreatic adenocarcinoma, an MSI-H pancreatic cancer, a pancreatic ductal adenocarcinoma, a metastatic pancreatic ductal adenocarcinoma) in a human patient, comprising administering to the human patient. (i) an anti-GFRAL antibody at a dose of about 30 mg once about every 4 weeks or a dose of about 100 mg once about every 4 weeks; (ii) paclitaxel (e.g., nab-paclitaxel); and (iii) gemcitabine; wherein the anti-GFRAL antibody inhibits GFRAL binding to RET. In some cases, the antibody is administered subcutaneously and the paclitaxel and gemcitabine are each administered intravenously. In some cases, the antibody, paclitaxel, and gemcitabine are each administered intravenously. In some instances, the paclitaxel (e.g., nab-paclitaxel) is administered to the human patient at a dose of 125 mg/m² once every week. In some instances, the gemcitabine is administered to the human patient at a dose of 1000 mg/m² gemcitabine once every week.

The term “treat” or “treatment” or “treating” or “to treat” as used herein refers to therapeutic measures that aim to relieve, slow down progression of, lessen symptoms of, and/or halt progression of a pathologic condition or disorder. Thus, those in need of treatment include those already with the disorder.

Unless explained otherwise, all technical and scientific terms used herein have the same meaning as commonly understood to one of ordinary skill in the art to which this disclosure belongs.

A. GFRAL, GDF15, and RET

Human GDNF Family Receptor Alpha Like (GFRAL, also known in the art as GDF15 receptor, C6orf144, Chromosome 6 Open reading Frame 144, BA360D14.1, IVF19356, and UNQ9356) is a 375-amino acid (excluding the signal peptide) protein that acts as a receptor for GDF15 signaling.

The amino acid sequence of a full-length precursor human GFRAL is provided below and includes a signal peptide sequence (lowercase residues):

(SEQ ID NO: 1797)
mivfiflamglsleneytsQTNNCTYLREQCLRDA
NGCKHAWRVMEDACNDSDPGDPCKMRNSSYCNLSI
QYLVESNFQFKECLCTDDFYCTVNKLLGKKCINKS
DNVKEDKFKWNLTTRSHHGFKGMWSCLEVAEACVG
DVVCNAQLASYLKACSANGNPCDLKQCQAAIRFFY
QNIPFNIAQMLAFCDCAQSDIPCQQSKEALHSKTC
AVNMVPPPTCLSVIRSCQNDELCRRHYRTFQSKCW
QRVTRKCHEDENCISTLSKQDLTCSGSDDCKAAYI
DILGTVLQVQCTCRTITQSEESLCKIFQHMLHRKS
CFNYPTLSNVKGMALYTRKHANKITLTGFHSPFNG
EVIYAAMCMTVTCGILLLVMVKLRTSRISSKARDP
SSIQIPGEL.

The amino acid sequence of a mature human GFRAL polypeptide is provided below:

(SEQ ID NO: 1798)
QTNNCTYLREQCLRDANGCKHAWRVMEDACNDSDP
GDPCKMRNSSYCNLSIQYLVESNFQFKECLCTDDF
YCTVNKLLGKKCINKSDNVKEDKFKWNLTTRSHHG
FKGMWSCLEVAEACVGDVVCNAQLASYLKACSANG
NPCDLKQCQAAIRFFYQNIPFNIAQMLAFCDCAQS
DIPCQQSKEALHSKTCAVNMVPPPTCLSVIRSCQN
DELCRRHYRTFQSKCWQRVTRKCHEDENCISTLSK
QDLTCSGSDDCKAAYIDILGTVLQVQCTCRTITQS
EESLCKIFQHMLHRKSCFNYPTLSNVKGMALYTRK
HANKITLTGFHSPFNGEVIYAAMCMTVTCGILLLV
MVKLRTSRISSKARDPSSIQIPGEI.

Human GFRAL has an extracellular domain (e.g., residues 20-351 of the amino acid sequence set forth in SEQ ID NO: 1797), a transmembrane domain (e.g., residues 352-371 of the amino acid sequence set forth in SEQ ID NO: 1797) and a cytoplasmic domain (e.g., residues 372-394 of the amino acid sequence set forth in SEQ ID NO: 1797).

GDF15 (also known in the art as MIC-1 (macrophage inhibitory cytokine-1), PDF (prostate differentiation factor), PLAB (placental bone morphogenetic protein), NAG-1 (non-steroidal anti-inflammatory drugs (NSAIDs) activated gene), TGF-PL, and PTGFB), is a member of the transforming growth factor β (TGF-β) super-family. GDF15, which is synthesized as a 62 kDa intracellular precursor protein that is subsequently cleaved by a furin-like protease, is secreted as a 25 kDa disulfide-linked protein (see, e.g., Fairlie et al., J. Leukoc. Biol 65:2-5 (1999)). GDF15 mRNA is seen in several tissues, including liver, kidney, pancreas, colon and placenta, and GDF15 expression in liver can be significantly up-regulated during injury of organs such as the liver, kidneys, heart and lungs.

The GDF15 precursor is a 308 amino acid polypeptide (NCBI Ref. Seq. NP_004855.2; GI:153792495) containing a 29 amino acid signal peptide, a 167 amino acid pro-domain, and a mature domain of 112 amino acids which is excised from the pro-domain by furin-like proteases.

An amino acid sequence of a precursor human GDF15 polypeptide is provided below:

(SEQ ID NO: 1810)
MPGQELRTVNGSQMLLVLLVLSWLPHGGALSLAEA
SRASFPGPSELHSEDSRFRELRKRYEDLLTRLRAN
QSWEDSNTDLVPAPAVRILTPEVRLGSGGHLHLRI
SRAALPEGLPEASRLHRALFRLSPTASRSWDVTRP
LRRQLSLARPQAPALHLRLSPPPSQSDQLLAESSS
ARPQLELHLRPQAARGRRRARARNGDHCPLGPGRC
CRLHTVRASLEDLGWADWVLSPREVQVTMCIGACP
SQFRAANMHAQIKTSLHRLKPDTVPAPCCVPASYN
PMVLIQKTDTGVSLQTYDDLLAKDCHCI.

Such a 308-amino acid GDF15 polypeptide is referred to as a “full-length” GDF15 polypeptide; a 112-amino acid GDF15 polypeptide (amino acids 197-308 of “full-length” GDF15) is a “mature” GDF15 polypeptide. An amino acid sequence of a mature human GDF15 polypeptide is provided below:

(SEQ ID NO: 1811)
ARNGDHCPLGPGRCCRLHTVRASLEDLGWADWVLS
PREVQVTMCIGACPSQFRAANMHAQIKTSLHRLKP
DTVPAPCCVPASYNPMVLIQKTDTGVSLQTYDDLL
AKDCHCI.

“RET” (also known in the art as Ret Proto-Oncogene, Cadherin-Related Family Member 16. Rearranged During Transfection, RET Receptor Tyrosine Kinase, Cadherin Family Member 12, Proto-Oncogene C-Ret, EC 2.7.10.1, CDHF12, CDHR16, RET51, PTC, Hydroxyaryl-Protein Kinase, RET Transforming Sequence, and Receptor Tyrosine Kinase) is one of the receptor tyrosine kinases, cell-surface molecules that transduce signals for cell growth and differentiation. RET acts as a co-receptor and is known as a primary signaling receptor for glial-cell-line-derived neurotrophic factor (GDNF) ligands (in human, GDNF, artemin, neurturin, and persephin) when bound to members of the GDNF receptor alpha (GFRα) co-receptors. A RET protein (e.g., a RET-ECD) comprises 4 consecutive cadherin-like domains (CLD1-CLD4) followed by a membrane proximal cysteine rich domain (CRD). RET protein is a co-receptor with a GFRAL protein and a GDF15 protein (e.g., acting as a co-receptor with a RET protein). A receptor complex includes a GFRAL protein, such as a RET/GFRAL complex, a GFRAL/GDF15 complex, and a RET/GFRAL/GDF15 complex.

RET is distinct from TGFβ RI and TGFβ RII. SEQ ID NO: 1812 is the sequence of a mature human RET9 that lacks a signal peptide:

(SEQ ID NO: 1812)
KVALGLYFSRDAYWEKLYVDQAAGTPLLYVHALRD
APEEVPSFRLGQHLYGTYRTRLHENNWICIQEDTG
LLYLNRSLDHSSWEKLSVRNRGFPLLTVYLKVFLS
PTSLREGECQWPGCARVYFSFFNTSFPACSSLKPR
ELCFPETRPSFRIRENRPPGTFHQFRLLPVQFLCP
NISVAYRLLEGEGLPFRCAPDSLEVSTRWALDREQ
REKYELVAVCTVHAGAREEVVMVPFPVTVYDEDDS
APTFPAGVDTASAVVEFKRKEDTVVATLRVFDADV
VPASGELVRRYTSTLLPGDTWAQQTFRVEHWPNET
SVQANGSFVRATVHDYRLVLNRNLSISENRTMQLA
VLVNDSDFQGPGAGVLLLHFNVSVLPVSLHLPSTY
SLSVSRRARRFAQIGKVCVENCQAFSGINVQYKLH
SSGANCSTLGVVTSAEDTSGILFVNDTKALRRPKC
AELHYMVVATDQQTSRQAQAQLLVTVEGSYVAEEA
GCPLSCAVSKRRLECEECGGLGSPTGRCEWRQGDG
KGITRNFSTCSPSTKTCPDGHCDVVETQDINICPQ
DCLRGSIVGGHEPGEPRGIKAGYGTCNCFPEEEKC
FCEPEDIQDPLCDELCRTVIAAAVLFSFIVSVLLS
AFCIHCYHKFAHKPPISSAEMTFRRPAQAFPVSYS
SSGARRPSLDSMENQVSVDAFKILEDPKWEFPRKN
LVLGKTLGEGEFGKVVKATAFHLKGRAGYTTVAVK
MLKENASPSELRDLLSEFNVLKQVNHPHVIKLYGA
CSQDGPLLLIVEYAKYGSLRGFLRESRKVGPGYLG
SGGSRNSSSLDHPDERALTMGDLISFAWQISQGM
QYLAEMKLVHRDLAARNILVAEGRKMKISDFGLSR
DVYEEDSYVKRSQGRIPVKWMAIESLFDHIYTTQ
SDVWSFGVLLWEIVTLGGNPYPGIPPERLFNLLKT
GHRMERPDNCSEEMYRLMLQCWKQEPDKRPVFAD
ISKDLEKMMVKRRDYLDLAASTPSDSLIYDDGLS
EEETPLVDCNNAPLPRALPSTWIENKLYGRISHA
FTRF

The amino acid sequence of a full-length precursor human RET protein is provided below and includes a signal peptide sequence (lowercase residues):

(SEQ ID NO: 1813)
makatsgaaglrlllllllpllgkvalgLYFSRDA
YWEKLYVDQAAGTPLLYVHALRDAPEEVPSFRLGQ
HLYGTYRTRLHENNWICIQEDTGLLYLNRSLDHSS
WEKLSVRNRGFPLLTVYLKVFLSPTSLREGECQWP
GCARVYFSFFNTSFPACSSLKPRELCFPETRPSFR
IRENRPPGTFHQFRLLPVQFLCPNISVAYRLLEGE
GLPFRCAPDSLEVSTRWALDREQREKYELVAVCTV
HAGAREEVVMVPFPVTVYDEDDSAPTFPAGVDTAS
AVVEFKRKEDTVVATLRVFDADVVPASGELVRRYT
STLLPGDTWAQQTFRVEHWPNETSVQANGSFVRAT
VHDYRLVLNRNLSISENRTMQLAVLVNDSDFQGPG
AGVLLLHFNVSVLPVSLHLPSTYSLSVSRRARRFA
QIGKVCVENCQAFSGINVQYKLHSSGANCSTLGVV
TSAEDTSGILFVNDTKALRRPKCAELHYMVVATDQ
QTSRQAQAQLLVTVEGSYVAEEAGCPLSCAVSKRR
LECEECGGLGSPTGRCEWRQGDGKGITRNFSTCSP
STKTCPDGHCDVVETQDINICPQDCLRGSIVGGHE
PGEPRGIKAGYGTCNCFPEEEKCFCEPEDIQDPLC
DELCRTVIAAAVLFSFIVSVLLSAFCIHCYHKFAH
KPPISSAEMTFRRPAQAFPVSYSSSGARRPSLDSM
ENQVSVDAFKILEDPKWEFPRKNLVLGKTLGEGEF
GKVVKATAFHLKGRAGYTTVAVKMLKENASPSELR
DLLSEFNVLKQVNHPHVIKLYGACSQDGPLLLIVE
YAKYGSLRGFLRESRKVGPGYLGSGGSRNSSSLDH
PDERALTMGDLISFAWQISQGMQYLAEMKLVHRDL
AARNILVAEGRKMKISDFGLSRDVYEEDSYVKRSQ
GRIPVKWMAIESLFDHIYTTQSDVWSFGVLLWEIV
TLGGNPYPGIPPERLFNLLKTGHRMERPDNCSEEM
YRLMLQCWKQEPDKRPVFADISKDLEKMMVKRRDY
LDLAASTPSDSLIYDDGLSEEETPLVDCNNAPLPR
ALPSTWIENKLYGRISHAFTRF

B. Anti-GFRAL Antibodies that Bind to a GFRAL Protein

The present disclosure provides antibodies that bind to GFRAL (e.g., human GFRAL) (also referred to herein as “anti-GFRAL antibodies”) and may be used in the methods of treating described herein. In some instances, an anti-GFRAL antibody described herein inhibits GFRAL binding to RET. Methods of determining whether an anti-GFRAL antibody inhibits GFRAL binding to RET are known in the art, such as, e.g., co-immunoprecipitation, immunohistochemistry, and ELISA (see, e.g., International Patent Application Publication No. WO 2017/172260, which is incorporated by reference herein in its entirety).

In some instances, an anti-GFRAL antibody comprises a VH comprising a VH CDR1, a VH CDR2, a VH CDR3, and a VL comprising a VL CDR1, a VL CDR2, and a VL CDR3, wherein the VH CDR1, VH CDR2, VH CDR3, VL CDR1, VL CDR2 and VL CDR3 are from any one of the VH and VL sequences of the antibodies described herein (e.g., 3P10, 5F12, 2I23, 6N16, 1B3, 6G9, or 2B11), such as the amino acid sequences depicted in Tables 1-7. In some instances, the anti-GFRAL antibody is a humanized version of an antibody described herein, (e.g., 3P10, 5F12, 2I23, 6N16, 1B3, 6G9, or 2B11). The antibody designated 3P10 comprises a VH sequence that is set forth in SEQ ID NO: 3 and a VL sequence that is set forth in SEQ ID NO: 4 (see Table 1). In some instances, a humanized 3P10 antibody comprises a VH comprising a VH CDR1, a VH CDR2, a VH CDR3, and a VL comprising a VL CDR1, a VL CDR2, and a VL CDR3, wherein the VH CDR1, VH CDR2, and VH CDR3 are from the amino acid sequence of SEQ ID NO:3, and the VL CDR1, VL CDR2 and VL CDR3 are from the amino acid sequence of SEQ ID NO:4. The antibody designated 5F12 comprises a VH sequence that is set forth in SEQ ID NO: 7 and a VL sequence that is set forth in SEQ ID NO: 8 (see Table 2). In some instances, a humanized 5F12 comprises a VH comprising a VH CDR1, a VH CDR2, a VH CDR3, and a VL comprising a VL CDR1, a VL CDR2, and a VL CDR3, wherein the VH CDR1, VH CDR2, and VH CDR3 are from the amino acid sequence of SEQ ID NO:7, and the VL CDR1, VL CDR2 and VL CDR3 are from the amino acid sequence of SEQ ID NO:8 The antibody designated 2I23 comprises a VH sequence that is set forth in SEQ ID NO: 21 and a VL sequence that is set forth in SEQ ID NO: 22 (see Table 3). In some instances, a humanized 2I23 comprises a VH comprising a VH CDR1, a VH CDR2, a VH CDR3, and a VL comprising a VL CDR1, a VL CDR2, and a VL CDR3, wherein the VH CDR1. VH CDR2, and VH CDR3 are from the amino acid sequence of SEQ ID NO:21, and the VL CDR1, VL CDR2 and VL CDR3 are from the amino acid sequence of SEQ ID NO:22. The antibody designated 6N16 comprises a VH sequence that is set forth in SEQ ID NO: 23 and a VL sequence that is set forth in SEQ ID NO: 24 (see Table 4). In some instances, a humanized 6N16 antibody comprises a VH comprising a VH CDR1, a VH CDR2, a VH CDR3, and a VL comprising a VL CDR1, a VL CDR2, and a VL CDR3, wherein the VH CDR1, VH CDR2, and VH CDR3 are from the amino acid sequence of SEQ ID NO:23, and the VL CDR1, VL CDR2 and VL CDR3 are from the amino acid sequence of SEQ ID NO:24. The antibody designated 1B3 comprises a VH sequence that is set forth in SEQ ID NO: 25 and a VL sequence that is set forth in SEQ ID NO: 26 (see Table 5). In some instances, a humanized 1B3 antibody comprises a VH comprising a VH CDR1, a VH CDR2, a VH CDR3, and a VL comprising a VL CDR1, a VL CDR2, and a VL CDR3, wherein the VH CDR1, VH CDR2, and VH CDR3 are from the amino acid sequence of SEQ ID NO:25, and the VL CDR1, VL CDR2 and VL CDR3 are from the amino acid sequence of SEQ ID NO:26. The antibody designated 609 comprises a VH sequence that is set forth in SEQ ID NO: 37 and a VL sequence that is set forth in SEQ ID NO: 38 (see Table 6). In some instances, a humanized 6G9 antibody comprises a VH comprising a VH CDR1, a VH CDR2, a VH CDR3, and a VL comprising a VL CDR1, a VL CDR2, and a VL CDR3, wherein the VH CDR1, VH CDR2, and VH CDR3 are from the amino acid sequence of SEQ ID NO:37, and the VL CDR1, VL CDR2 and VL CDR3 are from the amino acid sequence of SEQ ID NO:38. The antibody designated 2B11 comprises a VH sequence that is set forth in SEQ ID NO: 39 and a VL sequence that is set forth in SEQ ID NO: 40 (see Table 7). In some instances, a humanized 2B11 antibody comprises a VH comprising a VH CDR1, a VH CDR2, a VH CDR3, and a VL comprising a VL CDR1, a VL CDR2, and a VL CDR3, wherein the VH CDR1, VH CDR2, and VH CDR3 are from the amino acid sequence of SEQ ID NO:39, and the VL CDR1, VL CDR2 and VL CDR3 are from the amino acid sequence of SEQ ID NO:40.

TABLE 1
Antibody 3P10 CDR Sequences

| | | Exemplary | IMGT | Kabat | Chothia | Contact | AbM |
|---|---|---|---|---|---|---|---|
| VH CDR | VHCDR1 | GYTFTDY | GYTFTDY | DYGVI | GYTFTDY | TDYGVI | GYTFT |
| Seq. | | GVI | G | (SEQ ID | (SEQ ID | (SEQ ID | DYGVI |
| | | (SEQ ID | (SEQ ID | NO: 48) | NO: 49) | NO: 50) | (SEQ ID |
| | | NO: 46) | NO: 47) | | | | NO: 46) |
| | VH CDR2 | WINTYTG | INTYTGE | WINTYTG | TYTG | WMGWINT | WINTY |
| | | EPTYAD | P | EPTYAD | (SEQ ID | YTGEP | TGEPT |
| | | DLKG | (SEQ ID | DLKG | NO: 139) | T | (SEQ ID |
| | | (SEQ ID | NO: 138) | (SEQ ID | | (SEQ ID | NO: 141) |
| | | NO: 137) | | NO: 137) | | NO: 140) | |
| | VH CDR3 | RYGPEDI | ARRYGPE | RYGPEDI | YGPEDID | ARRYGPE | RYGPE |
| | | DY | DIDY | DY | (SEQ ID | DID | DIDY |
| | | (SEQ ID | (SEQ ID | (SEQ ID | NO: 227) | (SEQ ID | (SEQ ID |
| | | NO: 225) | NO: 226) | NO: 225) | | NO: 228) | NO: 225) |
| VL CDR | VL CDR1 | RASESVD | ESVDNYG | RASESVD | SESVDNY | DNYGISF | RASESV |
| Seq. | | NYGISF | ISF | NYGISF | GISF | MSWF | DNYGIS |
| | | MS | (SEQ ID | MS | (SEQ ID | (SEQ IDF | MS |
| | | (SEQ ID | NO: 302) | (SEQ ID | NO: 303) | NO: 304) | (SEQ ID |
| | | NO: 301) | | NO: 301) | | | NO: 301) |
| | VL CDR2 | AASHQGS | AAS | AASHQGS | AAS | LLIYAAS | AASHQGS |
| | | (SEQ ID | (SEQ ID | (SEQ ID | (SEQ ID | HQG | |
| | | NO: 376) | NO: 377) | NO: 376) | NO: 377) | (SEQ ID | (SEQ ID |
| | | | | | | NO: 378) | NO: 376) |
| | VL CDR3 | LQSKEVP | LQSKEVP | LQSKEVP | SKEVP | LQSKEVP | LQSKE |
| | | WT | WT | WT | W | W | VPWT |
| | | (SEQ ID | (SEQ ID | (SEQ ID | (SEQ ID | (SEQ ID | (SEQ ID |
| | | NO: 426) | NO: 426) | NO: 426) | NO: 427) | NO: 428) | NO: 426) |

VH Sequence:
QIQLVQSGPELKKPGETVKISCKASGYTFTDYGVIWVKQAPGKALKWMGWINTYT
GEPTYADDLKGRFAFSLETSASSASLQINNLKNEDTATYPCA
RRYGPEDIDYWGQGTTLTVSS (SEQ ID NO: 3)
VL Sequence:
DIVLTQSPVSLAVSLGQRATISCRASESVDNYGISFMSWFQQKPGQPPKLLIYAA
SHQGSGVPARFSGSGSGTDFSLNIHPMEEDDSAMYFCLQSKEVP
WTFGGGTKLEIK (SEQ ID NO: 4)

TABLE 1A
Hz3P10 VH, VL, heavy chain (HC), and
light chain (LC) Sequences

| | |
|---|---|
| VH | QVQLVQSGAEVKKPGSSVKVSCKAS |
| | GYTFTDYGVIWVRQAPGQGLEWMGW |
| | INTYTGEPTYADDLKGRVTFTADES |
| | TSTAYMELSSLRSEDTAVYYCARRY |
| | GPEDIDYWGQGTTVTVSS |
| | (SEQ ID NO: 1982) |
| VL | DVVLTQSPLSLPVTLGQPASISCRA |
| | SESVDNYGISFMSWFQQRPGQSPRL |
| | LIYAASHQGSGVPDRFSGSGSGIDF |
| | ILKISRVEAEDVGVYFCLQSKEVPW |
| | TFGGGTKVEIK |
| | (SEQ ID NO: 1997) |
| HC (with signal | MDMRVPAQLLGLLLLWLRGARCQVQ |
| sequence in | LVQSGAEVKKPGSSVKVSCKASGYT |
| bold; VH + heavy | FTDYGVIWVRQAPGQGLEWMGWINT |
| chain | YTGEPTYADDLKGRVTFTADEST |
| constant region) | STAYMELSSLRSEDTAVYYCARRYG |
| | PEDIDYWGQGTTVTVSSASTKGPSV |
| | FPLAPSSKSTSGGTAALGCLVKDYF |
| | PEPVTVSWNSGALTSGVHTFPAVLQ |
| | SSGLYSLSSVVTVPSSSLGTQTYIC |
| | NVNHKPSNTKVDKKVEPKSCDKTHT |
| | CPPCPAPALAGGPSVFLFPPKPKDT |
| | LMISRTPEVTCVVVDVSHEDPEVKF |
| | NWYVDGVEVHNAKTKPREEQYNSTY |
| | RVVSVLTVLHQDWLNGKEYKCKVSN |
| | KALPAPIEKTISKAKGQPREPQVYT |
| | LPPSREEMTKNQVSLTCLVKGFYPS |
| | DIAVEWESNGQPENNYKTTPPVLDS |
| | DGSFFLYSKLTVDKSRWQQGNVFSC |
| | SVMHEALHNHYTQKSLSLSPGK |
| | (SEQ ID NO: 2009) |
| HC (without | QVQLVQSGAEVKKPGSSVKVSCKAS |
| signal sequence; | GYIHDYGVIWVRQAPGQGLEWMGWI |
| VH + heavy | NTYTGEPTYADDLKGRVTFTADEST |
| chain constant | STAYMELSSLRSEDTAVYYCARRYG |
| region) | PEDIDYWGQGTTVTVSSASTKGPSV |
| | FPLAPSSKSTSGGTAALGCLVKDYF |
| | PEPVTVSWNSGALTSGVHTFPAVLQ |
| | SSGLYSLSSVVTVPSSSLGTQTYIC |
| | NVNHKPSNTKVDKKVEPKSCDKTHT |
| | CPPCPAPALAGGPSVFLFPPKPKDT |
| | LMISRTPEVTCVVVDVSHEDPEVKF |
| | NWYVDGVEVHNAKTKPREEQYNSTY |
| | RVVSVLTVLHQDWLNGKEYKCKVSN |
| | KALPAPIEKTISKAKGQPREPQVYT |
| | LPPSREEMTKNQVSLTCLVKGFYPS |
| | DIAVEWESNGQPENNYKTTPPVLDS |
| | DGSFFLYSKLTVDKSRWQQGNVFSC |
| | SVMHEALHNHYTQKSLSLSPGK |
| | (SEQ ID NO: 2010) |
| LC (with signal | MDMRVPAQLLGLLLLWLRGARCDVV |
| sequence in | LTQSPLSLPVTLGQPASISCRASES |
| bold; VL + kappa | VDNYGISFMSWFQQRPGQSPRLLIY |
| light chain | AASHQGSGVPDRFSGSGSGTDFTLK |
| constant region) | ISRVEAEDVGVYFCLQSKEVPWTFG |
| | GGTKVEIKRTVAAPSVFIFPPSDEQ |
| | LKSGTASVVCLLNNFYPREAKVQWK |
| | VDNALQSGNSQESVTEQDSKDSTYS |
| | LSSTLTLSKADYEKHKVYACEVTHQ |
| | GLSSPVTKSFNRGEC |
| | (SEQ ID NO: 2011) |
| LC (without signal | DVVLTQSPLSLPVTLGQPASISCRA |
| sequence; | SESVDNYGISFMSWFQQRPGQSPRL |
| VL + kappa light | LIYAASHQGSGVPDRFSGSGSGTDF |
| chain | TLKISRVEAEDVGVYFCLQSKEVPW |
| constant region) | TFGGGTKVEIKRTVAAPSVFIFPPS |
| | DEQLKSGTASVVCLLNNFYPREAKV |
| | QWKVDNALQSGNSQESVTEQDSKDS |
| | TYSLSSTLTLSKADYEKHKVYACEV |
| | THQGLSSPVTKSFNRGEC |
| | (SEQ ID NO: 2012) |

TABLE 2
Antibody 5F12 CDR Sequences

| | | Exemplary | IMGT | Kabat | Chothia | Contact | AbM |
|---|---|---|---|---|---|---|---|
| VH CDR | VH CDR1 | GYTFTDY | GYTFTDY | DYYIN | GYTFTDY | TDYYIN | GYTFT |
| Seq. | | YIN | Y | (SEQ ID | (SEQ ID | (SEQ ID | DYYIN |
| | | (SEQ ID | (SEQ ID | NO: 58) | NO: 49) | NO: 59) | (SEQ ID |
| | | NO: 56) | NO: 57) | | | | NO: 56) |
| | VH CDR2 | RIYPGNG | IYPGNGN | RIYPGNG | PGNG | WIARIYP | RIYPG |
| | | NTYHNE | T | NTYHNE | (SEQ ID | GNGNTY | NGNT |
| | | KFKG | (SEQ ID | KFKG | NO: 149) | (SEQ ID | Y |
| | | (SEQ ID | NO: 148) | (SEQ ID | | NO: 150) | (SEQ ID |
| | | NO: 147) | | NO: 147) | | | NO: 151) |
| | VH CDR3 | EGLYYDY | AREGLYY | EGLYYDY | GLYYDYD | AREGLYY | EGLYYDY |
| | | DRYFD | DYDRY | DRYFD | RYFD | DYDRY | DRYFD |
| | | Y | FDY | Y | (SEQ ID | FD | Y |
| | | (SEQ ID | (SEQ ID | (SEQ ID | NO: 235) | (SEQ ID | (SEQ ID |
| | | NO: 233) | NO: 234) | NO: 233) | | NO: 236) | NO: 233) |
| VL CDR | VL CDR1 | RASESVD | ESVDTYG | RASESVD | SESVDTY | DTYGNSF | RASESVD |
| Seq. | | TYGNSF | NSF | TYGNSF | GNSF | MHWY | TYGNSF |
| | | MH | (SEQ ID | MH | (SEQ ID | (SEQ ID | MH |
| | | (SEQ ID | NO: 310) | (SEQ ID | NO: 311) | NO: 312) | (SEQ ID |
| | | NO: 309) | | NO: 309) | | | NO: 309) |
| | VL CDR2 | LASNLES | LAS | LASNLES | LAS | LLIYLAS | LASNLES |
| | | (SEQ ID | (SEQ ID | (SEQ ID | (SEQ ID | NLE | (SEQ ID |
| | | NO: 382) | NO: 383) | NO: 382) | NO: 383) | (SEQ ID | NO: 382) |
| | | | | | | NO: 384) | |
| | VL CDR3 | HQNNEDP | HQNNEDP | HQNNEDP | NNEDPP | HQNNEDP | HQNNEDP |
| | | PA | PA | PA | (SEQ ID | P | PA |
| | | (SEQ ID | (SEQ ID | (SEQ ID | NO: 433) | (SEQ ID | (SEQ ID |
| | | NO: 432) | NO: 432) | NO: 432) | | NO: 434) | NO: 432) |

VH Sequence:
QVQLKQSGTELVRPGASVKLSCKASGYTFTDYYINWVKQRPGQG
LEWIARIYPUNGNTYHNEKFKGKATLTAEKSSSTAYMQLSSLTS
EDSAVYFCAREGLYYDYDRYFDYWGQGTALTVSS
(SEQ ID NO: 7)
VL Sequence:
NIVLTQSPASLAVSLGQRATISCRASESVDTYGNSFMHWYQQKP
GQPPKLLIYLASNLESGVPARFSGSGSRTDFTLTIDPVEADDAA
TYYCHQNNEDPPAFGGGTKLEIK (SEQ ID NO: 8)

TABLE 3
Antibody 2123 CDR Sequences

| | | Exemplary | IMGT | Kabat | Chothia | Contact | AbM |
|---|---|---|---|---|---|---|---|
| VH | VH | GYSFTSY | GYSFTSY | SYNID | GYSFTSY | TSYNID | GYSFTSY |
| CDR | CDR1 | NID | N | (SEQ ID | (SEQ ID | (SEQ ID | NID |
| Seq. | | (SEQ ID | (SEQ ID | NO: 88) | NO: 89) | NO: 90) | (SEQ ID |
| | | NO: 86) | NO: 87) | | | | NO: 86) |
| | VH | WIFPGDG | IFPGDGS | WIFPGDG | PGDG | WIGWIFP | WIFPGDG |
| | CDR2 | ST | T | STKYNE | (SEQ ID | GDGSTK | STK |
| | | (SEQ ID | (SEQ ID | KFKG | NO: 168) | (SEQ ID | (SEQ ID |
| | | NO: 177) | NO: 178) | (SEQ ID | | NO: 180) | NO: 181) |
| | | | | NO: 179) | | | |
| | VH | SGIYYGS | ARSGIYY | SGIYYGS | GIYYGSH | ARSGIYY | SGIYYGS |
| | CDR3 | HFVY | GSHFVY | HFVY | FV | GSHFV | HFVY |
| | | (SEQ ID | (SEQ ID | (SEQ ID | (SEQ ID | (SEQ ID | (SEQ ID |
| | | NO: 261) | NO: 262) | NO: 261) | NO: 263) | NO: 264) | NO: 261) |
| VL | VL | RSSQSLL | QSLLDSD | RSSQSLL | SQSLLDS | LDSDGKT | RSSQSLL |
| CDR | CDR1 | DSDGKT | GKTY | DSDGKT | DGKTY | YLNWL | DSDGKT |
| Seq. | | YEN | (SEQ ID | YEN | (SEQ ID | (SEQ ID | YEN |
| | | (SEQ ID | NO: 338) | (SEQ ID | NO: 339) | NO: 340) | (SEQ ID |
| | | NO: 337) | | NO: 337) | | | NO: 337) |
| | VL | LVSKVDS | LVS | LVSKVDS | LVS | RLIYLVS | LVSKVDS |
| | CDR2 | (SEQ ID | (SEQ ID | (SEQ ID | (SEQ ID | KVD | (SEQ ID |
| | | NO: 401) | NO: 386) | NO: 401) | NO: 386) | (SEQ ID | NO: 401) |
| | | | | | | NO: 402) | |
| | VL | WQGTHFP | WQGTHFP | WQGTHFP | GTHFPL | WQGTHFP | WQGTHFP |
| | CDR3 | LT | LT | LT | (SEQ ID | L | LT |
| | | (SEQ ID | (SEQ ID | (SEQ ID | NO: 454) | (SEQ ID | (SEQ ID |
| | | NO: 453) | NO: 453) | NO: 453) | | NO: 455) | NO: 453) |

VH Sequence:
QAQLQQSGAELVKPGASVKLSCKASGYSFTSYNIDWVRQRPEQGLE
WIGWIFPGDGSTKYNEKFKGQATLTTDKSSSTTYIHLSRLTSEDSA
VYFCARSGIYYGSHFVYWGQGTLVTVSA (SEQ ID NO: 21)
VL Sequence:
DVVMTQTPLTLSVTIGQSASISCRSSQSLLDSDGKTYLNWLLQRPG
QSPKRLIYLVSKVDSGVPDRFTGSGSGTDFTLKISRVEAEDLGVYF
CWQGTHFPLTFGAGTKLELK (SEQ ID NO: 22)

TABLE 4
Antibody 6N16 CDR Sequences

| | | Exemplary | IMGT | Kabat | Chethla | Contact | AbM |
|---|---|---|---|---|---|---|---|
| VH CDR | VH CDR1 | GYTFTSY | GYTFTSY | SYNIN | GYTPTSY | TSY | GYTFTSY |
| Seq. | | NIN | N | (SEQ ID | (SEQ ID | NIN | NIN |
| | | (SEQ ID | (SEQ ID | NO: 93) | NO: 75) | (SEQ ID | (SEQ ID |
| | | NO: 91) | NO: 92) | | | NO: 94) | NO: 91) |
| | VH CDR2 | WIFPGDD | IFPGDDS | WIFPGDD | PGDD | WIGWIFP | WIFPGDD |
| | | SIKYNE | I | SIKYNE | (SEQ ID | GDDSIK | SIK |
| | | NFRG | (SEQ ID | NFRG | NO: 184) | (SEQ ID | (SEQ ID |
| | | (SEQ ID | NO: 183) | (SEQ ID | | NO: 185) | NO: 186) |
| | | NO: 182) | | NO: 182) | | | |
| | VH CDR3 | SGIFY | ARSGIFY | SGIFYGN | GIYGN | ARSGIFY | SGIFY |
| | | GNNFAY | GNNFAY | NFAY | NFA | GNNFA | GNNFAY |
| | | (SEQ ID | (SEQ ID | (SEQ ID | (SEQ ID | (SEQ ID | (SEQ ID |
| | | NO: 265) | NO: 266) | NO: 265) | NO: 267) | NO: 268) | NO: 265) |
| VL CDR | VL CDR1 | KSSQSLL | QSLLDGD | KSSQSLL | SQSLLDG | LDGDGET | KSSQSLL |
| Seq. | | DGDGET | GETY | DGDGET | DGETY | YLSWL | DGDGET |
| | | YLS | (SEQ ID | YLS | (SEQ ID | (SEQ ID | YLS |
| | | (SEQ ID | NO: 342) | (SEQ ID | NO: 343) | NO: 344) | (SEQ ID |
| | | NO: 341) | | NO: 341) | | | NO: 341) |
| | VL CDR2 | LVSKLDS | LVS | LVSKLDS | LVS | RLIYLVS | LVSKLDS |
| | | (SEQ ID | (SEQ ID | (SEQ ID | (SEQ ID | KLD | (SEQ ID |
| | | NO: 385) | NO: 386) | NO: 385) | NO: 386) | (SEQ ID | NO: 385) |
| | | | | | | NO: 403) | |
| | VL CDR3 | CQSTHFP | CQSTHFP | CQSTHFP | STHFPL | CQSTHFP | CQSTHFP |
| | | LT | LT | LT | (SEQ ID | L | LT |
| | | (SEQ ID | (SEQ ID | (SEQ ID | NO: 457) | (SEQ ID | (SEQ ID |
| | | NO: 456) | NO: 456) | NO: 456) | | NO: 458) | NO: 456) |

VH Sequence:
SELVKPGTSMKLSCKASGYTF TSYNINWVRLRPEQGLEWIGW
IFPGDDSIKYNENFRGKATLTTDKSSSTAYMHLSRLTSDDSAV
YFCAQVQLQQSGFAYWGQGTLVTVSA (SEQ ID NO: 23)
RSGIFYGNN
VL Sequence:
DVVMTQAPLILSVTIGQPASISCKSSQSLLDGDGETYLSWLLQ
RPGQSPKRLIYLVSKLDSGVPDRFTGSGSGTDFTLKISRVEAE
DLGVYYCCQSTHFPLTFGAGTKLELK
(SEQ ID NO: 24)

TABLE 5
Antibody 1B3 CDR Sequences

| | | Exemplary | IMGT | Kabat | Chothia | Contact | AbM |
|---|---|---|---|---|---|---|---|
| VH CDR | VH CDR1 | GFTFTGY | GFTFTGY | GYNIN | GFTFTGY | TGYNIN | GFTFTGY |
| Seq. | | NIN | N | (SEQ ID | (SEQ ID | (SEQ ID | NIN |
| | | (SEQ ID | (SEQ ID | NO: 97) | NO: 98) | NO: 99) | (SEQ ID |
| | | NO: 95) | NO: 96) | | | | NO: 95) |
| | VH CDR2 | WIFPGDD | IFPGDDN | WIFPGDD | PGDD | WIGWIFP | WIFPGDD |
| | | NAKYNE | A | NAKYNE | (SEQ ID | GDDNAK | NAK |
| | | KFKG | (SEQ ID | KFKG | NO: 184) | (SEQ ID | (SEQ ID |
| | | (SEQ ID | NO: 188) | (SEQ ID | | NO: 189) | NO: 190) |
| | | NO: 187) | | NO: 187) | | | |
| | VH CDR3 | TPVLSNY | ARTPVLS | TPVLSNY | PVLSNYF | ARTPVLS | TPVLSNY |
| | | FDY | NYFDY | FDY | D | NYFD | FDY |
| | | (SEQ ID | (SEQ ID | (SEQ ID | (SEQ ID | (SEQ ID | (SEQ ID |
| | | NO: 269) | NO: 270) | NO: 269) | NO: 271) | NO: 272) | NO: 269) |
| VL CDR | VL CDR1 | KASQDIS | QDISKY | KASQDIS | SQDISKY | SKYISWY | KASQDIS |
| Seq. | | KYIS | (SEQ ID | KYIS | (SEQ ID | (SEQ ID | KYIS |
| | | (SEQ ID | NO: 346) | (SEQ ID | NO: 347) | NO: 348) | (SEQ ID |
| | | NO: 345) | | NO: 345) | | | NO: 345) |
| | VL CDR2 | YTSTLQP | YTS | YTSTLQP | YTS | LLIHYTS | YTSTLQP |
| | | (SEQ ID | (SEQ ID | (SEQ ID | (SEQ ID | TLQ | (SEQ ID |
| | | NO: 404) | NO: 405) | NO: 404) | NO: 405) | (SEQ ID | NO: 404) |
| | | | | | | NO: 406) | |
| | VL CDR3 | LQYDNLY | LQYDNLY | LQYDNLY | YDNLY | LQYDNLY | LQYDNLY |
| | | T | T | T | (SEQ ID | (SEQ ID | T |
| | | (SEQ ID | (SEQ ID | (SEQ ID | NO: 460) | NO: 461) | (SEQ ID |
| | | NO: 459) | NO: 459) | NO: 459) | | | NO: 459) |

VH Sequence:
QVHLQQPGAELVKPGASVKLSCKASGFTFTGYNINWVRLRPEQGLEWI
GWIFPGDDNAKYNEKFKGKATLTTDKSSNTAYMQLSRLTSEDSAVYFC
ARTPVLSNYFDYWGQGTTLTVSS (SEQ ID NO: 25)
VL Sequence:
DIQMTQSPSSLSASLGGKVTITCKASQDISKYISWYQHKPGKSPRLLI
HYTSTLQPGIPSRFSGSGSGRDYSFSISNLEPEDIATYYCLQYDNLYT
FGGGTKLEIK (SEQ ID NO: 26)

TABLE 6
Antibody 6G9 CDR Sequences

| | | Exemplary | IMGT | Kabat | Chothia | Contact | AbM |
|---|---|---|---|---|---|---|---|
| VH CDR | VH CDR1 | GYTFTSY | GYTFTSY | SYWMQ | GYTFTSY | TSYWMQ | GYTFTSY |
| Seq. | | WMQ | W | (SEQ ID | (SEQ ID | (SEQ ID | WMQ |
| | | (SEQ ID | (SEQ ID | NO: 125) | NO: 75) | NO: 126) | (SEQ ID |
| | | NO: 124) | NO: 73) | | | | NO: 124) |
| | VH CDR2 | EIDPSDS | IDPSDSY | EIDPSDS | PSDS | WIGEIDP | EIDPSDS |
| | | YTNYNQ | T | YTNYNQ | (SEQ ID | SDSYTN | YTN |
| | | KFKG | (SEQ ID | KFKG | NO: 212) | (SEQ ID | (SEQ ID |
| | | (SEQ ID | NO: 211) | (SEQ ID | | NO: 213) | NO: 214) |
| | | NO: 210) | | NO: 210) | | | |
| | VH CDR3 | PLDRSAY | ARPLDRS | PLDRSAY | LDRSAYY | ARPLDRS | PLDRSAY |
| | | Y IDY | AYYFDY | YFDY | FD | AYYFD | YFDY |
| | | (SEQ ID | (SEQ ID | (SEQ ID | (SEQ ID | (SEQ ID | (SEQ ID |
| | | NO: 289) | NO: 290) | NO: 289) | NO: 291) | NO: 292) | NO: 289) |
| VL CDR | VL CDR1 | RASES | ESVDFS | RASESVD | SESVDFS | DFSGNSF | RASESVD |
| Seq. | | VDFSG | GNS | FSGNSF | GNSF | MHWY | FSGNSF |
| | | NSF | F | MH | (SEQ ID | (SEQ ID | MH |
| | | MH | (SEQ ID | (SEQ ID | NO: 367) | NO: 368) | (SEQ ID |
| | | (SEQ ID | NO: 366) | NO: 365) | | | NO: 365) |
| | | NO: 365) | | | | | |
| | VL CDR2 | RASNLDS | RAS | RASNLDS | RAS | LLIYRAS | RASNLDS |
| | | (SEQ ID | (SEQ ID | (SEQ ID | (SEQ ID | NLD | (SEQ ID |
| | | NO: 418) | NO: 419) | NO: 418) | NO: 419) | (SEQ ID | NO: 418) |
| | | | | | | NO: 420) | |
| | VL CDR3 | QQSNEDP | QQSNEDP | QQSNEDP | SNEDPY | QQSNEDP | QQSNEDP |
| | | YT | YT | YT | (SEQ ID | Y | YT |
| | | (SEQ ID | (SEQ ID | (SEQ ID | NO: 475) | (SEQ ID | (SEQ ID |
| | | NO: 474) | NO: 474) | NO: 474) | | NO: 476) | NO: 474) |

VH Sequence:
QVQLHQPGAELVKPGASVKLSCKTSGYTFTSYWMQWVKQRPGQGLEWI
GEIDPSDSYTNYNQKFKGKATLTVDTSSTTAYMQLSSLTSEDSAVYYC
ARPLDRSAYYFDYWGQGTTLTVSS (SEQ ID NO: 37)
VL Sequence:
DIVLTQSPASLAVSLGQRATISCRASESVDFSGNSFMHWYQQKPGQPP
KLLIYRASNLDSGIPARFSGVGSRTDFTLTINPVEADDVATYYCQQSN
EDPYTFGGGTKLEIE (SEQ ID NO: 38)

TABLE 7
Antibody 2B11 CDR Sequences

| | | Exemplary | IMGT | Kabat | Chothia | Contact | AbM |
|---|---|---|---|---|---|---|---|
| VH CDR | VH CDR1 | GYSITSG | GYSITSG | SGYYW | GYSITSG | TSGY | GYSITSG |
| Seq. | | YYWN | YY | N | Y | YWN | YYWN |
| | | (SEQ ID | (SEQ ID | (SEQ ID | (SEQ ID | (SEQ ID | (SEQ ID |
| | | NO: 127) | NO: 128) | NO: 129) | NO: 130) | NO: 131) | NO: 127) |
| | VH CDR2 | HIANDGS | IANDGSN | HIANDGS | NDG | WMGHIAN | HIANDGS |
| | | NYYNPF | (SEQ ID | NYYNPF | (SEQ ID | DGSNY | NY |
| | | LKH | NO: 216) | LKH | NO: 217) | (SEQ ID | (SEQ ID |
| | | (SEQ ID | | (SEQ ID | | NO: 218) | NO: 219) |
| | | NO: 215) | | NO: 215) | | | |
| | VH CDR3 | GGSYFDY | ARGGSYF | GGSYFDY | GSYFDYV | ARGGSYF | GGSYFDY |
| | | VDY | DYVDY | VDY | D | DYVD | VDY |
| | | (SEQ ID | (SEQ ID | (SEQ ID | (SEQ ID | (SEQ ID | (SEQ ID |
| | | NO: 293) | NO: 294) | NO: 293) | NO: 295) | NO: 296) | NO: 293) |
| VL CDR | VL CDR1 | RASQDIS | QDISNY | RASQDIS | SQDISNY | SNYLNWY | RASQDIS |
| Seq. | | NYLN | (SEQ ID | NYLN | (SEQ ID | (SEQ ID | NYLN |
| | | (SEQ ID | NO: 370) | (SEQ ID | NO: 371) | NO: 372) | (SEQ ID |
| | | NO: 369) | | NO: 369) | | | NO: 369) |
| | VL CDR2 | YTSRLHS | YTS | YTSRLHS | YTS | LLIYYTS | YTSRLHS |
| | | (SEQ ID | (SEQ ID | (SEQ ID | (SEQ ID | RLH | (SEQ ID |
| | | NO: 421) | NO: 405) | NO: 421) | NO: 405) | (SEQ ID | NO: 421) |
| | | | | | | NO: 422) | |
| | VL CDR3 | QQGNTLP | QQGNTLP | QQGNTLP | GNTLPF | QQGNTLP | QQGNTLP |
| | | FT | FT | FT | (SEQ ID | F | FT |
| | | (SEQ ID | (SEQ ID | (SEQ ID | NO: 478) | (SEQ ID | (SEQ ID |
| | | NO: 477) | NO: 477) | NO: 477) | | NO: 479) | NO: 477) |

VH Sequence:
DVQLQESGPGLVKPSQSLSLTCSVTGYSITSGYYWNWIRQFPGNKL
EWMGHIANDGSNYYNPFLKHRVSITRDTSKNQFFLKLNSVTIQDTA
TYYCARGGSYFDYVDYWGQGTTLTVSS (SEQ ID NO: 39)
VL Sequence:
DIQMTQTTSSLSASLGDRVTINCRASQDISNYLNWYQQKPDGTVKL
LIYYTSRLHSGVPSRFSGSGSGTDYSLTITNLEQEDIATYFCQQGN
TLPFTFGSGTKLEIK (SEQ ID NO: 40)

CDRs of an antibody are defined using a variety of methods/systems by those skilled in the art. These systems and/or definitions have been developed and refined over a number of years and include Kabat, Chothia, IMGT, AbM, and Contact. The Kabat definition is based on sequence variability and is commonly used. The Chothia definition is based on the location of the structural loop regions. The IMGT system is based on sequence variability and location within the structure of the variable domain. The AbM definition is a compromise between Kabat and Chothia. The Contact definition is based on analyses of the available antibody crystal structures. An Exemplary system is a combination of Kabat and Chothia. Software programs (e.g., abYsis) are available and known to those of skill in the art for analysis of antibody sequences and determination of CDRs. It will be understood that reference in this disclosure to a VH CDR or VH CDRs and/or a VL CDR or VL CDRs of a specific antibody, including a specific VH amino acid sequence and/or a VL amino acid sequence, will encompass all CDR definitions as known to those of skill in the art.

In some instances, the anti-GFRAL antibody comprises (i) a VH comprising a VH CDR1, a VH CDR2, and a VH CDR3 defined by any one of the CDR definitions from Table 1; and (ii) a VL comprising a VL CDR1, a VL CDR2, and a VL CDR3 defined by any one of the CDR definitions from Table 1.

In some instances, the anti-GFRAL antibody comprises (i) a VH comprising a VH CDR1, a VH CDR2, and a VH CDR3 defined by any one of the CDR definitions from Table 2; and (ii) a VL comprising a VL CDR1, a VL CDR2, and a VL CDR3 defined by any one of the CDR definitions from Table 2.

In some instances, the anti-GFRAL antibody comprises (i) a VH comprising a VH CDR1, a VH CDR2, and a VH CDR3 defined by any one of the CDR definitions from Table 3; and (ii) a VL comprising a VL CDR1, a VL CDR2, and a VL CDR3 defined by any one of the CDR definitions from Table 3.

In some instances, the anti-GFRAL antibody comprises (i) a VH comprising a VH CDR1, a VH CDR2, and a VH CDR3 defined by any one of the CDR definitions from Table 4; and (ii) a VL comprising a VL CDR1, a VL CDR2, and a VL CDR3 defined by any one of the CDR definitions from Table 4.

In some instances, the anti-GFRAL antibody comprises (i) a VH comprising a VH CDR1, a VH CDR2, and a VH CDR3 defined by any one of the CDR definitions from Table 5; and (ii) a VL comprising a VL CDR1, a VL CDR2, and a VL CDR3 defined by any one of the CDR definitions from Table 5.

In some instances, the anti-GFRAL antibody comprises (i) a VH comprising a VH CDR1, a VH CDR2, and a VH CDR3 defined by any one of the CDR definitions from Table 6; and (ii) a VL comprising a VL CDR1, a VL CDR2, and a VL CDR3 defined by any one of the CDR definitions from Table 6.

In some instances, the anti-GFRAL antibody comprises (i) a VH comprising a VH CDR1, a VH CDR2, and a VH CDR3 defined by any one of the CDR definitions from Table 7; and (ii) a VL comprising a VL CDR1, a VL CDR2, and a VL CDR3 defined by any one of the CDR definitions from Table 7.

In some instances, the anti-GFRAL antibody comprises (i) a VH comprising the VH CDR1, the VH CDR2, the VH CDR3 from the amino acid sequence set forth in SEQ ID NO:3; and (ii) a VL comprising the VL CDR1, the VL CDR2, and the VL CDR3 from the amino acid sequence set forth in SEQ ID NO:4 (i.e., the six CDRs of antibody 3P10 of Table 1). In some instances, the anti-GFRAL antibody comprises (i) a VH comprising the VH CDR1, the VH CDR2, the VH CDR3 from the amino acid sequence set forth in SEQ ID NO:1982, and (ii) a VL comprising the VL CDR1, the VL CDR2, and the VL CDR3 from the amino acid sequence set forth in SEQ ID NO:1997 (i.e., the six CDRs of a humanized 3P10 antibody of Table 1A).

In some instances, the anti-GFRAL antibody comprises (i) a VH comprising the VH CDR1, the VH CDR2, the VH CDR3 from the amino acid sequence set forth in SEQ ID NO: 7; and (ii) a VL comprising the VL CDR1, the VL CDR2, and the VL CDR3 from the amino acid sequence set forth in SEQ ID NO: 8 (i.e., the six CDRs of antibody 5F12).

In some instances, the anti-GFRAL antibody comprises (i) a VH comprising the VH CDR1, the VH CDR2, the VH CDR3 from the amino acid sequence set forth in SEQ ID NO: 21; and (ii) a VL comprising the VL CDR1, the VL CDR2, and the VL CDR3 from the amino acid sequence set forth in EQ ID NO: 22 (i.e., the six CDRs of antibody 2I23).

In some instances, the anti-GFRAL antibody comprises (i) a VH comprising the VH CDR1, the VH CDR2, the VH CDR3 from the amino acid sequence set forth in SEQ ID NO: 23; and (ii) a VL comprising the VL CDR1, the VL CDR2, and the VL CDR3 from the amino acid sequence set forth in SEQ ID NO: 24 (i.e., the six CDRs of antibody 6N16).

In some instances, the anti-GFRAL antibody comprises (i) a VH comprising the VH CDR1, the VH CDR2, the VH CDR3 from the amino acid sequence set forth in SEQ ID NO: 25; and (ii) a VL comprising the VL CDR1, the VL CDR2, and the VL CDR3 from the amino acid sequence set forth in SEQ ID NO: 26 (i.e., the six CDRs of antibody 1B3).

In some instances, the anti-GFRAL antibody comprises (i) a VH comprising the VH CDR1, the VH CDR2, the VH CDR3 from the amino acid sequence set forth in SEQ ID NO: 37; and (ii) a VL comprising the VL CDR1, the VL CDR2, and the VL CDR3 from the amino acid sequence set forth in SEQ ID NO: 38 (i.e., the six CDRs of antibody 6G9).

In some instances, the anti-GFRAL antibody comprises (i) a VH comprising the VH CDR1, the VH CDR2, the VH CDR3 from the amino acid sequence set forth in SEQ ID NO: 39, and 1958-1965; and (ii) a VL comprising the VL CDR1, the VL CDR2, and the VL CDR3 from the amino acid sequence set forth in SEQ ID NO: 38 (i.e., the six CDRs of antibody 2B11).

In some instances, the anti-GFRAL antibody comprises (i) a VH comprising the VH CDR1, the VH CDR2, the VH CDR3 from the amino acid sequence set forth in any one of SEQ ID NOs: 1958-1965; and (ii) a VL comprising the VL CDR1, the VL CDR2, and the VL CDR3 from the amino acid sequence set forth in any one of SEQ ID NOs: 1967-1976 (i.e., the six CDRs of a humanized 5F12 antibody).

In some instances, the CDRs of the anti-GFRAL antibody are according to the Exemplary designation. In some instances, the anti-GFRAL antibody comprises: (i) a VH comprising a VH CDR1 comprising the amino acid sequence set forth in SEQ ID NO:46, a VH CDR2 comprising the amino acid sequence set forth in SEQ ID NO:137, and a VH CDR3 comprising the amino acid sequence set forth in SEQ ID NO:225; and (ii) a VL comprising a VL CDR1 comprising the amino acid sequence set forth in SEQ ID NO:301, a VL CDR2 comprising the amino acid sequence set forth in SEQ ID NO:376, and a VL CDR3 comprising the amino acid sequence set forth in SEQ ID NO:426.

In some instances, the anti-GFRAL antibody comprises: (i) a VH comprising the VH CDR1, the VH CDR2, and the VH CDR3 according to the Exemplary designation in Table 2; and (ii) a VL comprising the VL CDR1, the VL CDR2, and the VL CDR3 according to the Exemplary designation in any one of Table 2.

In some instances, the anti-GFRAL antibody comprises: (i) a VH comprising the VH CDR1, the VH CDR2, and the VH CDR3 according to the Exemplary designation in Table 3: and (ii) a VL comprising the VL CDR1, the VL CDR2, and the VL CDR3 according to the Exemplary designation in Table 3.

In some instances, the anti-GFRAL antibody comprises: (i) a VH comprising the VH CDR1, the VH CDR2, and the VH CDR3 according to the Exemplary designation in Table 4; and (ii) a VL comprising the VL CDR1, the VL CDR2, and the VL CDR3 according to the Exemplary designation in Table 4.

In some instances, the anti-GFRAL antibody comprises: (i) a VH comprising the VH CDR1, the VH CDR2, and the VH CDR3 according to the Exemplary designation in Table 5; and (ii) a VL comprising the VL CDR1, the VL CDR2, and the VL CDR3 according to the Exemplary designation in Table 5.

In some instances, the anti-GFRAL antibody comprises: (i) a VH comprising the VH CDR1, the VH CDR2, and the VH CDR3 according to the Exemplary designation in Table 6; and (ii) a VL comprising the VL CDR1, the VL CDR2, and the VL CDR3 according to the Exemplary designation in Table 6.

In some instances, the anti-GFRAL antibody comprises: (i) a VH comprising the VH CDR1, the VH CDR2, and the VH CDR3 according to the Exemplary designation in Table 7; and (ii) a VL comprising the VL CDR1, the VL CDR2, and the VL CDR3 according to the Exemplary designation in Table 7.

In some instances, the CDRs of the anti-GFRAL antibody are according to the IMGT designation. In some instances, the anti-GFRAL antibody comprises: (i) a VH comprising a VH CDR1 comprising the amino acid sequence set forth in SEQ ID NO:47, a VH CDR2 comprising the amino acid sequence set forth in SEQ ID NO:138, and a VH CDR3 comprising the amino acid sequence set forth in SEQ ID NO:226; and (ii) a VL comprising a VL CDR1 comprising the amino acid sequence set forth in SEQ ID NO:302, a VL CDR2 comprising the amino acid sequence set forth in SEQ ID NO:377, and a VL CDR3 comprising the amino acid sequence set forth in SEQ ID NO:426.

In some instances, the anti-GFRAL antibody comprises: (i) a VH comprising the VH CDR1, the VH CDR2, and the VH CDR3 according to the IMGT designation in Table 2; and (ii) a VL comprising the VL CDR1, the VL CDR2, and the VL CDR3 according to the IMGT designation in Table 2.

In some instances, the anti-GFRAL antibody comprises: (i) a VH comprising the VH CDR1, the VH CDR2, and the VH CDR3 according to the IMGT designation in Table 3; and (ii) a VL comprising the VL CDR1, the VL CDR2, and the VL CDR3 according to the IMGT designation in Table 3.

In some instances, the anti-GFRAL antibody comprises: (i) a VH comprising the VH CDR1, the VH CDR2, and the VH CDR3 according to the IMGT designation in Table 4; and (ii) a VL comprising the VL CDR1, the VL CDR2, and the VL CDR3 according to the IMGT designation in Table 4.

In some instances, the anti-GFRAL antibody comprises: (i) a VH comprising the VH CDR1, the VH CDR2, and the VH CDR3 according to the IMGT designation in Table 5; and (ii) a VL comprising the VL CDR1, the VL CDR2, and the VL CDR3 according to the IMGT designation in Table 5.

In some instances, the anti-GFRAL antibody comprises: (i) a VH comprising the VH CDR1, the VH CDR2, and the VH CDR3 according to the IMGT designation in Table 6; and (ii) a VL comprising the VL CDR1, the VL CDR2, and the VL CDR3 according to the IMGT designation in Table 6.

In some instances, the anti-GFRAL antibody comprises: (i) a VH comprising the VH CDR1, the VH CDR2, and the VH CDR3 according to the IMGT designation in Table 7; and (ii) a VL comprising the VL CDR1, the VL CDR2, and the VL CDR3 according to the IMGT designation in Table 7.

In some instances, the CDRs of the anti-GFRAL antibody are according to the Kabat designation. In some instances, the anti-GFRAL antibody comprises: (i) a VH comprising a VH CDR1 comprising the amino acid sequence set forth in SEQ ID NO:48, a VH CDR2 comprising the amino acid sequence set forth in SEQ ID NO:137, and a VH CDR3 comprising the amino acid sequence set forth in SEQ ID NO:225; and (ii) a VL comprising a VL CDR1 comprising the amino acid sequence set forth in SEQ ID NO:301, a VL CDR2 comprising the amino acid sequence set forth in SEQ ID NO:376, and a VL CDR3 comprising the amino acid sequence set forth in SEQ ID NO:426.

In some instances, the anti-GFRAL antibody comprises: (i) a VH comprising the VH CDR1, the VH CDR2, and the VH CDR3 according to the Kabat designation in Table 2; and (ii) a VL comprising the VL CDR1, the VL CDR2, and the VL CDR3 according to the Kabat designation in Table 2.

In some instances, the anti-GFRAL antibody comprises: (i) a VH comprising the VH CDR1, the VH CDR2, and the VH CDR3 according to the Kabat designation in Table 3; and (ii) a VL comprising the VL CDR1, the VL CDR2, and the VL CDR3 according to the Kabat designation in Table 3.

In some instances, the anti-GFRAL antibody comprises: (i) a VH comprising the VH CDR1, the VH CDR2, and the VH CDR3 according to the Kabat designation in Table 4; and (ii) a VL comprising the VL CDR1, the VL CDR2, and the VL CDR3 according to the Kabat designation in Table 4.

In some instances, the anti-GFRAL antibody comprises: (i) a VH comprising the VH CDR1, the VH CDR2, and the VH CDR3 according to the Kabat designation in Table 5; and (ii) a VL comprising the VL CDR1, the VL CDR2, and the VL CDR3 according to the Kabat designation in Table 5.

In some instances, the anti-GFRAL antibody comprises: (i) a VH comprising the VH CDR1, the VH CDR2, and the VH CDR3 according to the Kabat designation in Table 6; and (ii) a VL comprising the VL CDR1, the VL CDR2, and the VL CDR3 according to the Kabat designation in Table 6.

In some instances, the anti-GFRAL antibody comprises: (i) a VH comprising the VH CDR1, the VH CDR2, and the VH CDR3 according to the Kabat designation in Table 7; and (ii) a VL comprising the VL CDR1, the VL CDR2, and the VL CDR3 according to the Kabat designation in Table 7.

In some instances, the CDRs of the anti-GFRAL antibody are according to the Chothia designation. In some instances, the anti-GFRAL antibody comprises: (i) a VH comprising a VH CDR1 comprising the amino acid sequence set forth in SEQ ID NO:49, a VH CDR2 comprising the amino acid sequence set forth in SEQ ID NO:139, and a VH CDR3 comprising the amino acid sequence set forth in SEQ ID NO:227; and (ii) a VL comprising a VL CDR1 comprising the amino acid sequence set forth in SEQ ID NO:303, a VL CDR2 comprising the amino acid sequence set forth in SEQ ID NO:377, and a VL CDR3 comprising the amino acid sequence set forth in SEQ ID NO:427.

In some instances, the anti-GFRAL antibody comprises: (i) a VH comprising the VH CDR1, the VH CDR2, and the VH CDR3 according to the Chothia designation in Table 2; and (ii) a VL comprising the VL CDR1, the VL CDR2, and the VL CDR3 according to the Chothia designation in Table 2.

In some instances, the anti-GFRAL antibody comprises: (i) a VH comprising the VH CDR1, the VH CDR2, and the VH CDR3 according to the Chothia designation in Table 3; and (ii) a VL comprising the VL CDR1, the VL CDR2, and the VL CDR3 according to the Chothia designation in Table 3.

In some instances, the anti-GFRAL antibody comprises: (i) a VH comprising the VH CDR1, the VH CDR2, and the VH CDR3 according to the Chothia designation in Table 4; and (ii) a VL comprising the VL CDR1, the VL CDR2, and the VL CDR3 according to the Chothia designation in Table 4.

In some instances, the anti-GFRAL antibody comprises: (i) a VH comprising the VH CDR1, the VH CDR2, and the VH CDR3 according to the Chothia designation in Table 5; and (ii) a VL comprising the VL CDR1, the VL CDR2, and the VL CDR3 according to the Chothia designation in Table 5.

In some instances, the anti-GFRAL antibody comprises: (i) a VH comprising the VH CDR1, the VH CDR2, and the VH CDR3 according to the Chothia designation in Table 6; and (ii) a VL comprising the VL CDR1, the VL CDR2, and the VL CDR3 according to the Chothia designation in Table 6.

In some instances, the anti-GFRAL antibody comprises: (i) a VH comprising the VH CDR1, the VH CDR2, and the VH CDR3 according to the Chothia designation in Table 7; and (ii) a VL comprising the VL CDR1, the VL CDR2, and the VL CDR3 according to the Chothia designation in Table 7.

In some instances, the CDRs of the anti-GFRAL antibody are according to the Contact designation. In some instances, the anti-GFRAL antibody comprises: (i) a VH comprising a VH CDR1 comprising the amino acid sequence set forth in SEQ ID NO:50, a VH CDR2 comprising the amino acid sequence set forth in SEQ ID NO:140, and a VH CDR3 comprising the amino acid sequence set forth in SEQ ID NO:228; and (ii) a VL comprising a VL CDR1 comprising the amino acid sequence set forth in SEQ ID NO:304, a VL CDR2 comprising the amino acid sequence set forth in SEQ ID NO:378, and a VL CDR3 comprising the amino acid sequence set forth in SEQ ID NO:428.

In some instances, the anti-GFRAL antibody comprises: (i) a VH comprising the VH CDR1, the VH CDR2, and the VH CDR3 according to the Contact designation in Table 2; and (ii) a VL comprising the VL CDR1, the VL CDR2, and the VL CDR3 according to the Contact designation in Table 2.

In some instances, the anti-GFRAL antibody comprises: (i) a VH comprising the VH CDR1, the VH CDR2, and the VH CDR3 according to the Contact designation in Table 3; and (ii) a VL comprising the VL CDR1, the VL CDR2, and the VL CDR3 according to the Contact designation in Table 3.

In some instances, the anti-GFRAL antibody comprises: (i) a VH comprising the VH CDR1, the VH CDR2, and the VH CDR3 according to the Contact designation in Table 4; and (ii) a VL comprising the VL CDR1, the VL CDR2, and the VL CDR3 according to the Contact designation in Table 4.

In some instances, the anti-GFRAL antibody comprises: (i) a VH comprising the VH CDR1, the VH CDR2, and the VH CDR3 according to the Contact designation in Table 5; and (ii) a VL comprising the VL CDR1, the VL CDR2, and the VL CDR3 according to the Contact designation in Table 5.

In some instances, the anti-GFRAL antibody comprises: (i) a VH comprising the VH CDR1, the VH CDR2, and the VH CDR3 according to the Contact designation in Table 6; and (ii) a VL comprising the VL CDR1, the VL CDR2, and the VL CDR3 according to the Contact designation in Table 6.

In some instances, the anti-GFRAL antibody comprises: (i) a VH comprising the VH CDR1, the VH CDR2, and the VH CDR3 according to the Contact designation in Table 7; and (ii) a VL comprising the VL CDR1, the VL CDR2, and the VL CDR3 according to the Contact designation in Table 7.

In some instances, the CDRs of the anti-GFRAL antibody are according to the AbM designation. In some instances, the anti-GFRAL antibody comprises: (i) a VH comprising a VH CDR1 comprising the amino acid sequence set forth in SEQ ID NO:46, a VH CDR2 comprising the amino acid sequence set forth in SEQ ID NO:141, and a VH CDR3 comprising the amino acid sequence set forth in SEQ ID NO:225; and (ii) a VL comprising a VL CDR1 comprising the amino acid sequence set forth in SEQ ID NO:301, a VL CDR2 comprising the amino acid sequence set forth in SEQ ID NO:376, and a VL CDR3 comprising the amino acid sequence set forth in SEQ ID NO:426.

In some instances, the anti-GFRAL antibody comprises: (i) a VH comprising the VH CDR1, the VH CDR2, and the VH CDR3 according to the AbM designation in Table 2; and (ii) a VL comprising the VL CDR1, the VL CDR2, and the VL CDR3 according to the AbM designation in Table 2.

In some instances, the anti-GFRAL antibody comprises: (i) a VH comprising the VH CDR1, the VH CDR2, and the VH CDR3 according to the AbM designation in Table 3; and (ii) a VL comprising the VL CDR1, the VL CDR2, and the VL CDR3 according to the AbM designation in Table 3.

In some instances, the anti-GFRAL antibody comprises: (i) a VH comprising the VH CDR1, the VH CDR2, and the VH CDR3 according to the AbM designation in Table 4; and (ii) a VL comprising the VL CDR1, the VL CDR2, and the VL CDR3 according to the AbM designation in Table 4.

In some instances, the anti-GFRAL antibody comprises: (i) a VH comprising the VH CDR1, the VH CDR2, and the VH CDR3 according to the AbM designation in Table 5; and (ii) a VL comprising the VL CDR1, the VL CDR2, and the VL CDR3 according to the AbM designation in Table 5.

In some instances, the anti-GFRAL antibody comprises: (i) a VH comprising the VH CDR1, the VH CDR2, and the VH CDR3 according to the AbM designation in Table 6; and (ii) a VL comprising the VL CDR1, the VL CDR2, and the VL CDR3 according to the AbM designation in Table 6.

In some instances, the anti-GFRAL antibody comprises: (i) a VH comprising the VH CDR1, the VH CDR2, and the VH CDR3 according to the AbM designation in Table 7; and (ii) a VL comprising the VL CDR1, the VL CDR2, and the VL CDR3 according to the AbM designation in Table 7.

In certain embodiments, the anti-GFRAL antibody comprises a humanized framework region (FR) sequence, e.g., as described herein. In certain embodiments, the anti-GFRAL antibody described herein comprises a VH comprising a VH FR1, a VH FR2, a VH FR3 and a VH FR4 amino acid sequence described in International Patent Application Publication No. WO 2017/172260, which is incorporated by reference herein in its entirety; and/or (b) a VL comprising a VL FR1, a VL FR2, a VL FR3 and a VL FR4 amino acid sequence described in International Patent Application Publication No. WO 2017/172260.

In some instances, the anti-GFRAL antibody comprises a VH comprising the VH CDR1, the VH CDR2, and the VH CDR3 of antibody 3P10 (or a humanized version thereof), and wherein the VH comprises an amino acid sequence having at least at least 75%, at least 80%, at least 85%, at least 90%, at least 95%, at least 96%, at least 97%, at least 98%, or at least 99% identity to the amino acid sequence set forth in SEQ ID NO:3. In some instances, the anti-GFRAL antibody comprises a VH comprising the VH CDR1, the VH CDR2, and the VH CDR3 of antibody 3P10 (or a humanized version thereof), and wherein the VH comprises an amino acid sequence having at least at least 75%, at least 80%, at least 85%, at least 90%, at least 95%, at least 96%, at least 97%, at least 98%, at least 99%, or 100% identity to the amino acid sequence set forth in SEQ ID NO:1982.

In some instances, the anti-GFRAL antibody comprises a VL comprising the VL CDR1, the VL CDR2, and the VL CDR3 of antibody 3P10 (or a humanized version thereof), and wherein the VL comprises an amino acid sequence having at least at least 75%, at least 80%, at least 85%, at least 90%, at least 95%, at least 96%, at least 97%, at least 98%, or at least 99% identity to the amino acid sequence set forth in SEQ ID NO:4. In some instances, the anti-GFRAL antibody comprises a VL comprising the VL CDR1, the VL CDR2, and the VL CDR3 of antibody 3P10 (or a humanized version thereof), and wherein the VL comprises an amino acid sequence having at least at least 75%, at least 80%, at least 85%, at least 90%, at least 95%, at least 96%, at least 97%, at least 98%, at least 99%, or 100% identity to the amino acid sequence set forth in SEQ ID NO. 1997.

In some instances, the anti-GFRAL antibody comprises: (i) a VH comprising the VH CDR1, the VH CDR2, and the VH CDR3 of antibody 3P10 (or a humanized version thereof), and wherein the VH comprises an amino acid sequence having at least at least 75%, at least 80%, at least 85%, at least 90%, at least 95%, at least 96%, at least 97%, at least 98%, or at least 99% identity to the amino acid sequence set forth in SEQ ID NO:3; and a VL comprising the VL CDR1, the VL CDR2, and the VL CDR3 of antibody 3P10 (or a humanized version thereof), and wherein the VL comprises an amino acid sequence having at least at least 75%, at least 80%, at least 85%, at least 90%, at least 95%, at least 96%, at least 97%, at least 98%, or at least 99% identity to the amino acid sequence set forth in SEQ ID NO:4. In some instances, the anti-GFRAL antibody comprises: (i) a VH comprising the VH CDR1, the VH CDR2, and the VH CDR3 of antibody 3P10 (or a humanized version thereof), and wherein the VH comprises an amino acid sequence having at least at least 75%, at least 80%, at least 85%, at least 90%, at least 95%, at least 96%, at least 97%, at least 98%, at least 99%, or 100% identity to the amino acid sequence set forth in SEQ ID NO:1982; and (ii) a VL comprising the VL CDR1, the VL CDR2, and the VL CDR3 of antibody 3P10 (or a humanized version thereof), and wherein the VL comprises an amino acid sequence having at least at least 75%, at least 80%, at least 85%, at least 90%, at least 95%, at least 96%, at least 97%, at least 98%, at least 99%, or 100% identity to the amino acid sequence set forth in SEQ ID NO:1997.

In some instances, the anti-GFRAL antibody comprises a VH comprising the amino acid sequence set forth in any one of SEQ ID NOs:1978-1988. In some instances, the anti-GFRAL antibody comprises a VH comprising the amino acid sequence set forth in SEQ ID NO:1982.

In some instances, the anti-GFRAL antibody comprises a VL comprising the amino acid sequence set forth in any one of SEQ ID NOs:1990-2000. In some instances, the anti-GFRAL antibody comprises a VL comprising the amino acid sequence set forth in SEQ ID NO:1997.

In some instances, the anti-GFRAL antibody comprises: (i) a VH comprising the amino acid sequence set forth in any one of SEQ ID NOs:1978-1988; and (ii) a VL comprising the amino acid sequence set forth in any one of SEQ ID NOs:1990-2000. In some instances, the anti-GFRAL antibody comprises: (i) a VH comprising the amino acid sequence set forth in SEQ ID NO: 1982; and (ii) a VL comprising the amino acid sequence set forth in SEQ ID NO:1997.

In some instances, the anti-GFRAL antibody comprises a VH comprising the VH CDR1, the VH CDR2, and the VH CDR3 of antibody 5F12 (or a humanized version thereof), and wherein the VH comprises an amino acid sequence having at least at least 75%, at least 80%, at least 85%, at least 90%, at least 95%, at least 96%, at least 97%, at least 98%, or at least 99% identity to the amino acid sequence set forth in SEQ ID NO:7.

In some instances, the anti-GFRAL antibody comprises a VH comprising the VH CDR1, the VH CDR2, and the VH CDR3 of antibody 2I23 (or a humanized version thereof), and wherein the VH comprises an amino acid sequence having at least at least 75%, at least 80%, at least 85%, at least 90%, at least 95%, at least 96%, at least 97%, at least 98%, or at least 99% identity to the amino acid sequence set forth in SEQ ID NO: 21.

In some instances, the anti-GFRAL antibody comprises a VH comprising the VH CDR1, the VH CDR2, and the VH CDR3 of antibody 6N16 (or a humanized version thereof), and wherein the VH comprises an amino acid sequence having at least at least 75%, at least 80%, at least 85%, at least 90%, at least 95%, at least 96%, at least 97%, at least 98%, or at least 99% identity to the amino acid sequence set forth in SEQ ID NO: 23.

In some instances, the anti-GFRAL antibody comprises a VH comprising the VH CDR1, the VH CDR2, and the VH CDR3 of antibody 1B3 (or a humanized version thereof), and wherein the VH comprises an amino acid sequence having at least at least 75%, at least 80%, at least 85%, at least 90%, at least 95%, at least 96%, at least 97%, at least 98%, or at least 99% identity to the amino acid sequence set forth in SEQ ID NO: 25.

In some instances, the anti-GFRAL antibody comprises a VH comprising the VH CDR1, the VH CDR2, and the VH CDR3 of antibody 6G9 (or a humanized version thereof), and wherein the VH comprises an amino acid sequence having at least at least 75%, at least 80%, at least 85%, at least 90%, at least 95%, at least 96%, at least 97%, at least 98%, or at least 99% identity to the amino acid sequence set forth in SEQ ID NO: 37.

In some instances, the anti-GFRAL antibody comprises a VH comprising the VH CDR1, the VH CDR2, and the VH CDR3 of antibody 2B11 (or a humanized version thereof), and wherein the VH comprises an amino acid sequence having at least at least 75%, at least 80%, at least 85%, at least 90%, at least 95%, at least 96%, at least 97%, at least 98%, or at least 99% identity to the amino acid sequence set forth in SEQ ID NO: 39.

In some instances, the anti-GFRAL antibody comprises a VL comprising the VL CDR1, the VL CDR2, and the VL CDR3 of antibody 5F12 (or a humanized version thereof), and wherein the VL comprises an amino acid sequence having at least at least 75%, at least 80%, at least 85%, at least 90%, at least 95%, at least 96%, at least 97%, at least 98%, or at least 99% identity to the amino acid sequence set forth in SEQ ID NO:8.

In some instances, the anti-GFRAL antibody comprises a VL comprising the VL CDR1, the VL CDR2, and the VL CDR3 of antibody 2I23 (or a humanized version thereof), and wherein the VL comprises an amino acid sequence having at least at least 75%, at least 80%, at least 85%, at least 90%, at least 95%, at least 96%, at least 97%, at least 98%, or at least 99% identity to the amino acid sequence set forth in SEQ ID NO: 22.

In some instances, the anti-GFRAL antibody comprises a VL comprising the VL CDR1, the VL CDR2, and the VL CDR3 of antibody 6N16 (or a humanized version thereof), and wherein the VL comprises an amino acid sequence having at least at least 75%, at least 80%, at least 85%, at least 90%, at least 95%, at least 96%, at least 97%, at least 98%, or at least 99% identity to the amino acid sequence set forth in SEQ ID NO: 24.

In some instances, the anti-GFRAL antibody comprises a VL comprising the VL CDR1, the VL CDR2, and the VL CDR3 of antibody 1B3 (or a humanized version thereof), and wherein the VL comprises an amino acid sequence having at least at least 75%, at least 80%, at least 85%, at least 90%, at least 95%, at least 96%, at least 97%, at least 98%, or at least 99% identity to the amino acid sequence set forth in SEQ ID NO: 26.

In some instances, the anti-GFRAL antibody comprises a VL comprising the VL CDR1, the VL CDR2, and the VL CDR3 of antibody 6G9 (or a humanized version thereof), and wherein the VL comprises an amino acid sequence having at least at least 75%, at least 80%, at least 85%, at least 90%, at least 95%, at least 96%, at least 97%, at least 98%, or at least 99% identity to the amino acid sequence set forth in SEQ ID NO: 38.

In some instances, the anti-GFRAL antibody comprises a VL comprising the VL CDR1, the VL CDR2, and the VL CDR3 of antibody 2B11 (or a humanized version thereof), and wherein the VL comprises an amino acid sequence having at least at least 75%, at least 80%, at least 85%, at least 90%, at least 95%, at least 96%, at least 97%, at least 98%, or at least 99% identity to the amino acid sequence set forth in SEQ ID NO: 40.

In some instances, the anti-GFRAL antibody comprises: (i) a VH comprising the VH CDR1, the VH CDR2, and the VH CDR3 of antibody 5F12 (or a humanized version thereof), and wherein the VH comprises an amino acid sequence having at least at least 75%, at least 80%, at least 85%, at least 90%, at least 95%, at least 96%, at least 97%, at least 98%, or at least 99% identity to the amino acid sequence set forth in SEQ ID NO:7; and a VL comprising the VL CDR1, the VL CDR2, and the VL CDR3 of antibody 5F12 (or a humanized version thereof), and wherein the VL comprises an amino acid sequence having at least at least 75%, at least 80%, at least 85%, at least 90%, at least 95%, at least 96%, at least 97%, at least 98%, or at least 99% identity to the amino acid sequence set forth in SEQ ID NO:8.

In some instances, the anti-GFRAL antibody comprises: (i) a VH comprising the VH CDR1, the VH CDR2, and the VH CDR3 of antibody 2I23 (or a humanized version thereof), and wherein the VH comprises an amino acid sequence having at least at least 75%, at least 80%, at least 85%, at least 90%, at least 95%, at least 96%, at least 97%, at least 98%, or at least 99% identity to the amino acid sequence set forth in SEQ ID NO: 21; and a VL comprising the VL CDR1, the VL CDR2, and the VL CDR3 of antibody 2I23 (or a humanized version thereof), and wherein the VL comprises an amino acid sequence having at least at least 75%, at least 80%, at least 85%, at least 90%, at least 95%, at least 96%, at least 97%, at least 98%, or at least 99% identity to the amino acid sequence set forth in SEQ ID NO: 22.

In some instances, the anti-GFRAL antibody comprises: (i) a VH comprising the VH CDR1, the VH CDR2, and the VH CDR3 of antibody 6N16 (or a humanized version thereof), and wherein the VH comprises an amino acid sequence having at least at least 75%, at least 80%, at least 85%, at least 90%, at least 95%, at least 96%, at least 97%, at least 98%, or at least 99% identity to the amino acid sequence set forth in SEQ ID NO: 23; and a VL comprising the VL CDR1, the VL CDR2, and the VL CDR3 of antibody 6N16 (or a humanized version thereof), and wherein the VL comprises an amino acid sequence having at least at least 75%, at least 80%, at least 85%, at least 90%, at least 95%, at least 96%, at least 97%, at least 98%, or at least 99% identity to the amino acid sequence set forth in SEQ ID NO: 24.

In some instances, the anti-GFRAL antibody comprises: (i) a VH comprising the VH CDR1, the VH CDR2, and the VH CDR3 of antibody 1B3 (or a humanized version thereof), and wherein the VH comprises an amino acid sequence having at least at least 75%, at least 80%, at least 85%, at least 90%, at least 95%, at least 96%, at least 97%, at least 98%, or at least 99% identity to the amino acid sequence set forth in SEQ ID NO: 25; and a VL comprising the VL CDR1, the VL CDR2, and the VL CDR3 of antibody 1B3 (or a humanized version thereof), and wherein the VL comprises an amino acid sequence having at least at least 75%, at least 80%, at least 85%, at least 90%, at least 95%, at least 96%, at least 97%, at least 98%, or at least 99% identity to the amino acid sequence set forth in SEQ ID NO: 26.

In some instances, the anti-GFRAL antibody comprises: (i) a VH comprising the VH CDR1, the VH CDR2, and the VH CDR3 of antibody 6G9 (or a humanized version thereof), and wherein the VH comprises an amino acid sequence having at least at least 75%, at least 80%, at least 85%, at least 90%, at least 95%, at least 96%, at least 97%, at least 98%, or at least 99% identity to the amino acid sequence set forth in SEQ ID NO: 37; and a VL comprising the VL CDR1, the VL CDR2, and the VL CDR3 of antibody 6G9 (or a humanized version thereof), and wherein the VL comprises an amino acid sequence having at least at least 75%, at least 80%, at least 85%, at least 90%, at least 95%, at least 96%, at least 97%, at least 98%, or at least 99% identity to the amino acid sequence set forth in SEQ ID NO: 38.

In some instances, the anti-GFRAL antibody comprises: (i) a VH comprising the VH CDR1, the VH CDR2, and the VH CDR3 of antibody 2B11 (or a humanized version thereof), and wherein the VH comprises an amino acid sequence having at least at least 75%, at least 80%, at least 85%, at least 90%, at least 95%, at least 96%, at least 97%, at least 98%, or at least 99% identity to the amino acid sequence set forth in SEQ ID NO: 39; and a VL comprising the VL CDR1, the VL CDR2, and the VL CDR3 of antibody 2B11 (or a humanized version thereof), and wherein the VL comprises an amino acid sequence having at least at least 75%, at least 80%, at least 85%, at least 90%, at least 95%, at least 96%, at least 97%, at least 98%, or at least 99% identity to the amino acid sequence set forth in SEQ ID NO: 40.

In some instances, the anti-GFRAL antibody comprises a VH comprising a humanized version of the amino acid sequence set forth in SEQ ID NO:7. In some instances, the anti-GFRAL antibody comprises a VH comprising the amino acid sequence set forth in any one of SEQ ID NOs:1958-1965.

In some instances, the anti-GFRAL antibody comprises a VH comprising a humanized version of the amino acid sequence set forth in SEQ ID NO:21.

In some instances, the anti-GFRAL antibody comprises a VH comprising a humanized version of the amino acid sequence set forth in SEQ ID NO: 23.

In some instances, the anti-GFRAL antibody comprises a VH comprising a humanized version of the amino acid sequence set forth in SEQ ID NO: 25.

In some instances, the anti-GFRAL antibody comprises a VH comprising a humanized version of the amino acid sequence set forth in SEQ ID NO: 37.

In some instances, the anti-GFRAL antibody comprises a VH comprising a humanized version of the amino acid sequence set forth in SEQ ID NO: 39.

In some instances, the anti-GFRAL antibody comprises a VL comprising a humanized version of the amino acid sequence set forth in SEQ ID NO:8. In some instances, the anti-GFRAL antibody comprises a VL comprising the amino acid sequence set forth in any one of SEQ ID NOs:1967-1976.

In some instances, the anti-GFRAL antibody comprises a VL comprising a humanized version of the amino acid sequence set forth in SEQ ID NO: 22.

In some instances, the anti-GFRAL antibody comprises a VL comprising a humanized version of the amino acid sequence set forth in SEQ ID NO: 24.

In some instances, the anti-GFRAL antibody comprises a VL comprising a humanized version of the amino acid sequence set forth in SEQ ID NO: 26.

In some instances, the anti-GFRAL antibody comprises a VL comprising a humanized version of the amino acid sequence set forth in SEQ ID NO: 38.

In some instances, the anti-GFRAL antibody comprises a VL comprising a humanized version of the amino acid sequence set forth in SEQ ID NO: 40.

In some instances, the anti-GFRAL antibody comprises: (i) a VH comprising a humanized version of the amino acid sequence set forth in SEQ ID NO:7; and (ii) a VL comprising a humanized version of the amino acid sequence set forth in SEQ ID NO:8. In some instances, the anti-GFRAL antibody comprises: (i) a VH comprising the amino acid sequence set forth in any one of SEQ ID NOs:1958-1965; and (ii) a VL comprising the amino acid sequence set forth in any one of SEQ ID NOs:1967-1976.

In some instances, the anti-GFRAL antibody comprises: (i) a VH comprising a humanized version of the amino acid sequence set forth in SEQ ID NO: 21; and (ii) a VL comprising a humanized version of the amino acid sequence set forth in SEQ ID NO:22.

In some instances, the anti-GFRAL antibody comprises: (i) a VH comprising a humanized version of the amino acid sequence set forth in SEQ ID NO: 23; and (ii) a VL comprising a humanized version of the amino acid sequence set forth in SEQ ID NO: 24.

In some instances, the anti-GFRAL antibody comprises: (i) a VH comprising a humanized version of the amino acid sequence set forth in SEQ ID NO: 25; and (ii) a VL comprising a humanized version of the amino acid sequence set forth in SEQ ID NO: 26.

In some instances, the anti-GFRAL antibody comprises: (i) a VH comprising a humanized version of the amino acid sequence set forth in SEQ ID NO: 37; and (ii) a VL comprising a humanized version of the amino acid sequence set forth in SEQ ID NO: 38.

In some instances, the anti-GFRAL antibody comprises: (i) a VH comprising a humanized version of the amino acid sequence set forth in SEQ ID NO: 39; and (ii) a VL comprising a humanized version of the amino acid sequence set forth in SEQ ID NO: 40.

In some instances, the anti-GFRAL antibody comprises a heavy chain comprising a VH comprising the VH CDR1, the VH CDR2, and the VH CDR3 from the amino acid sequence set forth in SEQ ID NO: 1982, and wherein the heavy chain comprises an amino acid sequence having at least at least 75%, at least 80%, at least 85%, at least 90%, at least 95%, at least 96%, at least 97%, at least 98%, at least 99%, or 100% identity to the amino acid sequence set forth in SEQ ID NO:2010.

In some instances, the anti-GFRAL antibody comprises a light chain comprising a VL comprising the VL CDR1, the VL CDR2, and the VL CDR3 from the amino acid sequence set forth in SEQ ID NO:1997, and wherein the light chain comprises an amino acid sequence having at least at least 75%, at least 80%, at least 85%, at least 90%, at least 95%, at least 96%, at least 97%, at least 98%, at least 99%, or 100% identity to the amino acid sequence set forth in SEQ ID NO:2012.

In some instances, the anti-GFRAL antibody comprises: (i) a heavy chain comprising a VH comprising the VH CDR1, the VH CDR2, and the VH CDR3 from the amino acid sequence set forth in SEQ ID NO: 1982, and wherein the heavy chain comprises an amino acid sequence having at least at least 75%, at least 80%, at least 85%, at least 90%, at least 95%, at least 96%, at least 97%, at least 98%, at least 99%, or 100% identity to the amino acid sequence set forth in SEQ ID NO:2010; and (ii) a light chain comprising a VL comprising the VL CDR1, the VL CDR2, and the VL CDR3 from the amino acid sequence set forth in SEQ ID NO:1997, and wherein the light chain comprises an amino acid sequence having at least at least 75%, at least 80%, at least 85%, at least 90%, at least 95%, at least 96%, at least 97%, at least 98%, at least 99%, or 100% identity to the amino acid sequence set forth in SEQ ID NO:2012.

In some instances, the anti-GFRAL antibody comprises a heavy chain comprising the amino acid sequence set forth in SEQ ID NO:2010. In some instances, the anti-GFRAL antibody comprises a heavy chain consisting of the amino acid sequence set forth in SEQ ID NO:2010.

In some instances, the anti-GFRAL antibody comprises a light chain comprising the amino acid sequence set forth in SEQ ID NO:2012. In some instances, the anti-GFRAL antibody comprises a light chain consisting of the amino acid sequence set forth in SEQ ID NO:2012.

In some instances, the anti-GFRAL antibody comprises: (i) a heavy chain comprising the amino acid sequence set forth in SEQ ID NO:2010; and (ii) a light chain comprising the amino acid sequence set forth in SEQ ID NO:2012. In some instances, the anti-GFRAL antibody comprises: (i) a heavy chain consisting of the amino acid sequence set forth in SEQ ID NO:2010; and (ii) a light chain consisting of the amino acid sequence set forth in SEQ ID NO:2012.

In certain instances, the anti-GFRAL antibody is an antibody described in International Patent Application Publication No. WO 2017/172260, which is incorporated by reference herein in its entirety. In certain instances, the anti-GFRAL antibody is a humanized version of an antibody described in International Patent Application Publication No. WO 2017/172260, which is incorporated by reference herein in its entirety.

In some instances, the anti-GFRAL antibody binds to the same epitope as an antibody having the VH and VL of any one of Tables 1-7. In some instances, the anti-GFRAL antibody binds to the same epitope as antibody Hz3P10 (see Table 1A).

In some instances, the anti-GFRAL antibody specifically binds to human GFRAL within amino acid residues 220-316 of the amino acid sequence set forth in SEQ ID NO: 1797. In some instances, the anti-GFRAL antibody specifically binds to human GFRAL at one or more (e.g., 1, 2, 3, 4, 5, 6, 7, 8, 9, 10, 11, 12, 13, 14, 15, 16, 17, 18, 19) residues selected from the group consisting of. Met214, Pro216, Pro217, Gln290, Cys291, Thr292, Cys293, Arg294, Thr295, Ile296, Thr297, Gln298, Ser299, Glu301, Lys305, Gln308, His309, His312, and Ser315 of the amino acid sequence set forth in SEQ ID NO: 1797. In some instances, the anti-GFRAL antibody specifically binds to human GFRAL at Thr297. Gln298, and Ser299 of the amino acid sequence set forth in SEQ ID NO:1797.

In some instances, the anti-GFRAL antibody is a humanized antibody.

Various methods for generating humanized antibodies are known in the art. In some embodiments, a humanized antibody comprises one or more amino acid residues that have been introduced into it from a source that is non-human. In some embodiments, humanization is performed by substituting one or more non-human CDR sequences for the corresponding CDR sequences of a human antibody. In some embodiments, the humanized antibodies are constructed by substituting all six CDRs of a non-human antibody (e.g., a mouse antibody) for the corresponding CDRs of a human antibody.

The choice of which human heavy chain variable region and/or light chain variable region is used for generating humanized antibodies can be made based on a variety of factors and by a variety of methods known in the art. In some embodiments, the “best-fit” method is used where the sequence of the variable region of a non-human (e.g., rodent) antibody is screened against the entire library of known human variable region sequences. The human sequence that is most similar to that of the non-human (e.g., rodent) sequence is selected as the human variable region framework for the humanized antibody. In some embodiments, a particular variable region framework derived from a consensus sequence of all human antibodies of a particular subgroup of light or heavy chains is selected as the variable region framework. In some embodiments, the variable region framework sequence is derived from the consensus sequences of the most abundant human subclasses. In some embodiments, human germline genes are used as the source of the variable region framework sequences.

Other methods for humanization include, but are not limited to, a method called “superhumanization” which is described as the direct transfer of CDRs to a human germline framework, a method termed Human String Content (HSC) which is based on a metric of “antibody humanness”, methods based on generation of large libraries of humanized variants (including phage, ribosomal, and yeast display libraries), and methods based on framework region shuffling.

In some instances, the anti-GFRAL antibody is a human IgG1 antibody. In some instances, the anti-GFRAL antibody is a human IgG2 antibody. In some instances, the anti-GFRAL antibody is a human IgG4 antibody. In some instances, the anti-GFRAL antibody comprises a human kappa light chain constant region. In some instances, the anti-GFRAL antibody comprises a human lambda light chain constant region.

In some instances, the anti-GFRAL antibody is an antibody fragment comprising at least one antigen-binding site. In some embodiments, the antibody or antibody fragment is a Fab, Fab′, F(ab′)₂, Fv, scFv, (scFv)₂, single chain antibody, dual variable region antibody, single variable region antibody, linear antibody, diabody, nanobody, or a V region antibody.

The anti-GFRAL antibodies described herein can be produced by any suitable method known in the art. Such methods range from direct protein synthesis methods to constructing a DNA sequence encoding polypeptide sequences and expressing those sequences in a suitable host. See, e.g., International Patent Application Publication No. WO 2017/172260, which is incorporated by reference herein in its entirety, for a description of various methods for producing antibodies.

Also provided herein are pharmaceutical compositions comprising an anti-GFRAL antibody described herein. In some instances, the pharmaceutical composition comprises an anti-GFRAL antibody described herein and a pharmaceutically acceptable carrier. In some instances, the pharmaceutical composition comprises, an anti-GFRAL antibody comprising a VH comprising the VH CDR1, the VH CDR2, and the VH CDR3 of the antibody 3P10 or Hz3P10 (see Table 1 and Table 1A) and a VL comprising the VL CDR1, the VL CDR2, and the VL CDR3 of the antibody 3P10 or Hz3P10 (see Table 1 and Table 1A); and a pharmaceutically acceptable carrier. In some instances, the pharmaceutical composition comprises: an anti-GFRAL antibody comprising a VH comprising the amino acid sequence set forth in SEQ ID NO: 1982 and a VL comprising the amino acid sequence set forth in SEQ ID NO: 1997; and a pharmaceutically acceptable carrier. In some instances, the pharmaceutical composition comprises: an anti-GFRAL antibody comprising a heavy chain comprising the amino acid sequence set forth in SEQ ID NO:2010 and a light comprising the amino acid sequence set forth in SEQ ID NO:2012; and a pharmaceutically acceptable carrier.

C. Paclitaxel

Paclitaxel (TAXOL) is an antimicrotubule agent indicated: (i) as first-line and subsequent therapy for the treatment of advanced carcinoma of the ovary, (ii) for the adjuvant treatment of node-positive breast cancer administered sequentially to standard doxorubicin-containing combination chemotherapy; (iii) for the treatment of breast cancer after failure of combination chemotherapy for metastatic disease or relapse within 6 months of adjuvant chemotherapy; (iv) in combination with cisplatin, for the first-line treatment of non-small cell lung cancer in patients who are not candidates for potentially curative surgery and/or radiation therapy; and (v) the second-line treatment of AIDS-related Kaposi's sarcoma.

The chemical name for paclitaxel is 5β,20-Epoxy-1,2α,4,7β,10β,13α-hexahydroxytax-11-en-9-one 4,10-diacetate 2-benzoate 13-ester with (2R,3S)—N-benzoyl-3-phenylisoserine.

Paclitaxel may be bound to protein (e.g., albumin). For instance, nanoparticle albumin-bound paclitaxel (referred to as “nab-paclitaxel”; ABRAXANE) is indicated for the treatment of: (i) metastatic breast cancer, after failure of combination chemotherapy for metastatic disease or relapse within 6 months of adjuvant chemotherapy; (ii) locally advanced or metastatic non-small cell lung cancer (NSCLC), as first-line treatment in combination with carboplatin, in patients who are not candidates for curative surgery or radiation therapy; and (iii) metastatic adenocarcinoma of the pancreas as first-line treatment, in combination with gemcitabine.

In some instances, paclitaxel (e.g., nab-paclitaxel) is administered to a human patient at a dose of about 50 mg/m², about 60 mg/m², about 65 mg/m², about 70 mg/m², about 75 mg/m², about 80 mg/m², about 90 mg/m², about 95 mg/m², about 100 mg/m², about 115 mg/m², about 125 mg/m², about 135 mg/m², about 150 mg/m², 50-150 mg/m², 50-125 mg/m², 50-100 mg/m², 50-75 mg/m², 75-150 mg/m², 75-125 mg/m², 75-100 mg/m², or 100-125 mg/m². In some instances, paclitaxel (e.g., nab-paclitaxel) is administered to a human patient at a dose of 125 mg/m². In some instances, paclitaxel (e.g., nab-paclitaxel) is administered to a human patient at a dose of 125 mg/m² once a week. In some instances, the paclitaxel is nab-paclitaxel. In some instances, the nab-paclitaxel is administered to a human patient at a dose of 125 mg/m². In some instances, the nab-paclitaxel is administered to a human patient at a dose of 125 mg/m² once a week for 3 weeks during a 4 week interval during which an anti-GFRAL antibody is administered to the human patient. In some instances, paclitaxel (e.g., nab-paclitaxel) is administered to the human patient intravenously. In some instances, paclitaxel (e.g., nab-paclitaxel) is administered to a human patient via intravenous infusion over 30-40 minutes on days 1, 8, and 15 during a 4 week interval during which an anti-GFRAL antibody is administered to the human patient.

D. Gemcitabine

Gemcitabine (GEMZAR) is a nucleoside metabolic inhibitor indicated: (i) in combination with carboplatin, for the treatment of advanced ovarian cancer that has relapsed at least 6 months after completion of platinum-based therapy; (ii) in combination with paclitaxel, for first-line treatment of metastatic breast cancer after failure of prior anthracycline-containing adjuvant chemotherapy, unless anthracyclines were clinically contraindicated, and (iii) in combination with cisplatin for the treatment of non-small cell lung cancer as a single agent for the treatment of pancreatic cancer.

The chemical name for gemcitabine is 4-amino-1-[(2R,4R,5R)-3,3-difluoro-4-hydroxy-5-(hydroxymethyl)oxolan-2-yl]pyrimidin-2-one.

In some instances, gemcitabine is administered to a human patient at a dose of about 400 mg/m², about 450 mg/m², about 500 mg/m², about 550 mg/m², about 600 mg/m², about 650 mg/m², about 700 mg/m², about 750 mg/m², about 800 mg/m², about 850 mg/m², about 900 mg/m², about 950 mg/m², about 1000 mg/m², 400-1000 mg/m², 400-800 mg/m², 400-600 mg/m², 500-1000 mg/m², 500-900 mg/m², 500-800 mg/m², or 500-700 mg/m². In some instances, gemcitabine is administered to a human patient at a dose of 1000 mg/m². In some instances, gemcitabine is administered to a human patient at a dose of 1000 mg/m² once a week for 3 weeks during a 4 week interval during which an anti-GFRAL antibody is administered to the human patient. In some instances, gemcitabine is administered to a human patient intravenously. In some instances, gemcitabine is administered to a human patient via intravenous infusion over 30-40 minutes on days 1, 8, and 15 during a 4 week interval during which an anti-GFRAL antibody is administered to the human patient.

E. Methods of Treatment

1. Methods of Treating a Cancer

An anti-GFRAL antibody described herein or a pharmaceutical composition comprising the same can be used to treat a human patient having a tumor (e.g., a tumor, an advanced solid tumor, a cancer).

Thus, provided herein is a method for treating a tumor (e.g., a tumor, an advanced solid tumor, a cancer) in a human patient, the method comprising administering to the human patient an anti-GFRAL antibody described herein at a dose of about 30 mg (or a pharmaceutical composition comprising the same) once about every 3 weeks. In some instances, the dose is 30 mg. In some instances, the dose is administered once every 3 weeks. In some instances, the dose is 30 mg and the dose is administered once every 3 weeks. In some instances, the anti-GFRAL antibody is administered subcutaneously. In some instances, the anti-GFRAL antibody is administered subcutaneously at a dose of about 30 mg about once every 3 weeks. In some instances, the anti-GFRAL antibody is administered subcutaneously at a dose of 30 mg once every 3 weeks. In some instances, the anti-GFRAL antibody is administered intravenously. In some instances, the tumor is a solid tumor. In some instances, the tumor is an advanced solid tumor. In some instances, the tumor is a cancer. In some instances, the cancer is metastatic. In some instances, the cancer is advanced. In some instances, the cancer is an advanced solid tumor. In some instances, the cancer is a prostate cancer, a bladder cancer, a melanoma, a small cell lung cancer (SCLC) (including an extensive-stage SCLC), a non-small cell lung cancer (NSCLC), a pancreatic cancer, a colorectal cancer, a gastric cancer, an esophageal cancer, an ovarian cancer, a head and neck squamous cell carcinoma, or an MSI-H (microsatellite instability high) cancer. The prostate cancer can include, for example, a castration-sensitive prostate cancer, a castration-resistant prostate cancer, an MSI-H prostate cancer, a metastatic prostate cancer, a chemo-refractory prostate cancer, a prostate cancer where the patient has previously received one or more, two or more, or one or more (e.g., 1, 2, 3, or more) lines of standard prostate cancer therapies, or an advanced prostate cancer where the patient has exhausted all standard lines of therapies. A pancreatic cancer can include, for example, an advanced pancreatic cancer, a metastatic pancreatic cancer, a pancreatic adenocarcinoma, a metastatic pancreatic adenocarcinoma, an MSI-H pancreatic cancer, a pancreatic ductal adenocarcinoma, or a metastatic pancreatic ductal adenocarcinoma. An MSI-H cancer can include, for example, an MSI-H prostate cancer, an MSI-H bladder cancer, an MSI-H melanoma, an MSI-H small cell lung cancer (SCLC) (including an extensive-stage SCLC), an MSI-H non-small cell lung cancer (NSCLC), an MSI-H pancreatic cancer, an MSI-H colorectal cancer, an MSI-H gastric cancer, an MSI-H esophageal cancer, an MSI-H ovarian cancer, or an MSI-H head and neck squamous cell carcinoma. In some instances, the patient has an elevated level of GDF15 in the serum compared to the baseline level of GDF15 thereof without the tumor. In some instances, the cancer is a pancreatic cancer (e.g., an advanced pancreatic cancer, a metastatic pancreatic cancer, a pancreatic adenocarcinoma, a metastatic pancreatic adenocarcinoma, an MSI-H pancreatic cancer, a pancreatic ductal adenocarcinoma, a metastatic pancreatic ductal adenocarcinoma). In some instances, the cancer is a pancreatic adenocarcinoma. In some instances, the cancer is a metastatic pancreatic cancer. In some instances, the cancer is a metastatic pancreatic adenocarcinoma. In some instances, the pancreatic cancer is an advanced pancreatic cancer. In some instances, the pancreatic cancer is an MSI-H pancreatic cancer. In some instances, the pancreatic cancer is a pancreatic ductal adenocarcinoma. In some instances, the pancreatic cancer is a metastatic pancreatic ductal adenocarcinoma. In some instances, the human patient has a prostate cancer (e.g., an mCRPC, a castration-sensitive prostate cancer, a castration-resistant prostate cancer, an MSI-H prostate cancer, a metastatic prostate cancer, a metastatic castrate-resistant, chemo-refractory prostate cancer; an advanced prostate cancer where the patient has exhausted all standard therapies with elevated baseline serum GDF15; an advanced prostate cancer where the patient has previously received one or more (e.g., 1, 2, 3, or more) lines of cancer therapies; a chemo-refractory prostate cancer; a metastatic chemo-refractory prostate cancer). In some instances, the cancer is advanced prostate cancer. In some instances, the human patient has prostate cancer and poor survival. In some instances, the cancer is an mCRPC. In some instances, the cancer is chemo-refractory prostate cancer. In some instances, the cancer is metastatic chemo-refractory prostate cancer. In some instances, the cancer is metastatic castrate-resistant, chemo-refractory prostate cancer. In some instances, the cancer is advanced prostate cancer, wherein the human patient has exhausted all standard therapies, and wherein the human patient has an elevated baseline serum GDF15 (compared to a human or a panel of humans not having prostate cancer). In some instances, the cancer is advanced prostate cancer (e.g., castration-resistant prostate cancer), wherein the human patient has previously received one or more (e.g., 1, 2, 3, or more) lines of therapies (e.g., leuprolide acetate (LUPRON), zoledronic acid (ZOMETA), docetaxel, cabazitaxel, radium 223, mitoxantrone, Oxaliplatin and pemetrexed).

Also provided herein is a method for treating a tumor (e.g., a solid tumor, an advanced solid tumor, a cancer) in a human patient, the method comprising administering to the human patient an anti-GFRAL antibody described herein at a dose of about 100 mg (or a pharmaceutical composition comprising the same) once about every 3 weeks. In some instances, the dose is 100 mg. In some instances, the dose is administered once every 3 weeks. In some instances, the dose is 100 mg and the dose is administered once every 3 weeks. In some instances, the anti-GFRAL antibody is administered subcutaneously. In some instances, the anti-GFRAL antibody is administered subcutaneously at a dose of about 100 mg about once every 3 weeks. In some instances, the anti-GFRAL antibody is administered subcutaneously at a dose of 100 mg once every 3 weeks. In some instances, the anti-GFRAL antibody is administered intravenously. In some instances, the tumor is a solid tumor. In some instances, the tumor is an advanced solid tumor. In some instances, the tumor is a cancer. In some instances, the cancer is metastatic. In some instances, the cancer is advanced. In some instances, the cancer is an advanced solid tumor. In some instances, the cancer is a prostate cancer, a bladder cancer, a melanoma, a small cell lung cancer (SCLC) (including an extensive-stage SCLC), a non-small cell lung cancer (NSCLC), a pancreatic cancer, a colorectal cancer, a gastric cancer, an esophageal cancer, an ovarian cancer, a head and neck squamous cell carcinoma, or an MSI-H (microsatellite instability high) cancer. The prostate cancer can include, for example, a castration-sensitive prostate cancer, a castration-resistant prostate cancer, an MSI-H prostate cancer, a metastatic prostate cancer, a chemo-refractory prostate cancer, a prostate cancer where the patient has previously received one or more, two or more, or one or more (e.g., 1, 2, 3, or more) lines of standard prostate cancer therapies, or an advanced prostate cancer where the patient has exhausted all standard lines of therapies. A pancreatic cancer can include, for example, an advanced pancreatic cancer, a metastatic pancreatic cancer, a pancreatic adenocarcinoma, a metastatic pancreatic adenocarcinoma, an MSI-H pancreatic cancer, a pancreatic ductal adenocarcinoma, or a metastatic pancreatic ductal adenocarcinoma. An MSI-H cancer can include, for example, an MSI-H prostate cancer, an MSI-H bladder cancer, an MSI-H melanoma, an MSI-H small cell lung cancer (SCLC) (including an extensive-stage SCLC), an MSI-H non-small cell lung cancer (NSCLC), an MSI-H pancreatic cancer, an MSI-H colorectal cancer, an MSI-H gastric cancer, an MSI-H esophageal cancer, an MSI-H ovarian cancer, or an MSI-H head and neck squamous cell carcinoma. In some instances, the patient has an elevated level of GDF15 in the serum compared to the baseline level of GDF15 thereof without the tumor. In some instances, the cancer is a pancreatic cancer (e.g., an advanced pancreatic cancer, a metastatic pancreatic cancer, a pancreatic adenocarcinoma, a metastatic pancreatic adenocarcinoma, an MSI-H pancreatic cancer, a pancreatic ductal adenocarcinoma, a metastatic pancreatic ductal adenocarcinoma). In some instances, the cancer is a pancreatic adenocarcinoma. In some instances, the cancer is a metastatic pancreatic cancer. In some instances, the cancer is a metastatic pancreatic adenocarcinoma. In some instances, the pancreatic cancer is an advanced pancreatic cancer. In some instances, the pancreatic cancer is an MSI-H pancreatic cancer. In some instances, the pancreatic cancer is a pancreatic ductal adenocarcinoma. In some instances, the pancreatic cancer is a metastatic pancreatic ductal adenocarcinoma. In some instances, the human patient has a prostate cancer (e.g., an mCRPC, a castration-sensitive prostate cancer, a castration-resistant prostate cancer, an MSI-H prostate cancer, a metastatic prostate cancer, a metastatic castrate-resistant, chemo-refractory prostate cancer; an advanced prostate cancer where the patient has exhausted all standard therapies with elevated baseline serum GDF15; an advanced prostate cancer where the patient has previously received one or more (e.g., 1, 2, 3, or more) lines of cancer therapies; a chemo-refractory prostate cancer; a metastatic chemo-refractory prostate cancer). In some instances, the cancer is advanced prostate cancer. In some instances, the human patient has prostate cancer and poor survival. In some instances, the cancer is an mCRPC. In some instances, the cancer is chemo-refractory prostate cancer. In some instances, the cancer is metastatic chemo-refractory prostate cancer. In some instances, the cancer is metastatic castrate-resistant, chemo-refractory prostate cancer. In some instances, the cancer is advanced prostate cancer, wherein the human patient has exhausted all standard therapies, and wherein the human patient has an elevated baseline serum GDF15 (compared to a human or a panel of humans not having prostate cancer). In some instances, the cancer is advanced prostate cancer (e.g., castration-resistant prostate cancer), wherein the human patient has previously received one or more (e.g., 1, 2, 3, or more) lines of therapies (e.g., leuprolide acetate (LUPRON), zoledronic acid (ZOMETA), docetaxel, cabazitaxel, radium 223, mitoxantrone, Oxaliplatin and pemetrexed).

In some cases, the human patient has an elevated serum level of GDF15 prior to the administering of the anti-GFRAL antibody compared to the serum level of GDF15 in said human prior to developing the cancer. In some instances, the human patient has an elevated level of GDF15 prior to the administering of the anti-GFRAL antibody. In some instances, the elevated level of GDF15 is an elevated level of circulating GDF15. In some instances, the elevated level of GDF15 is an elevated level of serum GDF15. In some cases, the elevated level of GDF15 is in the cancer tissue relative to the same non-cancerous tissue (e.g., in a cancerous prostate tissue relative to a non-cancerous prostate tissue). In some instances, the elevated level of GDF15 is as compared to a level of GDF15 in a human not having the cancer or a panel of humans (e.g., 2, 3, 4, 5, 10, 15, 20, 25, or more) not having the cancer. In some instances, the human patient has a level of GDF15 that is at least 500 pg/mL (e.g., at least 500 pg/mL, at least 550 pg/mL, at least 600 pg/mL, at least 650 pg/mL, at least 700 pg/mL, at least 750 pg/mL, at least 800 pg/mL, at least 850 pg·mL, at least 900 pg/mL, at least 950 pg/mL, at least 1000 pg/mL, at least 1100 pg/mL, at least 1200 pg/mL, at least 1290 pg/mL, at least 1300 pg/mL, at least 1400 pg/mL, at least 1500 pg/mL, at least 1600 pg/mL, at least 1700 pg/mL, at least 1800 pg/mL, at least 1900 pg/mL, at least 2000 pg/mL, at least 3000 pg/mL, at least 4,000 pg/mL, at least 5,000 pg/mL, at least 10,000 pg/mL, at least 20,000 pg/mL, at least 30,000 pg/mL, at least 40,000 pg/mL, at least 50,000 pg/mL). In some instances, the human patient has a level of GDF between 1,000 and 60,000 pg/mL, inclusive. In some instances, the human patient has a level of GDF15 that is between 500 and 60,000 pg/mL, inclusive. In some instances, the human patient has a level of GDF15 that is between 800 and 60,000 pg/mL, inclusive. In some instances, the human patient has a level of GDF15 that is between 1000 and 60,000 pg/mL, inclusive. In some instances, the human patient has a level of GDF15 that is between 1,100 and 60,000 pg/mL, inclusive. In some instances, the human patient has a level of GDF15 that is between 1,300 and 60,000 pg/mL, inclusive. In some instances, the human patient has a level of GDF15 that is between 1,500 and 60,000 pg/mL, inclusive. In some instances, the human patient has a level of GDF15 that is between 1,000 and 50,000 pg/mL, inclusive. In some instances, the human patient has a level of GDF15 that is between 1,000 and 40,000 pg/mL, inclusive. In some instances, the human patient has a level of GDF15 that is between 1,000 and 30,000 pg/mL, inclusive. In some instances, the human patient has a level of GDF15 that is between 1,000 and 20,000 pg/mL, inclusive. In some instances, the human patient has a level of GDF15 that is between 1,000 and 10,000 pg/mL, inclusive. In some instances, the human patient has a level of GDF15 that is between 1,000 and 5,000 pg/mL, inclusive. In some instances, the human patient has a level of GDF15 that is between 1,000 and 3,000 pg/mL, inclusive. In some instances, the human patient has a serum GDF15 level of at least 950 pg/ml. In some instances, the human patient has a serum GDF15 level of at least 1300 pg/ml. In some instances, the level of GDF15 is a level of circulating GDF15. In some instances, the level of GDF15 is a level of serum GDF15.

In some instances, the patient is selected for treatment has elevated serum level of GDF15 in comparison with his or her own level before developing the cancer.

In some instances, the human patient has a reduced serum GDF15 level after the administering of the anti-GFRAL antibody as compared to the serum GDF15 level in the human patient prior to the administering of the anti-GFRAL antibody. In some instances, the method reduces the level of GDF15 (e.g., the level of GDF15 is decreased by at least 10%, at least 20%, at least 30%, at least 40%, at least 50%, at least 60%, at least 70%, at least 80%, at least 90%, at least 98%, 10-98%, 20-98%, 30-98%, 40-98%, 50-98%, 60-98%, 70-98%, 80-98%, or 90-98% after being administered the anti-GFRAL antibody (e.g., within 1, 2, 3, 4, 5, 6, 7, 8, 9, 10, 11, 12, 13, 14, 15, 16, 17, 18, 19, 20, 21, 22, 23, 24 or more weeks of being administered 1, 2, 3, 4, 5, 6, or more doses of the anti-GFRAL antibody) compared to the level of GDF15 before being administered the anti-GFRAL antibody. Thus, in some instances, the level of GDF15 may be determined in a method of assessing effectiveness of administering the anti-GFRAL antibody to treat a human patient having cancer. In some instances, the level of GDF15 is a blood level of GDF15. In some instances, the level of GDF15 is a serum level of GDF15.

In some instances, the method further comprises determining a first level of GDF15. In some instances, the determining of a first level of GDF15 is before administering a first dose of the anti-GFRAL antibody. In some instances, the first level of GDF15 is a blood level of GDF15. In some instances, the first level of GDF15 is a serum level of GDF15. In some instances, the method further comprises determining a second level of GDF15 after administering the anti-GFRAL antibody. In some instances, the second level of GDF15 is a blood level of GDF15. In some instances, the second level of GDF15 is a serum level of GDF15. In some instances, the determining of the second level of GDF15 is after 2, 3, 4, 5, 6, 7, 8, 9, 10, or more doses of the anti-GFRAL antibody are administered to the human subject. In some instances, the second level of GDF15 is at least 10%, at least 20%, at least 30%, at least 40%, at least 50%, at least 60%, at least 70%, at least 80%, at least 90%, at least 98%, 10-98%, 20-98%, 30-98%, 40-98%, 50-98%, 60-98%, 70-98%, 80-98%, or 90-98% less than the first level of GDF15.

In some instances, the method for treating the tumor stabilizes progression of the tumor. In some instances, the method for treating the cancer slows down progression of the tumor. In some instances, the method for treating the tumor halts progression of the cancer. In some instances, the method for treating the tumor shrinks a tumor size. In some instances, the method for treating the tumor increases overall survival of the patient. Methods for assessing the progression of a cancer are known in the art and include, e.g., evaluation of target lesions (e.g., measurement of tumor or lesion, using, e.g., X-ray, computerized tomography scan, magnetic resonance imaging, caliper measurement, or positron emission tomography scan), cytology or histology, or evaluation of tumor marker(s) (see, e.g., Eisenhauer et al., 2009, European Journal of Cancer 45:228-247 and Schwartz et al., 2016, European Journal of Cancer 62:132-137; each of which is incorporated by reference herein in its entirety).

In some instances, the human patient has tumor-related weight loss. In some instances, the method reduces tumor-related weight loss (e.g., the human patient loses less than 5%, less than 10%, less than 15%, less than 20%, less than 25%, less than 30%, less than 40%, or less than 50%, or does not lose more than 5%, more than 10%, more than 15%, more than 20%, more than 25%, more than 30%, more than 40%, or more than 50% of weight after being administered the anti-GFRAL antibody (e.g., within 1, 2, 3, 4, 5, 6, 7, 8, 9, 10 or more weeks of being administered 1, 2, 3, 4, 5, or more doses of the anti-GFRAL antibody)). In some instances, the method increases lean body mass in the human patient (e.g., the human patient has an increase of at least 1%, at least 2%, at least 3%, at least 4%, at least 5%, at least 6%, at least 7%, at least 10%, at least 15%, at least 20%, at least 25%, 1-5%, 1-10%, 1-15%, 1-20%, 1-25%, 2-5%, 2-10%, 2-15%, 2-20%, 3-5%, 3-10%, 3-15%, 3-20%, 3-25%, 4-5%, 4-10%, 4-15%, 4-20%, 4-25%, 4-5%, 5-10%, 5-15%, 5-20%, 5-25%, 6-10%, 6-15%, 6-20%, 6-25%, 7-10%, 7-15%, 7-20%, 7-25%, 8-10%, 8-15%, 8-20%, 8-25%, 10-15%, 10-20%, 10-25%, 15-20%, 15-25%, 20%-25% of lean body mass after being administered the anti-GFRAL antibody (e.g., within 1, 2, 3, 4, 5, 6, 7, 8, 9, 10 or more weeks of being administered 1, 2, 3, 4, 5, or more doses of the anti-GFRAL antibody)).

In some instances, the human patient has tumor-related cachexia. Cachexia is a wasting syndrome that is marked with loss of weight, muscle atrophy, fatigue, weakness, and significant loss of appetite in someone who is not actively trying to lose weight. Cachexia can greatly contribute to morbidity of patients suffering from some chronic diseases (e.g., cancer, chronic renal disease, chronic inflammatory disease, muscle wasting, such as muscular dystrophy, and anorexia nervosa). For example, in late stage cancer, cachexia is common (occurring in most terminally ill cancer patients), and is responsible for about a quarter of all cancer-related deaths. In some instances, the method of treating the tumor reduces one or more (e.g., 1, 2, 3, 4, 5) symptoms of cachexia. Reduction of cachexia may be measured by, e.g., evaluating weight loss, muscle atrophy, fatigue, weakness, or appetite.

In some instances, the human patient previously failed to be treated with one or more (e.g., 1, 2, 3, 4, 5) other cancer therapies (i.e., other than an anti-GFRAL antibody described herein) prior to the administering of the anti-GFRAL antibody. In some instances, the human patient previously failed to be treated with one other cancer therapy prior to the administering of the anti-GFRAL antibody. In some instances, the human patient previously failed to be treated with two other cancer therapies prior to the administering of the anti-GFRAL antibody. In some instances, the human patient previously failed to be treated with three other cancer therapies prior to the administering of the anti-GFRAL antibody.

In some instances, the human patient has a prostate cancer (e.g., an mCRPC, a castration-sensitive prostate cancer, a castration-resistant prostate cancer, an MSI-H prostate cancer, a metastatic prostate cancer, a metastatic castrate-resistant, chemo-refractory prostate cancer; an advanced prostate cancer where the patient has exhausted all standard therapies with elevated baseline serum GDF15; an advanced prostate cancer where the patient has previously received one or more (e.g., 1, 2, 3, or more) lines of cancer therapies; a chemo-refractory prostate cancer; a metastatic chemo-refractory prostate cancer). In some instances, the cancer is advanced prostate cancer. In some instances, the human patient has prostate cancer and poor survival. In some instances, the cancer is an mCRPC. In some instances, the cancer is chemo-refractory prostate cancer. In some instances, the cancer is metastatic chemo-refractory prostate cancer. In some instances, the cancer is metastatic castrate-resistant, chemo-refractory prostate cancer. In some instances, the cancer is advanced prostate cancer, wherein the human patient has exhausted all standard therapies, and wherein the human patient has an elevated baseline serum GDF15 (compared to a human or a panel of humans not having prostate cancer). In some cases, the human patient has castration-resistant prostate cancer, and has received one or more lines of hormone therapies (e.g., leuprolide acetate (LUPRON), zoledronic acid (ZOMETA), docetaxel, cabazitaxel, radium 223, mitoxantrone, Oxaliplatin and pemetrexed). In some instances, the human patient has castration-resistant prostate cancer and previously failed after one or more lines of hormone therapies (e.g., leuprolide acetate (LUPRON), zoledronic acid (ZOMETA), docetaxel, cabazitaxel, radium 223, mitoxantrone, Oxaliplatin and pemetrexed). In some instances, the cancer is advanced prostate cancer (e.g., castration-resistant prostate cancer), wherein the human patient has previously received one or more (e.g., 1, 2, 3, or more) lines of therapies (e.g., leuprolide acetate (LUPRON), zoledronic acid (ZOMETA), docetaxel, cabazitaxel, radium 223, mitoxantrone, Oxaliplatin and pemetrexed).

Standard therapies for prostate cancer are known in the art. Non-limiting examples of standard therapy for prostate cancer include surgery, androgen-deprivation therapy, abiraterone, abiraterone acetate, apalutamide, bicalutamide, cabazitaxel, bicalutamide, darolutamide, degarelix, docetaxel, leuprolide acetate (LUPRON), enzalutamide, apalutamide, degarelix, flutamide, goserelin acetate, cabazitaxel, lutetium Lu 177 vipivotide tetraxetan, olaparib, mitoxantrone, mitoxantrone hydrochloride, nilutamide, relugolix, sipuleucel-t, radium 223, radium 223 dichloride, rucaparib camsylate, goserelin acetate, zoledronic acid (ZOMETA, and Oxaliplatin and pemetrexed.

In some instances in which the cancer is prostate cancer (e.g., an mCRPC, a castration-resistant prostate cancer tumor, a metastatic castrate-resistant, chemo-refractory prostate cancer, an advanced prostate cancer where the patient has exhausted all standard therapies with elevated baseline serum GDF15; an advanced prostate cancer where the patient has previously received one or more (e.g., 1, 2, 3, or more) lines of cancer therapies; a chemo-refractory prostate cancer; a metastatic chemo-refractory prostate cancer) the cancer is PSA positive and the method reduces the level of PSA (e.g., the level of PSA is decreased by at least 10%, at least 20%, at least 30%, at least 40%, at least 50%, at least 60%, at least 70%, at least 80%, at least 90%, at least 98%, 10-98%, 20-98%, 30-98%, 40-98%, 50-98%, 60-98%, 70-98%, 80-98%, or 90-98% after being administered the anti-GFRAL antibody (e.g., within 1, 2, 3, 4, 5, 6, 7, 8, 9, 10, 11, 12, 13, 14, 15, 16, 17, 18, 19, 20, 21, 22, 23, 24 or more weeks of being administered 1, 2, 3, 4, 5, 6, or more doses of the anti-GFRAL antibody) compared to the level of PSA before being administered the anti-GFRAL antibody. Thus, in some instances, the level of PSA may be determined in a method of assessing effectiveness of administering the anti-GFRAL antibody to treat a human patient having prostate cancer (e.g., an mCRPC, a castration-resistant prostate cancer tumor, a metastatic castrate-resistant, chemo-refractory prostate cancer; an advanced prostate cancer where the patient has exhausted all standard therapies with elevated baseline serum GDF15; an advanced prostate cancer where the patient has previously received one or more (e.g., 1, 2, 3, or more) lines of cancer therapies; a chemo-refractory prostate cancer; a metastatic chemo-refractory prostate cancer). In some instances, the level of PSA is a blood level of PSA. In some instances, the level of PSA is a serum level of PSA.

In some instances in which the cancer is prostate cancer (e.g., an mCRPC, a castration-resistant prostate cancer tumor, a metastatic castrate-resistant, chemo-refractory prostate cancer; an advanced prostate cancer where the patient has exhausted all standard therapies with elevated baseline serum GDF15; an advanced prostate cancer where the patient has previously received one or more (e.g., 1, 2, 3, or more) lines of cancer therapies; a chemo-refractory prostate cancer; a metastatic chemo-refractory prostate cancer), the method further comprises determining a first level of PSA. In some instances, the determining of a first level of PSA is before administering a first dose of the anti-GFRAL antibody. In some instances, the first level of PSA is a blood level of PSA. In some instances, the first level of PSA is a serum level of PSA. In some instances, the method further comprises determining a second level of PSA after administering the anti-GFRAL antibody. In some instances, the second level of PSA is a blood level of PSA. In some instances, the second level of PSA is a serum level of PSA. In some instances, the determining of the second level of PSA is after 2, 3, 4, 5, 6, 7, 8, 9, 10, or more doses of the anti-GFRAL antibody are administered to the human subject. In some instances, the second level of PSA is at least 10%, at least 20%, at least 30%, at least 40%, at least 50%, at least 60%, at least 70%, at least 80%, at least 90%, at least 98%, 10-98%, 20-98%, 30-98%, 40-98%, 50-98%, 60-98%, 70-98%, 80-98%, or 90-98% less than the first level of PSA.

In some instances in which the cancer is a prostate cancer, administering of the anti-GFRAL antibody is in combination with one or more (e.g., 1, 2, 3, or more) anti-prostate cancer therapies (e.g., a chemotherapy, a radiation therapy, a hormonal therapy, or a surgical therapy). In some instances, the anti-prostate cancer therapy is surgery. In some instances, the anti-prostate cancer therapy is a radiation therapy. In some instances, the anti-prostate cancer therapy is androgen-deprivation therapy. In some instances, the anti-prostate cancer is a chemotherapy. In some instances, the anti-GFRAL antibody is administered prior to the anti-prostate cancer therapy (e.g., within 1 week, within 1 month, within 2 months, within 3 months, within 6 months prior to the anti-prostate cancer therapy). In some instances, the anti-GFRAL antibody is administered after the anti-prostate cancer therapy (e.g., within 1 week, within 1 month, within 2 months, within 3 months, within 6 months after the anti-prostate cancer therapy). In some instances, the anti-GFRAL antibody is administered simultaneously with the anti-prostate cancer therapy (e.g., within one day). In some instances, the anti-GFRAL antibody is administered after androgen-deprivation therapy. In some instances, the anti-GFRAL antibody is administered before chemotherapy.

In some instances, the anti-GFRAL antibody is a first-line treatment. In some instances, the human patient has not previously been treated with any other cancer therapy (i.e., other than an anti-GFRAL antibody described herein) prior to the administering of the anti-GFRAL antibody.

In some instances, the human patient has previously received one or more (e.g., 1, 2, 3, or more) anti-cancer therapies prior to the administering of the anti-GFRAL antibody.

In some instances, the human patient is receiving a concomitant anti-cancer therapy. In some instances, the method further comprises administering to the human patient an anti-cancer therapy. In some instances, the anti-cancer therapy is a standard of care anti-cancer therapy for the tumor (e.g., cancer) being treated. In certain cases, the anti-cancer therapy is a chemotherapy, a radiation therapy, a hormonal therapy, or a surgical therapy.

In some instances, the human patient is about 35 years of age, about 40 years of age, about 45 years of age, about 50 years of age, about 55 years of age, about 60 years of age, about 65 years of age, about 70 years of age, about 75 years of age, about 80 years of age, or about 85 years of age. In some instances, the human patient is at least 35 years of age, at least 40 years of age, at least 45 years of age, at least 50 years of age, at least 55 years of age, at least 60 years of age, at least 65 years of age, at least 70 years of age, at least 75 years of age, at least 80 years of age, or at least 85 years of age. In some instances, the human patient is 35-90 years of age, 35-80 years of age, 35-70 years of age, 45-90 years of age, 45-80 years of age, 45-70 years of age, 55-90 years of age, 55-80 years of age, 55 to 75 years of age, or 55-65 years of age.

In some instances, the human patient is administered at least 2, at least 3, at least 4, at least 5, at least 6, at least 7 at least 8, at least 9, at least 10 doses, at least 11 doses, at least 12 doses, or 2, 3, 4, 5, 6, 7, 8, 9, 10, 11, 12, 13, 14, 15, 16, 17, 18, 19, 20, 21, 22, 23, 24, 25, 26, 27, 28, 29, 30 or more doses of the anti-GFRAL antibody.

In some instances, each of the at least 2, at least 3, at least 4, at least 5, at least 6, at least 7 at least 8, at least 9, at least 10 doses, at least 11 doses, at least 12 doses, or 2, 3, 4, 5, 6, 7, 8, 9, 10, 11, 12, 13, 14, 15, 16, 17, 18, 19, 20, 21, 22, 23, 24, 25, 26, 27, 28, 29, 30 or more doses of the anti-GFRAL antibody is administered about 3 weeks apart (i.e., once about every 3 weeks). In some instances, each of the doses of the anti-GFRAL antibody is administered 3 weeks apart (i.e., once every 3 weeks).

In some instances, each of the at least 2, at least 3, at least 4, at least 5, at least 6, at least 7 at least 8, at least 9, at least 10 doses, at least 11 doses, at least 12 doses, or 2, 3, 4, 5, 6, 7, 8, 9, 10, 11, 12, 13, 14, 15, 16, 17, 18, 19, 20, 21, 22, 23, 24, 25, 26, 27, 28, 29, 30 or more doses of the anti-GFRAL antibody is about 30 mg and is administered about 3 weeks apart (i.e., once about every 3 weeks). In some instances, each of the doses of the anti-GFRAL antibody is 30 mg and is administered 3 weeks apart (i.e., once every 3 weeks). In some instances, each of the at least 2, at least 3, at least 4, at least 5, at least 6, at least 7 at least 8, at least 9, at least 10 doses, at least 11 doses, at least 12 doses, or 2, 3, 4, 5, 6, 7, 8, 9, 10, 11, 12, 13, 14, 15, 16, 17, 18, 19, 20, 21, 22, 23, 24, 25, 26, 27, 28, 29, 30 or more doses of the anti-GFRAL antibody is about 100 mg and is administered about 3 weeks apart (i.e., once about every 3 weeks). In some instances, each of the doses of the anti-GFRAL antibody is 100 mg and is administered 3 weeks apart (i.e., once every 3 weeks).

In some instances, the anti-GFRAL antibody comprises (i) a VH comprising the VH CDR1, the VH CDR2, the VH CDR3 from the amino acid sequence set forth in SEQ ID NO:3 or 1982; and (ii) a VL comprising the VL CDR1, the VL CDR2, and the VL CDR3 from the amino acid sequence set forth in SEQ ID NO:4 or 1997. In some instances, the anti-GFRAL antibody comprises: (i) a VH comprising a VH CDR1 comprising the amino acid sequence set forth in SEQ ID NO:46, a VH CDR2 comprising the amino acid sequence set forth in SEQ ID NO:137, and a VH CDR3 comprising the amino acid sequence set forth in SEQ ID NO:225, and (ii) a VL comprising a VL CDR1 comprising the amino acid sequence set forth in SEQ ID NO:301, a VL CDR2 comprising the amino acid sequence set forth in SEQ ID NO:376, and a VL CDR3 comprising the amino acid sequence set forth in SEQ ID NO:426. In some instances, the anti-GFRAL antibody comprises: (i) a VH comprising a VH CDR1 comprising the amino acid sequence set forth in SEQ ID NO:48, a VH CDR2 comprising the amino acid sequence set forth in SEQ ID NO:137, and a VH CDR3 comprising the amino acid sequence set forth in SEQ ID NO:225; and (ii) a VL comprising a VL CDR1 comprising the amino acid sequence set forth in SEQ ID NO:301, a VL CDR2 comprising the amino acid sequence set forth in SEQ ID NO:376, and a VL CDR3 comprising the amino acid sequence set forth in SEQ ID NO:426. In some instances, the anti-GFRAL antibody comprises: (i) a VH comprising an amino acid sequence having at least at least 75%, at least 80%, at least 85%, at least 90%, at least 95%, at least 96%, at least 97%, at least 98%, at least 99%, or 100% identity to the amino acid sequence set forth in SEQ ID NO:1982; and (ii) a VL comprising an amino acid sequence having at least at least 75%, at least 80%, at least 85%, at least 9(0%, at least 95%, at least 96%, at least 97%, at least 98%, at least 99%, or 100% identity to the amino acid sequence set forth in SEQ ID NO:1997. In some instances, the anti-GFRAL antibody comprises a VH comprising the amino acid sequence set forth in SEQ ID NO: 1982. In some instances, the anti-GFRAL antibody comprises a VL comprising the amino acid sequence set forth in SEQ ID NO:1997. In some instances, the anti-GFRAL antibody comprises: (i) a VH comprising the amino acid sequence set forth in SEQ ID NO:1982; and (ii) a VL comprising the amino acid sequence set forth in SEQ ID NO:1997. In some instances, the anti-GFRAL antibody comprises a heavy chain comprising an amino acid sequence having at least at least 75%, at least 80%, at least 85%, at least 90%, at least 95%, at least 96%, at least 97%, at least 98%, at least 99%, or 100% identity to the amino acid sequence set forth in SEQ ID NO:2010. In some instances, the anti-GFRAL antibody comprises a light chain comprising an amino acid sequence having at least at least 75%, at least 80%, at least 85%, at least 90%, at least 95%, at least 96%, at least 97%, at least 98%, at least 99%, or 100% identity to the amino acid sequence set forth in SEQ ID NO:2012. In some instances, the anti-GFRAL antibody comprises: (i) a heavy chain comprising an amino acid sequence having at least at least 75%, at least 80%, at least 85%, at least 90%, at least 95%, at least 96%, at least 97%, at least 98%, at least 99%, or 100% identity to the amino acid sequence set forth in SEQ ID NO:2010; and (ii) a light chain comprising an amino acid sequence having at least at least 75%, at least 80%, at least 85%, at least 90%, at least 95%, at least 96%, at least 97%, at least 98%, at least 99%, or 100% identity to the amino acid sequence set forth in SEQ ID NO:2012. In some instances, the anti-GFRAL antibody comprises a heavy chain comprising the amino acid sequence set forth in SEQ ID NO:2010. In some instances, the anti-GFRAL antibody comprises a heavy chain consisting of the amino acid sequence set forth in SEQ ID NO:2010. In some instances, the anti-GFRAL antibody comprises a light chain comprising the amino acid sequence set forth in SEQ ID NO:2012. In some instances, the anti-GFRAL antibody comprises a light chain consisting of the amino acid sequence set forth in SEQ ID NO:2012. In some instances, the anti-GFRAL antibody comprises: (i) a heavy chain comprising the amino acid sequence set forth in SEQ ID NO:2010; and (ii) a light chain comprising the amino acid sequence set forth in SEQ ID NO:2012. In some instances, the anti-GFRAL antibody comprises: (i) a heavy chain consisting of the amino acid sequence set forth in SEQ ID NO:2010; and (ii) a light chain consisting of the amino acid sequence set forth in SEQ ID NO:2012. In some instances, the dose of the anti-GFRAL antibody is about 30 mg. In some instances, the dose of the anti-GFRAL antibody is 30 mg. In some instances, the dose of the anti-GFRAL antibody is about 100 mg. In some instances, the dose of the anti-GFRAL antibody is 100 mg. In some instances, the dose of the anti-GFRAL antibody is administered once about every 3 weeks. In some instances, the dose of the anti-GFRAL antibody is administered once every 3 weeks. In some instances, the dose of the anti-GFRAL antibody is about 30 mg administered once about every 3 weeks. In some instances, the dose of the anti-GFRAL antibody is 30 mg administered once every 3 weeks. In some instances, the dose of the anti-GFRAL antibody is about 100 mg administered once about every 3 weeks. In some instances, the dose of the anti-GFRAL antibody is 100 mg administered once every 3 weeks. In some instances, the anti-GFRAL antibody is administered subcutaneously. In some instances, the anti-GFRAL antibody is administered subcutaneously at a dose of 30 mg once every 3 weeks. In some instances, the anti-GFRAL antibody is administered subcutaneously at a dose of 100 mg once every 3 weeks. In some instances, the anti-GFRAL antibody is administered intravenously. In some instances, the human patient previously failed to be treated with one or more (e.g., 1, 2, 3, 4, 5) other cancer therapies (i.e., other than an anti-GFRAL antibody described herein) prior to the administering of the anti-GFRAL antibody. In some instances, the cancer is metastatic. In some instances, the cancer is advanced. In some instances, the cancer is a prostate cancer (e.g., an mCRPC, a castration-sensitive prostate cancer, a castration-resistant prostate cancer, an MSI-H prostate cancer, a metastatic prostate cancer, a metastatic castrate-resistant, chemo-refractory prostate cancer; an advanced prostate cancer where the patient has exhausted all standard therapies with elevated baseline serum GDF15; an advanced prostate cancer where the patient has previously received one or more (e.g., 1, 2, 3, or more) lines of cancer therapies; a chemo-refractory prostate cancer; a metastatic chemo-refractory prostate cancer), a bladder cancer, a melanoma, an SCLC (e.g., an extensive-stage SCLC), an NSCLC, a pancreatic cancer (e.g., an advanced pancreatic cancer, a metastatic pancreatic cancer, a pancreatic adenocarcinoma, a metastatic pancreatic adenocarcinoma, an MSI-H pancreatic cancer, a pancreatic ductal adenocarcinoma, a metastatic pancreatic ductal adenocarcinoma), a colorectal cancer, a gastric cancer, an esophageal cancer, an ovarian cancer, a head neck squamous cell carcinoma tumor or an MSI-H cancer (e.g., an MSI-H prostate cancer, an MSI-H ovarian cancer, an MSI-H ovarian cancer, an MSI-H HNSCC, an MSI-H SLCLC (e.g., an MSI-H extensive-stage SCLC). In some instances, the cancer is an MSI-H cancer selected from a castration-resistant prostate cancer, a bladder cancer, a melanoma, a SCLC (e.g., an extensive-stage SCLC), a pancreatic cancer (e.g., an advanced pancreatic cancer, a metastatic pancreatic cancer, a pancreatic adenocarcinoma, a metastatic pancreatic adenocarcinoma, an MSI-H pancreatic cancer, a pancreatic ductal adenocarcinoma, a metastatic pancreatic ductal adenocarcinoma), a colorectal cancer, a gastric cancer, an esophageal cancer, an ovarian cancer, a head and neck squamous cell carcinoma). In some instances, the cancer is a pancreatic cancer (e.g., an advanced pancreatic cancer, a metastatic pancreatic cancer, a pancreatic adenocarcinoma, a metastatic pancreatic adenocarcinoma, an MSI-H pancreatic cancer, a pancreatic ductal adenocarcinoma, a metastatic pancreatic ductal adenocarcinoma). In some instances, the cancer is a pancreatic adenocarcinoma. In some instances, the cancer is a metastatic pancreatic cancer. In some instances, the cancer is a metastatic pancreatic adenocarcinoma. In some instances, the pancreatic cancer is an advanced pancreatic cancer. In some instances, the pancreatic cancer is an MSI-H pancreatic cancer. In some instances, the pancreatic cancer is a pancreatic ductal adenocarcinoma. In some instances, the pancreatic cancer is a metastatic pancreatic ductal adenocarcinoma. In some instances, the human patient has a prostate cancer (e.g., an mCRPC, a castration-sensitive prostate cancer, a castration-resistant prostate cancer, an MSI-H prostate cancer, a metastatic prostate cancer, a metastatic castrate-resistant, chemo-refractory prostate cancer; an advanced prostate cancer where the patient has exhausted all standard therapies with elevated baseline serum GDF15; an advanced prostate cancer where the patient has previously received one or more (e.g., 1, 2, 3, or more) lines of cancer therapies; a chemo-refractory prostate cancer; a metastatic chemo-refractory prostate cancer). In some instances, the human patient has castration-resistant prostate cancer. In some instances, the human patient has castration-resistant prostate cancer and previously failed after one or more lines of hormone therapies. In some cases, the human patient has an advanced prostate cancer (e.g., castration-resistant prostate cancer) and the patient has previously received one or more (e.g., 1, 2, 3, or more) lines of cancer therapies (e.g., leuprolide acetate (LUPRON), zoledronic acid (ZOMETA), docetaxel, cabazitaxel, radium 223, mitoxantrone. Oxaliplatin and pemetrexed).

In some instances, the anti-GFRAL antibody is administered subcutaneously. In some instances, the anti-GFRAL antibody is administered intravenously.

2. Methods of Treating Tumor-Related Weight Loss or Cachexia

An anti-GFRAL antibody described herein or a pharmaceutical composition comprising the same can also be used to treat a human patient having tumor-related weight loss or cachexia.

Thus, provided herein is a method for treating tumor-related weight loss or cachexia in a human patient, the method comprising administering to the human patient an anti-GFRAL antibody described herein at a dose of about 30 mg (or a pharmaceutical composition comprising the same) once about every 3 weeks. In some instances, the method is for treating tumor-related weight loss. In some instances, the method is for treating tumor-related cachexia. In some instances, the dose is 30 mg. In some instances, the dose is administered once every 3 weeks. In some instances, the dose is 30 mg and the dose is administered once every 3 weeks. In some instances, the anti-GFRAL antibody is administered subcutaneously. In some instances, the anti-GFRAL antibody is administered subcutaneously at a dose of about 30 mg once about every 3 weeks. In some instances, the anti-GFRAL antibody is administered subcutaneously at a dose of 30 mg once every 3 weeks. In some instances, the anti-GFRAL antibody is administered intravenously.

Also provided herein is a method for treating tumor-related weight loss or cachexia in a human patient, comprising administering to the human patient an anti-GFRAL antibody described herein at a dose of about 100 mg (or a pharmaceutical composition comprising the same) once about every 3 weeks. In some instances, the method is for treating tumor-related weight loss. In some instances, the method is for treating tumor-related cachexia. In some instances, the dose is 100 mg. In some instances, the dose is administered once every 3 weeks. In some instances, the dose is 100 mg and the dose is administered once every 3 weeks. In some instances, the anti-GFRAL antibody is administered subcutaneously. In some instances, the anti-GFRAL antibody is administered subcutaneously at a dose of about 100 mg once about every 3 weeks. In some instances, the anti-GFRAL antibody is administered subcutaneously at a dose of 100 mg once every 3 weeks. In some instances, the anti-GFRAL antibody is administered intravenously.

In some cases, the human patient has an elevated serum level of GDF15 prior to the administering of the anti-GFRAL antibody compared to the serum level of GDF15 in said human prior to developing the cancer. In some instances, the human patient has an elevated level of GDF15 prior to the administering of the anti-GFRAL antibody. In some instances, the elevated level of GDF15 is an elevated level of circulating GDF15. In some instances, the elevated level of GDF15 is an elevated level of serum GDF15. In some cases, the elevated level of GDF15 is in the cancer tissue relative to the same non-cancerous tissue (e.g., in a cancerous prostate tissue relative to a non-cancerous prostate tissue). In some instances, the elevated level of GDF15 is as compared to a level of GDF15 in a human not having a cancer or a panel of humans (e.g., 2, 3, 4, 5, 10, 15, 20, 25, or more) not having a cancer. In some instances, the human patient has a level of GDF15 that is at least 500 pg/mL (e.g., at least 500 pg/mL, at least 550 pg/mL, at least 600 pg/mL, at least 650 pg/mL, at least 700 pg/mL, at least 750 pg/mL, at least 800 pg/mL, at least 850 pg·mL, at least 900 pg/mL, at least 950 pg/mL, at least 1000 pg/mL, at least 1100 pg/mL, at least 1200 pg/mL, at least 1290 pg/mL, at least 1300 pg/mL, at least 1400 pg/mL, at least 1500 pg/mL, at least 1600 pg/mL, at least 1700 pg/mL, at least 1800 pg/mL, at least 1900 pg/mL, at least 2000 pg/mL, at least 3000 pg/mL, at least 4,000 pg/mL, at least 5,000 pg/mL, at least 10,000 pg/mL, at least 20,000 pg/mL, at least 30,000 pg/mL, at least 40,000 pg/mL, at least 50,000 pg/mL). In some instances, the human patient has a level of GDF between 1,000 and 60,000 pg/mL, inclusive. In some instances, the human patient has a level of GDF15 that is between 500 and 60,000 pg/mL, inclusive. In some instances, the human patient has a level of GDF15 that is between 800 and 60,000 pg/mL, inclusive. In some instances, the human patient has a level of GDF15 that is between 1000 and 60,000 pg/mL, inclusive. In some instances, the human patient has a level of GDF15 that is between 1,100 and 60.000 pg/mL, inclusive. In some instances, the human patient has a level of GDF15 that is between 1,300 and 60,000 pg/mL, inclusive. In some instances, the human patient has a level of GDF15 that is between 1,500 and 60,000 pg/mL, inclusive. In some instances, the human patient has a level of GDF15 that is between 1,000 and 50,000 pg/mL, inclusive. In some instances, the human patient has a level of GDF15 that is between 1,000 and 40,000 pg/mL, inclusive. In some instances, the human patient has a level of GDF15 that is between 1,000 and 30,000 pg/mL, inclusive. In some instances, the human patient has a level of GDF15 that is between 1,000 and 20,000 pg/mL, inclusive. In some instances, the human patient has a level of GDF15 that is between 1,000 and 10,000 pg/mL, inclusive. In some instances, the human patient has a level of GDF15 that is between 1,000 and 5,000 pg/mL, inclusive. In some instances, the human patient has a level of GDF15 that is between 1,000 and 3,000 pg/mL, inclusive. In some instances, the human patient has a serum GDF15 level of at least 950 pg/ml. In some instances, the human patient has a serum GDF15 level of at least 1300 pg/ml. In some instances, the level of GDF15 is a level of circulating GDF15. In some instances, the level of GDF15 is a level of serum GDF15. In some instances, the human patient has a reduced serum GDF15 level after the administering of the anti-GFRAL antibody as compared to the serum GDF15 level in the human patient prior to the administering of the anti-GFRAL antibody.

In some instances, the patient is selected for treatment has elevated serum level of GDF15 in comparison with his or her own level before developing the cancer.

In some instances, the method is for treating tumor-related weight loss. In some instances, the method reduces tumor-related weight loss (e.g., the human patient loses less than 5%, less than 10%, less than 15%, less than 20%, less than 25%, less than 30%, less than 40%, or less than 50%, or does not lose more than 5%, more than 10%, more than 15%, more than 20%, more than 25%, more than 30%, more than 40%, or more than 50% of weight after being administered the anti-GFRAL antibody (e.g., within 1, 2, 3, 4, 5, 6, 7, 8, 9, 10 or more weeks of being administered 2, 3, 4, 5, or more doses of the anti-GFRAL antibody)). In some instances, the method increases lean body mass in the human patient (e.g., the human patient has an increase of at least 1%, at least 2%, at least 3%, at least 4%, at least 5%, at least 6%, at least 7%, at least 10%, at least 15%, at least 20%, at least 25%, 1-5%, 1-10%, 1-15%, 1-20%, 1-25%, 2-5%, 2-10%, 2-15%, 2-20%, 3-5%, 3-10%, 3-15%, 3-20%, 3-25%, 4-5%, 4-10%, 4-15%, 4-20%, 4-25%, 4-5%, 5-10%, 5-15%, 5-20%, 5-25%, 6-10%, 6-15%, 6-20%, 6-25%, 7-10%, 7-15%, 7-20%, 7-25%, 8-10%, 8-15%, 8-20%, 8-25%, 10-15%, 10-20%, 10-25%, 15%-20%, 15-25%, 20%-25% of lean body mass after being administered the anti-GFRAL antibody (e.g., within 1, 2, 3, 4, 5, 6, 7, 8, 9, 10 or more weeks of being administered 1, 2, 3, 4, 5, or more doses of the anti-GFRAL antibody)). In some instances, the tumor is a solid tumor. In some instances, the tumor is an advanced solid tumor. In some instances, the tumor is a cancer. In some instances, the cancer is metastatic. In some instances, the cancer is advanced. In some instances, the cancer is a prostate cancer (e.g., an mCRPC, a castration-sensitive prostate cancer, a castration-resistant prostate cancer, an MSI-H prostate cancer, a metastatic prostate cancer, a metastatic castrate-resistant, chemo-refractory prostate cancer; an advanced prostate cancer where the patient has exhausted all standard therapies with elevated baseline serum GDF15; an advanced prostate cancer where the patient has previously received one or more (e.g., 1, 2, 3, or more) lines of cancer therapies; a chemo-refractory prostate cancer; a metastatic chemo-refractory prostate cancer), a bladder cancer, a melanoma, an SCLC (e.g., an extensive-stage SCLC), an NSCLC, a pancreatic cancer (e.g., an advanced pancreatic cancer, a metastatic pancreatic cancer, a pancreatic adenocarcinoma, a metastatic pancreatic adenocarcinoma, an MSI-H pancreatic cancer, a pancreatic ductal adenocarcinoma, a metastatic pancreatic ductal adenocarcinoma), a colorectal cancer, a gastric cancer, an esophageal cancer, an ovarian cancer, a head neck squamous cell carcinoma or an MSI-H cancer (e.g., an MSI-H prostate cancer, an MSI-H ovarian cancer, an MSI-H ovarian cancer, an MSI-H HNSCC, an MSI-H SLCLC (e.g., an MSI-H extensive-stage SCLC). In some instances, the cancer is an MSI-H cancer selected from a castration-resistant prostate cancer, a bladder cancer, a melanoma, a SCLC (e.g., an extensive-stage SCLC), a pancreatic cancer (e.g., an advanced pancreatic cancer, a metastatic pancreatic cancer, a pancreatic adenocarcinoma, a metastatic pancreatic adenocarcinoma, an MSI-H pancreatic cancer, a pancreatic ductal adenocarcinoma, a metastatic pancreatic ductal adenocarcinoma), a colorectal cancer, a gastric cancer, an esophageal cancer, an ovarian cancer, a head and neck squamous cell carcinoma). In some instances, the cancer is a pancreatic cancer (e.g., an advanced pancreatic cancer, a metastatic pancreatic cancer, a pancreatic adenocarcinoma, a metastatic pancreatic adenocarcinoma, an MSI-H pancreatic cancer, a pancreatic ductal adenocarcinoma, a metastatic pancreatic ductal adenocarcinoma). In some instances, the cancer is a pancreatic adenocarcinoma. In some instances, the cancer is a metastatic pancreatic cancer. In some instances, the cancer is a metastatic pancreatic adenocarcinoma. In some instances, the pancreatic cancer is an advanced pancreatic cancer. In some instances, the pancreatic cancer is an MSI-H pancreatic cancer. In some instances, the pancreatic cancer is a pancreatic ductal adenocarcinoma. In some instances, the pancreatic cancer is a metastatic pancreatic ductal adenocarcinoma. In some instances, the human patient has a prostate cancer (e.g., an mCRPC, a castration-sensitive prostate cancer, a castration-resistant prostate cancer, an MSI-H prostate cancer, a metastatic prostate cancer, a metastatic castrate-resistant, chemo-refractory prostate cancer; an advanced prostate cancer where the patient has exhausted all standard therapies with elevated baseline serum GDF15; an advanced prostate cancer where the patient has previously received one or more (e.g., 1, 2, 3, or more) lines of cancer therapies; a chemo-refractory prostate cancer; a metastatic chemo-refractory prostate cancer). In some instances, the cancer is advanced prostate cancer. In some instances, the human patient has prostate cancer and poor survival. In some instances, the cancer is an mCRPC. In some instances, the cancer is chemo-refractory prostate cancer. In some instances, the cancer is metastatic chemo-refractory prostate cancer. In some instances, the cancer is metastatic castrate-resistant, chemo-refractory prostate cancer. In some instances, the cancer is advanced prostate cancer, wherein the human patient has exhausted all standard therapies, and wherein the human patient has an elevated baseline serum GDF15 (compared to a human or a panel of humans not having prostate cancer). In some instances, the cancer is advanced prostate cancer (e.g., castration-resistant prostate cancer), wherein the human patient has previously received one or more (e.g., 1, 2, 3, or more) lines of therapies (e.g., leuprolide acetate (LUPRON), zoledronic acid (ZOMETA), docetaxel, cabazitaxel, radium 223, mitoxantrone, Oxaliplatin and pemetrexed).

In some instances, method is for treating tumor-related cachexia. In some instances, the method reduces one or more (e.g., 1, 2, 3, 4, 5) symptoms of cachexia. Reduction of cachexia may be measured by, e.g., evaluating weight loss, muscle atrophy, fatigue, weakness, or appetite in a human patient.

In some instances, the human patient has a cancer. In some instances, the cancer is a solid tumor cancer. In some instances, the cancer is an advanced cancer. In some instances, the cancer is an advanced solid tumor. In some instances, the cancer is metastatic. In some instances, the cancer is advanced. In some instances, the cancer is a prostate cancer (e.g., an mCRPC, a castration-sensitive prostate cancer, a castration-resistant prostate cancer, an MSI-H prostate cancer, a metastatic prostate cancer, a metastatic castrate-resistant, chemo-refractory prostate cancer; an advanced prostate cancer where the patient has exhausted all standard therapies with elevated baseline serum GDF15; an advanced prostate cancer where the patient has previously received one or more (e.g., 1, 2, 3, or more) lines of cancer therapies; a chemo-refractory prostate cancer; a metastatic chemo-refractory prostate cancer), a bladder cancer, a melanoma, an SCLC (e.g., an extensive-stage SCLC), an NSCLC, a pancreatic cancer (e.g., an advanced pancreatic cancer, a metastatic pancreatic cancer, a pancreatic adenocarcinoma, a metastatic pancreatic adenocarcinoma, an MSI-H pancreatic cancer, a pancreatic ductal adenocarcinoma, a metastatic pancreatic ductal adenocarcinoma), a colorectal cancer, a gastric cancer, an esophageal cancer, an ovarian cancer, a head neck squamous cell carcinoma or an MSI-H cancer (e.g., an MSI-H prostate cancer, an MSI-H ovarian cancer, an MSI-H ovarian cancer, an MSI-H HNSCC, an MSI-H SLCLC (e.g., an MSI-H extensive-stage SCLC). In some instances, the cancer is an MSI-H cancer selected from a castration-resistant prostate cancer, a bladder cancer, a melanoma, a SCLC (e.g., an extensive-stage SCLC), a pancreatic cancer (e.g., an advanced pancreatic cancer, a metastatic pancreatic cancer, a pancreatic adenocarcinoma, a metastatic pancreatic adenocarcinoma, an MSI-H pancreatic cancer, a pancreatic ductal adenocarcinoma, a metastatic pancreatic ductal adenocarcinoma), a colorectal cancer, a gastric cancer, an esophageal cancer, an ovarian cancer, a head and neck squamous cell carcinoma). In some instances, the cancer is a pancreatic cancer (e.g., an advanced pancreatic cancer, a metastatic pancreatic cancer, a pancreatic adenocarcinoma, a metastatic pancreatic adenocarcinoma, an MSI-H pancreatic cancer, a pancreatic ductal adenocarcinoma, a metastatic pancreatic ductal adenocarcinoma). In some instances, the cancer is a pancreatic adenocarcinoma. In some instances, the cancer is a metastatic pancreatic cancer. In some instances, the cancer is a metastatic pancreatic adenocarcinoma. In some instances, the pancreatic cancer is an advanced pancreatic cancer. In some instances, the pancreatic cancer is an MSI-H pancreatic cancer. In some instances, the pancreatic cancer is a pancreatic ductal adenocarcinoma. In some instances, the pancreatic cancer is a metastatic pancreatic ductal adenocarcinoma. In some instances, the human patient has a prostate cancer (e.g., an mCRPC, a castration-sensitive prostate cancer, a castration-resistant prostate cancer, an MSI-H prostate cancer, a metastatic prostate cancer, a metastatic castrate-resistant, chemo-refractory prostate cancer; an advanced prostate cancer where the patient has exhausted all standard therapies with elevated baseline serum GDF15; an advanced prostate cancer where the patient has previously received one or more (e.g., 1, 2, 3, or more) lines of cancer therapies; a chemo-refractory prostate cancer; a metastatic chemo-refractory prostate cancer). In some instances, the cancer is advanced prostate cancer. In some instances, the human patient has prostate cancer and poor survival. In some instances, the cancer is an mCRPC. In some instances, the cancer is chemo-refractory prostate cancer. In some instances, the cancer is metastatic chemo-refractory prostate cancer. In some instances, the cancer is metastatic castrate-resistant, chemo-refractory prostate cancer. In some instances, the cancer is advanced prostate cancer, wherein the human patient has exhausted all standard therapies, and wherein the human patient has an elevated baseline serum GDF15 (compared to a human or a panel of humans not having prostate cancer). In some instances, the cancer is advanced prostate cancer (e.g., castration-resistant prostate cancer), wherein the human patient has previously received one or more (e.g., 1, 2, 3, or more) lines of therapies (e.g., leuprolide acetate (LUPRON), zoledronic acid (ZOMETA), docetaxel, cabazitaxel, radium 223, mitoxantrone, Oxaliplatin and pemetrexed).

In some instances, the human patient previously failed to be treated with one or more (e.g., 1, 2, 3, or more) other cancer therapies (i.e., other than an anti-GFRAL antibody described herein) prior to the administering of the anti-GFRAL antibody. In some instances, the human patient previously failed to be treated with one other cancer therapy prior to the administering of the anti-GFRAL antibody. In some instances, the human patient previously failed to be treated with two other cancer therapies prior to the administering of the anti-GFRAL antibody. In some instances, the human patient previously failed to be treated with three other cancer therapies prior to the administering of the anti-GFRAL antibody.

In some instances, the human patient has castration-resistant prostate cancer and previously failed or relapsed after one or more lines of hormone therapies. In some cases, the human patient has an advanced prostate cancer (e.g., castration-resistant prostate cancer) and the patient has previously received one or more (e.g., 1, 2, 3, or more) lines of cancer therapies (e.g., leuprolide acetate (LUPRON), zoledronic acid (ZOMETA), docetaxel, cabazitaxel, radium 223, mitoxantrone, Oxaliplatin and pemetrexed). In some instances, the human patient previously failed to be treated with or relapsed from one or more (e.g., 1, 2, 3, 4, 5) other cancer therapies (i.e., other than an anti-GFRAL antibody described herein) prior to the administering of the anti-GFRAL antibody.

In some instances, the anti-GFRAL antibody is a first-line treatment. In some instances, the human patient has not previously been treated with any other cancer therapy (i.e., other than an anti-GFRAL antibody described herein) prior to the administering of the anti-GFRAL antibody.

In some instances, the human patient has previously received one or more (e.g., 1, 2, 3, or more) anti-cancer therapies prior to the administering of the anti-GFRAL antibody.

In some instances, the human patient is receiving a concomitant anti-cancer therapy. In some instances, the method further comprises administering to the human patient an anti-cancer therapy. In certain cases, the anti-cancer therapy is a chemotherapy, a radiation therapy, a hormonal therapy, or a surgical therapy.

In some instances, the human patient is about 35 years of age, about 40 years of age, about 45 years of age, about 50 years of age, about 55 years of age, about 60 years of age, about 65 years of age, about 70 years of age, about 75 years of age, about 80 years of age, or about 85 years of age. In some instances, the human patient is at least 35 years of age, at least 40 years of age, at least 45 years of age, at least 50 years of age, at least 55 years of age, at least 60 years of age, at least 65 years of age, at least 70 years of age, at least 75 years of age, at least 80 years of age, or at least 85 years of age. In some instances, the human patient is 35-90 years of age, 35-80 years of age, 35-70 years of age, 45-90 years of age, 45-80 years of age, 45-70 years of age, 55-90 years of age, 55-80 years of age, 55 to 75 years of age, or 55-65 years of age.

In some instances, the human patient is administered at least 2, at least 3, at least 4, at least 5, at least 6, at least 7 at least 8, at least 9, at least 10 doses, at least 11 doses, at least 12 doses, or 2, 3, 4, 5, 6, 7, 8, 9, 10, 11, 12, 13, 14, 15, 16, 17, 18, 19, 20, 21, 22, 23, 24, 25, 26, 27, 28, 29, 30 or more doses of the anti-GFRAL antibody.

In some instances, each of the at least 2, at least 3, at least 4, at least 5, at least 6, at least 7 at least 8, at least 9, at least 10 doses, at least 11 doses, at least 12 doses, or 2, 3, 4, 5, 6, 7, 8, 9, 10, 11, 12, 13, 14, 15, 16, 17, 18, 19, 20, 21, 22, 23, 24, 25, 26, 27, 28, 29, 30 or more doses of the anti-GFRAL antibody is administered about 3 weeks apart (i.e., once about every 3 weeks). In some instances, each of the doses of the anti-GFRAL antibody is administered 3 weeks apart (i.e., once every 3 weeks).

In some instances, each of the at least 2, at least 3, at least 4, at least 5, at least 6, at least 7 at least 8, at least 9, at least 10 doses, at least 11 doses, at least 12 doses, or 2, 3, 4, 5, 6, 7, 8, 9, 10, 11, 12, 13, 14, 15, 16, 17, 18, 19, 20, 21, 22, 23, 24, 25, 26, 27, 28, 29, 30 or more doses of the anti-GFRAL antibody is about 30 mg and is administered about 3 weeks apart (i.e., once about every 3 weeks). In some instances, each of the doses of the anti-GFRAL antibody is 30 mg and is administered 3 weeks apart (i.e., once every 3 weeks). In some instances, each of the at least 2, at least 3, at least 4, at least 5, at least 6, at least 7 at least 8, at least 9, at least 10 doses, at least 11 doses, at least 12 doses, or 2, 3, 4, 5, 6, 7, 8, 9, 10, 11, 12, 13, 14, 15, 16, 17, 18, 19, 20, 21, 22, 23, 24, 25, 26, 27, 28, 29, 30 or more doses of the anti-GFRAL antibody is about 100 mg and is administered about 3 weeks apart (i.e., once about every 3 weeks). In some instances, each of the doses of the anti-GFRAL antibody is 100 mg and is administered 3 weeks apart (i.e., once every 3 weeks).

In some instances, the anti-GFRAL antibody comprises (i) a VH comprising the VH CDR1, the VH CDR2, the VH CDR3 from the amino acid sequence set forth in SEQ ID NO:3 or 1982, and (ii) a VL comprising the VL CDR1, the VL CDR2, and the VL CDR3 from the amino acid sequence set forth in SEQ ID NO:4 or 1997. In some instances, the anti-GFRAL antibody comprises: (i) a VH comprising a VH CDR1 comprising the amino acid sequence set forth in SEQ ID NO:46, a VH CDR2 comprising the amino acid sequence set forth in SEQ ID NO:137, and a VH CDR3 comprising the amino acid sequence set forth in SEQ ID NO:225; and (ii) a VL comprising a VL CDR1 comprising the amino acid sequence set forth in SEQ ID NO:301, a VL CDR2 comprising the amino acid sequence set forth in SEQ ID NO:376, and a VL CDR3 comprising the amino acid sequence set forth in SEQ ID NO:426. In some instances, the anti-GFRAL antibody comprises: (i) a VH comprising a VH CDR1 comprising the amino acid sequence set forth in SEQ ID NO:48, a VH CDR2 comprising the amino acid sequence set forth in SEQ ID NO:137, and a VH CDR3 comprising the amino acid sequence set forth in SEQ ID NO:225; and (ii) a VL comprising a VL CDR1 comprising the amino acid sequence set forth in SEQ ID NO:301, a VL CDR2 comprising the amino acid sequence set forth in SEQ ID NO:376, and a VL CDR3 comprising the amino acid sequence set forth in SEQ ID NO:426. In some instances, the anti-GFRAL antibody comprises: (i) a VH comprising an amino acid sequence having at least at least 75%, at least 80%, at least 85%, at least 90%, at least 95%, at least 96%, at least 97%, at least 98%, at least 99%, or 100% identity to the amino acid sequence set forth in SEQ ID NO:1982; and (ii) a VL comprising an amino acid sequence having at least at least 75%, at least 80%, at least 85%, at least 90%, at least 95%, at least 96%, at least 97%, at least 98%, at least 99%, or 100% identity to the amino acid sequence set forth in SEQ ID NO:1997. In some instances, the anti-GFRAL antibody comprises a VH comprising the amino acid sequence set forth in SEQ ID NO:1982. In some instances, the anti-GFRAL antibody comprises a VL comprising the amino acid sequence set forth in SEQ ID NO: 1997. In some instances, the anti-GFRAL antibody comprises: (i) a VH comprising the amino acid sequence set forth in SEQ ID NO:1982; and (ii) a VL comprising the amino acid sequence set forth in SEQ ID NO: 1997. In some instances, the anti-GFRAL antibody comprises a heavy chain comprising an amino acid sequence having at least at least 75%, at least 80%, at least 85%, at least 90%, at least 95%, at least 96%, at least 97%, at least 98%, at least 99%, or 100% identity to the amino acid sequence set forth in SEQ ID NO:2010. In some instances, the anti-GFRAL antibody comprises a light chain comprising an amino acid sequence having at least at least 75%, at least 80%, at least 85%, at least 90%, at least 95%, at least 96%, at least 97%, at least 98%, at least 99%, or 100% identity to the amino acid sequence set forth in SEQ ID NO:2012. In some instances, the anti-GFRAL antibody comprises: (i) a heavy chain comprising an amino acid sequence having at least at least 75%, at least 80%, at least 85%, at least 90%, at least 95%, at least 96%, at least 97%, at least 98%, at least 99%, or 100% identity to the amino acid sequence set forth in SEQ ID NO:2010; and (ii) a light chain comprising an amino acid sequence having at least at least 75%, at least 80%, at least 85%, at least 90%, at least 95%, at least 96%, at least 97%, at least 98%, at least 99%, or 100% identity to the amino acid sequence set forth in SEQ ID NO:2012. In some instances, the anti-GFRAL antibody comprises a heavy chain comprising the amino acid sequence set forth in SEQ ID NO:2010. In some instances, the anti-GFRAL antibody comprises a heavy chain consisting of the amino acid sequence set forth in SEQ ID NO:2010. In some instances, the anti-GFRAL antibody comprises a light chain comprising the amino acid sequence set forth in SEQ ID NO:2012. In some instances, the anti-GFRAL antibody comprises a light chain consisting of the amino acid sequence set forth in SEQ ID NO:2012. In some instances, the anti-GFRAL antibody comprises: (i) a heavy chain comprising the amino acid sequence set forth in SEQ ID NO:2010; and (ii) a light chain comprising the amino acid sequence set forth in SEQ ID NO:2012. In some instances, the anti-GFRAL antibody comprises: (i) a heavy chain consisting of the amino acid sequence set forth in SEQ ID NO:2010; and (ii) a light chain consisting of the amino acid sequence set forth in SEQ ID NO:2012. In some instances, the dose of the anti-GFRAL antibody is about 30 mg. In some instances, the dose of the anti-GFRAL antibody is 30 mg. In some instances, the dose of the anti-GFRAL antibody is about 100 mg. In some instances, the dose of the anti-GFRAL antibody is 100 mg. In some instances, the dose of the anti-GFRAL antibody is administered once about every 3 weeks. In some instances, the dose of the anti-GFRAL antibody is administered once every 3 weeks. In some instances, the dose of the anti-GFRAL antibody is about 30 mg administered once about every 3 weeks. In some instances, the dose of the anti-GFRAL antibody is 30 mg administered once every 3 weeks. In some instances, the dose of the anti-GFRAL antibody is about 100 mg administered once about every 3 weeks. In some instances, the dose of the anti-GFRAL antibody is 100 mg administered once every 3 weeks. In some instances, the anti-GFRAL antibody is administered subcutaneously. In some instances, the anti-GFRAL antibody is administered subcutaneously at a dose of 30 mg once every 3 weeks. In some instances, the anti-GFRAL antibody is administered subcutaneously at a dose of 100 mg once every 3 weeks. In some instances, the anti-GFRAL antibody is administered intravenously. In some instances, the human patient previously failed to be treated with one or more (e.g., 1, 2, 3, 4, 5) other cancer therapies (i.e., other than an anti-GFRAL antibody described herein) prior to the administering of the anti-GFRAL antibody. In some instances, the tumor is a solid tumor. In some instances, the tumor is an advanced solid tumor. In some instances, the tumor is a cancer. In some instances, the cancer is metastatic. In some instances, the cancer is advanced. In some instances, the cancer is a prostate cancer (e.g., an mCRPC, a castration-sensitive prostate cancer, a castration-resistant prostate cancer, an MSI-H prostate cancer, a metastatic prostate cancer, a metastatic castration-resistant prostate cancer, a chemo-refractory prostate cancer; an advanced prostate cancer where the patient has exhausted all standard therapies with elevated baseline serum GDF15; an advanced prostate cancer where the patient has previously received one or more (e.g., 1, 2, 3, or more) lines of cancer therapies; a metastatic chemo-refractory prostate cancer), a bladder cancer, a melanoma, an SCLC (e.g., an extensive-stage SCLC), an NSCLC, a pancreatic cancer (e.g., an advanced pancreatic cancer, a metastatic pancreatic cancer, a pancreatic adenocarcinoma, a metastatic pancreatic adenocarcinoma, an MSI-H pancreatic cancer, a pancreatic ductal adenocarcinoma, a metastatic pancreatic ductal adenocarcinoma), a colorectal cancer, a gastric cancer, an esophageal cancer, an ovarian cancer, a head neck squamous cell carcinoma or an MSI-H cancer (e.g., an MSI-H prostate cancer, an MSI-H pancreatic cancer, an MSI-H ovarian cancer, an MSI-H ovarian cancer, an MSI-H HNSCC, an MSI-H SLCLC (e.g., an MSI-H extensive-stage SCLC). In some instances, the cancer is an MSI-H cancer selected from a castration-resistant prostate cancer, a bladder cancer, a melanoma, a SCLC (e.g., an extensive-stage SCLC), a pancreatic cancer (e.g., an advanced pancreatic cancer, a metastatic pancreatic cancer, a pancreatic adenocarcinoma, a metastatic pancreatic adenocarcinoma, an MSI-H pancreatic cancer, a pancreatic ductal adenocarcinoma, a metastatic pancreatic ductal adenocarcinoma), a colorectal cancer, a gastric cancer, an esophageal cancer, an ovarian cancer, a head and neck squamous cell carcinoma). In some instances, the cancer is a pancreatic cancer (e.g., an advanced pancreatic cancer, a metastatic pancreatic cancer, a pancreatic adenocarcinoma, a metastatic pancreatic adenocarcinoma, an MSI-H pancreatic cancer, a pancreatic ductal adenocarcinoma, a metastatic pancreatic ductal adenocarcinoma). In some instances, the cancer is a pancreatic adenocarcinoma. In some instances, the cancer is a metastatic pancreatic cancer. In some instances, the cancer is a metastatic pancreatic adenocarcinoma. In some instances, the pancreatic cancer is an advanced pancreatic cancer. In some instances, the pancreatic cancer is an MSI-H pancreatic cancer. In some instances, the pancreatic cancer is a pancreatic ductal adenocarcinoma. In some instances, the pancreatic cancer is a metastatic pancreatic ductal adenocarcinoma. In some instances, the human patient has a prostate cancer (e.g., an mCRPC, a castration-sensitive prostate cancer, a castration-resistant prostate cancer, an MSI-H prostate cancer, a metastatic prostate cancer, a metastatic castrate-resistant prostate cancer, a chemo-refractory prostate cancer; an advanced prostate cancer where the patient has exhausted all standard therapies with elevated baseline serum GDF15; an advanced prostate cancer where the patient has previously received one or more (e.g., 1, 2, 3, or more) lines of cancer therapies; a metastatic chemo-refractory prostate cancer). In some instances, the cancer is advanced prostate cancer. In some instances, the human patient has prostate cancer and poor survival. In some instances, the cancer is an mCRPC. In some instances, the cancer is chemo-refractory prostate cancer. In some instances, the cancer is metastatic chemo-refractory prostate cancer. In some instances, the cancer is metastatic castrate-resistant, chemo-refractory prostate cancer. In some instances, the cancer is advanced prostate cancer, wherein the human patient has exhausted all standard therapies, and wherein the human patient has an elevated baseline serum GDF15 (compared to a human or a panel of humans not having prostate cancer). In some instances, the cancer is advanced prostate cancer, wherein the human patient has previously received more than one line of therapies. In some instances, the cancer is advanced prostate cancer, wherein the human patient has previously received more than two lines of therapies. In some instances, the cancer is advanced prostate cancer, wherein the human patient has previously received more than three linel of therapies. In some instances, the cancer is advanced prostate cancer, wherein the human patient has relapsed from one or more lines of previous therapies. The one or more lines of therapies can include surgery, radiation, androgen-deprivation therapy or a chemotherapy (e.g., leuprolide acetate (LUPRON), zoledronic acid (ZOMETA), docetaxel, cabazitaxel, radium 223, mitoxantrone. Oxaliplatin and pemetrexed).

In some instances, the anti-GFRAL antibody is administered subcutaneously. In some instances, the anti-GFRAL antibody is administered intravenously.

3. Methods of Treating Pancreatic Cancer

An anti-GFRAL antibody described herein or a pharmaceutical composition comprising the same can also be used to treat a human patient having pancreatic cancer (e.g., an advanced pancreatic cancer, a metastatic pancreatic cancer, a pancreatic adenocarcinoma, a metastatic pancreatic adenocarcinoma, an MSI-H pancreatic cancer, a pancreatic ductal adenocarcinoma, a metastatic pancreatic ductal adenocarcinoma), in combination with paclitaxel (e.g., nab-paclitaxel) and gemcitabine.

Thus, provided herein is a method for treating pancreatic cancer (e.g., an advanced pancreatic cancer, a metastatic pancreatic cancer, a pancreatic adenocarcinoma, a metastatic pancreatic adenocarcinoma, an MSI-H pancreatic cancer, a pancreatic ductal adenocarcinoma, a metastatic pancreatic ductal adenocarcinoma) in a human patient, the method comprising administering to the human patient: (a) an anti-GFRAL antibody at a dose of about 30 mg (or a pharmaceutical composition comprising the same) once about every 4 weeks, wherein the anti-GFRAL antibody inhibits GFRAL binding to RET; (b) paclitaxel (e.g., nab-paclitaxel); and (c) gemcitabine. In some instances, the dose is 30 mg. In some instances, the dose is administered once every 4 weeks. In some instances, the dose is 30 mg and the dose is administered once every 4 weeks. In some instances, the anti-GFRAL antibody is administered subcutaneously. In some instances, the anti-GFRAL antibody is administered subcutaneously at a dose of about 30 mg once about every 4 weeks. In some instances, the anti-GFRAL antibody is administered subcutaneously at a dose of 30 mg once every 4 weeks. In some instances, the anti-GFRAL antibody is administered intravenously. In some instances, the pancreatic cancer is advanced pancreatic cancer. In some instances, the pancreatic cancer is metastatic pancreatic cancer. In some instances, the pancreatic cancer is pancreatic adenocarcinoma. In some instances, the pancreatic cancer is metastatic pancreatic adenocarcinoma. In some instances, the pancreatic cancer is an MSI-H pancreatic cancer. In some instances, the pancreatic cancer is a pancreatic ductal adenocarcinoma. In some instances, the pancreatic cancer is a metastatic pancreatic ductal adenocarcinoma.

Thus, provided herein is a method for treating pancreatic cancer (e.g., an advanced pancreatic cancer, a metastatic pancreatic cancer, a pancreatic adenocarcinoma, a metastatic pancreatic adenocarcinoma, an MSI-H pancreatic cancer, a pancreatic ductal adenocarcinoma, a metastatic pancreatic ductal adenocarcinoma) in a human patient, comprising administering to the human patient: (a) an anti-GFRAL antibody at a dose of about 100 mg (or a pharmaceutical composition comprising the same) once about every 4 weeks, wherein the anti-GFRAL antibody inhibits GFRAL binding to RET; (b) paclitaxel (e.g., nab-paclitaxel); and (c) gemcitabine. In some instances, the dose is 100 mg. In some instances, the dose is administered once every 4 weeks. In some instances, the dose is 100 mg and the dose is administered once every 4 weeks. In some instances, the anti-GFRAL antibody is administered subcutaneously. In some instances, the anti-GFRAL antibody is administered subcutaneously at a dose of about 100 mg once about every 4 weeks. In some instances, the anti-GFRAL antibody is administered subcutaneously at a dose of 100 mg once every 4 weeks. In some instances, the anti-GFRAL antibody is administered intravenously. In some instances, the pancreatic cancer is advanced pancreatic cancer. In some instances, the pancreatic cancer is metastatic. In some instances, the pancreatic cancer is pancreatic adenocarcinoma. In some instances, the pancreatic cancer is metastatic pancreatic adenocarcinoma. In some instances, the pancreatic cancer is an MSI-H pancreatic cancer. In some instances, the pancreatic cancer is a pancreatic ductal adenocarcinoma. In some instances, the pancreatic cancer is a metastatic pancreatic ductal adenocarcinoma.

In some instances, the human patient is administered at least 2, at least 3, at least 4, at least 5, at least 6, at least 7 at least 8, at least 9, at least 10 doses, at least 11 doses, at least 12 doses, at least 15 doses, at least 20 doses, at least 30 doses, at least 40 doses, at least 50 doses, 1-50 doses, 10-50 doses, 10-40 doses, or 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more doses of paclitaxel (e.g., nab-paclitaxel). In some instances, the paclitaxel (e.g., nab-paclitaxel) is administered to the human patient once a week, once every 2 weeks, once every 3 weeks, once every 4 weeks, once every 5 weeks, once every 6 weeks, once every 7 weeks, or once every 8 weeks. In some instances, the paclitaxel (e.g., nab-paclitaxel) is administered to the human patient once a week. In some instances, the paclitaxel (e.g., nab-paclitaxel) is administered to the human patient once every 2 weeks. In some instances, the paclitaxel (e.g., nab-paclitaxel) is administered to the human patient once, twice, 3 times, 4 times, 5 times, 6 times, 7 times, or 8 times during the 4 week interval during which the anti-GFRAL antibody is administered to the human patient. For instance, if the anti-GFRAL antibody is administered to the human patient once every 4 weeks, then the paclitaxel (e.g., nab-paclitaxel) is administered to the human patient once, twice, 3 times, 4 times, 5 times, 6 times, 7 times, or 8 times in between two doses (4 weeks apart) of the anti-GFRAL antibody, inclusive. In some instances, the paclitaxel (e.g., nab-paclitaxel) is administered to the human patient twice during the 4 week interval during which the anti-GFRAL antibody is administered to the human patient. In some instances, the paclitaxel (e.g., nab-paclitaxel) is administered to the human patient 3 times during the 4 week interval during which the anti-GFRAL antibody is administered to the human patient. In some instances, the paclitaxel (e.g., nab-paclitaxel) is administered to the human patient 4 times during the 4 week interval during which the anti-GFRAL antibody is administered to the human patient. In some instances, the paclitaxel (e.g., nab-paclitaxel) is administered to the human patient at a dose of about 50 mg/m², about 60 mg/m², about 65 mg/m², about 70 mg/m², about 75 mg/m², about 80 mg/m², about 90 mg/m², about 95 mg/m², about 100 mg/m², about 115 mg/m², about 125 mg/m², about 135 mg/m², about 150 mg/m², 50-150 mg/m², 50-125 mg/m², 50-100 mg/m², 50-75 mg/m², 75-150 mg/m², 75-125 mg/m², 75-100 mg/m², or 100-125 mg/m². In some instances, the paclitaxel (e.g., nab-paclitaxel) is administered to the human patient at a dose of 125 mg/m². In some instances, the paclitaxel (e.g., nab-paclitaxel) is administered to the human patient at a dose of 125 mg/m² once a week. In some instances, the paclitaxel is nab-paclitaxel. In some instances, the nab-paclitaxel is administered to the human patient at a dose of 125 mg/m². In some instances, the nab-paclitaxel is administered to the human patient at a dose of 125 mg/m² once a week for 3 weeks during the 4 week interval during which the anti-GFRAL antibody is administered to the human patient. In some instances, the paclitaxel (e.g., nab-paclitaxel) is administered to the human patient intravenously. In some instances, the paclitaxel (e.g., nab-paclitaxel) is administered to the human patient via intravenous infusion over 30-40 minutes on days 1, 8, and 15 during the 4 week interval during which the anti-GFRAL antibody is administered to the human patient.

In some instances, the human patient is administered at least 2, at least 3, at least 4, at least 5, at least 6, at least 7 at least 8, at least 9, at least 10 doses, at least 11 doses, at least 12 doses, at least 15 doses, at least 20 doses, at least 30 doses, at least 40 doses, at least 50 doses, 1-50 doses, 10-50 doses, 10-40 doses, or 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more of gemcitabine. In some instances, the gemcitabine is administered to the human patient once a week, once every 2 weeks, once every 3 weeks, once every 4 weeks, once every 5 weeks, once every 6 weeks, once every 7 weeks, or once every 8 weeks. In some instances, the gemcitabine is administered to the human patient once a week. In some instances, the gemcitabine is administered to the human patient once every 2 weeks. In some instances, the gemcitabine is administered to the human patient once, twice, 3 times, 4 times, 5 times, 6 times, 7 times, or 8 times during the 4 week interval during which the anti-GFRAL antibody is administered to the human patient. For instance, if the anti-GFRAL antibody is administered to the human patient once every 4 weeks, then the gemcitabine is administered to the human patient once, twice, 3 times, 4 times, 5 times, 6 times, 7 times, or 8 times in between two doses (4 weeks apart) of the anti-GFRAL antibody, inclusive. In some instances, the gemcitabine is administered to the human patient twice during the 4 week interval during which the anti-GFRAL antibody is administered to the human patient. In some instances, the gemcitabine is administered to the human patient 3 times during the 4 week interval during which the anti-GFRAL antibody is administered to the human patient. In some instances, the gemcitabine is administered to the human patient 4 times during the 4 week interval during which the anti-GFRAL antibody is administered to the human patient. In some instances, the gemcitabine is administered to the human patient at a dose of about 400 mg/m², about 450 mg/m², about 500 mg/m², about 550 mg/m², about 600 mg/m², about 650 mg/m², about 700 mg/m², about 750 mg/m², about 800 mg/m², about 850 mg/m², about 900 mg/m², about 950 mg/m², about 1000 mg/m², 400-1000 mg/m², 400-800 mg/m², 400-600 mg/m², 500-1000 mg/m², 500-900 mg/m², 500-800 mg/m², or 500-700 mg/m². In some instances, the gemcitabine is administered to the human patient at a dose of 1000 mg/m². In some instances, the gemcitabine is administered to the human patient at a dose of 1000 mg/m² once a week for 3 weeks during the 4 week interval during which the anti-GFRAL antibody is administered to the human patient. In some instances, the gemcitabine is administered to the human patient intravenously. In some instances, the gemcitabine is administered to the human patient via intravenous infusion over 30-40 minutes on days 1, 8, and 15 during the 4 week interval during which the anti-GFRAL antibody is administered to the human patient.

In some instances, the paclitaxel (e.g., nab-paclitaxel) and gemcitabine are administered to the human patient concurrently (or within 1 minute, within 2 minutes, within 5 minutes, within 10 minutes, or within 1 hour of each other). In some instances, the paclitaxel (e.g., nab-paclitaxel) and gemcitabine are administered to the human patient sequentially (e.g., within 1, 2, 3, 4, 5, 6, 7, 8, 9, 10, 11, 12 13, 14, 15, 16, 17, 18, 19, 20, or 21 days of each other). In some instances, the paclitaxel (e.g., nab-paclitaxel) is administered to the human patient prior to (e.g., within 1, 2, 3, 4, 5, 6, 7, 8, 9, 10, 11, 12, 13, 14, 15, 16, 17, 18, 19, 20, or 21 days prior to) administering to the human patient the gemcitabine. In some instances, the gemcitabine is administered to the human patient prior to (e.g., within 1, 2, 3, 4, 5, 6, 7, 8, 9, 10, 11, 12 13, 14, 15, 16, 17, 18, 19, 20, or 21 days prior to) administering to the human patient the paclitaxel (e.g., nab-paclitaxel). In some instances, the first dose of paclitaxel (e.g., nab-paclitaxel) and/or the gemcitabine is/are administered to the human patient prior to (e.g., within 1, 2, 3, 4, 5, 6, 7, 8, 9, 10, 11, 12 13, 14, 15, 16, 17, 18, 19, 20, or 21 days prior to) administering to the human patient a first dose of the human patient the anti-GFRAL antibody. In some instances, the first dose of paclitaxel (e.g., nab-paclitaxel) and/or the gemcitabine is/are administered to the human patient after (e.g., within 1, 2, 3, 4, 5, 6, 7, 8, 9, 10, 11, 12 13, 14, 15, 16, 17, 18, 19, 20, or 21 days after) administering to the human patient a first dose of the human patient the anti-GFRAL antibody. In some instances, the first dose of paclitaxel (e.g., nab-paclitaxel) and/or the gemcitabine is/are administered to the human patient concurrently (or within 1 minute, within 2 minutes, within 5 minutes, within 10 minutes, or within 1 hour of each other) with administering to the human patient a first dose of the human patient the anti-GFRAL antibody.

In some cases, the human patient has an elevated serum level of GDF15 prior to the administering of the anti-GFRAL antibody compared to the serum level of GDF15 in said human prior to developing the cancer. In some instances, the human patient has an elevated level of GDF15 prior to the administering of the anti-GFRAL antibody. In some instances, the elevated level of GDF15 is an elevated level of circulating GDF15. In some instances, the elevated level of GDF15 is an elevated level of serum GDF15. In some cases, the elevated level of GDF15 is in the cancer tissue relative to the same non-cancerous tissue (e.g., in a cancerous pancreatic tissue relative to a non-cancerous pancreatic tissue). In some instances, the elevated level of GDF15 is as compared to a level of GDF15 in a human not having the cancer or a panel of humans (e.g., 2, 3, 4, 5, 10, 15, 20, 25, or more) not having the cancer. In some instances, the human patient has a level of GDF15 that is at least 500 pg/mL (e.g., at least 500 pg/mL, at least 550 pg/mL, at least 600 pg/mL, at least 650 pg/mL, at least 700 pg/mL, at least 750 pg/mL, at least 800 pg/mL, at least 850 pg·mL, at least 900 pg/mL, at least 950 pg/mL, at least 1000 pg/mL, at least 1100 pg/mL, at least 1200 pg/mL, at least 1290 pg/mL, at least 1300 pg/mL, at least 1400 pg/mL, at least 1500 pg/mL, at least 1600 pg/mL, at least 1700 pg/mL, at least 1800 pg/mL, at least 1900 pg/mL, at least 2000 pg/mL, at least 3000 pg/mL, at least 4,000 pg/mL, at least 5,000 pg/mL, at least 10,000 pg/mL, at least 20,000 pg/mL, at least 30,000 pg/mL, at least 40,000 pg/mL, at least 50,000 pg/mL). In some instances, the human patient has a level of GDF between 1,000 and 60,000 pg/mL, inclusive. In some instances, the human patient has a level of GDF15 that is between 500 and 60,000 pg/mL, inclusive. In some instances, the human patient has a level of GDF15 that is between 800 and 60,000 pg/mL, inclusive. In some instances, the human patient has a level of GDF15 that is between 1000 and 60,000 pg/mL, inclusive. In some instances, the human patient has a level of GDF15 that is between 1,100 and 60,000 pg/mL, inclusive. In some instances, the human patient has a level of GDF15 that is between 1,300 and 60,000 pg/mL, inclusive. In some instances, the human patient has a level of GDF15 that is between 1,500 and 60,000 pg/mL, inclusive. In some instances, the human patient has a level of GDF15 that is between 1,000 and 50,000 pg/mL, inclusive. In some instances, the human patient has a level of GDF15 that is between 1,000 and 40,000 pg/mL, inclusive. In some instances, the human patient has a level of GDF15 that is between 1,000 and 30,000 pg/mL, inclusive. In some instances, the human patient has a level of GDF15 that is between 1,000 and 20,000 pg/mL, inclusive. In some instances, the human patient has a level of GDF15 that is between 1,000 and 10,000 pg/mL, inclusive. In some instances, the human patient has a level of GDF15 that is between 1,000 and 5,000 pg/mL, inclusive. In some instances, the human patient has a level of GDF15 that is between 1,000 and 3,000 pg/mL, inclusive. In some instances, the human patient has a serum GDF15 level of at least 950 pg/ml. In some instances, the human patient has a serum GDF15 level of at least 1300 pg/ml. In some instances, the level of GDF15 is a level of circulating GDF15. In some instances, the level of GDF15 is a level of serum GDF15. In some instances, the human patient has a reduced serum GDF15 level after the administering of the anti-GFRAL antibody as compared to the serum GDF15 level in the human patient prior to the administering of the anti-GFRAL antibody.

In some instances, the patient is selected for treatment has elevated serum level of GDF15 in comparison with his or her own level before developing the cancer.

In some instances, the method for treating pancreatic cancer (e.g., an advanced pancreatic cancer, a metastatic pancreatic cancer, a pancreatic adenocarcinoma, a metastatic pancreatic adenocarcinoma, an MSI-H pancreatic cancer, a pancreatic ductal adenocarcinoma, a metastatic pancreatic ductal adenocarcinoma) stabilizes progression of the cancer. In some instances, the method for treating pancreatic cancer (e.g., an advanced pancreatic cancer, a metastatic pancreatic cancer, a pancreatic adenocarcinoma, a metastatic pancreatic adenocarcinoma, an MSI-H pancreatic cancer, a pancreatic ductal adenocarcinoma, a metastatic pancreatic ductal adenocarcinoma) slows down progression of the cancer. In some instances, the method for treating pancreatic cancer (e.g., an advanced pancreatic cancer, a metastatic pancreatic cancer, a pancreatic adenocarcinoma, a metastatic pancreatic adenocarcinoma, an MSI-H pancreatic cancer, a pancreatic ductal adenocarcinoma, a metastatic pancreatic ductal adenocarcinoma) halts progression of the cancer. In some instances, the method for treating pancreatic cancer (e.g., an advanced pancreatic cancer, a metastatic pancreatic cancer, a pancreatic adenocarcinoma, a metastatic pancreatic adenocarcinoma, an MSI-H pancreatic cancer, a pancreatic ductal adenocarcinoma, a metastatic pancreatic ductal adenocarcinoma) shrinks the size of a tumor of the cancer. In some instances, the method for treating pancreatic cancer (e.g., an advanced pancreatic cancer, a metastatic pancreatic cancer, a pancreatic adenocarcinoma, a metastatic pancreatic adenocarcinoma, an MSI-H pancreatic cancer, a pancreatic ductal adenocarcinoma, a metastatic pancreatic ductal adenocarcinoma) increases overall survival of the patient. Methods for assessing the progression of a cancer are known in the art and include, e.g., evaluation of target lesions (e.g., measurement of tumor or lesion, using, e.g., X-ray, computerized tomography scan, magnetic resonance imaging, caliper measurement, or positron emission tomography scan), cytology or histology, or evaluation of tumor marker(s) (see, e.g., Eisenhauer et al., 2009, European Journal of Cancer 45:228-247 and Schwartz et al., 2016, European Journal of Cancer 62:132-137; each of which is incorporated by reference herein in its entirety).

In some instances, the human patient has pancreatic tumor-related weight loss. In some instances, the method reduces pancreatic tumor-related weight loss (e.g., the human patient loses less than 5%, less than 10%, less than 15%, less than 20%, less than 25%, less than 30%, less than 40%, or less than 50%, or does not lose more than 5%, more than 10%, more than 15%, more than 20%, more than 25%, more than 30%, more than 40%, or more than 50% of weight after being administered the anti-GFRAL antibody (e.g., within 1, 2, 3, 4, 5, 6, 7, 8, 9, 10 or more weeks of being administered 1, 2, 3, 4, 5, or more doses of the anti-GFRAL antibody)). In some instances, the method increases lean body mass in the human patient (e.g., the human patient has an increase of at least 1%, at least 2%, at least 3%, at least 4%, at least 5%, at least 6%, at least 7%, at least 10%, at least 15%, at least 20%, at least 25%, 1-5%, 1-10%, 1-15%, 1-20%, 1-25%, 2-5%, 2-10%, 2-15%, 2-20%, 3-5%, 3-10%, 3-15%, 3-20%, 3-25%, 4-5%, 4-10%, 4-15%, 4-20%, 4-25%, 4-5%, 5-10%, 5-15%, 5-20%, 5-25%, 6-10%, 6-15%, 6-20%, 6-25%, 7-10%, 7-15%, 7-20%, 7-25%, 8-10%, 8-15%, 8-20%, 8-25%, 10-15%, 10-20%, 10-25%, 15%-20%, 15-25%, 20%-25% of lean body mass after being administered the anti-GFRAL antibody (e.g., within 1, 2, 3, 4, 5, 6, 7, 8, 9, 10 or more weeks of being administered 1, 2, 3, 4, 5, or more doses of the anti-GFRAL antibody, paclitaxel, and gemcitabine)).

In some instances, the human patient has tumor-related cachexia. In some instances, the method reduces one or more (e.g., 1, 2, 3, 4, 5) symptoms of cachexia. Reduction of cachexia may be measured by, e.g., evaluating weight loss, muscle atrophy, fatigue, weakness, or appetite.

In some instances, the human patient previously failed to be treated with one or more (e.g., 1, 2, 3, or more) other cancer (e.g., pancreatic cancer, e.g., an advanced pancreatic cancer, a metastatic pancreatic cancer, a pancreatic adenocarcinoma, a metastatic pancreatic adenocarcinoma, an MSI-H pancreatic cancer, a pancreatic ductal adenocarcinoma, a metastatic pancreatic ductal adenocarcinoma.) therapies (i.e., other than an anti-GFRAL antibody described herein) prior to the administering of the anti-GFRAL antibody. In some instances, the human patient previously failed to be treated with one other cancer (e.g., pancreatic cancer, e.g., an advanced pancreatic cancer, a metastatic pancreatic cancer, a pancreatic adenocarcinoma, a metastatic pancreatic adenocarcinoma, an MSI-H pancreatic cancer, a pancreatic ductal adenocarcinoma, a metastatic pancreatic ductal adenocarcinoma) therapy prior to the administering of the anti-GFRAL antibody. In some instances, the human patient previously failed to be treated with two other cancer (e.g., pancreatic cancer, e.g., an advanced pancreatic cancer, a metastatic pancreatic cancer, a pancreatic adenocarcinoma, a metastatic pancreatic adenocarcinoma, an MSI-H pancreatic cancer, a pancreatic ductal adenocarcinoma, a metastatic pancreatic ductal adenocarcinoma) therapies prior to the administering of the anti-GFRAL antibody. In some instances, the human patient previously failed to be treated with three other cancer (e.g., pancreatic cancer, e.g., an advanced pancreatic cancer, a metastatic pancreatic cancer, a pancreatic adenocarcinoma, a metastatic pancreatic adenocarcinoma, an MSI-H pancreatic cancer, a pancreatic ductal adenocarcinoma, a metastatic pancreatic ductal adenocarcinoma) therapies prior to the administering of the anti-GFRAL antibody.

In some instances, the anti-GFRAL antibody is a first-line treatment. In some instances, the human patient has not previously been treated with any other cancer (e.g., pancreatic cancer, e.g., an advanced pancreatic cancer, a metastatic pancreatic cancer, a pancreatic adenocarcinoma, a metastatic pancreatic adenocarcinoma, an MSI-H pancreatic cancer, a pancreatic ductal adenocarcinoma, a metastatic pancreatic ductal adenocarcinoma) therapy (i.e., other than an anti-GFRAL antibody described herein) prior to the administering of the anti-GFRAL antibody.

In some instances, the human patient has previously received one or more (e.g., 1, 2, 3, or more) anti-pancreatic cancer therapies prior to the administering of the anti-GFRAL antibody.

In some instances, the human patient is about 35 years of age, about 40 years of age, about 45 years of age, about 50 years of age, about 55 years of age, about 60 years of age, about 65 years of age, about 70 years of age, about 75 years of age, about 80 years of age, or about 85 years of age. In some instances, the human patient is at least 35 years of age, at least 40 years of age, at least 45 years of age, at least 50 years of age, at least 55 years of age, at least 60 years of age, at least 65 years of age, at least 70 years of age, at least 75 years of age, at least 80 years of age, or at least 85 years of age. In some instances, the human patient is 35-90 years of age, 35-80 years of age, 35-70 years of age, 45-90 years of age, 45-80 years of age, 45-70 years of age, 55-90 years of age, 55-80 years of age, 55 to 75 years of age, or 55-65 years of age.

In some instances, the human patient is administered at least 2, at least 3, at least 4, at least 5, at least 6, at least 7 at least 8, at least 9, at least 10 doses, at least 11 doses, at least 12 doses, or 2, 3, 4, 5, 6, 7, 8, 9, 10, 11, 12, 13, 14, 15, 16, 17, 18, 19, 20, 21, 22, 23, 24, 25, 26, 27, 28, 29, 30 or more doses of the anti-GFRAL antibody.

In some instances, each of the at least 2, at least 3, at least 4, at least 5, at least 6, at least 7 at least 8, at least 9, at least 10 doses, at least 11 doses, at least 12 doses, or 2, 3, 4, 5, 6, 7, 8, 9, 10, 11, 12, 13, 14, 15, 16, 17, 18, 19, 20, 21, 22, 23, 24, 25, 26, 27, 28, 29, 30 or more doses of the anti-GFRAL antibody is administered about 4 weeks apart (i.e., once about every 4 weeks). In some instances, each of the doses of the anti-GFRAL antibody is administered 4 weeks apart (i.e., once every 4 weeks).

In some instances, each of the at least 2, at least 3, at least 4, at least 5, at least 6, at least 7 at least 8, at least 9, at least 10 doses, at least 11 doses, at least 12 doses, or 2, 3, 4, 5, 6, 7, 8, 9, 10, 11, 12, 13, 14, 15, 16, 17, 18, 19, 20, 21, 22, 23, 24, 25, 26, 27, 28, 29, 30 or more of the anti-GFRAL antibody is about 30 mg and is administered about 4 weeks apart (i.e., once about every 4 weeks). In some instances, each of the doses of the anti-GFRAL antibody is 30 mg and is administered 4 weeks apart (i.e., once every 4 weeks). In some instances, each of the at least 2, at least 3, at least 4, at least 5, at least 6, at least 7 at least 8, at least 9, at least 10 doses, at least 11 doses, at least 12 doses, or 2, 3, 4, 5, 6, 7, 8, 9, 10, 11, 12, 13, 14, 15, 16, 17, 18, 19, 20, 21, 22, 23, 24, 25, 26, 27, 28, 29, 30 or more doses of the anti-GFRAL antibody is about 100 mg and is administered about 4 weeks apart (i.e., once about every 4 weeks). In some instances, each of the doses of the anti-GFRAL antibody is 100 mg and is administered 4 weeks apart (i.e., once every 4 weeks).

In some instances in which the pancreatic cancer (e.g., an advanced pancreatic cancer, a metastatic pancreatic cancer, a pancreatic adenocarcinoma, a metastatic pancreatic adenocarcinoma, an MSI-H pancreatic cancer, a pancreatic ductal adenocarcinoma, a metastatic pancreatic ductal adenocarcinoma) is CA19-9-positive, the method reduces the level of CA19-9 (e.g., the level of CA19-9 is decreased by at least 10%, at least 20%, at least 30%, at least 40%, at least 50%, at least 60%, at least 70%, at least 80%, at least 90%, at least 98%, 10-98%, 20-98%, 30-98%, 40-98%, 50-98%, 60-98%, 70-98%, 80-98%, or 90-98% after being administered the anti-GFRAL antibody (e.g., within 1, 2, 3, 4, 5, 6, 7, 8, 9, 10, 11, 12, 13, 14, 15, 16, 17, 18, 19, 20, 21, 22, 23, 24 or more weeks of being administered 1, 2, 3, 4, 5, 6, or more doses of the anti-GFRAL antibody) compared to the level of CA19-9 before being administered the anti-GFRAL antibody. Thus, in some instances, the level of CA19-9 may be determined in a method of assessing effectiveness of administering the anti-GFRAL antibody, paclitaxel (e.g., nab-paclitaxel), and gemcitabine to treat a human patient having pancreatic cancer (e.g., an advanced pancreatic cancer, a metastatic pancreatic cancer, a pancreatic adenocarcinoma, a metastatic pancreatic adenocarcinoma, an MSI-H pancreatic cancer, a pancreatic ductal adenocarcinoma, a metastatic pancreatic ductal adenocarcinoma). In some instances, the level of CA19-9 is a blood level of CA19-9. In some instances, the level of CA19-9 is a serum level of CA19-9. In some instances, a pancreatic cancer (e.g., an advanced pancreatic cancer, a metastatic pancreatic cancer, a pancreatic adenocarcinoma, a metastatic pancreatic adenocarcinoma, an MSI-H pancreatic cancer, a pancreatic ductal adenocarcinoma, a metastatic pancreatic ductal adenocarcinoma) is CA19-9-positive if blood levels are greater than or equal to 37 U/mL.

In some instances, the method further comprises determining a first level of CA19-9. In some instances, the determining of a first level of CA19-9 is before administering a first dose of the anti-GFRAL antibody, before administering a first dose of paclitaxel (e.g., nab-paclitaxel), and before administering a first dose of gemcitabine. In some instances, the first level of CA19-9 is a blood level of CA19-9. In some instances, the first level of CA19-9 is a serum level of CA19-9. In some instances, the method further comprises determining a second level of CA19-9 after administering the anti-GFRAL antibody, paclitaxel (e.g., nab-paclitaxel), and gemcitabine. In some instances, the second level of CA19-9 is a blood level of CA19-9. In some instances, the second level of CA19-9 is a serum level of CA19-9. In some instances, the determining of the second level of CA19-9 is after 2, 3, 4, 5, 6, 7, 8, 9, 10, or more doses of the anti-GFRAL antibody are administered to the human subject. In some instances, the second level of CA19-9 is at least 10%, at least 20%, at least 30%, at least 40%, at least 50%, at least 60%, at least 70%, at least 80%, at least 90%, at least 98%, 10-98%, 20-98%, 30-98%, 40-98%, 50-98%, 60-98%, 70-98%, 80-98%, or 90-98% less than the first level of CA19-9.

In some instances, the method reduces the level of GDF15 (e.g., the level of GDF15 is decreased by at least 10%, at least 20%, at least 30%, at least 40%, at least 50%, at least 60%, at least 70%, at least 80%, at least 90%, at least 98%, 10-98%, 20-98%, 30-98%, 40-98%, 50-98%, 60-98%, 70-98%, 80-98%, or 90-98% after being administered the anti-GFRAL antibody (e.g., within 1, 2, 3, 4, 5, 6, 7, 8, 9, 10, 11, 12, 13, 14, 15, 16, 17, 18, 19, 20, 21, 22, 23, 24 or more weeks of being administered 1, 2, 3, 4, 5, 6, or more doses of the anti-GFRAL antibody) compared to the level of GDF15 before being administered the anti-GFRAL antibody. Thus, in some instances, the level of GDF15 may be determined in a method of assessing effectiveness of administering the anti-GFRAL antibody to treat a human patient having cancer. In some instances, the level of GDF15 is a blood level of GDF15. In some instances, the level of GDF15 is a serum level of GDF15.

In some instances, the method of assessing effectiveness of administering the anti-GFRAL antibody to treat a human patient having cancer further comprises determining a first level of GDF15. In some instances, the determining of a first level of GDF15 is before administering a first dose of the anti-GFRAL antibody. In some instances, the first level of GDF15 is a blood level of GDF15. In some instances, the first level of GDF15 is a serum level of GDF15. In some instances, the method further comprises determining a second level of GDF15 after administering the anti-GFRAL antibody. In some instances, the second level of GDF15 is a blood level of GDF15. In some instances, the second level of GDF15 is a serum level of GDF15. In some instances, the determining of the second level of GDF15 is after 2, 3, 4, 5, 6, 7, 8, 9, 10, or more doses of the anti-GFRAL antibody are administered to the human subject. In some instances, the second level of GDF15 is at least 10%, at least 20%, at least 30%, at least 40%, at least 50%, at least 60%, at least 70%, at least 80%, at least 90%, at least 98%, 10-98%, 20-98%, 30-98%, 40-98%, 50-98%, 60-98%, 70-98%, 80-98%, or 90-98% less than the first level of GDF15.

In some instances of the combination therapy of the anti-GFRAL antibody, paclitaxel (e.g., nab-paclitaxel), and gemcitabine, the anti-GFRAL antibody comprises (i) a VH comprising the VH CDR1, the VH CDR2, the VH CDR3 from the amino acid sequence set forth in SEQ ID NO:3 or 1982; and (ii) a VL comprising the VL CDR1, the VL CDR2, and the VL CDR3 from the amino acid sequence set forth in SEQ ID NO:4 or 1997. In some instances, the anti-GFRAL antibody comprises: (i) a VH comprising a VH CDR1 comprising the amino acid sequence set forth in SEQ ID NO:46, a VH CDR2 comprising the amino acid sequence set forth in SEQ ID NO:137, and a VH CDR3 comprising the amino acid sequence set forth in SEQ ID NO:225; and (ii) a VL comprising a VL CDR1 comprising the amino acid sequence set forth in SEQ ID NO:301, a VL CDR2 comprising the amino acid sequence set forth in SEQ ID NO:376, and a VL CDR3 comprising the amino acid sequence set forth in SEQ ID NO:426. In some instances, the anti-GFRAL antibody comprises: (i) a VH comprising a VH CDR1 comprising the amino acid sequence set forth in SEQ ID NO:48, a VH CDR2 comprising the amino acid sequence set forth in SEQ ID NO:137, and a VH CDR3 comprising the amino acid sequence set forth in SEQ ID NO:225; and (ii) a VL comprising a VL CDR1 comprising the amino acid sequence set forth in SEQ ID NO:301, a VL CDR2 comprising the amino acid sequence set forth in SEQ ID NO:376, and a VL CDR3 comprising the amino acid sequence set forth in SEQ ID NO:426. In some instances, the anti-GFRAL antibody comprises: (i) a VH comprising an amino acid sequence having at least at least 75%, at least 80%, at least 85%, at least 90%, at least 95%, at least 96%, at least 97%, at least 98%, at least 99%, or 100% identity to the amino acid sequence set forth in SEQ ID NO:1982; and (ii) a VL comprising an amino acid sequence having at least at least 75%, at least 80%, at least 85%, at least 90° %, at least 95%, at least 96%, at least 97%, at least 98%, at least 99%, or 10(0% identity to the amino acid sequence set forth in SEQ ID NO:1997. In some instances, the anti-GFRAL antibody comprises a VH comprising the amino acid sequence set forth in SEQ ID NO:1982. In some instances, the anti-GFRAL antibody comprises a VL comprising the amino acid sequence set forth in SEQ ID NO:1997. In some instances, the anti-GFRAL antibody comprises: (i) a VH comprising the amino acid sequence set forth in SEQ ID NO:1982; and (ii) a VL comprising the amino acid sequence set forth in SEQ ID NO:1997. In some instances, the anti-GFRAL antibody comprises a heavy chain comprising an amino acid sequence having at least at least 75%, at least 80%, at least 85%, at least 90%, at least 95%, at least 96%, at least 97%, at least 98%, at least 99%, or 100% identity to the amino acid sequence set forth in SEQ ID NO:2010. In some instances, the anti-GFRAL antibody comprises a light chain comprising an amino acid sequence having at least at least 75%, at least 80%, at least 85%, at least 90%, at least 95%, at least 96%, at least 97%, at least 98%, at least 99%, or 100% identity to the amino acid sequence set forth in SEQ ID NO:2012. In some instances, the anti-GFRAL antibody comprises: (i) a heavy chain comprising an amino acid sequence having at least at least 75%, at least 80%, at least 85%, at least 90%, at least 95%, at least 96%, at least 97%, at least 98%, at least 99%, or 100% identity to the amino acid sequence set forth in SEQ ID NO:2010; and (ii) a light chain comprising an amino acid sequence having at least at least 75%, at least 80%, at least 85%, at least 90%, at least 95%, at least 96%, at least 97%, at least 98%, at least 99%, or 100% identity to the amino acid sequence set forth in SEQ ID NO:2012. In some instances, the anti-GFRAL antibody comprises a heavy chain comprising the amino acid sequence set forth in SEQ ID NO:2010. In some instances, the anti-GFRAL antibody comprises a heavy chain consisting of the amino acid sequence set forth in SEQ ID NO:2010. In some instances, the anti-GFRAL antibody comprises a light chain comprising the amino acid sequence set forth in SEQ ID NO:2012. In some instances, the anti-GFRAL antibody comprises a light chain consisting of the amino acid sequence set forth in SEQ ID NO:2012. In some instances, the anti-GFRAL antibody comprises: (i) a heavy chain comprising the amino acid sequence set forth in SEQ ID NO:2010; and (ii) a light chain comprising the amino acid sequence set forth in SEQ ID NO:2012. In some instances, the anti-GFRAL antibody comprises: (i) a heavy chain consisting of the amino acid sequence set forth in SEQ ID NO:2010; and (ii) a light chain consisting of the amino acid sequence set forth in SEQ ID NO:2012. In some instances, the dose of the anti-GFRAL antibody is about 30 mg. In some instances, the dose of the anti-GFRAL antibody is 30 mg. In some instances, the dose of the anti-GFRAL antibody is about 100 mg. In some instances, the dose of the anti-GFRAL antibody is 100 mg. In some instances, the dose of the anti-GFRAL antibody is administered once about every 4 weeks. In some instances, the dose of the anti-GFRAL antibody is administered once every 4 weeks. In some instances, the dose of the anti-GFRAL antibody is about 30 mg administered once about every 4 weeks. In some instances, the dose of the anti-GFRAL antibody is 30 mg administered once every 4 weeks. In some instances, the dose of the anti-GFRAL antibody is about 100 mg administered once about every 4 weeks. In some instances, the dose of the anti-GFRAL antibody is 100 mg administered once every 4 weeks. In some instances, the anti-GFRAL antibody is administered subcutaneously. In some instances, the anti-GFRAL antibody is administered subcutaneously at a dose of about 30 mg once about every 4 weeks. In some instances, the anti-GFRAL antibody is administered subcutaneously at a dose of 30 mg once every 4 weeks. In some instances, the anti-GFRAL antibody is administered subcutaneously at a dose of about 100 mg once about every 4 weeks. In some instances, the anti-GFRAL antibody is administered subcutaneously at a dose of 100 mg once every 4 weeks. In some instances, the anti-GFRAL antibody is administered intravenously. In some instances, the pancreatic cancer is advanced pancreatic cancer. In some instances, the pancreatic cancer is metastatic pancreatic cancer. In some instances, the pancreatic cancer is an MSI-H pancreatic cancer. In some instances, the pancreatic cancer is pancreatic adenocarcinoma. In some instances, the pancreatic cancer is metastatic pancreatic adenocarcinoma. In some instances, the pancreatic cancer is an MSI-H pancreatic adenocarcinoma. In some instances, the pancreatic cancer is a pancreatic ductal adenocarcinoma. In some instances, the pancreatic cancer is a metastatic pancreatic ductal adenocarcinoma

In some instances of the combination therapy of the anti-GFRAL antibody, paclitaxel (e.g., nab-paclitaxel), and gemcitabine, the anti-GFRAL antibody is administered to the human patient subcutaneously at a dose of 30 mg once every 4 weeks, the paclitaxel (e.g., nab-paclitaxel) is administered to the human patient via intravenous infusion (e.g., over 30-40 minutes) once every week for three weeks during the 4 week interval during which the anti-GFRAL antibody is administered to the human patient, and the gemcitabine is administered to the human patient via intravenous infusion (e.g., over 30-40 minutes) once every week for 3 weeks during the 4 week interval during which the anti-GFRAL antibody is administered to the human patient, wherein the anti-GFRAL antibody comprises (i) a VH comprising the VH CDR1, the VH CDR2, the VH CDR3 from the amino acid sequence set forth in SEQ ID NO:3 or 1982; and (ii) a VL comprising the VL CDR1, the VL CDR2, and the VL CDR3 from the amino acid sequence set forth in SEQ ID NO:4 or 1997. In some instances, the anti-GFRAL antibody comprises: (i) a VH comprising a VH CDR1 comprising the amino acid sequence set forth in SEQ ID NO:46, a VH CDR2 comprising the amino acid sequence set forth in SEQ ID NO:137, and a VH CDR3 comprising the amino acid sequence set forth in SEQ ID NO:225; and (ii) a VL comprising a VL CDR1 comprising the amino acid sequence set forth in SEQ ID NO:301, a VL CDR2 comprising the amino acid sequence set forth in SEQ ID NO:376, and a VL CDR3 comprising the amino acid sequence set forth in SEQ ID NO:426. In some instances, the anti-GFRAL antibody comprises: (i) a VH comprising a VH CDR1 comprising the amino acid sequence set forth in SEQ ID NO:48, a VH CDR2 comprising the amino acid sequence set forth in SEQ ID NO:137, and a VH CDR3 comprising the amino acid sequence set forth in SEQ ID NO:225; and (ii) a VL comprising a VL CDR1 comprising the amino acid sequence set forth in SEQ ID NO:301, a VL CDR2 comprising the amino acid sequence set forth in SEQ ID NO:376, and a VL CDR3 comprising the amino acid sequence set forth in SEQ ID NO:426. In some instances, the anti-GFRAL antibody comprises; (i) a VH comprising an amino acid sequence having at least at least 75%, at least 80%, at least 85%, at least 90%, at least 95%, at least 96%, at least 97%, at least 98%, at least 99%, or 100% identity to the amino acid sequence set forth in SEQ ID NO:1982; and (ii) a VL comprising an amino acid sequence having at least at least 75%, at least 80%, at least 85%, at least 9(0%, at least 95%, at least 96%, at least 97%, at least 98%, at least 99%, or 100% identity to the amino acid sequence set forth in SEQ ID NO:1997. In some instances, the anti-GFRAL antibody comprises a VH comprising the amino acid sequence set forth in SEQ ID NO:1982. In some instances, the anti-GFRAL antibody comprises a VL comprising the amino acid sequence set forth in SEQ ID NO:1997. In some instances, the anti-GFRAL antibody comprises: (i) a VH comprising the amino acid sequence set forth in SEQ ID NO:1982; and (ii) a VL comprising the amino acid sequence set forth in SEQ ID NO: 1997. In some instances, the anti-GFRAL antibody comprises a heavy chain comprising an amino acid sequence having at least at least 75%, at least 80%, at least 85%, at least 90%, at least 95%, at least 96%, at least 97%, at least 98%, at least 99%, or 100% identity to the amino acid sequence set forth in SEQ ID NO:2010. In some instances, the anti-GFRAL antibody comprises a light chain comprising an amino acid sequence having at least at least 75%, at least 80%, at least 85%, at least 90%, at least 95%, at least 96%, at least 97%, at least 98%, at least 99%, or 100% identity to the amino acid sequence set forth in SEQ ID NO:2012. In some instances, the anti-GFRAL antibody comprises: (i) a heavy chain comprising an amino acid sequence having at least at least 75%, at least 80%, at least 85%, at least 90%, at least 95%, at least 96%, at least 97%, at least 98%, at least 99%, or 100% identity to the amino acid sequence set forth in SEQ ID NO:2010; and (ii) a light chain comprising an amino acid sequence having at least at least 75%, at least 80%, at least 85%, at least 90%, at least 95%, at least 96%, at least 97%, at least 98%, at least 99%, or 100% identity to the amino acid sequence set forth in SEQ ID NO:2012. In some instances, the anti-GFRAL antibody comprises a heavy chain comprising the amino acid sequence set forth in SEQ ID NO:2010. In some instances, the anti-GFRAL antibody comprises a heavy chain consisting of the amino acid sequence set forth in SEQ ID NO:2010. In some instances, the anti-GFRAL antibody comprises a light chain comprising the amino acid sequence set forth in SEQ ID NO:2012. In some instances, the anti-GFRAL antibody comprises a light chain consisting of the amino acid sequence set forth in SEQ ID NO:2012. In some instances, the anti-GFRAL antibody comprises: (i) a heavy chain comprising the amino acid sequence set forth in SEQ ID NO:2010; and (ii) a light chain comprising the amino acid sequence set forth in SEQ ID NO:2012. In some instances, the anti-GFRAL antibody comprises: (i) a heavy chain consisting of the amino acid sequence set forth in SEQ ID NO:2010; and (ii) a light chain consisting of the amino acid sequence set forth in SEQ ID NO:2012. In some instances, the paclitaxel (e.g., nab-paclitaxel) is administered to the human patient on days 1, 8, and 15 during the 4 week interval during which the anti-GFRAL antibody is administered to the human patient. In some instances, the gemcitabine is administered to the human patient on days 1, 8, and 15 during the 4 week interval during which the anti-GFRAL antibody is administered to the human patient. In some instances, the anti-GFRAL antibody is administered subcutaneously. In some instances, the anti-GFRAL antibody is administered intravenously. In some instances, the pancreatic cancer is advanced pancreatic cancer. In some instances, the pancreatic cancer is metastatic pancreatic cancer. In some instances, the pancreatic cancer is an MSI-H pancreatic cancer. In some instances, the pancreatic cancer is pancreatic adenocarcinoma. In some instances, the pancreatic cancer is metastatic pancreatic adenocarcinoma. In some instances, the pancreatic cancer is an MSI-H pancreatic adenocarcinoma. In some instances, the pancreatic cancer is a pancreatic ductal adenocarcinoma. In some instances, the pancreatic cancer is a metastatic pancreatic ductal adenocarcinoma.

In some instances of the combination therapy of the anti-GFRAL antibody, paclitaxel (e.g., nab-paclitaxel), and gemcitabine, the anti-GFRAL antibody is administered to the human patient subcutaneously at a dose of 100 mg once every 4 weeks, the paclitaxel (e.g., nab-paclitaxel) is administered to the human patient via intravenous infusion (e.g., over 30-40 minutes) once every week for three weeks during the 4 week interval during which the anti-GFRAL antibody is administered to the human patient, and the gemcitabine is administered to the human patient via intravenous infusion (e.g., over 30-40 minutes) once every week for 3 weeks during the 4 week interval during which the anti-GFRAL antibody is administered to the human patient, wherein the anti-GFRAL antibody comprises (i) a VH comprising the VH CDR1, the VH CDR2, the VH CDR3 from the amino acid sequence set forth in SEQ ID NO:3 or 1982; and (ii) a VL comprising the VL CDR1, the VL CDR2, and the VL CDR3 from the amino acid sequence set forth in SEQ ID NO:4 or 1997. In some instances, the anti-GFRAL antibody comprises: (i) a VH comprising a VH CDR1 comprising the amino acid sequence set forth in SEQ ID NO:46, a VH CDR2 comprising the amino acid sequence set forth in SEQ ID NO: 137, and a VH CDR3 comprising the amino acid sequence set forth in SEQ ID NO:225; and (ii) a VL comprising a VL CDR1 comprising the amino acid sequence set forth in SEQ ID NO:301, a VL CDR2 comprising the amino acid sequence set forth in SEQ ID NO:376, and a VL CDR3 comprising the amino acid sequence set forth in SEQ ID NO:426. In some instances, the anti-GFRAL antibody comprises: (i) a VH comprising a VH CDR1 comprising the amino acid sequence set forth in SEQ ID NO:48, a VH CDR2 comprising the amino acid sequence set forth in SEQ ID NO:137, and a VH CDR3 comprising the amino acid sequence set forth in SEQ ID NO:225; and (ii) a VL comprising a VL CDR1 comprising the amino acid sequence set forth in SEQ ID NO:301, a VL CDR2 comprising the amino acid sequence set forth in SEQ ID NO:376, and a VL CDR3 comprising the amino acid sequence set forth in SEQ ID NO:426. In some instances, the anti-GFRAL antibody comprises; (i) a VH comprising an amino acid sequence having at least at least 75%, at least 80%, at least 85%, at least 90%, at least 95%, at least 96%, at least 97%, at least 98%, at least 99%, or 100% identity to the amino acid sequence set forth in SEQ ID NO:1982; and (ii) a VL comprising an amino acid sequence having at least at least 75%, at least 80%, at least 85%, at least 90%, at least 95%, at least 96%, at least 97%, at least 98%, at least 99%, or 100% identity to the amino acid sequence set forth in SEQ ID NO:1997. In some instances, the anti-GFRAL antibody comprises a VH comprising the amino acid sequence set forth in SEQ ID NO:1982. In some instances, the anti-GFRAL antibody comprises a VL comprising the amino acid sequence set forth in SEQ ID NO:1997. In some instances, the anti-GFRAL antibody comprises: (i) a VH comprising the amino acid sequence set forth in SEQ ID NO:1982; and (ii) a VL comprising the amino acid sequence set forth in SEQ ID NO:1997. In some instances, the anti-GFRAL antibody comprises a heavy chain comprising an amino acid sequence having at least at least 75%, at least 80%, at least 85%, at least 90%, at least 95%, at least 96%, at least 97%, at least 98%, at least 99%, or 100% identity to the amino acid sequence set forth in SEQ ID NO:2010. In some instances, the anti-GFRAL antibody comprises a light chain comprising an amino acid sequence having at least at least 75%, at least 80%, at least 85%, at least 90%, at least 95%, at least 96%, at least 97%, at least 98%, at least 99%, or 100% identity to the amino acid sequence set forth in SEQ ID NO:2012. In some instances, the anti-GFRAL antibody comprises; (i) a heavy chain comprising an amino acid sequence having at least at least 75%, at least 80%, at least 85%, at least 90%, at least 95%, at least 96%, at least 97%, at least 98%, at least 99%, or 100% identity to the amino acid sequence set forth in SEQ ID NO:2010; and (ii) a light chain comprising an amino acid sequence having at least at least 75%, at least 80%, at least 85%, at least 90%, at least 95%, at least 96%, at least 97%, at least 98%, at least 99%, or 100% identity to the amino acid sequence set forth in SEQ ID NO:2012. In some instances, the anti-GFRAL antibody comprises a heavy chain comprising the amino acid sequence set forth in SEQ ID NO:2010. In some instances, the anti-GFRAL antibody comprises a heavy chain consisting of the amino acid sequence set forth in SEQ ID NO:2010. In some instances, the anti-GFRAL antibody comprises a light chain comprising the amino acid sequence set forth in SEQ ID NO:2012. In some instances, the anti-GFRAL antibody comprises a light chain consisting of the amino acid sequence set forth in SEQ ID NO:2012. In some instances, the anti-GFRAL antibody comprises: (i) a heavy chain comprising the amino acid sequence set forth in SEQ ID NO:2010; and (ii) a light chain comprising the amino acid sequence set forth in SEQ ID NO:2012. In some instances, the anti-GFRAL antibody comprises; (i) a heavy chain consisting of the amino acid sequence set forth in SEQ ID NO:2010; and (ii) a light chain consisting of the amino acid sequence set forth in SEQ ID NO:2012. In some instances, the paclitaxel (e.g., nab-paclitaxel) is administered to the human patient on days 1, 8, and 15 during the 4 week interval during which the anti-GFRAL antibody is administered to the human patient. In some instances, the gemcitabine is administered to the human patient on days 1, 8, and 15 during the 4 week interval during which the anti-GFRAL antibody is administered to the human patient. In some instances, the anti-GFRAL antibody is administered subcutaneously. In some instances, the anti-GFRAL antibody is administered intravenously. In some instances, the pancreatic cancer is advanced pancreatic adenocarcinoma. In some instances, the pancreatic cancer is metastatic pancreatic cancer. In some instances, the pancreatic cancer is an MSI-H pancreatic cancer. In some instances, the pancreatic cancer is pancreatic adenocarcinoma. In some instances, the pancreatic cancer is metastatic pancreatic adenocarcinoma. In some instances, the pancreatic cancer is an MSI-H pancreatic adenocarcinoma. In some instances, the pancreatic cancer is a pancreatic ductal adenocarcinoma. In some instances, the pancreatic cancer is a metastatic pancreatic ductal adenocarcinoma.

In some instances, the human patient previously failed to be treated with one or more (e.g., 1, 2, 3, 4, 5) other cancer therapies including any one of the standard of care therapies for a pancreatic cancer (i.e., other than an anti-GFRAL antibody described herein) prior to the administering of the anti-GFRAL antibody. In some instances, the human patient has relapsed from one or more (e.g., 1, 2, 3, 4, 5) other cancer therapies including any one of the standard of care therapies for a pancreatic cancer patient (i.e., other than an anti-GFRAL antibody described herein) prior to the administering of the anti-GFRAL antibody. In some instances, the pancreatic cancer is metastatic pancreatic cancer. In some instances, the pancreatic cancer is an MSI-H pancreatic cancer. In some instances, the pancreatic cancer is pancreatic adenocarcinoma. In some instances, the pancreatic cancer is metastatic pancreatic adenocarcinoma. In some instances, the pancreatic cancer is an MSI-H pancreatic adenocarcinoma. In some instances, the pancreatic cancer is a pancreatic ductal adenocarcinoma. In some instances, the pancreatic cancer is a metastatic pancreatic ductal adenocarcinoma.

In some instances, the anti-GFRAL antibody is administered subcutaneously. In some instances, the anti-GFRAL antibody is administered intravenously.

### VIII. EXAMPLES

### Example 1: Doses of an Anti-GFRAL Antibody Tested in Animal Model

In order to ascertain the effective dose(s) of an anti-GFRAL antibody as a therapy, a wide range of doses were first tested in an animal model. In this model, rats were administered with cisplatin to induced body weight loss, and the effect of various doses of an anti-GFRAL antibody on preventing or reducing the body weight loss of the rats were assessed. Briefly, rats were administered subcutaneously with a humanized anti-GFRAL antibody 3P10 (comprising a heavy chain of SEQ ID NO:2010, and a light chain of SEQ ID NO:2012) at a dose of 0.3 mg/kg, 1.0 mg/kg, 3.0 mg/kg, or 10 mg/kg once per week for 3 weeks. 24 hours after each administration, the rats were administered subcutaneously with cisplatin at 4 mg/kg. Body weight was determined for each animal (n=8 per group) every 24 hours during the 21-day treatment. Day 0 measurement was performed 24 hours after the first administration of the anti-GFRAL antibody and before the first administration of cisplatin.

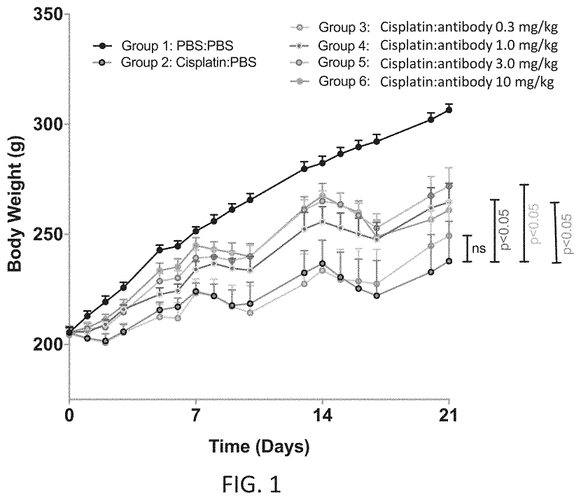

As shown in FIG. 1, the rats in the control group (Group 1) that received PBS instead of cisplatin and the anti-GFRAL antibody gained weight throughout the experiment with an average increase in weight of 33% (from 205 f 2 g to 306 t 3 g) from day 0 to day 21. In contrast, the rats that received cisplatin only (Group 2) increased weight by an average of only 13% (from 205 t 3 g to 237 f 18 g) day 0 to day 21, 0.3 mg/kg of the anti-GFRAL antibody (in Group 3 rats) did not prevent the cisplatin-induced weight loss in rats significantly. However, 1.0 mg/kg, 3.0 mg/kg and 10 mg/kg of the anti-GFRAL antibody reduced the cisplatin-induced weight loss significantly and to a similar extent. These results suggest that 1.0 mg/kg, 3.0 mg/kg and 10 mg/kg of the anti-GFRAL antibody worked at a similar efficacy in rats.

The dose-response relationship for the anti-GFRAL antibody on food intake was also measured (data not shown), and the results are consistent with the observed reduction of body weight loss in the cisplatin-treated rats, confirming that 1.0 mg/kg, 3.0 mg/kg and 10 mg/kg of the anti-GFRAL antibody work at a similar efficacy in an animal model.

### Example 2: Doses of an Anti-GFRAL Antibody Tested in Human

Based on the effective doses of the anti-GFRAL antibody determined from the rat model as described in Example 1, a roughly corresponding range of doses of the anti-GFRAL antibody for human was tested.

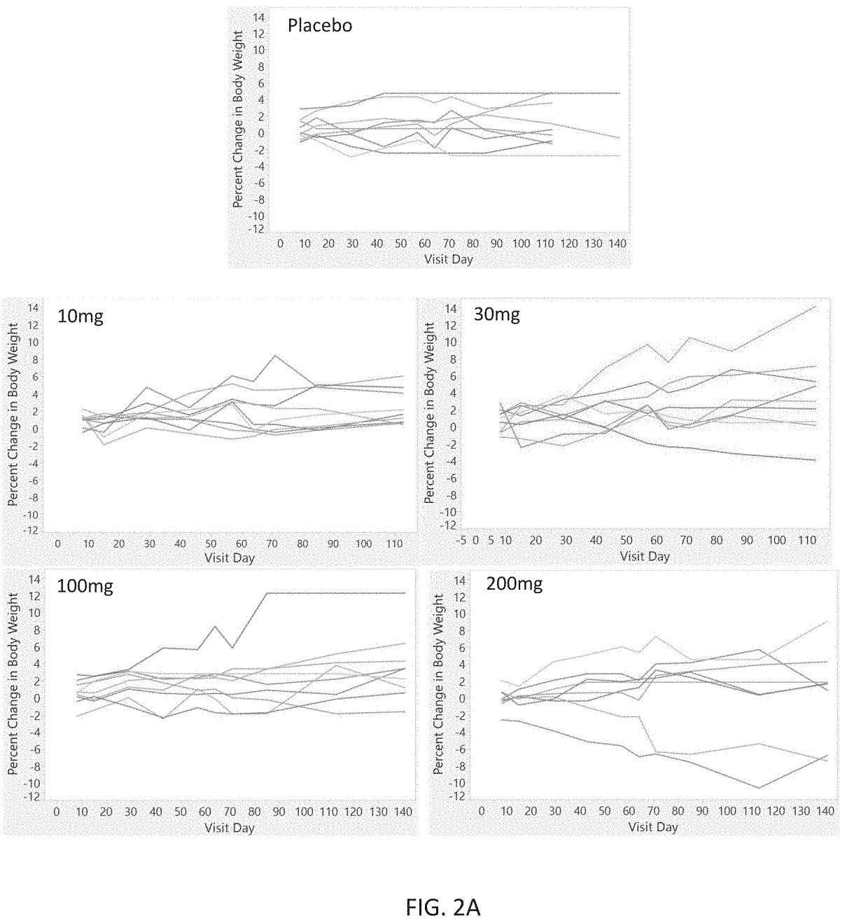

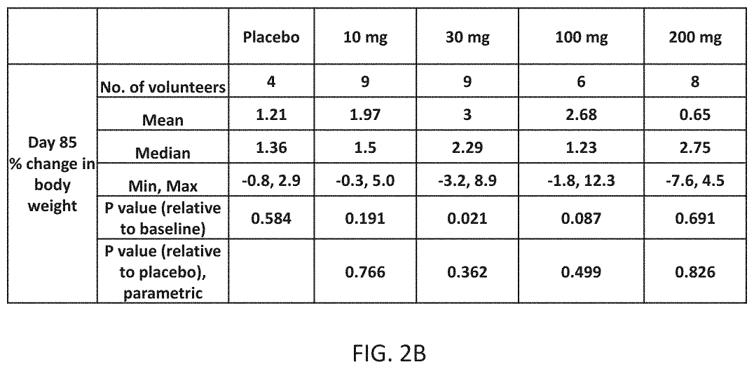

Healthy human volunteers were administered subcutaneously with the humanized anti-GFRAL antibody 3P10 (comprising a heavy chain of SEQ ID NO:2010, and a light chain of SEQ ID NO:2012) at a dose of 10 mg, 30 mg, 100 mg or 200 mg once every four weeks for 12 weeks. The body weight in every volunteer was measured weekly for 16 weeks. The body weight measured on the day prior to the first administration of the antibody was used as a baseline weight for each subject. Percentage changes of the body weight relative to the baseline for each subject were calculated and plotted (FIG. 2A). Percentage changes in body weight on day 85 are shown as examples in FIG. 2B. Surprisingly, 30 mg and 100 mg showed the most prominent changes in body weight among all the doses tested, even more so than the highest dose of 200 mg. These results indicate that 30 mg and 100 mg are effective doses of the anti-GFRAL antibody to be used in humans as a therapy.

### Example 3: Monotherapy Using an Anti-GFRAL Antibody

Based on the data from Examples 1 and 2, a human trial was designed to test the humanized anti-GFRAL antibody 3P10 (comprising a heavy chain of SEQ ID NO:2010, and a light chain of SEQ ID NO:2012) as a therapy for cancer. 19 human subjects with various cancers were administered subcutaneously once every 3 weeks for the total of 18 weeks (6 cycles) with 30 mg or 100 mg of the antibody. Among the 19 subjects, 7 subjects were diagnosed with colorectal cancer, 4 subjects were diagnosed with pancreatic cancer, and 6 subjects were diagnosed with prostate cancer, 1 patient is diagnosed bladder cancer, and 1 patient is diagnosed with esophageal cancer. The serum GDF15 levels in these subjects ranged from 1290-59600 pg/ml.

1. Safety

During the treatment, dose limiting toxicities, adverse events, serious adverse events, discontinuation of investigational product due to toxicity, and changes from baseline in laboratory parameters, electrocardiograms, vital signs, and local injection-site symptoms were assessed in the subjects. Overall, no dose-limiting toxicities were observed in all the subjects treated with 30 mg or 100 mg of the antibody. Most of the adverse events observed in these subjects were mild or moderate, and were not attributed to the administration of the antibody. Most frequent adverse events included fatigue (observed in 30% of the subjects), insomnia (observed in 25% of the subjects), GGT increase (observed in 20% of the subjects), and nausea (observed in 20% of the subjects). 7 subjects experienced 11 severe adverse events. However, none of them were attributed to the administration of the antibody. Instead, they were attributed to the underlying diseases of the subjects.

2. Pharmacokinetics

During the treatment with the antibody, blood samples were collected from the subjects for measurement of the serum concentrations of the antibody on day 1 (pre-dosing) and day 15 (post-dosing) for the first three cycles and day 1 (pre-dosing) of all the subsequent cycles. The concentrations were determined using an electrochemiluminescence immunoassay on the Meso Scale Diagnostics (MSD) platform. Briefly, a biotinylated monoclonal anti-idiotype antibody of the anti-GFRAL antibody was captured onto a streptavidin coated plate. The plate was then incubated with the serum of the subjects. After thorough washing of the plate to remove the unbound antibodies, a ruthenylated anti-idiotype antibody was added to the plate. Electrochemiluminescence signals were measured after adding the substrate to the plate. The concentration of the anti-GFRAFL antibody in the serum of each subject was calculated based on the standard curve generated using the same platform.

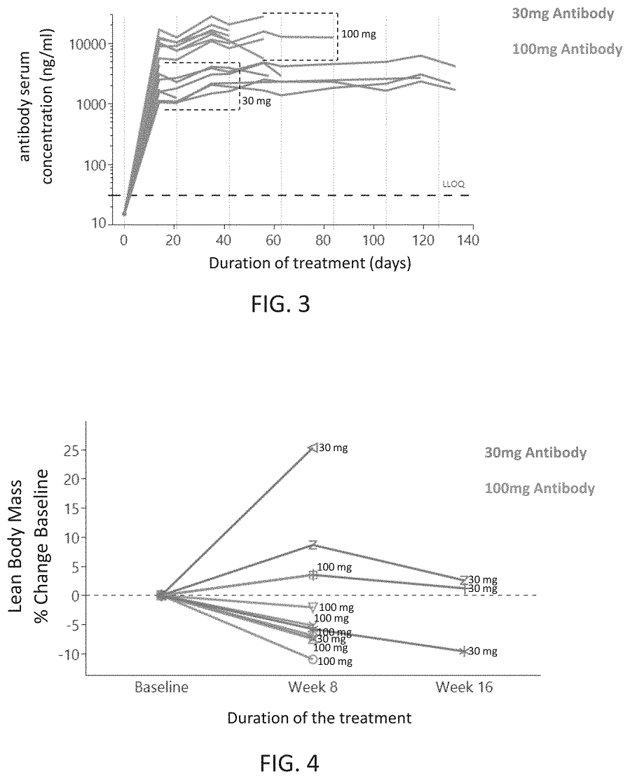

The antibody concentration-time profiles are shown in FIG. 3. From the 10 subjects receiving 30 mg of the antibody, the mean (±standard deviation) trough concentration following the 1^{st} and 2^{nd} doses were 1.58 (±0.659) pg/mL and 2.98 (±0.987) pg/mL, respectively. The accumulation ratios based on the trough concentration following the 1^{st} and 2^{nd} doses were between 1.26 to 1.73. From the 10 subjects receiving 100 mg of the antibody, the mean (±standard deviation) trough concentration following the 1^{st} and 2^{nd} doses were 8.88 (±2.35) μg/mL and 13.2 (±4.43) μg/mL, respectively. The accumulation ratios based on the trough concentration following the 1^{st} and 2^{nd} doses were between 1.33 to 1.64.

The serum half-life of the antibody determined from healthy subjects ranges from 28 to 40 days.

3. Reduction of Ketone Body Production

GDF15 induces lipolysis, thereby increasing the production of ketone bodies. Blocking the GDF15-GFRAL-RET signaling pathway decreases lipolysis and as a result reducing ketone body production. The serum level of beta hydroxybutyrate (BHB), a form of ketone bodies, was measured in the subjects on day 14 and day 28 after the antibody administration for the first 4 cycles and then day 28 after the antibody administration for all subsequent cycles. Serum samples were taken from the subjects who had fast for 12 hours, and were sent to a commercial diagnostic lab for measurement of the BHB level.

Both 30 mg and 100 mg of the antibody led to a reduced level of BHB in serum during the 18 week treatment period. In comparison with subjects treated with 30 mg, subjects treated with 100 mg showed a further reduction of BHB in the serum.

4. Lean Body Mass Gain and Tumor Stabilization

Lean body mass (LBM) and tumor response based on CT or MRI scans were performed during the study. A baseline measurement within 28 days before the first administration was obtained and post-treatment measurement every 6 weeks was carried out as long as the patients were on the study. From the 10 patients who obtained at least one scan post-treatment, 4 patients (3 in the 30 mg cohort and 1 in the 100 mg cohort) had increased LBM based on maximum change from baseline.

Tumor progression in the 16 patients was assessed according to the criteria set out from RECIST 1.1. 3 out of the 7 patients in the 30 mg cohort had Stable Disease and 1 out of the 9 patients in the 100 mg cohorts had Partial Response based on their best response.

5. Anti-GFRAL Antibody for Treating Prostate Cancer

Five patients with advanced prostate cancer were treated with 30 or 100 mg of humanized anti-GFRAL antibody 3P10 (comprising a heavy chain of SEQ ID NO:2010, and a light chain of SEQ ID NO:2012) subcutaneously once every 3 weeks. The patients had exhausted all standard therapies and had elevated baseline serum GDF15. All of the patients had previously received three or more lines of therapies. The median age was 65.6 years (range: 53.2 to 81.2 years). Primary endpoints including safety and tolerability, secondary endpoints including pharmacokinetics (PK), tumor progression using RECIST measurement, serum prostate specific antigen (PSA) level, and anti-cachexia effects were assessed in these patients.

Among the patients, their serum GDF15 levels ranged 2070 to 27900 pg/ml; and their serum PSA level ranged from 15 to 1728 ng/ml before the treatment. During the treatment, the anti-GFRAL antibody was well tolerated in all patients with no dose-limiting toxicities. Most of the adverse events observed were grade 1 to 2 and not attributed to the anti-GFRAL antibody treatment. Two out of the five patients had serum PSA reduction, one was −31.7% and the other was over −99% (from 76 ng/ml to <0.1 ng/ml). For the latter patient, the PSA reduction has been durable and sustained for 50 weeks and tumor partial response (PR) has been observed with RECIST assessment. Furthermore, the latter patient significantly elevated serum GDF15 level and high GDF15 expression in the tumor cells. After the treatment, the patient's serum GDF15 level was reduced up to 90% from baseline and body weight increased by up to 9.7%. Analysis of the circulating immune cell composition in the same patient showed an increase of CD8⁺ Tcm (central memory) cells and CD8⁺ T cells with proliferative capacity (Ki67+); and a decrease of immuno-suppressive T-regulatory cells (Treg) and naïve Treg cells. It is noted that this patient had high levels of MicroSatellie Instability (MSI-H), a genotype that-without being bound by any particular theory—may be more likely to respond to therapy of immune modulation.

### Example 4: Combination Therapy Using Anti-GFRAL Antibody

Another human trial was designed to test the humanized anti-GFRAL antibody 3P10 (comprising a heavy chain of SEQ ID NO:2010, and a light chain of SEQ ID NO:2012) in combination abraxane (paclitaxel protein-bound particles, also known as “nab-paclitaxel”) and gemcitabine as a therapy for pancreatic cancer.

Eight human subjects diagnosed with metastatic pancreatic adenocarcinoma were administered with the anti-GFRAL antibody in combination with abraxane (nab-paclitaxel) and gemcitabine. The median age was 67 years old. Seven patients were at stage IV disease and previously received FOLFERINOX or gemcitabine-based therapies. The antibody was administered subcutaneously at 30 mg or 100 mg once every 4 weeks. Abraxane was administered at a dose of 125 mg/m² weekly via intravenous infusion over 30-40 minutes for the first 3 weeks during the 4-week cycle (on day 1, day 8, and day 15). Gemcitabine was administered at a dose of 1000 mg/m² via intravenous infusion over 30-40 minutes for the first 3 weeks of the 4-week cycle (on day 1, day 8, and day 15). The serum GDF15 levels in these subjects ranged from 1350-3540 pg/mL.

1. Safety

During the treatment, dose limiting toxicities, adverse events, serious adverse events, discontinuation of investigational product due to toxicity, and changes from baseline in laboratory parameters, electrocardiograms, vital signs, and local injection-site symptoms were assessed in the subjects.

The anti-GFRAL antibody was well-tolerated. Overall, no dose-limiting toxicities of the antibody were observed in the tested subjects. Most of the observed adverse events and severe adverse events were not attributed to the antibody itself. The most frequent adverse events were diarrhea (observed in 50% of the subjects), nausea (observed in 50% of the subjects), and fatigue (observed in 50% of the subjects), which are commonly seen in the context of gemcitabine/nab-paclitaxel therapy. 5 patients experienced multiple severe adverse events, including sepsis, febrile neutropenia, pancreatitis, diarrhea, vomiting, retroperitoneal hemorrhage, acute kidney injury, cardiac arrest, and encephalopathy. However, none of these events were deemed related to the treatment of antibody itself after medical safety review. Instead, these events were attributed to gemcitabine and abraxane.

2. Pharmacokinetics

During the combination therapy, blood samples were collected from the subjects for measurement of serum concentrations of the antibody on day 1 (pre-dosing), day 8 (post-dosing) and day 15 (post-dosing) during the first four cycles and day 1 (pre-dosing) in all the subsequent cycles. The concentrations were determined in the same manner as described in Example 3, Section 2.

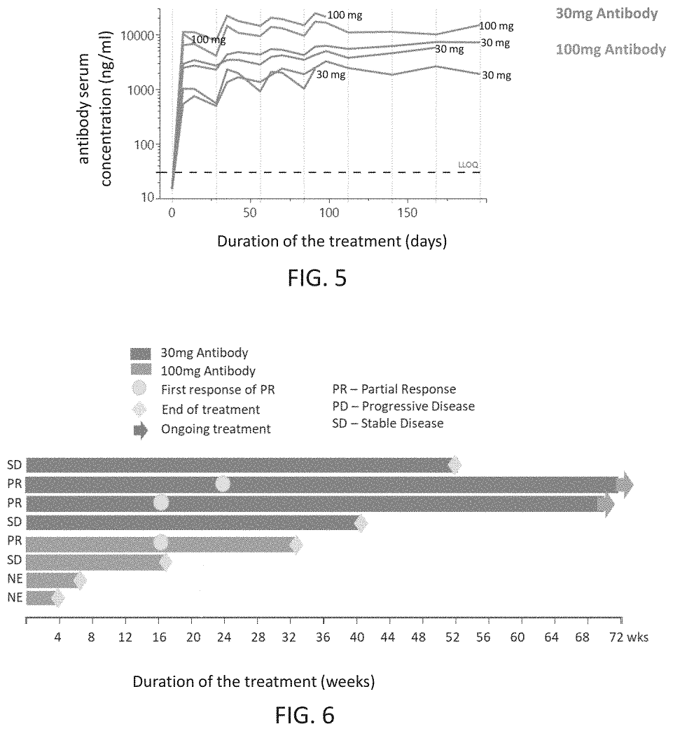

The antibody concentration-time profiles are shown in FIG. 5. From the 4 subjects receiving 30 mg of the antibody in combination with gemcitabine and abraxane, the mean (±standard deviation) trough concentration following the 1^{st} and 3^{rd} doses were 1.51 (±1.15) μg/mL and 2.67 (±1.16) μg/mL, respectively. The accumulation ratios based on trough concentration following the 1^{st} and 3^{rd} doses were between 1.28 to 3.81. From the 4 subjects receiving 100 mg of the antibody in combination with gemcitabine and abraxane, the mean (±standard deviation) trough concentration following the 1^{st} and 3^{rd} doses were 5.40 (±1.62) μg/mL and 10.1 (±4.61) μg/mL, respectively. The accumulation ratios based on trough concentration following the 1^{st} and 3^{rd} doses were between 1.17 to 2.32.

3. Tumor Progression

RECIST assessment based on CT or MRI scans were performed during the study to evaluate the patient's tumor progression. A baseline measurement within 28 days before the first administration of the combination therapy was obtained and post-treatment measurement every 8 weeks was carried out as long as the patients were on the study. As shown in FIG. 6, out of the 8 patients who received the combination therapy, 2 patients experienced gemcitabine/nab-paclitaxel related SAEs and discontinued from the study before obtaining their first post-treatment CT scan and, thus, RECIST assessment was not conducted and they were excluded from the analysis. Among the 6 evaluable patients, RECIST assessment showed 3 patients with Partial Response (extending >32 weeks) and 3 patients with Stable Disease. Overall, the disease control rate (DCR) was 100%, medium progression free survival (mPFS) was not reached (2/8 deaths), 12-month survival rate (12-month OS rate) was 83.3%. As a comparison, historic DCR is 50%, mPFS is 5.5 months, 12-month OS rate is 35% and medium overall survival is 8.5 months in metastatic pancreatic cancer (Von Hoff D D, et al. N Engl J Med. 2013, which is incorporated by reference herein in its entirety).

In addition to the CT scan, tumor antigen CA19-9 levels in patients were also assessed. Although not all pancreatic cancers express CA19-9, the antigen is expressed on the surface of many pancreatic cancers and is shed into the circulation. Tests measuring the levels of CA19-9 are therefore used as a less-invasive measurement of tumor burden. CA19-9 is expressed at very low levels in the normal pancreas and the normal CA19-9 range in serum is considered to be <37 U/mL. Blood samples of the patients were collected and CA19-9 levels were measured in a standard assay.

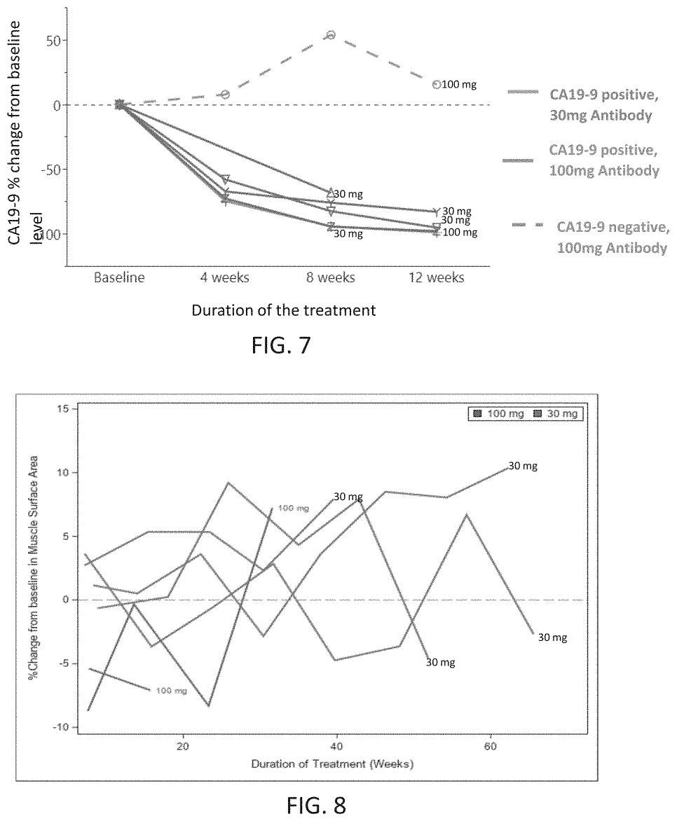

Of the 6 RECIST-evaluable patients, 1 patient was CA19-9 negative (CA19-9 levels <37 U/mL, dotted line in FIG. 7), and 5 patients were CA19-9 positive (CA19-9 levels between 480 and 16,000 U/mL, solid lines in FIG. 7). All 5 CA19-9 positive patients achieved at least a 68% reduction of CA19-9 levels in circulation during the treatment, wherein 4 out of these 5 patients achieved >80% reduction and 3 out of these 5 patients achieved >95% reduction.

4. Lean Body Mass Gain and Body Weight Gain

Cachexia (weight loss of at least 5% during the past 6 months) is common in patients with metastatic pancreatic cancer. Furthermore, up to 30% of follow-up cachexia may be observed within 12 weeks after the start of first-line chemotherapy (Mitsunaga S, et al., Supportive Care in Cancer, 2020). Out of the 8 patients, 6 patients had post-treatment measurements of both lean body mass and body weight.

As shown in FIG. 8, 5 out of 6 patients gained or maintained their LBM based on maximum change from baseline. The average maximal change of lean body mass in the 6 patients was an 4% increase compared to the baseline level.

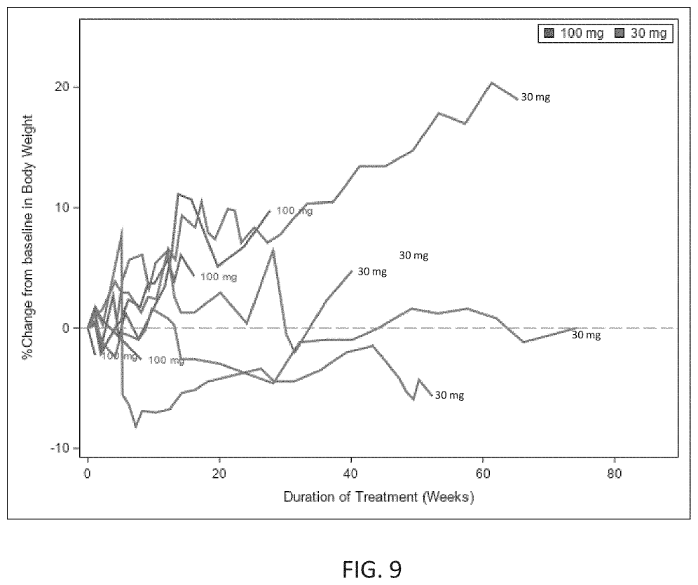

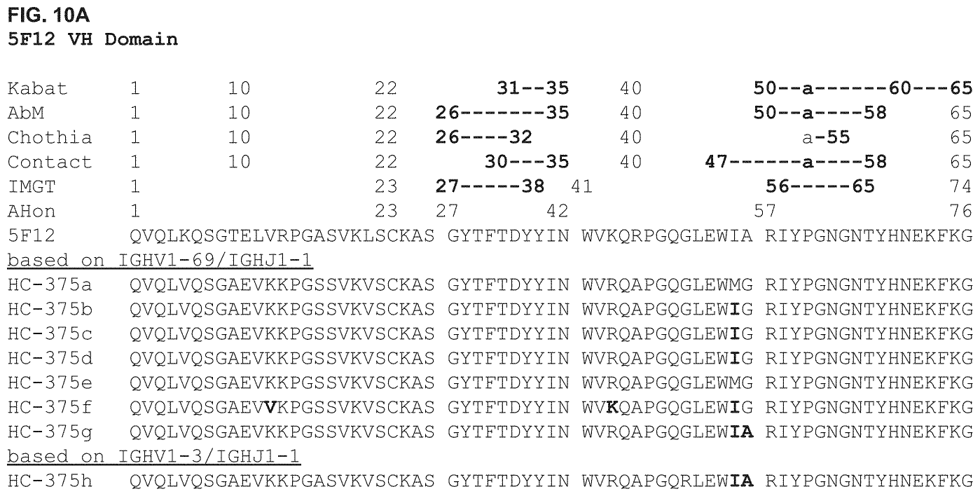

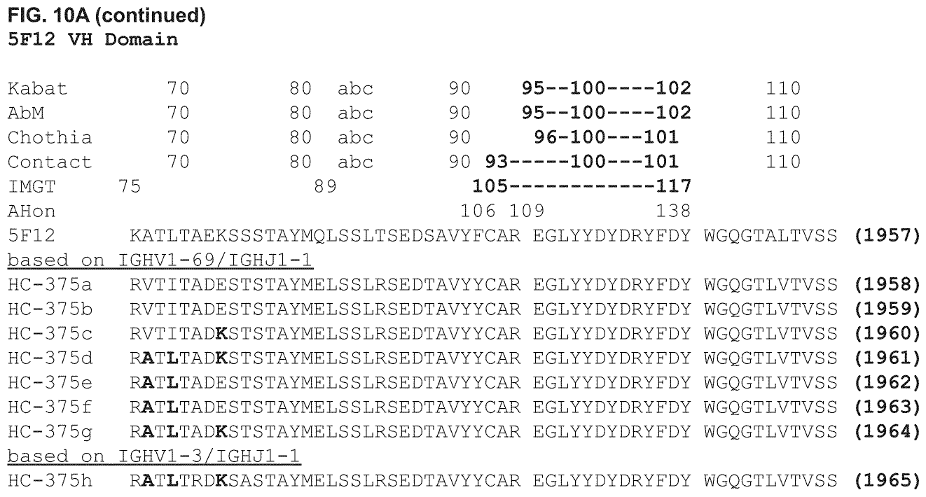

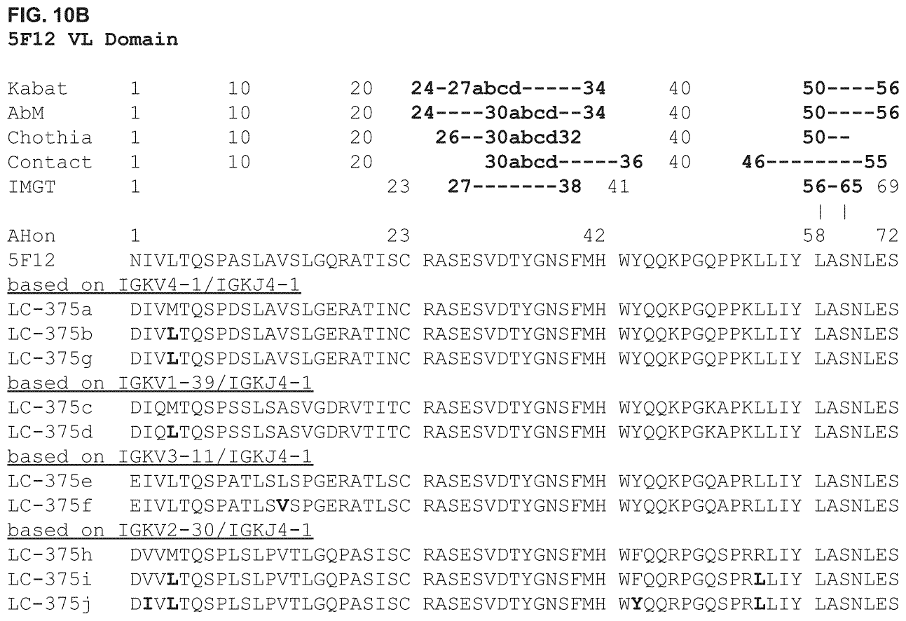

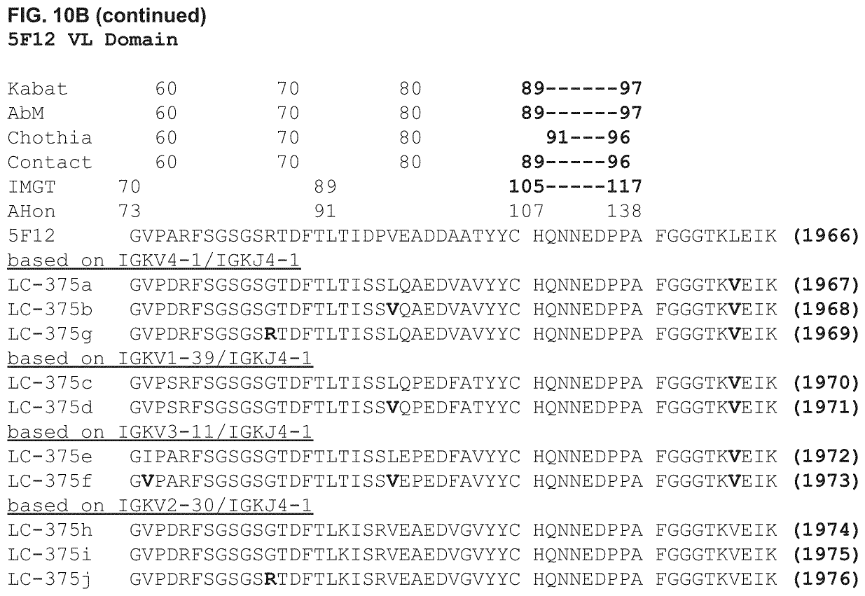

As shown in FIG. 9, over the course of the study, 5 out of 6 patients gained or maintained their body weight based on maximum change from baseline, achieving a 5-10% increase during the treatment. The average maximal body weight gain across all 6 patients was 6.2%. One patient who initially lost significant weight rebounded to almost the baseline weight during the treatment.

Overall, the data in the Examples show that humanized anti-GFRAL antibody that inhibits GFRAL binding to RET (e.g., humanized 3P10 antibody) can decrease or reverse body weight loss in cancer patients undergoing chemotherapies, suggesting an anti-cachexia effect of anti-GFRAL antibodies.

1. A method for treating a tumor in a human patient, the method comprising administering to the human patient an anti-GDNF Family Receptor Alpha-Like (GFRAL) antibody at a dose of about 30 mg or about 100 mg once about every 3 weeks, wherein the anti-GFRAL antibody inhibits GFRAL binding to RET.

2.-6. (canceled)

7. A method for treating tumor-related weight loss or cachexia in a human patient, the method comprising administering to the human patient an anti-GFRAL antibody at a dose of about 30 mg or about 100 mg once about every 3 weeks, wherein the antibody inhibits GFRAL binding to RET.

8.-11. (canceled)

12. The method of claim 1, wherein the tumor is a cancer selected from the group consisting of a prostate cancer, a bladder cancer, melanoma, a small cell lung cancer, a non-small cell lung cancer, a pancreatic cancer, a colorectal cancer, a gastric cancer, an esophageal cancer, an ovarian cancer, and a head neck squamous cell carcinoma.

13.-24. (canceled)

25. A method for treating pancreatic cancer in a human patient, the method comprising administering to the human patient:
(a) an anti-GFRAL antibody at a dose of about 30 mg or about 100 mg once about every 4 weeks, wherein the anti-GFRAL antibody inhibits GFRAL binding to RET,
(b) paclitaxel; and
(c) gemcitabine.

26.-42. (canceled)

43. The method of claim 1, wherein the anti-GFRAL antibody;
(a) specifically binds within amino acid residues 220-316 of the amino acid sequence set forth in SEQ ID NO: 1797;
(b) specifically binds to one or more residues selected from the group consisting of: Met214, Pro216, Pro217, Gln290, Cys291, Thr292, Cys293, Arg294, Thr295, Ile296, Thr297, Gln298, Ser299, Glu301, Lys305, Gln308, His309, His312, and Ser315 of the amino acid sequence set forth in SEQ ID NO: 1797; or
(c) specifically binds to Thr297, Gln298, and Ser299 of the amino acid sequence set forth in SEQ ID NO:1797.

44. The method of claim 7, wherein the anti-GFRAL antibody;
(a) specifically binds within amino acid residues 220-316 of the amino acid sequence set forth in SEQ ID NO: 1797;
(b) specifically binds to one or more residues selected from the group consisting of: Met214, Pro216, Pro217, Gln290, Cys291, Thr292, Cys293, Arg294, Thr295, Ile296, Thr297, Gln298, Ser299, Glu301, Lys305, Gln308, His309, His312, and Ser315 of the amino acid sequence set forth in SEQ ID NO: 1797; or
(c) specifically binds to Thr297, Gln298, and Ser299 of the amino acid sequence set forth in SEQ ID NO:1797.

45. The method of claim 25, wherein the anti-GFRAL antibody;
(a) specifically binds within amino acid residues 220-316 of the amino acid sequence set forth in SEQ ID NO: 1797;
(b) specifically binds to one or more residues selected from the group consisting of: Met214, Pro216, Pro217, Gln290, Cys291, Thr292, Cys293, Arg294, Thr295, Ile296, Thr297, Gln298, Ser299, Glu301, Lys305, Gln308, His309, His312, and Ser315 of the amino acid sequence set forth in SEQ ID NO: 1797; or
(c) specifically binds to Thr297, Gln298, and Ser299 of the amino acid sequence set forth in SEQ ID NO:1797.

46. The method of claim 1, wherein the antibody comprises a heavy chain variable region (VH) and a light chain variable region (VL), and wherein the VH comprises a VH complementarity determining region (CDR)1, a VH CDR2, and a VH CDR3 from the amino acid sequence set forth in SEQ ID NO:1982, and the VL comprises a VL CDR1, a VL CDR2, and a VL CDR3 from the amino acid sequence set forth in SEQ ID NO:1997.

47. The method of claim 1, wherein the antibody comprises a VH and a VL, and wherein
(a) the VH comprises a VH CDR1 comprising the amino acid sequence set forth in SEQ ID NO:46, a VH CDR2 comprising the amino acid sequence set forth in SEQ ID NO:137, a VH CDR3 comprising the amino acid sequence set forth in SEQ ID NO:225; and the VL comprising a VL CDR1 comprising the amino acid sequence set forth in SEQ ID NO:301, a VL CDR2 comprising the amino acid sequence set forth in SEQ ID NO:376, and a VL CDR3 comprising the amino acid sequence set forth in SEQ ID NO:426;
(b) the VH comprises a VH CDR1 comprising the amino acid sequence set forth in SEQ ID NO:47, a VH CDR2 comprising the amino acid sequence set forth in SEQ ID NO:138, a VH CDR3 comprising the amino acid sequence set forth in SEQ ID NO:226; and the VL comprising a VL CDR1 comprising the amino acid sequence set forth in SEQ ID NO:302, a VL CDR2 comprising the amino acid sequence set forth in SEQ ID NO:377, and a VL CDR3 comprising the amino acid sequence set forth in SEQ ID NO:426;
(c) the VH comprises a VH CDR1 comprising the amino acid sequence set forth in SEQ ID NO:48, a VH CDR2 comprising the amino acid sequence set forth in SEQ ID NO:137, a VH CDR3 comprising the amino acid sequence set forth in SEQ ID NO:225; and the VL comprising a VL CDR1 comprising the amino acid sequence set forth in SEQ ID NO:301, a VL CDR2 comprising the amino acid sequence set forth in SEQ ID NO:376, and a VL CDR3 comprising the amino acid sequence set forth in SEQ ID NO:426;
(d) the VH comprises a VH CDR1 comprising the amino acid sequence set forth in SEQ ID NO:49, a VH CDR2 comprising the amino acid sequence set forth in SEQ ID NO:139, a VH CDR3 comprising the amino acid sequence set forth in SEQ ID NO:227; and the VL comprising a VL CDR1 comprising the amino acid sequence set forth in SEQ ID NO:303, a VL CDR2 comprising the amino acid sequence set forth in SEQ ID NO:377, and a VL CDR3 comprising the amino acid sequence set forth in SEQ ID NO:427;
(e) the VH comprises a VH CDR1 comprising the amino acid sequence set forth in SEQ ID NO:50, a VH CDR2 comprising the amino acid sequence set forth in SEQ ID NO:140, a VH CDR3 comprising the amino acid sequence set forth in SEQ ID NO:228; and the VL comprising a VL CDR1 comprising the amino acid sequence set forth in SEQ ID NO:304, a VL CDR2 comprising the amino acid sequence set forth in SEQ ID NO:378, and a VL CDR3 comprising the amino acid sequence set forth in SEQ ID NO:428;
(f) the VH comprises a VH CDR1 comprising the amino acid sequence set forth in SEQ ID NO:46, a VH CDR2 comprising the amino acid sequence set forth in SEQ ID NO:141, a VH CDR3 comprising the amino acid sequence set forth in SEQ ID NO:225; and the VL comprising a VL CDR1 comprising the amino acid sequence set forth in SEQ ID NO:301, a VL CDR2 comprising the amino acid sequence set forth in SEQ ID NO:376, and a VL CDR3 comprising the amino acid sequence set forth in SEQ ID NO:426.

48.-50. (canceled)

51. The method of claim 1, wherein antibody comprises a VH and a VL, wherein the VH comprises the amino acid sequence set forth in SEQ ID NO:1982 and the VL comprises the amino acid sequence set forth in SEQ ID NO:1997.

52.-58. (canceled)

59. The method of claim 1, wherein the antibody comprises a heavy chain comprising the amino acid sequence set forth in SEQ ID NO: 2010 and a light chain comprising the amino acid sequence set forth in SEQ ID NO: 2012.

60. (canceled)

61. (canceled)

62. The method of claim 7, wherein the antibody comprises a VH and a VL, and wherein the VH comprises a VH CDR1, a VH CDR2, and a VH CDR3 from the amino acid sequence set forth in SEQ ID NO:1982, and the VL comprises a VL CDR1, a VL CDR2, and a VL CDR3 from the amino acid sequence set forth in SEQ ID NO:1997.

63. The method of claim 25, wherein the antibody comprises a VH and a VL, and wherein the VH comprises a VH CDR1, a VH CDR2, and a VH CDR3 from the amino acid sequence set forth in SEQ ID NO:1982, and the VL comprises a VL CDR1, a VL CDR2, and a VL CDR3 from the amino acid sequence set forth in SEQ ID NO:1997.

64. The method of claim 7, wherein the antibody comprises a VH and a VL, and wherein
(a) the VH comprises a VH CDR1 comprising the amino acid sequence set forth in SEQ ID NO:46, a VH CDR2 comprising the amino acid sequence set forth in SEQ ID NO:137, a VH CDR3 comprising the amino acid sequence set forth in SEQ ID NO:225; and the VL comprising a VL CDR1 comprising the amino acid sequence set forth in SEQ ID NO:301, a VL CDR2 comprising the amino acid sequence set forth in SEQ ID NO:376, and a VL CDR3 comprising the amino acid sequence set forth in SEQ ID NO:426;
(b) the VH comprises a VH CDR1 comprising the amino acid sequence set forth in SEQ ID NO:47, a VH CDR2 comprising the amino acid sequence set forth in SEQ ID NO:138, a VH CDR3 comprising the amino acid sequence set forth in SEQ ID NO:226; and the VL comprising a VL CDR1 comprising the amino acid sequence set forth in SEQ ID NO:302, a VL CDR2 comprising the amino acid sequence set forth in SEQ ID NO:377, and a VL CDR3 comprising the amino acid sequence set forth in SEQ ID NO:426;
(c) the VH comprises a VH CDR1 comprising the amino acid sequence set forth in SEQ ID NO:48, a VH CDR2 comprising the amino acid sequence set forth in SEQ ID NO:137, a VH CDR3 comprising the amino acid sequence set forth in SEQ ID NO:225; and the VL comprising a VL CDR1 comprising the amino acid sequence set forth in SEQ ID NO:301, a VL CDR2 comprising the amino acid sequence set forth in SEQ ID NO:376, and a VL CDR3 comprising the amino acid sequence set forth in SEQ ID NO:426;
(d) the VH comprises a VH CDR1 comprising the amino acid sequence set forth in SEQ ID NO:49, a VH CDR2 comprising the amino acid sequence set forth in SEQ ID NO:139, a VH CDR3 comprising the amino acid sequence set forth in SEQ ID NO:227; and the VL comprising a VL CDR1 comprising the amino acid sequence set forth in SEQ ID NO:303, a VL CDR2 comprising the amino acid sequence set forth in SEQ ID NO:377, and a VL CDR3 comprising the amino acid sequence set forth in SEQ ID NO:427;
(e) the VH comprises a VH CDR1 comprising the amino acid sequence set forth in SEQ ID NO:50, a VH CDR2 comprising the amino acid sequence set forth in SEQ ID NO:140, a VH CDR3 comprising the amino acid sequence set forth in SEQ ID NO:228; and the VL comprising a VL CDR1 comprising the amino acid sequence set forth in SEQ ID NO:304, a VL CDR2 comprising the amino acid sequence set forth in SEQ ID NO:378, and a VL CDR3 comprising the amino acid sequence set forth in SEQ ID NO:428;
(f) the VH comprises a VH CDR1 comprising the amino acid sequence set forth in SEQ ID NO:46, a VH CDR2 comprising the amino acid sequence set forth in SEQ ID NO:141, a VH CDR3 comprising the amino acid sequence set forth in SEQ ID NO:225; and the VL comprising a VL CDR1 comprising the amino acid sequence set forth in SEQ ID NO:301, a VL CDR2 comprising the amino acid sequence set forth in SEQ ID NO:376, and a VL CDR3 comprising the amino acid sequence set forth in SEQ ID NO:426.

65. The method of claim 25, wherein the antibody comprises a VH and a VL, and wherein
(a) the VH comprises a VH CDR1 comprising the amino acid sequence set forth in SEQ ID NO:46, a VH CDR2 comprising the amino acid sequence set forth in SEQ ID NO:137, a VH CDR3 comprising the amino acid sequence set forth in SEQ ID NO:225; and the VL comprising a VL CDR1 comprising the amino acid sequence set forth in SEQ ID NO:301, a VL CDR2 comprising the amino acid sequence set forth in SEQ ID NO:376, and a VL CDR3 comprising the amino acid sequence set forth in SEQ ID NO:426;
(b) the VH comprises a VH CDR1 comprising the amino acid sequence set forth in SEQ ID NO:47, a VH CDR2 comprising the amino acid sequence set forth in SEQ ID NO:138, a VH CDR3 comprising the amino acid sequence set forth in SEQ ID NO:226; and the VL comprising a VL CDR1 comprising the amino acid sequence set forth in SEQ ID NO:302, a VL CDR2 comprising the amino acid sequence set forth in SEQ ID NO:377, and a VL CDR3 comprising the amino acid sequence set forth in SEQ ID NO:426;
(c) the VH comprises a VH CDR1 comprising the amino acid sequence set forth in SEQ ID NO:48, a VH CDR2 comprising the amino acid sequence set forth in SEQ ID NO:137, a VH CDR3 comprising the amino acid sequence set forth in SEQ ID NO:225; and the VL comprising a VL CDR1 comprising the amino acid sequence set forth in SEQ ID NO:301, a VL CDR2 comprising the amino acid sequence set forth in SEQ ID NO:376, and a VL CDR3 comprising the amino acid sequence set forth in SEQ ID NO:426;
(d) the VH comprises a VH CDR1 comprising the amino acid sequence set forth in SEQ ID NO:49, a VH CDR2 comprising the amino acid sequence set forth in SEQ ID NO:139, a VH CDR3 comprising the amino acid sequence set forth in SEQ ID NO:227; and the VL comprising a VL CDR1 comprising the amino acid sequence set forth in SEQ ID NO:303, a VL CDR2 comprising the amino acid sequence set forth in SEQ ID NO:377, and a VL CDR3 comprising the amino acid sequence set forth in SEQ ID NO:427;
(e) the VH comprises a VH CDR1 comprising the amino acid sequence set forth in SEQ ID NO:50, a VH CDR2 comprising the amino acid sequence set forth in SEQ ID NO:140, a VH CDR3 comprising the amino acid sequence set forth in SEQ ID NO:228; and the VL comprising a VL CDR1 comprising the amino acid sequence set forth in SEQ ID NO:304, a VL CDR2 comprising the amino acid sequence set forth in SEQ ID NO:378, and a VL CDR3 comprising the amino acid sequence set forth in SEQ ID NO:428;
(f) the VH comprises a VH CDR1 comprising the amino acid sequence set forth in SEQ ID NO:46, a VH CDR2 comprising the amino acid sequence set forth in SEQ ID NO:141, a VH CDR3 comprising the amino acid sequence set forth in SEQ ID NO:225; and the VL comprising a VL CDR1 comprising the amino acid sequence set forth in SEQ ID NO:301, a VL CDR2 comprising the amino acid sequence set forth in SEQ ID NO:376, and a VL CDR3 comprising the amino acid sequence set forth in SEQ ID NO:426.

66. The method of claim 7, wherein the antibody comprises a VH and a VL, and wherein the VH comprises the amino acid sequence set forth in SEQ ID NO:1982 and the VL comprises the amino acid sequence set forth in SEQ ID NO:1997.

67. The method of claim 25, wherein the antibody comprises a VH and a VL, and wherein the VH comprises the amino acid sequence set forth in SEQ ID NO:1982 and the VL comprises the amino acid sequence set forth in SEQ ID NO:1997.

68. The method of claim 7, wherein the antibody comprises a heavy chain comprising the amino acid sequence set forth in SEQ ID NO: 2010 and a light chain comprising the amino acid sequence set forth in SEQ ID NO: 2012.

69. The method of claim 25, wherein the antibody comprises a heavy chain comprising the amino acid sequence set forth in SEQ ID NO: 2010 and a light chain comprising the amino acid sequence set forth in SEQ ID NO: 2012.


SEQUENCE LISTING (2012 sequences)

SEQ ID NO: 1-2 - skipped (2 entries)

SEQ ID NO: 3 - protein, 118 aa, synthetic construct
QIQLVQSGPELKKPGETVKISCKASGYTFTDYGVIWVKQAPGKALKWMGWINTYTGEPTYADDLKGRFAFSLETSASSASLQINNLKNEDTATYFCARRYGPEDIDYWGQGTTLTVSS

SEQ ID NO: 4 - protein, 111 aa, synthetic construct
DIVLTQSPVSLAVSLGQRATISCRASESVDNYGISFMSWFQQKPGQPPKLLIYAASHQGSGVPARFSGSGSGTDFSLNIHPMEEDDSAMYFCLQSKEVPWTFGGGTKLEIK

SEQ ID NO: 5-6 - skipped (2 entries)

SEQ ID NO: 7 - protein, 122 aa, synthetic construct
QVQLKQSGTELVRPGASVKLSCKASGYTFTDYYINWVKQRPGQGLEWIARIYPGNGNTYHNEKFKGKATLTAEKSSSTAYMQLSSLTSEDSAVYFCAREGLYYDYDRYFDYWGQGTALTVSS

SEQ ID NO: 8 - protein, 111 aa, synthetic construct
NIVLTQSPASLAVSLGQRATISCRASESVDTYGNSFMHWYQQKPGQPPKLLIYLASNLESGVPARFSGSGSRTDFTLTIDPVEADDAATYYCHQNNEDPPAFGGGTKLEIK

SEQ ID NO: 9-20 - skipped (12 entries)

SEQ ID NO: 21 - protein, 120 aa, synthetic construct
QAQLQQSGAELVKPGASVKLSCKASGYSFTSYNIDWVRQRPEQGLEWIGWIFPGDGSTKYNEKFKGQATLTTDKSSSTTYIHLSRLTSEDSAVYFCARSGIYYGSHFVYWGQGTLVTVSA

SEQ ID NO: 22 - protein, 112 aa, synthetic construct
DVVMTQTPLTLSVTIGQSASISCRSSQSLLDSDGKTYLNWLLQRPGQSPKRLIYLVSKVDSGVPDRFTGSGSGTDFTLKISRVEAEDLGVYFCWQGTHFPLTFGAGTKLELK

SEQ ID NO: 23 - protein, 120 aa, synthetic construct
QVQLQQSGSELVKPGTSMKLSCKASGYTFTSYNINWVRLRPEQGLEWIGWIFPGDDSIKYNENFRGKATLTTDKSSSTAYMHLSRLTSDDSAVYFCARSGIFYGNNFAYWGQGTLVTVSA

SEQ ID NO: 24 - protein, 112 aa, synthetic construct
DVVMTQAPLILSVTIGQPASISCKSSQSLLDGDGETYLSWLLQRPGQSPKRLIYLVSKLDSGVPDRFTGSGSGTDFTLKISRVEAEDLGVYYCCQSTHFPLTFGAGTKLELK

SEQ ID NO: 25 - protein, 119 aa, synthetic construct
QVHLQQPGAELVKPGASVKLSCKASGFTFTGYNINWVRLRPEQGLEWIGWIFPGDDNAKYNEKFKGKATLTTDKSSNTAYMQLSRLTSEDSAVYFCARTPVLSNYFDYWGQGTTLTVSS

SEQ ID NO: 26 - protein, 106 aa, synthetic construct
DIQMTQSPSSLSASLGGKVTITCKASQDISKYISWYQHKPGKSPRLLIHYTSTLQPGIPSRFSGSGSGRDYSFSISNLEPEDIATYYCLQYDNLYTFGGGTKLEIK

SEQ ID NO: 27-36 - skipped (10 entries)

SEQ ID NO: 37 - protein, 120 aa, synthetic construct
QVQLHQPGAELVKPGASVKLSCKTSGYTFTSYWMQWVKQRPGQGLEWIGEIDPSDSYTNYNQKFKGKATLTVDTSSTTAYMQLSSLTSEDSAVYYCARPLDRSAYYFDYWGQGTTLTVSS

SEQ ID NO: 38 - protein, 111 aa, synthetic construct
DIVLTQSPASLAVSLGQRATISCRASESVDFSGNSFMHWYQQKPGQPPKLLIYRASNLDSGIPARFSGVGSRTDFTLTINPVEADDVATYYCQQSNEDPYTFGGGTKLEIE

SEQ ID NO: 39 - protein, 119 aa, synthetic construct
DVQLQESGPGLVKPSQSLSLTCSVTGYSITSGYYWNWIRQFPGNKLEWMGHIANDGSNYYNPFLKHRVSITRDTSKNQFFLKLNSVTIQDTATYYCARGGSYFDYVDYWGQGTTLTVSS

SEQ ID NO: 40 - protein, 107 aa, synthetic construct
DIQMTQTTSSLSASLGDRVTINCRASQDISNYLNWYQQKPDGTVKLLIYYTSRLHSGVPSRFSGSGSGTDYSLTITNLEQEDIATYFCQQGNTLPFTFGSGTKLEIK

SEQ ID NO: 41-45 - skipped (5 entries)

SEQ ID NO: 46 - protein, 10 aa, synthetic construct
GYTFTDYGVI

SEQ ID NO: 47 - protein, 8 aa, synthetic construct
GYTFTDYG

SEQ ID NO: 48 - protein, 5 aa, synthetic construct
DYGVI

SEQ ID NO: 49 - protein, 7 aa, synthetic construct
GYTFTDY

SEQ ID NO: 50 - protein, 6 aa, synthetic construct
TDYGVI

SEQ ID NO: 51-55 - skipped (5 entries)

SEQ ID NO: 56 - protein, 10 aa, synthetic construct
GYTFTDYYIN

SEQ ID NO: 57 - protein, 8 aa, synthetic construct
GYTFTDYY

SEQ ID NO: 58 - protein, 5 aa, synthetic construct
DYYIN

SEQ ID NO: 59 - protein, 6 aa, synthetic construct
TDYYIN

SEQ ID NO: 60-72 - skipped (13 entries)

SEQ ID NO: 73 - protein, 8 aa, synthetic construct
GYTFTSYW

SEQ ID NO: 74 - skipped

SEQ ID NO: 75 - protein, 7 aa, synthetic construct
GYTFTSY

SEQ ID NO: 76-85 - skipped (10 entries)

SEQ ID NO: 86 - protein, 10 aa, synthetic construct
GYSFTSYNID

SEQ ID NO: 87 - protein, 8 aa, synthetic construct
GYSFTSYN

SEQ ID NO: 88 - protein, 5 aa, synthetic construct
SYNID

SEQ ID NO: 89 - protein, 7 aa, synthetic construct
GYSFTSY

SEQ ID NO: 90 - protein, 6 aa, synthetic construct
TSYNID

SEQ ID NO: 91 - protein, 10 aa, synthetic construct
GYTFTSYNIN

SEQ ID NO: 92 - protein, 8 aa, synthetic construct
GYTFTSYN

SEQ ID NO: 93 - protein, 5 aa, synthetic construct
SYNIN

SEQ ID NO: 94 - protein, 6 aa, synthetic construct
TSYNIN

SEQ ID NO: 95 - protein, 10 aa, synthetic construct
GFTFTGYNIN

SEQ ID NO: 96 - protein, 8 aa, synthetic construct
GFTFTGYN

SEQ ID NO: 97 - protein, 5 aa, synthetic construct
GYNIN

SEQ ID NO: 98 - protein, 7 aa, synthetic construct
GFTFTGY

SEQ ID NO: 99 - protein, 6 aa, synthetic construct
TGYNIN

SEQ ID NO: 100-123 - skipped (24 entries)

SEQ ID NO: 124 - protein, 10 aa, synthetic construct
GYTFTSYWMQ

SEQ ID NO: 125 - protein, 5 aa, synthetic construct
SYWMQ

SEQ ID NO: 126 - protein, 6 aa, synthetic construct
TSYWMQ

SEQ ID NO: 127 - protein, 11 aa, synthetic construct
GYSITSGYYWN

SEQ ID NO: 128 - protein, 9 aa, synthetic construct
GYSITSGYY

SEQ ID NO: 129 - protein, 6 aa, synthetic construct
SGYYWN

SEQ ID NO: 130 - protein, 8 aa, synthetic construct
GYSITSGY

SEQ ID NO: 131 - protein, 7 aa, synthetic construct
TSGYYWN

SEQ ID NO: 132-136 - skipped (5 entries)

SEQ ID NO: 137 - protein, 17 aa, synthetic construct
WINTYTGEPTYADDLKG

SEQ ID NO: 138 - protein, 8 aa, synthetic construct
INTYTGEP

SEQ ID NO: 139 - protein, 4 aa, synthetic construct
TYTG

SEQ ID NO: 140 - protein, 13 aa, synthetic construct
WMGWINTYTGEPT

SEQ ID NO: 141 - protein, 10 aa, synthetic construct
WINTYTGEPT

SEQ ID NO: 142-146 - skipped (5 entries)

SEQ ID NO: 147 - protein, 17 aa, synthetic construct
RIYPGNGNTYHNEKFKG

SEQ ID NO: 148 - protein, 8 aa, synthetic construct
IYPGNGNT

SEQ ID NO: 149 - protein, 4 aa, synthetic construct
PGNG

SEQ ID NO: 150 - protein, 13 aa, synthetic construct
WIARIYPGNGNTY

SEQ ID NO: 151 - protein, 10 aa, synthetic construct
RIYPGNGNTY

SEQ ID NO: 152-167 - skipped (16 entries)

SEQ ID NO: 168 - protein, 4 aa, synthetic construct
PGDG

SEQ ID NO: 169-176 - skipped (8 entries)

SEQ ID NO: 177 - protein, 9 aa, synthetic construct
WIFPGDGST

SEQ ID NO: 178 - protein, 8 aa, synthetic construct
IFPGDGST

SEQ ID NO: 179 - protein, 17 aa, synthetic construct
WIFPGDGSTKYNEKFKG

SEQ ID NO: 180 - protein, 13 aa, synthetic construct
WIGWIFPGDGSTK

SEQ ID NO: 181 - protein, 10 aa, synthetic construct
WIFPGDGSTK

SEQ ID NO: 182 - protein, 17 aa, synthetic construct
WIFPGDDSIKYNENFRG

SEQ ID NO: 183 - protein, 8 aa, synthetic construct
IFPGDDSI

SEQ ID NO: 184 - protein, 4 aa, synthetic construct
PGDD

SEQ ID NO: 185 - protein, 13 aa, synthetic construct
WIGWIFPGDDSIK

SEQ ID NO: 186 - protein, 10 aa, synthetic construct
WIFPGDDSIK

SEQ ID NO: 187 - protein, 17 aa, synthetic construct
WIFPGDDNAKYNEKFKG

SEQ ID NO: 188 - protein, 8 aa, synthetic construct
IFPGDDNA

SEQ ID NO: 189 - protein, 13 aa, synthetic construct
WIGWIFPGDDNAK

SEQ ID NO: 190 - protein, 10 aa, synthetic construct
WIFPGDDNAK

SEQ ID NO: 191-209 - skipped (19 entries)

SEQ ID NO: 210 - protein, 17 aa, synthetic construct
EIDPSDSYTNYNQKFKG

SEQ ID NO: 211 - protein, 8 aa, synthetic construct
IDPSDSYT

SEQ ID NO: 212 - protein, 4 aa, synthetic construct
PSDS

SEQ ID NO: 213 - protein, 13 aa, synthetic construct
WIGEIDPSDSYTN

SEQ ID NO: 214 - protein, 10 aa, synthetic construct
EIDPSDSYTN

SEQ ID NO: 215 - protein, 16 aa, synthetic construct
HIANDGSNYYNPFLKH

SEQ ID NO: 216 - protein, 7 aa, synthetic construct
IANDGSN

SEQ ID NO: 217 - skipped

SEQ ID NO: 218 - protein, 12 aa, synthetic construct
WMGHIANDGSNY

SEQ ID NO: 219 - protein, 9 aa, synthetic construct
HIANDGSNY

SEQ ID NO: 220-224 - skipped (5 entries)

SEQ ID NO: 225 - protein, 9 aa, synthetic construct
RYGPEDIDY

SEQ ID NO: 226 - protein, 11 aa, synthetic construct
ARRYGPEDIDY

SEQ ID NO: 227 - protein, 7 aa, synthetic construct
YGPEDID

SEQ ID NO: 228 - protein, 10 aa, synthetic construct
ARRYGPEDID

SEQ ID NO: 229-232 - skipped (4 entries)

SEQ ID NO: 233 - protein, 13 aa, synthetic construct
EGLYYDYDRYFDY

SEQ ID NO: 234 - protein, 15 aa, synthetic construct
AREGLYYDYDRYFDY

SEQ ID NO: 235 - protein, 11 aa, synthetic construct
GLYYDYDRYFD

SEQ ID NO: 236 - protein, 14 aa, synthetic construct
AREGLYYDYDRYFD

SEQ ID NO: 237-260 - skipped (24 entries)

SEQ ID NO: 261 - protein, 11 aa, synthetic construct
SGIYYGSHFVY

SEQ ID NO: 262 - protein, 13 aa, synthetic construct
ARSGIYYGSHFVY

SEQ ID NO: 263 - protein, 9 aa, synthetic construct
GIYYGSHFV

SEQ ID NO: 264 - protein, 12 aa, synthetic construct
ARSGIYYGSHFV

SEQ ID NO: 265 - protein, 11 aa, synthetic construct
SGIFYGNNFAY

SEQ ID NO: 266 - protein, 13 aa, synthetic construct
ARSGIFYGNNFAY

SEQ ID NO: 267 - protein, 9 aa, synthetic construct
GIFYGNNFA

SEQ ID NO: 268 - protein, 12 aa, synthetic construct
ARSGIFYGNNFA

SEQ ID NO: 269 - protein, 10 aa, synthetic construct
TPVLSNYFDY

SEQ ID NO: 270 - protein, 12 aa, synthetic construct
ARTPVLSNYFDY

SEQ ID NO: 271 - protein, 8 aa, synthetic construct
PVLSNYFD

SEQ ID NO: 272 - protein, 11 aa, synthetic construct
ARTPVLSNYFD

SEQ ID NO: 273-288 - skipped (16 entries)

SEQ ID NO: 289 - protein, 11 aa, synthetic construct
PLDRSAYYFDY

SEQ ID NO: 290 - protein, 13 aa, synthetic construct
ARPLDRSAYYFDY

SEQ ID NO: 291 - protein, 9 aa, synthetic construct
LDRSAYYFD

SEQ ID NO: 292 - protein, 12 aa, synthetic construct
ARPLDRSAYYFD

SEQ ID NO: 293 - protein, 10 aa, synthetic construct
GGSYFDYVDY

SEQ ID NO: 294 - protein, 12 aa, synthetic construct
ARGGSYFDYVDY

SEQ ID NO: 295 - protein, 8 aa, synthetic construct
GSYFDYVD

SEQ ID NO: 296 - protein, 11 aa, synthetic construct
ARGGSYFDYVD

SEQ ID NO: 297-300 - skipped (4 entries)

SEQ ID NO: 301 - protein, 15 aa, synthetic construct
RASESVDNYGISFMS

SEQ ID NO: 302 - protein, 10 aa, synthetic construct
ESVDNYGISF

SEQ ID NO: 303 - protein, 11 aa, synthetic construct
SESVDNYGISF

SEQ ID NO: 304 - protein, 11 aa, synthetic construct
DNYGISFMSWF

SEQ ID NO: 305-308 - skipped (4 entries)

SEQ ID NO: 309 - protein, 15 aa, synthetic construct
RASESVDTYGNSFMH

SEQ ID NO: 310 - protein, 10 aa, synthetic construct
ESVDTYGNSF

SEQ ID NO: 311 - protein, 11 aa, synthetic construct
SESVDTYGNSF

SEQ ID NO: 312 - protein, 11 aa, synthetic construct
DTYGNSFMHWY

SEQ ID NO: 313-336 - skipped (24 entries)

SEQ ID NO: 337 - protein, 16 aa, synthetic construct
RSSQSLLDSDGKTYLN

SEQ ID NO: 338 - protein, 11 aa, synthetic construct
QSLLDSDGKTY

SEQ ID NO: 339 - protein, 12 aa, synthetic construct
SQSLLDSDGKTY

SEQ ID NO: 340 - protein, 12 aa, synthetic construct
LDSDGKTYLNWL

SEQ ID NO: 341 - protein, 16 aa, synthetic construct
KSSQSLLDGDGETYLS

SEQ ID NO: 342 - protein, 11 aa, synthetic construct
QSLLDGDGETY

SEQ ID NO: 343 - protein, 12 aa, synthetic construct
SQSLLDGDGETY

SEQ ID NO: 344 - protein, 12 aa, synthetic construct
LDGDGETYLSWL

SEQ ID NO: 345 - protein, 11 aa, synthetic construct
KASQDISKYIS

SEQ ID NO: 346 - protein, 6 aa, synthetic construct
QDISKY

SEQ ID NO: 347 - protein, 7 aa, synthetic construct
SQDISKY

SEQ ID NO: 348 - protein, 7 aa, synthetic construct
SKYISWY

SEQ ID NO: 349-364 - skipped (16 entries)

SEQ ID NO: 365 - protein, 15 aa, synthetic construct
RASESVDFSGNSFMH

SEQ ID NO: 366 - protein, 10 aa, synthetic construct
ESVDFSGNSF

SEQ ID NO: 367 - protein, 11 aa, synthetic construct
SESVDFSGNSF

SEQ ID NO: 368 - protein, 11 aa, synthetic construct
DFSGNSFMHWY

SEQ ID NO: 369 - protein, 11 aa, synthetic construct
RASQDISNYLN

SEQ ID NO: 370 - protein, 6 aa, synthetic construct
QDISNY

SEQ ID NO: 371 - protein, 7 aa, synthetic construct
SQDISNY

SEQ ID NO: 372 - protein, 7 aa, synthetic construct
SNYLNWY

SEQ ID NO: 373-375 - skipped (3 entries)

SEQ ID NO: 376 - protein, 7 aa, synthetic construct
AASHQGS

SEQ ID NO: 377 - skipped

SEQ ID NO: 378 - protein, 10 aa, synthetic construct
LLIYAASHQG

SEQ ID NO: 379-381 - skipped (3 entries)

SEQ ID NO: 382 - protein, 7 aa, synthetic construct
LASNLES

SEQ ID NO: 383 - skipped

SEQ ID NO: 384 - protein, 10 aa, synthetic construct
LLIYLASNLE

SEQ ID NO: 385 - protein, 7 aa, synthetic construct
LVSKLDS

SEQ ID NO: 386-400 - skipped (15 entries)

SEQ ID NO: 401 - protein, 7 aa, synthetic construct
LVSKVDS

SEQ ID NO: 402 - protein, 10 aa, synthetic construct
RLIYLVSKVD

SEQ ID NO: 403 - protein, 10 aa, synthetic construct
RLIYLVSKLD

SEQ ID NO: 404 - protein, 7 aa, synthetic construct
YTSTLQP

SEQ ID NO: 405 - skipped

SEQ ID NO: 406 - protein, 10 aa, synthetic construct
LLIHYTSTLQ

SEQ ID NO: 407-417 - skipped (11 entries)

SEQ ID NO: 418 - protein, 7 aa, synthetic construct
RASNLDS

SEQ ID NO: 419 - skipped

SEQ ID NO: 420 - protein, 10 aa, synthetic construct
LLIYRASNLD

SEQ ID NO: 421 - protein, 7 aa, synthetic construct
YTSRLHS

SEQ ID NO: 422 - protein, 10 aa, synthetic construct
LLIYYTSRLH

SEQ ID NO: 423-425 - skipped (3 entries)

SEQ ID NO: 426 - protein, 9 aa, synthetic construct
LQSKEVPWT

SEQ ID NO: 427 - protein, 6 aa, synthetic construct
SKEVPW

SEQ ID NO: 428 - protein, 8 aa, synthetic construct
LQSKEVPW

SEQ ID NO: 429-431 - skipped (3 entries)

SEQ ID NO: 432 - protein, 9 aa, synthetic construct
HQNNEDPPA

SEQ ID NO: 433 - protein, 6 aa, synthetic construct
NNEDPP

SEQ ID NO: 434 - protein, 8 aa, synthetic construct
HQNNEDPP

SEQ ID NO: 435-452 - skipped (18 entries)

SEQ ID NO: 453 - protein, 9 aa, synthetic construct
WQGTHFPLT

SEQ ID NO: 454 - protein, 6 aa, synthetic construct
GTHFPL

SEQ ID NO: 455 - protein, 8 aa, synthetic construct
WQGTHFPL

SEQ ID NO: 456 - protein, 9 aa, synthetic construct
CQSTHFPLT

SEQ ID NO: 457 - protein, 6 aa, synthetic construct
STHFPL

SEQ ID NO: 458 - protein, 8 aa, synthetic construct
CQSTHFPL

SEQ ID NO: 459 - protein, 8 aa, synthetic construct
LQYDNLYT

SEQ ID NO: 460 - protein, 5 aa, synthetic construct
YDNLY

SEQ ID NO: 461 - protein, 7 aa, synthetic construct
LQYDNLY

SEQ ID NO: 462-473 - skipped (12 entries)

SEQ ID NO: 474 - protein, 9 aa, synthetic construct
QQSNEDPYT

SEQ ID NO: 475 - protein, 6 aa, synthetic construct
SNEDPY

SEQ ID NO: 476 - protein, 8 aa, synthetic construct
QQSNEDPY

SEQ ID NO: 477 - protein, 9 aa, synthetic construct
QQGNTLPFT

SEQ ID NO: 478 - protein, 6 aa, synthetic construct
GNTLPF

SEQ ID NO: 479 - protein, 8 aa, synthetic construct
QQGNTLPF

SEQ ID NO: 480-1796 - skipped (1317 entries)

SEQ ID NO: 1797 - protein, 394 aa, Homo sapiens
MIVFIFLAMGLSLENEYTSQTNNCTYLREQCLRDANGCKHAWRVMEDACNDSDPGDPCKMRNSSYCNLSIQYLVESNFQFKECLCTDDFYCTVNKLLGKKCINKSDNVKEDKFKWNLTTRSHHGFKGMWSCLEVAEACVGDVVCNAQLASYLKACSANGNPCDLKQCQAAIRFFYQNIPFNIAQMLAFCDCAQSDIPCQQSKEALHSKTCAVNMVPPPTCLSVIRSCQNDELCRRHYRTFQSKCWQRVTRKCHEDENCISTLSKQDLTCSGSDDCKAAYIDILGTVLQVQCTCRTITQSEESLCKIFQHMLHRKSCFNYPTLSNVKGMALYTRKHANKITLTGFHSPFNGEVIYAAMCMTVTCGILLLVMVKLRTSRISSKARDPSSIQIPGEL

SEQ ID NO: 1798 - protein, 375 aa, Homo sapiens
QTNNCTYLREQCLRDANGCKHAWRVMEDACNDSDPGDPCKMRNSSYCNLSIQYLVESNFQFKECLCTDDFYCTVNKLLGKKCINKSDNVKEDKFKWNLTTRSHHGFKGMWSCLEVAEACVGDVVCNAQLASYLKACSANGNPCDLKQCQAAIRFFYQNIPFNIAQMLAFCDCAQSDIPCQQSKEALHSKTCAVNMVPPPTCLSVIRSCQNDELCRRHYRTFQSKCWQRVTRKCHEDENCISTLSKQDLTCSGSDDCKAAYIDILGTVLQVQCTCRTITQSEESLCKIFQHMLHRKSCFNYPTLSNVKGMALYTRKHANKITLTGFHSPFNGEVIYAAMCMTVTCGILLLVMVKLRTSRISSKARDPSSIQIPGEL

SEQ ID NO: 1799-1809 - skipped (11 entries)

SEQ ID NO: 1810 - protein, 308 aa, Homo sapiens
MPGQELRTVNGSQMLLVLLVLSWLPHGGALSLAEASRASFPGPSELHSEDSRFRELRKRYEDLLTRLRANQSWEDSNTDLVPAPAVRILTPEVRLGSGGHLHLRISRAALPEGLPEASRLHRALFRLSPTASRSWDVTRPLRRQLSLARPQAPALHLRLSPPPSQSDQLLAESSSARPQLELHLRPQAARGRRRARARNGDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 1811 - protein, 112 aa, Homo sapiens
ARNGDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 1812 - protein, 1049 aa, Homo sapiens
KVALGLYFSRDAYWEKLYVDQAAGTPLLYVHALRDAPEEVPSFRLGQHLYGTYRTRLHENNWICIQEDTGLLYLNRSLDHSSWEKLSVRNRGFPLLTVYLKVFLSPTSLREGECQWPGCARVYFSFFNTSFPACSSLKPRELCFPETRPSFRIRENRPPGTFHQFRLLPVQFLCPNISVAYRLLEGEGLPFRCAPDSLEVSTRWALDREQREKYELVAVCTVHAGAREEVVMVPFPVTVYDEDDSAPTFPAGVDTASAVVEFKRKEDTVVATLRVFDADVVPASGELVRRYTSTLLPGDTWAQQTFRVEHWPNETSVQANGSFVRATVHDYRLVLNRNLSISENRTMQLAVLVNDSDFQGPGAGVLLLHFNVSVLPVSLHLPSTYSLSVSRRARRFAQIGKVCVENCQAFSGINVQYKLHSSGANCSTLGVVTSAEDTSGILFVNDTKALRRPKCAELHYMVVATDQQTSRQAQAQLLVTVEGSYVAEEAGCPLSCAVSKRRLECEECGGLGSPTGRCEWRQGDGKGITRNFSTCSPSTKTCPDGHCDVVETQDINICPQDCLRGSIVGGHEPGEPRGIKAGYGTCNCFPEEEKCFCEPEDIQDPLCDELCRTVIAAAVLFSFIVSVLLSAFCIHCYHKFAHKPPISSAEMTFRRPAQAFPVSYSSSGARRPSLDSMENQVSVDAFKILEDPKWEFPRKNLVLGKTLGEGEFGKVVKATAFHLKGRAGYTTVAVKMLKENASPSELRDLLSEFNVLKQVNHPHVIKLYGACSQDGPLLLIVEYAKYGSLRGFLRESRKVGPGYLGSGGSRNSSSLDHPDERALTMGDLISFAWQISQGMQYLAEMKLVHRDLAARNILVAEGRKMKISDFGLSRDVYEEDSYVKRSQGRIPVKWMAIESLFDHIYTTQSDVWSFGVLLWEIVTLGGNPYPGIPPERLFNLLKTGHRMERPDNCSEEMYRLMLQCWKQEPDKRPVFADISKDLEKMMVKRRDYLDLAASTPSDSLIYDDGLSEEETPLVDCNNAPLPRALPSTWIENKLYGRISHAFTRF

SEQ ID NO: 1813 - protein, 1072 aa, Homo sapiens
MAKATSGAAGLRLLLLLLLPLLGKVALGLYFSRDAYWEKLYVDQAAGTPLLYVHALRDAPEEVPSFRLGQHLYGTYRTRLHENNWICIQEDTGLLYLNRSLDHSSWEKLSVRNRGFPLLTVYLKVFLSPTSLREGECQWPGCARVYFSFFNTSFPACSSLKPRELCFPETRPSFRIRENRPPGTFHQFRLLPVQFLCPNISVAYRLLEGEGLPFRCAPDSLEVSTRWALDREQREKYELVAVCTVHAGAREEVVMVPFPVTVYDEDDSAPTFPAGVDTASAVVEFKRKEDTVVATLRVFDADVVPASGELVRRYTSTLLPGDTWAQQTFRVEHWPNETSVQANGSFVRATVHDYRLVLNRNLSISENRTMQLAVLVNDSDFQGPGAGVLLLHFNVSVLPVSLHLPSTYSLSVSRRARRFAQIGKVCVENCQAFSGINVQYKLHSSGANCSTLGVVTSAEDTSGILFVNDTKALRRPKCAELHYMVVATDQQTSRQAQAQLLVTVEGSYVAEEAGCPLSCAVSKRRLECEECGGLGSPTGRCEWRQGDGKGITRNFSTCSPSTKTCPDGHCDVVETQDINICPQDCLRGSIVGGHEPGEPRGIKAGYGTCNCFPEEEKCFCEPEDIQDPLCDELCRTVIAAAVLFSFIVSVLLSAFCIHCYHKFAHKPPISSAEMTFRRPAQAFPVSYSSSGARRPSLDSMENQVSVDAFKILEDPKWEFPRKNLVLGKTLGEGEFGKVVKATAFHLKGRAGYTTVAVKMLKENASPSELRDLLSEFNVLKQVNHPHVIKLYGACSQDGPLLLIVEYAKYGSLRGFLRESRKVGPGYLGSGGSRNSSSLDHPDERALTMGDLISFAWQISQGMQYLAEMKLVHRDLAARNILVAEGRKMKISDFGLSRDVYEEDSYVKRSQGRIPVKWMAIESLFDHIYTTQSDVWSFGVLLWEIVTLGGNPYPGIPPERLFNLLKTGHRMERPDNCSEEMYRLMLQCWKQEPDKRPVFADISKDLEKMMVKRRDYLDLAASTPSDSLIYDDGLSEEETPLVDCNNAPLPRALPSTWIENKLYGRISHAFTRF

SEQ ID NO: 1814-1956 - skipped (143 entries)

SEQ ID NO: 1957 - protein, 122 aa, synthetic construct; identical to SEQ ID NO: 7

SEQ ID NO: 1958 - protein, 122 aa, synthetic construct
QVQLVQSGAEVKKPGSSVKVSCKASGYTFTDYYINWVRQAPGQGLEWMGRIYPGNGNTYHNEKFKGRVTITADESTSTAYMELSSLRSEDTAVYYCAREGLYYDYDRYFDYWGQGTLVTVSS

SEQ ID NO: 1959 - protein, 122 aa, synthetic construct
QVQLVQSGAEVKKPGSSVKVSCKASGYTFTDYYINWVRQAPGQGLEWIGRIYPGNGNTYHNEKFKGRVTITADESTSTAYMELSSLRSEDTAVYYCAREGLYYDYDRYFDYWGQGTLVTVSS

SEQ ID NO: 1960 - protein, 122 aa, synthetic construct
QVQLVQSGAEVKKPGSSVKVSCKASGYTFTDYYINWVRQAPGQGLEWIGRIYPGNGNTYHNEKFKGRVTITADKSTSTAYMELSSLRSEDTAVYYCAREGLYYDYDRYFDYWGQGTLVTVSS

SEQ ID NO: 1961 - protein, 122 aa, synthetic construct
QVQLVQSGAEVKKPGSSVKVSCKASGYTFTDYYINWVRQAPGQGLEWIGRIYPGNGNTYHNEKFKGRATLTADKSTSTAYMELSSLRSEDTAVYYCAREGLYYDYDRYFDYWGQGTLVTVSS

SEQ ID NO: 1962 - protein, 122 aa, synthetic construct
QVQLVQSGAEVKKPGSSVKVSCKASGYTFTDYYINWVRQAPGQGLEWMGRIYPGNGNTYHNEKFKGRATLTADESTSTAYMELSSLRSEDTAVYYCAREGLYYDYDRYFDYWGQGTLVTVSS

SEQ ID NO: 1963 - protein, 122 aa, synthetic construct
QVQLVQSGAEVVKPGSSVKVSCKASGYTFTDYYINWVKQAPGQGLEWIGRIYPGNGNTYHNEKFKGRATLTADESTSTAYMELSSLRSEDTAVYYCAREGLYYDYDRYFDYWGQGTLVTVSS

SEQ ID NO: 1964 - protein, 122 aa, synthetic construct
QVQLVQSGAEVKKPGSSVKVSCKASGYTFTDYYINWVRQAPGQGLEWIARIYPGNGNTYHNEKFKGRATLTADKSTSTAYMELSSLRSEDTAVYYCAREGLYYDYDRYFDYWGQGTLVTVSS

SEQ ID NO: 1965 - protein, 122 aa, synthetic construct
QVQLVQSGAEVKKPGASVKVSCKASGYTFTDYYINWVRQAPGQRLEWIARIYPGNGNTYHNEKFKGRATLTRDKSASTAYMELSSLRSEDTAVYYCAREGLYYDYDRYFDYWGQGTLVTVSS

SEQ ID NO: 1966 - protein, 111 aa, synthetic construct; identical to SEQ ID NO: 8

SEQ ID NO: 1967 - protein, 111 aa, synthetic construct
DIVMTQSPDSLAVSLGERATINCRASESVDTYGNSFMHWYQQKPGQPPKLLIYLASNLESGVPDRFSGSGSGTDFTLTISSLQAEDVAVYYCHQNNEDPPAFGGGTKVEIK

SEQ ID NO: 1968 - protein, 111 aa, synthetic construct
DIVLTQSPDSLAVSLGERATINCRASESVDTYGNSFMHWYQQKPGQPPKLLIYLASNLESGVPDRFSGSGSGTDFTLTISSVQAEDVAVYYCHQNNEDPPAFGGGTKVEIK

SEQ ID NO: 1969 - protein, 111 aa, synthetic construct
DIVLTQSPDSLAVSLGERATINCRASESVDTYGNSFMHWYQQKPGQPPKLLIYLASNLESGVPDRFSGSGSRTDFTLTISSLQAEDVAVYYCHQNNEDPPAFGGGTKVEIK

SEQ ID NO: 1970 - protein, 111 aa, synthetic construct
DIQMTQSPSSLSASVGDRVTITCRASESVDTYGNSFMHWYQQKPGKAPKLLIYLASNLESGVPSRFSGSGSGTDFTLTISSLQPEDFATYYCHQNNEDPPAFGGGTKVEIK

SEQ ID NO: 1971 - protein, 111 aa, synthetic construct
DIQLTQSPSSLSASVGDRVTITCRASESVDTYGNSFMHWYQQKPGKAPKLLIYLASNLESGVPSRFSGSGSGTDFTLTISSVQPEDFATYYCHQNNEDPPAFGGGTKVEIK

SEQ ID NO: 1972 - protein, 111 aa, synthetic construct
EIVLTQSPATLSLSPGERATLSCRASESVDTYGNSFMHWYQQKPGQAPRLLIYLASNLESGIPARFSGSGSGTDFTLTISSLEPEDFAVYYCHQNNEDPPAFGGGTKVEIK

SEQ ID NO: 1973 - protein, 111 aa, synthetic construct
EIVLTQSPATLSVSPGERATLSCRASESVDTYGNSFMHWYQQKPGQAPRLLIYLASNLESGVPARFSGSGSGTDFTLTISSVEPEDFAVYYCHQNNEDPPAFGGGTKVEIK

SEQ ID NO: 1974 - protein, 111 aa, synthetic construct
DVVMTQSPLSLPVTLGQPASISCRASESVDTYGNSFMHWFQQRPGQSPRRLIYLASNLESGVPDRFSGSGSGTDFTLKISRVEAEDVGVYYCHQNNEDPPAFGGGTKVEIK

SEQ ID NO: 1975 - protein, 111 aa, synthetic construct
DVVLTQSPLSLPVTLGQPASISCRASESVDTYGNSFMHWFQQRPGQSPRLLIYLASNLESGVPDRFSGSGSGTDFTLKISRVEAEDVGVYYCHQNNEDPPAFGGGTKVEIK

SEQ ID NO: 1976 - protein, 111 aa, synthetic construct
DIVLTQSPLSLPVTLGQPASISCRASESVDTYGNSFMHWYQQRPGQSPRLLIYLASNLESGVPDRFSGSGSRTDFTLKISRVEAEDVGVYYCHQNNEDPPAFGGGTKVEIK

SEQ ID NO: 1977-1981 - skipped (5 entries)

SEQ ID NO: 1982 - protein, 118 aa, synthetic construct
QVQLVQSGAEVKKPGSSVKVSCKASGYTFTDYGVIWVRQAPGQGLEWMGWINTYTGEPTYADDLKGRVTFTADESTSTAYMELSSLRSEDTAVYYCARRYGPEDIDYWGQGTTVTVSS

SEQ ID NO: 1983-1996 - skipped (14 entries)

SEQ ID NO: 1997 - protein, 111 aa, synthetic construct
DVVLTQSPLSLPVTLGQPASISCRASESVDNYGISFMSWFQQRPGQSPRLLIYAASHQGSGVPDRFSGSGSGTDFTLKISRVEAEDVGVYFCLQSKEVPWTFGGGTKVEIK

SEQ ID NO: 1998-2008 - skipped (11 entries)

SEQ ID NO: 2009 - protein, 470 aa, synthetic construct
MDMRVPAQLLGLLLLWLRGARCQVQLVQSGAEVKKPGSSVKVSCKASGYTFTDYGVIWVRQAPGQGLEWMGWINTYTGEPTYADDLKGRVTFTADESTSTAYMELSSLRSEDTAVYYCARRYGPEDIDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPALAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2010 - protein, 448 aa, synthetic construct
QVQLVQSGAEVKKPGSSVKVSCKASGYTFTDYGVIWVRQAPGQGLEWMGWINTYTGEPTYADDLKGRVTFTADESTSTAYMELSSLRSEDTAVYYCARRYGPEDIDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPALAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2011 - protein, 240 aa, synthetic construct
MDMRVPAQLLGLLLLWLRGARCDVVLTQSPLSLPVTLGQPASISCRASESVDNYGISFMSWFQQRPGQSPRLLIYAASHQGSGVPDRFSGSGSGTDFTLKISRVEAEDVGVYFCLQSKEVPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2012 - protein, 218 aa, synthetic construct
DVVLTQSPLSLPVTLGQPASISCRASESVDNYGISFMSWFQQRPGQSPRLLIYAASHQGSGVPDRFSGSGSGTDFTLKISRVEAEDVGVYFCLQSKEVPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC